(19) United States
(12) Patent Application Publication
(10) Pub. No.: US 2024/0374735 A1
(43) Pub. Date: Nov. 14, 2024

(54) AROMATIC BORON-CONTAINING COMPOUNDS AND INSULIN ANALOGS

(71) Applicant: PROTOMER TECHNOLOGIES INC., Indianapolis, IN (US)

(72) Inventors: Ryan Kelly SPENCER, Porter Ranch, CA (US); Diao CHEN, Pasadena, CA (US); Sachitanand MALI, Pasadena, CA (US); Jack Joseph STEELE (nee HALE), Pasadena, CA (US); Jingxin LIANG, Covington, WA (US); Mirna Ekram Anwar SHAKER, Cypress, CA (US); Alborz MAHDAVI, Pasadena, CA (US)

(21) Appl. No.: 18/253,408

(22) PCT Filed: Nov. 18, 2021
(86) PCT No.: PCT/US2021/059802

§ 371 Date: Apr. 19, 2024

(30) Foreign Application Priority Data
Nov. 19, 2020 (US) 63116050
Dec. 7, 2020 (US) 63122338
Jun. 15, 2021 (US) 63210968
Sep. 29, 2021 (US) 63249868

(51) Int. Cl. A61K 47/54; A61K 38/22; A61K 38/26; A61K 38/28; A61K 38/31

(52) U.S. Cl. CPC A61K 47/545; A61K 38/22; A61K 38/26; A61K 38/28; A61K 38/31; A61K 47/542; A61K 47/543

(57) ABSTRACT

The present disclosure relates to novel compounds that include one or more aromatic boron-containing groups. The present disclosure further relates to pharmaceutical compositions containing such compounds, and their use in prevention and treatment of disorders, such as hyperglycemia, type 2 diabetes, impaired glucose tolerance, type 1 diabetes, obesity, metabolic syndrome X, or dyslipidemia, diabetes during pregnancy, pre-diabetes, Alzheimer's disease, MODY 1, MODY 2 or MODY 3 diabetes, mood disorders, and psychiatric disorders.

### CROSS REFERENCES TO RELATED APPLICATIONS

This application claims priority from U.S. Provisional Patent Application Nos. 63/116,050, filed Nov. 19, 2020, 63/122,338, filed Dec. 7, 2020, 63/210,968, filed Jun. 15, 2021, and 63/249,868, filed Sep. 29, 2021, all of which are incorporated herein by reference.

### FIELD OF THE DISCLOSURE

The present disclosure relates to novel compounds that include one or more aromatic boron-containing groups. The present disclosure further relates to kits and the use of the compounds and/or pharmaceutical compositions comprising the disclosed compounds for the treatment of disorders, such as hyperglycemia, type 2 diabetes, impaired glucose tolerance, type 1 diabetes, obesity, metabolic syndrome X, or dyslipidemia, diabetes during pregnancy, pre-diabetes, Alzheimer's disease, MODY 1, MODY 2 or MODY 3 diabetes, mood disorders, and psychiatric disorders.

### SEQUENCE LISTING

The present application is being filed along with a Sequence Listing in electronic format. The Sequence Listing is provided as a file titled “X23056 2Nov2021_ST25,” created Nov. 2, 2021, and is 4,084,000 bytes in size. The information in the electronic format of the Sequence Listing is incorporated herein by reference in its entirety.

### BACKGROUND OF THE DISCLOSURE

Boronic acids are generally considered Lewis acids that have a tendency to bind to hydroxyls, because, as Lewis acids, boronic acids can form complexes with Lewis bases such as, for example, hydroxide anions. Thus, molecules containing boronates including boronic acids have a general tendency to bind hydroxyl groups. This binding tendency can be used for detection of hydroxyl-containing groups by boronated labeling reagents wherein the boronate groups bind to the hydroxyls and, depending on the solvent and buffer conditions, the boronates can form hydrolysable boronate-ester bonds to the hydroxyl groups of hydroxyl containing molecules, such as the hydroxyl groups present in diols (e.g., glucose). Although boron-containing compounds can bind to diol containing molecules, achieving selectivity using boron-containing compounds has been challenging because of their ability to bind various diols, including cis diols, to varying degrees. While improved binding affinity of boron-containing compounds towards a specific vicinal diol of interest may be achieved, this may result in a loss of selectivity.

Glucose is the main fuel for the human body, and blood glucose values are tightly regulated in healthy individuals. For example, between meals, blood glucose is near 5 mmol/L (mM), and when blood glucose concentrations rise after a meal, the value is quickly adjusted back toward 5 mM by the action of insulin. The hormone insulin is secreted from pancreatic beta cells, and when insulin binds to insulin receptors on cells all over the body (for example muscle and fat), the cells are stimulated to absorb glucose by translocation of glucose transporters from storage vesicles to the cell surface (GLUT4). (Huang, S. H. et al. (2007) Cell Metabolism 5:237-252.)

People with diabetes may lose their ability to produce insulin due to autoimmunity against the beta cells (type 1) or have low sensitivity to insulin in combination with impaired insulin secretion (type 2). For example, those with type 1 diabetes may rely on multiple daily insulin injections, both for basal coverage, typically once a day, and with meals (bolus) to control their glucose levels. (Polonsky, K. S. et al. (1988) The Journal of Clinical Investigation 81: 442-448.) Because glucose values can fluctuate unpredictably, perfect insulin dosing day after day is extremely difficult. Indeed, despite many technological advances in diabetes treatment, researchers are currently seeing, partly due to lifestyle problems, a worsening of long-term glucose control and/or overall metabolic health.

Thus, there is an unmet medical need for novel compounds, such as glucose-responsive insulin analogues/conjugates, that can control blood glucose levels.

### SUMMARY OF THE DISCLOSURE

The present disclosure provides, according to some embodiments, novel Z1c compounds (e.g., FF1-FF224) comprising aromatic boron-containing functionalities (e.g., F1-F12). In some embodiments, the present disclosure provides compounds that comprise a drug substance (e.g., X1) and at least one Z1c with aromatic boron-containing functionalities (e.g., aromatic boron-containing groups, Z1c scaffolds comprising FF1-FF224 and F1-F12). In some embodiments, the compounds disclosed comprise one or more molecular scaffolds (e.g., FF scaffolds). In some embodiments, the compounds comprise a drug substance. In at least some embodiments, the drug substance is a polypeptide or a small-molecule.

In at least some embodiments, the disclosed compounds have at least two aromatic boron-containing functionalities. In some embodiments, the compounds are selective towards specific sugars, such as glucose, while showing reduced affinity for other sugars and wherein there is at least two aromatic boron-containing functionalities in the aromatic boron-containing portion of the compounds. In certain embodiments, the aromatic boron-containing portion may be covalently conjugated, directly or indirectly, to a drug substance comprising an amine, a drug substance that is covalently conjugated to an amine containing linker, an amine configured to be covalently conjugated to a drug substance, NH₂, or OH (e.g., X1). In some embodiments, the drug substance is covalently conjugated to an amine containing linker and the amine group is conjugated to the aromatic boron-containing portion (Z1c). In certain embodiments the aromatic boron-containing portion (Z1c) has an architecture comprising of tethering groups (FF formulae) and aromatic boron containing groups that collectively make the aromatic boron-containing portion of the disclosed compounds.

In some embodiments, one or more Z1c may be linked (e.g., conjugated, connected) to a drug substance (e.g., X1) via one or more small-molecule linkers (e.g., Z1b) and/or one or more amino acids connected together using amide or peptide bonds (e.g., Z1a). In at least some embodiments, rotational constraining of the boron functionalities via the tethering group (e.g., FF formulae) enhances the binding affinity of the compounds (e.g., conjugates) towards specific diols (such as glucose) and away from other diols in the body and thereby provides selectivity to specific diols. In some embodiments, the compounds may exhibit therapeutic pharmacokinetics and/or pharmacodynamics in response to endogenous and/or exogenous small molecules in the body, such as glucose. Changes in the physiological concentration of glucose, result in the activation and/or release of the drug molecule or conversely, deactivation and/or sequestering of the drug molecule (e.g., a peptide hormone), and/or modulation of the activity of the drug molecule, through interaction of glucose with the boron-containing architectures conjugated to the drug molecule.

In some embodiments, the present disclosure provides a compound comprising X1 and one or more Z1c, or a tautomer, stereoisomer or a mixture of stereoisomers, or a pharmaceutically acceptable salt, or hydrate, or isotopic derivative thereof, wherein:
X1 comprises:
   i. NH₂ or OH (e.g. X1 is NH₂ or OH);
   ii. a drug substance comprising an amine;
   iii. a drug substance that is covalently conjugated to an amine containing linker; or
   iv. an amine configured to be covalently conjugated to a drug substance;
wherein each Z1c is independently selected from Formulae FF1-FF224; and wherein each Z1c is covalently conjugated, directly or indirectly, to an amine in X1 or to OH when X1 is OH.

In some embodiments, the compound is a molecular conjugate represented by Formula I, or a stereoisomer or a mixture of stereoisomers, or pharmaceutically acceptable salts:

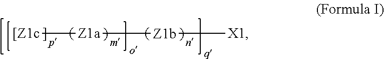

wherein X1 comprises:
   i. NH₂ or OH (e.g. X1 is NH₂ or OH);
   ii. a polypeptide drug substance comprising an amine;
   iii. a polypeptide drug substance that is covalently conjugated to an amine containing linker; or
   iv. an amine configured to be covalently conjugated to a polypeptide drug substance;
each Z1c is independently selected from Formulae FF1-FF224 and covalently conjugated either directly, or via Z1a and/or Z1b, to X1;
each Z1a comprises 1 to 50 amino acids connected together using amide or peptide bonds;
each Z1b is a small-molecule linker;
each m′ is independently 0 or 1;
each n′ is independently 0 or a positive integer;
each o′ is independently an integer of 1 or greater;
each p′ is a positive integer; and
q′ is a positive integer of at least 1 and not more than two times the total number of amine groups in X1, with the proviso that when any of n′, o′, p′, or q′ is 2 or more, the corresponding groups Z1a, Z1b, and Z1c are independently selected and may be the same or different;
wherein each Z1c is independently covalently conjugated, directly or indirectly, to an amine of Z1a, to an amine of Z1b, or to X1; and wherein optionally the molecular conjugate may comprise one or more isotopes at any position of the molecular conjugate of Formula I.

In some embodiments, X1 comprises human insulin or a human insulin analogue comprising an A-chain and a B-chain, wherein the A-chain comprises a sequence selected from SEQ ID NOs 1 and 3 to 33, and the B-chain comprises a sequence selected from SEQ ID NOs 2 and 34 to 74, 24047, and 24048;
each Z1c is independently selected from FF1, FF10, FF12, FF14, FF15, FF114, FF115, FF116, FF163, FF193, FF194, FF203, and FF221-FF224 and covalently conjugated either directly, or indirectly via a linker (an indirect linker), to Z1a and/or Z1b, or to X1;
each Z1a is independently absent or independently comprises a sequence selected from K, GK, KGSH (SEQ ID NO:24049), KGSHK (SEQ ID NO:4238), KNSTK (SEQ ID NO:5085), GKASHK (SEQ ID NO:12414), GKEEEK (SEQ ID NO:12677), GKEEHK (SEQ ID NO:12680), GKGHSK (SEQ ID NO:13120), GKGSH (SEQ ID NO:24050), GKGSHK (SEQ ID NO:13198), GKGSTK (SEQ ID NO:13205), GKHENK (SEQ ID NO:13271), GKNSHK (SEQ ID NO:13982), GKNSTK (SEQ ID NO:13989), GKQSSK (SEQ ID NO:14380), GKYQFK (SEQ ID NO:15128), GKGSKK (SEQ ID NO:24045), GKKPGKK (SEQ ID NO:24046), GKGPSK (SEQ ID NO:24044), GKPSHKP (SEQ ID NO:24043), and GSHKGSHK (SEQ ID NO:24042);
each said linker is selected from FL1, FL3, FL4, and FL5;
each m′ is independently 0 or 1;
each n′ is independently 0, 1, 2, or 3;
each o′ is independently 1, 2, 3, 4, or 5;
each p′ is 1, 2, 3, 4, or 5; and
q′ is 1, 2, 3, or 4, wherein when any of n′, o′, p′, or q′ is 2 or more, the corresponding groups Z1a, Z1b, and Z1c are independently selected and may be the same or different; and
wherein each Z1c is independently covalently conjugated, directly or indirectly, to an amine of Z1a, to an amine of Z1b, or to X1.

In some embodiments, the compound of Formula I is selected from:

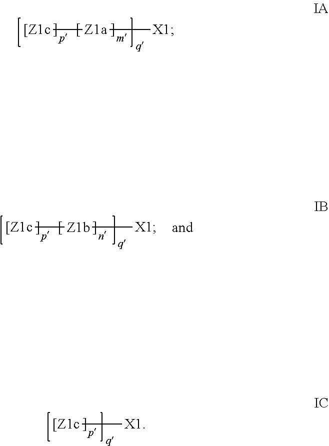

Also disclosed herein is a method of treating or preventing an endocrine and/or metabolic disease, in a subject in need thereof, comprising administering to said subject a compound of Formula (I), or a pharmaceutically acceptable salt thereof, or a pharmaceutical composition comprising a compound of Formula (I) or a pharmaceutically acceptable salt thereof. In some embodiments, the polypeptide drug substance is a polypeptide hormone such as insulin, an insulin analog, an incretin or an incretin analog. In some embodiments, the disease (e.g., disorder) is chosen from hyperglycemia, type 2 diabetes, impaired glucose tolerance, type 1 diabetes, obesity, metabolic syndrome X, or dyslipidemia, diabetes during pregnancy, pre-diabetes, Alzheimer's disease, MODY 1, MODY 2 or MODY 3 diabetes, mood disorders, and psychiatric disorders.

In at least one embodiment, the pharmaceutical composition of the present disclosure may be for use in (or in the manufacture of medicaments for) the treatment of diabetes in the subject. In at least one embodiment, a therapeutically-effective amount of a pharmaceutical composition of the present disclosure may be administered to a subject diagnosed with diabetes or metabolic disease. In at least one embodiment, the pharmaceutical composition of the present disclosure comprises at least one compound disclosed herein (e.g., Formula I, Z1c) and at least one additional component selected from pharmaceutically acceptable carriers, pharmaceutically acceptable vehicles, and pharmaceutically acceptable excipients.

In at least one embodiment, the present disclosure is directed to a human insulin analog comprising an A-chain and a B-chain, wherein the sequence of the A-chain comprises:

X_{aa′}X_{bb′}X_{cc′}X_{dd′}X_{ee′}X_{ff′}X_{gg′}VEQCCX_{hh′}X_{ii′}JCSLYQLENYCNX_{jj′}X_{kk′}X_{ll′}X_{mm′}X_{nn′}X_{oo′}X_{pp′} (SEQ ID NO:24015); and wherein the sequence of the B-chain comprises:
(i)

(SEQ ID NO: 24016)
XₐₐX_{bb}X_{cc}X_{dd}KXₑₑX_{ff}X_{gg}XₕₕXᵢᵢXⱼⱼKXₖₖXₗₗXₘₘXₙₙQHLCGSHLVEALYL
VCXₒₒXₚₚX_{qq}GFFYTXᵣᵣXₛₛXₜₜXᵤᵤXᵥᵥX_{ww},

wherein X_{aa′}, X_{bb′}, X_{cc′}, X_{dd′}, X_{ee′}, X_{ff′}, X_{gg′}, X_{hh′}, X_{ii′}, X_{jj′}, X_{kk′}, X_{ll′}, X_{mm′}, X_{nn′}, X_{oo′}, X_{pp′}, Xₐₐ, X_{bb}, X_{cc}, X_{dd}, Xₑₑ, X_{ff}, X_{gg}, Xₕₕ, Xᵢᵢ, Xⱼⱼ, Xₖₖ, Xₗₗ, Xₘₘ, Xₙₙ, Xₒₒ, Xₚₚ, X_{qq}, Xᵣᵣ, Xₛₛ, Xₜₜ, Xᵤᵤ, Xᵥᵥ, and X_{ww} are each independently either absent or selected from amino acid residues A, D, E, F, G, H, I, K, L, N, P, Q, R, S, T, V, Y and W,
(ii)

(SEQ ID NO: 24017)
XₐₐX_{bb}X_{cc}X_{dd}KPXₑₑX_{ff}X_{gg}XₕₕXᵢᵢXⱼⱼXₖₖXₗₗXₘₘXₙₙQHLCGSHLVEALYLV
CXₒₒXₚₚX_{qq}GFFYTXᵣᵣXₛₛXₜₜXᵤᵤXᵥᵥX_{ww},

wherein X_{aa′}, X_{bb′}, X_{cc′}, X_{dd′}, X_{ee′}, X_{ff′}, X_{gg′}, X_{hh′}, X_{ii′}, X_{jj′}, X_{kk′}, X_{ll′}, X_{mm′}, X_{nn′}, X_{oo′}, X_{pp′}, Xₐₐ, X_{bb}, X_{cc}, X_{dd}, X_{ff}, X_{gg}, Xₕₕ, Xᵢᵢ, Xⱼⱼ, Xₖₖ, Xₗₗ, Xₘₘ, Xₙₙ, Xₒₒ, Xₚₚ, X_{qq}, Xᵣᵣ, Xₛₛ, Xₜₜ, Xᵤᵤ, Xᵥᵥ, and X_{ww} are each independently either absent or selected from amino acid residues A, D, E, F, G, H, I, K, L, N, P, Q, R, S, T, V, Y and W, and wherein Xₑₑ is selected from amino acid residues A, E, F, H, I, K, L, N, P, Q, R, S, T, V, Y and W,
(iii)

(SEQ ID NO: 24018)
XₐₐX_{bb}X_{cc}X_{dd}KXₑₑX_{ff}X_{gg}XₕₕXᵢᵢXⱼⱼKXₖₖXₗₗXₘₘXₙₙQHLCGSHLVEALYLVC
XₒₒXₚₚX_{qq}GFFYTXᵣᵣXₛₛXₜₜXᵤᵤXᵥᵥX_{ww},

wherein X_{aa′}, X_{bb′}, X_{cc′}, X_{dd′}, X_{ee′}, X_{ff′}, X_{gg′}, X_{hh′}, X_{ii′}, X_{jj′}, X_{kk′}, X_{ll′}, X_{mm′}, X_{nn′}, X_{oo′}, X_{pp′}, Xₐₐ, X_{bb}, X_{cc}, X_{dd}, Xₑₑ, X_{ff}, X_{gg}, Xₕₕ, Xᵢᵢ, Xⱼⱼ, Xₖₖ, Xₗₗ, Xₘₘ, Xₙₙ, Xₒₒ, Xₚₚ, X_{qq}, Xᵣᵣ, Xₛₛ, Xₜₜ, Xᵤᵤ, Xᵥᵥ, and X_{ww}, are each independently either absent or selected from amino acid residues A, D, E, F, G, H, I, K, L, N, P, Q, R, S, T, V, Y, W and at least one of Xₑₑ, X_{ff}, X_{gg}, Xₕₕ, Xᵢᵢ, Xⱼⱼ is present and at least one of Xₑₑ, X_{ff}, X_{gg}, Xₕₕ, Xᵢᵢ, Xⱼⱼ is G,
(iv)

(SEQ ID NO: 24019)
XₐₐX_{bb}X_{cc}X_{dd}KXₑₑX_{ff}X_{gg}XₕₕXᵢᵢXⱼⱼKXₖₖXₗₗXₘₘXₙₙQHLCGSHLVEALYLVC
XₒₒXₚₚX_{qq}GFFYTXᵣᵣXₛₛXₜₜXᵤᵤXwX_{ww},

wherein X_{aa′}, X_{bb′}, X_{cc′}, X_{dd′}, X_{ee′}, X_{ff}, X_{gg′}, X_{hh′}, X_{ii′}, X_{jj′}, X_{kk′}, X_{ll′}, X_{mm′}, X_{nn′}, X_{oo′}, X_{pp′}, Xₐₐ, X_{bb}, X_{cc}, X_{dd}, Xₑₑ, X_{ff}, X_{gg}, Xₕₕ, Xᵢᵢ, Xⱼⱼ, Xₖₖ, Xₗₗ, Xₘₘ, Xₙₙ, Xₒₒ, Xₚₚ, X_{qq}, Xᵣᵣ, Xₛₛ, Xₜₜ, Xᵤᵤ, Xᵥᵥ, and X_{ww} are each independently either absent or selected from amino acid residues A, D, E, F, G, H, I, K, L, N, P, Q, R, S, T, V, Y, W and at least one of Xₑₑ, X_{ff}, X_{gg}, Xₕₕ, Xᵢᵢ, Xⱼⱼ is present and at least one of Xₑₑ, X_{ff}, X_{gg}, Xₕₕ, Xᵢᵢ, Xⱼⱼ is S, or
(v)

(SEQ ID NO: 24020)
XₐₐX_{bb}X_{cc}X_{dd}KXₑₑX_{ff}X_{gg}XₕₕXᵢᵢXⱼⱼKXₖₖXₗₗXₘₘXₙₙQHLCGSHLVEALYLVC
XₒₒXₚₚX_{gg}GFFYTXᵣᵣXₛₛXₜₜXᵤᵤXᵥᵥX_{ww},

wherein X_{aa′}, X_{bb′}, X_{cc′}, X_{dd′}, X_{ee′}, X_{ff′}, X_{gg′}, X_{hh′}, X_{ii′}, X_{jj′}, X_{kk′}, X_{ll′}, X_{mm′}, X_{nn′}, X_{oo′}, X_{pp′}, Xₐₐ, X_{bb}, X_{cc}, X_{dd}, Xₑₑ, X_{ff}, X_{gg}, Xₕₕ, Xᵢᵢ, Xⱼⱼ, Xₖₖ, Xₗₗ, Xₘₘ, Xₙₙ, Xₒₒ, Xₚₚ, X_{qq}, Xᵣᵣ, Xₛₛ, Xₜₜ, Xᵤᵤ, Xᵥᵥ, and X_{ww} are each independently either absent or selected from amino acid residues A, D, E, F, G, H, I, K, L, N, P, Q, R, S, T, V, Y, W and at least two of Xₑₑ, X_{ff}, X_{gg}, Xₕₕ, Xᵢᵢ, Xⱼⱼ are present and at least one of Xₑₑ, X_{ff}, X_{gg}, Xₕₕ, Xᵢᵢ, Xⱼⱼ is S, and another is G.

In some embodiments, one or more lysine residues and/or the N-terminus of the insulin A- or B-chain are covalently conjugated as described by Formula I. In some embodiments the insulins described herein are used as intermediate compounds for the manufacture of conjugates described by Formula I. In some embodiments, the insulins described herein are used in methods for preventing and/or treating an endocrine and/or metabolic disease, for example comprising administering any compound (e.g., modified insulin) of the embodiments described herein to a subject in need thereof, thereby treating the endocrine and/or metabolic disease.

### DETAILED DESCRIPTION OF THE DISCLOSURE

While aromatic boron-containing compounds (e.g., groups) can bind to diol containing molecules, achieving selectivity using aromatic boron-containing compounds (which can act as molecular sensors) is challenging because of their ability to bind various diols, including cis diols, to varying degrees. Improved binding affinity of aromatic boron-containing compounds (which can act as sensors) towards a specific vicinal diol of interest may result in a loss of selectivity.

Scaffolds that position the boron functionality (e.g., sensors) of the aromatic boron-containing compounds in a specific or particular ensemble of geometries can increase selectivity towards a specific vicinal diol while simultaneously maintaining affinity for the diol of interest. According to some embodiments, aromatic boron-containing compounds disclosed herein have different pendant groups on the aromatic boron-based scaffolds along with which specific scaffold geometries that impact binding to hydroxyl containing molecules.

According to some embodiments, the compounds of the present disclosure comprise aromatic boron-containing compounds that orient the boron functionalities in three dimensional space, so that the boron-containing compounds are spatially oriented to engage hexoses containing vicinal diols, such that the boron groups can appropriately engage the hydroxyls in the vicinal diol molecule and provide enhancement of selectivity. In some embodiments, the aromatic boron-containing compounds are modified with specific functional groups on the aromatic ring that, together with an appropriate or suitable scaffold, may provide higher selectivity and/or affinity for binding towards a vicinal diol of interest and away from other diols in the body.

In some embodiments, the aromatic boron-containing compounds are conjugated to a drug substance (e.g., small-molecule, polypeptide) wherein the aromatic boron-containing compounds provide intramolecular and intermolecular interactions with the drug substance and/or with proteins in the body, such as circulating proteins in the blood and/or plasma including albumin and/or globulins. In some embodiments, the selective binding of the sensors to specific vicinal diols changes the extent of those intramolecular and intermolecular bindings and thereby modulates the pharmacokinetics and overall activity of the drug substance in the body; this effect can be controlled by the level of the vicinal diols present.

In some embodiments the drug substance is a peptide hormone. In some embodiments, the peptide hormone is a human peptide hormone such as insulin, glucagon, or another incretin hormone. In some embodiments the sensors are selective towards the vicinal diols in glucose, and this selectivity is enhanced while maintaining affinity to glucose and simultaneously reducing affinity to other sugars in the blood. In some embodiments, the scaffolds as well as (e.g., in combination with) the pendant groups on the aromatic core of the boron-containing compounds enable controlling the overall activity and/or pharmacokinetics of the conjugated drug substances based on levels of glucose and/or other vicinal diols in the blood.

In some embodiments, the aromatic boron-containing compounds comprise specific scaffold molecules (i.e., FF structures) with conjugated boron functionalities (i.e., F1-F12 groups), wherein the scaffolds have been used to orient the boron functionalities in three dimensional geometries so that the boron functionalities are oriented near each other and within a distance that helps engage specific hydroxyl orientations of select hexoses such as glucose. Without wishing to be bound by theory, it is believed that the aromatic boron-containing compounds (e.g., molecules) disclosed herein enhance selectivity through at least one or more of the following three mechanisms: (1) the FF scaffold facilitates matching the orientation of the hydroxyl and/or alkoxy groups on boron groups in the aromatic boron-containing compounds and the hydroxyls in the vicinal diol molecule which enhances selectivity; (2) further selectivity gain is obtained by identifying specific functional groups attached to, or near, for example, the aromatic core of the boron-containing compound which impact the electronic structure of the aromatic boron-containing compound and thereby favor reversible binding to the vicinal diols at physiological pH; and (3) functional groups attached to the aromatic boron-containing compound (e.g., the sensor scaffold) help to provide steric hindrance to reduce binding to unwanted hexoses while maintaining binding to the sugar of interest such as glucose. These effects as combined together in the present disclosure provide desired or suitable selectivity of binding towards a vicinal diol-containing molecule of interest and away from other diols in the body.

In some embodiments, the aromatic boron-containing compounds are conjugated to a drug substance wherein the aromatic boron-containing compounds provide intramolecular and/or intermolecular interactions with proteins in the body. Such proteins may include circulating proteins in the blood and/or human plasma such as albumin, glycosylated proteins and/or immunoglobulins. In some embodiments the selective binding of the sensors to specific vicinal diols in a molecule of interest changes the extent of intramolecular and intermolecular bindings and thereby modulates the pharmacokinetics and overall activity of the drug substance in the body. In some embodiments the drug substance is a peptide hormone and in certain embodiments thereof the peptide hormone is an incretin hormone such as insulin and the vicinal diol containing molecule is glucose, but the present disclosure is not limited thereto.

### Definitions

Unless otherwise defined, all terms (including technical and scientific terms) used herein have the same meaning as commonly understood by one of ordinary skill in the art to which the present disclosure belongs. It will be further understood that terms, such as those defined in commonly used dictionaries, should be interpreted as having a meaning that is consistent with their meaning in the context of the relevant art and/or the present specification, and should not be interpreted in an idealized or overly formal sense, unless expressly so defined herein.

Unless specifically described herein, functional groups, functional moieties, and reactions referred to herein are understood to have meanings consistent with standard descriptions in and/or general principles of organic chemistry, for example, as described in Organic Chemistry, Thomas Sorrell, University Science Books, Sausalito, 1999; Larock, Comprehensive Organic Transformations, VCH Publishers, Inc., New York, 1989; Carruthers, Some Modern Methods of Organic Synthesis, 3^{rd} Edition, Cambridge University Press, Cambridge, 1987; Smith and March, March's Advanced Organic Chemistry, 5^{th} Edition, John Wiley & Sons, Inc., New York, 2001. Generic functional groups (such as alkyl, aryl, acetyl, etc.) encompass specific examples or species falling within those functional group categories as generally defined in the field of organic chemistry, and those having ordinary skill in the art are capable of identifying specific example embodiments of functional groups.

Unless specifically described herein, chemical terms, functional groups, and general terms used throughout the specification are identified in accordance with the Periodic Table of the Elements, CAS version, Handbook of Chemistry and Physics, 75^{th} Ed., inside cover. In certain embodiments the terms “a,” “an,” and “the” and similar referents used herein are to be construed to cover both the singular and the plural unless their usage in context indicates otherwise. The term “CAS #” as used herein is also referred to as CASRN or CAS Number, is a unique numerical identifier assigned by Chemical Abstracts Service (CAS) to every chemical substance described in the open scientific literature.

As used herein, nomenclature for compounds including organic compounds, can be given using common names, IUPAC, IUBMB, or CAS recommendations for nomenclature. One of skill in the art can readily ascertain the structure of a compound if given a name, either by systemic reduction of compound structure using naming conventions, or by commercially available software, such as CHEMDRAW™ (Cambridgesoft Corporation, U.S.A.).

The terminology used herein is for the purpose of describing embodiments and is not intended to be limiting of the present disclosure. It will be further understood that the terms “comprises,” “comprising,” “includes,” and “including,” when used in this specification, specify the presence of the stated features, integers, acts, operations, elements, and/or components, but do not preclude the presence or addition of one or more other features, integers, acts, operations, elements, components, and/or groups thereof. As used herein, the term “and/or” includes any and all combinations of one or more of the associated listed items. Expressions such as “at least one of,” when preceding a list of elements, modify the entire list of elements and do not modify the individual elements of the list.

As used herein, the terms “substantially,” “about,” and similar terms are used as terms of approximation and not as terms of degree, and are intended to account for the inherent deviations in measured or calculated values that would be recognized by those of ordinary skill in the art. The term “about” used throughout is used to describe and account for small variations. For instance, “about” may mean the numeric value may be modified by ±5%, ±4%, ±3%, ±2%, ±1%, ±0.5%, ±0.4%, ±0.3%, ±0.2%, ±0.1% or ±0.05%. Numeric values modified by the term “about” include the specific identified value. For example, “about 5.0” includes 5.0.

Further, the use of “may” when describing embodiments of the present disclosure refers to “one or more embodiments of the present disclosure.” As used herein, the terms “use,” “using,” and “used” may be considered synonymous with the terms “utilize,” “utilizing,” and “utilized,” respectively. Also, the term “exemplary” is intended to refer to an example or illustration.

Also, any numerical range recited herein is intended to include all sub-ranges of the same numerical precision subsumed within the recited range. For example, a range of “1 to 10” is intended to include all subranges between (and including) the recited minimum value of 1 and the recited maximum value of 10, that is, having a minimum value equal to or greater than 1 and a maximum value equal to or less than 10, such as, for example, 2 to 7. Any maximum numerical limitation recited herein is intended to include all lower numerical limitations subsumed therein, and any minimum numerical limitation recited in this specification is intended to include all higher numerical limitations subsumed therein. Accordingly, Applicant reserves the right to amend this specification, including the claims, to expressly recite any sub-range subsumed within the ranges expressly recited herein.

As used herein, “aromatic boron-containing group” refers to a compound having at least one boron atom covalently bonded to an aromatic group and/or a compound having at least one boron atom covalently incorporated within an aromatic group. The term “aromatic” as used herein may include “heterocycle,” “heterocyclyl,” or “heterocyclic.” As used herein the terms “heterocycle,” “heterocyclyl,” or “heterocyclic” each refer to an unsaturated 3- to 18-membered ring containing one, two, three, or four heteroatoms independently selected from nitrogen, oxygen, phosphorus, and sulfur. In some embodiments, the term “aromatic” may include an “aryl.” The term “aryl” as used herein refers to a mono-, bi-, or other multi carbocyclic, aromatic ring system with 5 to 14 ring atoms. The aryl group can optionally be fused to one or more rings selected from aryls, cycloalkyls, heteroaryls, and heterocyclyls. Exemplary aryl groups also include but are not limited to a monocyclic aromatic ring system, wherein the ring comprises 6 carbon atoms.

The term “heteroaryl” as used herein refers to a mono-, bi-, or multi-cyclic, aromatic ring system containing one or more heteroatoms, for example 1-3 heteroatoms, such as nitrogen, oxygen, and sulfur. Heteroaryls can be substituted with one or more substituents. Heteroaryls can also be fused to non-aromatic rings. Exemplary heteroaryl groups include, but are not limited to, a monocyclic aromatic ring, wherein the ring comprises 2-5 carbon atoms and 1-3 heteroatoms. In some embodiments, the aromatic boron-containing group may include but is not limited to aryl- and heteroaryl boronic acids, aryl and heteroaryl boronate esters, and/or boroxoles. Exemplary aromatic boron-containing groups useful according to certain embodiments, include, e.g., those described herein as FF1-FF224, F1-F10 and further include, e.g., those as disclosed in patent application PCT/US2021/025261 (filed Mar. 31, 2021) as compounds F1-F9, F12-F43, F500-F520; the disclosure of which is herein expressly incorporated by reference in its entirety.

The term “small-molecule linker” as used herein refers to a chemical group (e.g., scaffold, moiety) comprising a first attachment point toward X1 and a second attachment point toward Z1b, Z1a, or Z1c. In some embodiments, the first attachment point is toward X1 and the second attachment point is toward Z1c. In some embodiments, the first attachment point is toward X1 and the second attachment point is toward Zia. In some embodiments, the small molecule linker is a moiety/chemical group selected from Formulae IIa-IIai and Formulae IIIa-IIIai. In some embodiments, the small molecule linker is a moiety/chemical group selected from Formulae FL1-FL19 and an L- or D-amino acid comprising at least one amine group directly conjugated to Z1c, wherein an acid functional group of the amino acid is conjugated toward X1 in Formula I.

The term “indirect linker” as used herein refers to a chemical group (e.g., scaffold, moiety) comprising a first attachment point toward X1 and a second attachment point toward Z1b, Z1a, or Z1c. In some embodiments, the first attachment point is toward X1 and the second attachment point is toward Z1c. In some embodiments, the first attachment point is toward Z1a and the second attachment point is toward Z1c. In some embodiments, the indirect linker is a moiety/chemical group selected from Formulae FL1-FL19 and an L- or D-amino acid comprising at least one amine group directly conjugated to Z1c, wherein an acid functional group of the amino acid is conjugated toward Z1a or X1, independently, in Formula I.

The term “alkyl” as used herein refers to a saturated straight or branched hydrocarbon, such as a straight or branched group of 1-30 carbon atoms, referred to herein as C₁₋₃₀ alkyl. In some embodiments, the alkyl group is a C₁-C₂₂ alkyl group. In some embodiments, the alkyl group is a C₁-C₂₀ alkyl group. In some embodiments, the alkyl group is a C₁-C₁₈ alkyl group. In some embodiments, the alkyl group is a C₁-C₁₆ alkyl group. In some embodiments, the alkyl group is a C₁-C₁₄ alkyl group. In some embodiments, the alkyl group is a C₁-C₁₂ alkyl group. In some embodiments, the alkyl group is a C₁-C₁₀ alkyl group. In some embodiments, the alkyl group is a C₁-C₈ alkyl group. In some embodiments, the alkyl group is a C₁-C₆ alkyl group. In some embodiments, the alkyl group is a C₁-C₄ alkyl group. Exemplary alkyl groups include, but are not limited to, methyl, ethyl, propyl, isopropyl, 2-methyl-1-propyl, 2-methyl-2-propyl, 2-methyl-1-butyl, 3-methyl-1-butyl, 2-methyl-3-butyl, 2,2-dimethyl-1-propyl, 2-methyl-1-pentyl, 3-methyl-1-pentyl, 4-methyl-1-pentyl, 2-methyl-2-pentyl, 3-methyl-2-pentyl, 4-methyl-2-pentyl, 2,2-dimethyl-1-butyl, 3,3-dimethyl-1-butyl, 2-ethyl-1-butyl, butyl, isobutyl, t-butyl, pentyl, isopentyl, neopentyl, hexyl, heptyl, and octyl. In some embodiments, “alkyl” is a straight-chain hydrocarbon. In some embodiments, “alkyl” is a branched hydrocarbon.

The term “cycloalkyl” as used herein refers to a saturated or unsaturated cyclic, bicyclic, or bridged bicyclic hydrocarbon group of 3-16 carbons, or 3-8 carbons, referred to herein as “(C₃-C₅)cycloalkyl,” derived from a cycloalkane. Exemplary cycloalkyl groups include, but are not limited to, cyclohexanes, cyclohexenes, cyclopentanes, and cyclopentenes.

Cycloalkyl groups may be substituted with alkoxy, aryloxy, alkyl, alkenyl, alkynyl, amide, amino, aryl, arylalkyl, carbamate, carboxy, cyano, cycloalkyl, ester, ether, formyl, halogen, haloalkyl, heteroaryl, heterocyclyl, hydroxyl, ketone, nitro, phosphate, sulfide, sulfinyl, sulfonyl, sulfonic acid, sulfonamide and thioketone. Cycloalkyl groups can be fused to other cycloalkyl (saturated or partially unsaturated), aryl, or heterocyclyl groups, to form a bicycle, tetracycle, etc. The term “cycloalkyl” also includes bridged and spiro-fused cyclic structures which may or may not contain heteroatoms.

The term “acyl” as used herein refers to R—C(O)— groups such as, but not limited to, (alkyl)-C(O)—, (alkenyl)-C(O)—, (alkynyl)-C(O)—, (aryl)-C(O)—, (cycloalkyl)-C(O)—, (heteroaryl)-C(O)—, and (heterocyclyl)-C(O)—, wherein the group is attached to the parent molecular structure through the carbonyl functionality. In some embodiments, it is a C₁₋₁₀ acyl radical which refers to the total number of chain or ring atoms of the, for example, alkyl, alkenyl, alkynyl, aryl, cycloalkyl, or heteroaryl, portion plus the carbonyl carbon of acyl. For example, a C₄-acyl has three other ring or chain atoms plus carbonyl. In some embodiments, it is a C₁-C₂₂acyl group. In some embodiments, it is a C₁-C₂₀acyl group. In some embodiments, it is a C₁-C₁₈acyl group. In some embodiments, it is a C₁-C₁₆acyl group. In some embodiments, it is a C₁-C₁₄acyl group. In some embodiments, it is a C₁-C₁₂acyl group. In some embodiments, it is a C₁-C₁₀acyl group. In some embodiments, it is a C₁-C₈acyl group.

The term “haloalkyl” as used herein refers to an alkyl group substituted with one or more halogens. Examples of haloalkyl groups include, but are not limited to, trifluoromethyl, difluoromethyl, pentafluoroethyl, trichloromethyl, etc. In some embodiments, it is a C₁-C₂₂ haloalkyl group. In some embodiments, it is a C₁-C₂₀ haloalkyl group. In some embodiments, it is a C₁-C₁₈ haloalkyl group. In some embodiments, it is a C₁-C₁₆ haloalkyl group. In some embodiments, it is a C₁-C₁₄ haloalkyl group. In some embodiments, it is a C₁-C₁₂ haloalkyl group. In some embodiments, it is a C₁-C₁₀ haloalkyl group. In some embodiments, it is a C₁-C₈ haloalkyl group.

The term “aryl” as used herein refers to a mono-, bi-, or other multi-carbocyclic, aromatic ring system with 5 to 14 ring atoms. The aryl group can optionally be fused to one or more rings selected from aryls, cycloalkyls, heteroaryls, and heterocyclyls. The aryl groups of this present disclosure can be substituted with groups selected from alkoxy, aryloxy, alkyl, alkenyl, alkynyl, amide, amino, aryl, arylalkyl, carbamate, carboxy, cyano, cycloalkyl, ester, ether, formyl, halogen, haloalkyl, heteroaryl, heterocyclyl, hydroxyl, ketone, nitro, phosphate, sulfide, sulfinyl, sulfonyl, sulfonic acid, sulfonamide, and thioketone. Exemplary aryl groups include, but are not limited to, phenyl, tolyl, anthracenyl, fluorenyl, indenyl, azulenyl, and naphthyl, as well as benzo-fused carbocyclic moieties such as 5,6,7,8-tetrahydronaphthyl. Exemplary aryl groups also include but are not limited to a monocyclic aromatic ring system, wherein the ring comprises 6 carbon atoms.

“Isomers” means compounds having the same number and kind of atoms, and hence the same molecular weight, but differing with respect to the arrangement or configuration of the atoms in space.

“Stereoisomer” or “optical isomer” means a stable isomer that has at least one chiral atom or restricted rotation giving rise to perpendicular dissymmetric planes (e.g., certain biphenyls, allenes, and spiro compounds) and can rotate plane-polarized light. Because asymmetric centers and other chemical structure exist in the compounds of the disclosure which may give rise to stereoisomerism, the disclosure contemplates stereoisomers and mixtures thereof. The compounds of the disclosure and their salts include asymmetric carbon atoms and may therefore exist as single stereoisomers, racemates, and as mixtures of enantiomers and diastereomers. In some embodiments, such compounds will be prepared as a racemic mixture. In some embodiments, such compounds can be prepared or isolated as pure stereoisomers, e.g., as individual enantiomers or diastereomers, or as stereoisomer-enriched mixtures. As discussed in more detail below, individual stereoisomers of compounds may be prepared by synthesis from optically active starting materials containing the desired chiral centers or by preparation of mixtures of enantiomeric products followed by separation or resolution, such as conversion to a mixture of diastereomers followed by separation or recrystallization, chromatographic techniques, use of chiral resolving agents, or direct separation of the enantiomers on chiral chromatographic columns. Starting compounds of particular stereochemistry are either commercially available or are made by the methods described below and resolved by techniques well-known in the art.

The term “pharmaceutically acceptable salt(s)” refers to salts of acidic or basic groups that may be present in compounds used in the present compositions.

As used herein, “drug substance” refers to small-molecule compounds and/or polypeptide containing compounds. According to some embodiments, a drug substance suitable for use in the compounds and methods described herein is a therapeutically, prophylactically and/or diagnostically active drug substance.

It will be understood that, although terms such as “first,” “second,” “third,” etc., may be used herein to describe various elements (such as molecules, components, groups, and/or moieties, etc.), those elements should not be limited by these terms. These terms are merely used to distinguish one element from another element. Thus, a first element described below could be termed a second element without departing from the spirit and scope of the present disclosure. It will be understood that when an element or group is referred to as being “connected to,” “conjugated with,” “linked,” or “coupled to” another element or group, the two elements may be directly connected, or one or more intervening elements may be present. It will be understood that conjugations and linkages described herein have the option of being direct conjugations or direct linkages, unless expressly excluded or precluded by the context.

As used herein, the terms “directly” or “directly covalently conjugated” or “covalently conjugated directly” may be interchangeably used to indicate that a first group is “directly” or “directly covalently conjugated” or “covalently conjugated directly” to a second group, which means the first and second groups are covalently bonded together without additional intervening groups.

As used herein, the terms “indirectly” or “indirectly covalently conjugated” or “covalently conjugated indirectly” may be interchangeably used to indicate that a first group is “indirectly” or “indirectly covalently conjugated” or “covalently conjugated indirectly” to a second group, which means the first and second groups are covalently bonded together with at least one additional intervening group (e.g., a small-molecule, a linker, a spacer, a linear sequence of amino acids and/or nonlinear sequence of amino acids).

In at least some embodiments, one or more groups (e.g., X1a, Z1a, Z1b, Z1c) are covalently conjugated directly or indirectly to each other. For example, according to certain embodiments Z1c is covalently conjugated, directly or indirectly, to an amine in X1 or to OH when X1 is OH. As another example, according to certain embodiments one or more drug substances (X1) are covalently conjugated to one or more amine containing linkers. In some embodiments, X represents a point of covalent attachment either directly to an amine in X1 or to an amine that is covalently conjugated directly or indirectly to X1, or to OH when X1 is OH

In certain embodiment, each Z1c is independently covalently conjugated, directly or indirectly, to an amine of Z1a, to an amine of Z1b, or to X1.

As used herein, terms such as “attachment point toward [group],” “attachment to,” and “covalent linkage toward [group]” express that the indicated atom, attachment, or linkage is closer to the indicated group than the other attachment point or covalent linkage variables within the structure formula. In some embodiments an attachment point or covalent linkage may be directly adjacent to the indicated group, and in some embodiments other atoms or groups may be present therebetween.

As used herein, the term “percentage homology” refers to the percentage of sequence identity between two sequences after optimal alignment. Identical sequences have a percentage homology of 100%. Optimal alignment may be performed by homology alignment algorithms described by the search for similarity method of Pearson and Lipman, Proc. Natl. Acad. Sci. USA 85:2444 (1988), by general methods described for search for similarities by Neddleman and Wunsch, J. Mol. Biol. 48:443 (1970), including implementation of these algorithms or visual comparison. As used herein, “insulin A-chain” is the chain of insulin that has the highest percentage homology to the A-chain of wild-type human insulin. As used herein, “insulin B-chain” is the chain of insulin that has the highest percentage homology to the B-chain of wild-type human insulin.

In some embodiments, the terms “covalently connected,” “covalently conjugated,” or “through a covalent conjugation” may be interchangeably used to indicate that two or more atoms, groups, or chemical moieties are bonded or connected via a chemical linkage. In some embodiments, the chemical linkage (which in some embodiments may be referred to as a covalent linkage) may be (e.g., consist of) one or more shared electron pairs (e.g., in a single bond, a double bond, or a triple bond) between two atoms, groups, or chemical moieties.” In some embodiments, the chemical (covalent) linkage may further include one or more atoms or functional groups, and may be referred to using the corresponding name of that functional group in the art. For example, a covalent linkage including a —S—S— group may be referred to as a disulfide linkage; a covalent linkage including a —(C=O)— group may be referred to as a carbonyl linkage; a covalent linkage including a —(CF₂)— group may be referred to as a difluoromethylene linkage, etc. The type of linkage or functional group within the covalent bond is not limited unless expressly stated, for example when it is described as including or being selected from certain groups. The types or kinds of suitable covalent linkages will be understood from the description and/or context.

In some embodiments, side chains of amino acids may be covalently connected (e.g., linked or cross-linked) through any number of chemical bonds (e.g., bonding moieties) as generally described in Bioconjugate Techniques (Third edition), edited by Greg T. Hermanson, Academic Press, Boston, 2013. For example, the side chains may be covalently connected through an amide, ester, ether, thioether, isourea, imine, triazole, or any suitable covalent conjugation chemistry available in the art for covalently connecting one peptide, protein, or synthetic polymer to a second peptide, protein, or synthetic polymer. The term polymer includes polypeptide. The term “covalent conjugation chemistry” may refer to one or more functional groups included in the bonding moiety, and/or the chemical reactions used to form the bonding moiety.

The term “vicinal diol” refers to a group of molecules in which two hydroxyl groups occupy vicinal positions, that is, they are attached to adjacent atoms. Such molecules may include, but are not limited to, sugars such as hexoses, glucose, mannose and fructose.

In some embodiments, the term “albumin” means human serum albumin or a protein with at least 60% percentage homology to human serum albumin protein. It is to be understood that in some embodiments the albumin may be further chemically modified for the purposes of conjugation. In some embodiments, modifications may include one or more covalently connected linkers.

The term “treatment” is meant to include both the prevention and minimization of the referenced disease, disorder, or condition (i.e., “treatment” refers to both prophylactic and therapeutic administration of a compound of the present invention or a composition comprising a compound of the present invention unless otherwise indicated or clearly contradicted by context). The route of administration may be any route which effectively transports a compound of this disclosure to the desired or appropriate place in the body, such as parenterally, for example, subcutaneously, intramuscularly, orally, or intravenously. For parenterally administration, a compound of the disclosure is formulated analogously with the formulation of known insulins. Furthermore, for parenterally administration, a compound of this disclosure is administered analogously with the administration of known insulins and the physicians are familiar with this procedure. The amount of a compound of this disclosure to be administered, the determination of how frequently to administer a compound of this disclosure, and the election of which compound or compounds of this disclosure to administer, optionally together with another antidiabetic compound, is decided in consultation with a practitioner who is familiar with the treatment of the condition (e.g. diabetes) to be treated.

In some embodiments, “therapeutic composition” and “pharmaceutical composition” as used herein means a composition that is intended to have a therapeutic effect such as pharmaceutical compositions, genetic materials, biologics, and other substances. Pharmaceutical compositions may be configured to function in the body with therapeutic qualities; concentration may be altered to reduce the frequency of replenishment, and the like. In some embodiments “therapeutically effective amount” and “prophylactically effective amount” refer to an amount that provides a therapeutic benefit in the treatment, prevention, or management of a disease or an overt symptom of the disease. The therapeutically effective amount may treat a disease or condition, a symptom of disease, or a predisposition toward a disease, with the purpose to cure, heal, alleviate, relieve, alter, remedy, ameliorate, improve, or affect the disease, the symptoms of disease, or the predisposition toward disease. The set or specific amount that is therapeutically effective can be readily determined by an ordinary medical practitioner, and may vary depending on factors known in the art, such as, e.g. the type of disease, the patient's history and age, the stage of disease, and the administration of other therapeutic agents. In some embodiments, modified insulins described herein are delivered to the body by injection or inhalation, or by other routes, and can reversibly bind to soluble glucose in a non-depot form. In some embodiments, modified insulins described herein are released over an extended period of time from a local depot in the body or from bound forms to proteins in the serum such as albumin. In some embodiments, the release of modified insulin is accelerated at elevated glucose levels, and in some embodiments such release rate may be dependent on blood sugar levels or levels of other small molecules in the blood including diol containing molecules. In some embodiments the release, bioavailability, and/or solubility of modified insulins described herein is controlled as a function of blood or serum glucose concentrations or concentrations of other small molecules in the body.

Additionally, unless otherwise stated, structures described herein are also meant to include compounds that differ only in the presence of one or more isotopically enriched atoms. For example, compounds having the present structures except for the replacement of hydrogen by deuterium (²H) or tritium (³H), or the replacement of a carbon by a ¹³C- or ¹⁴C-carbon atom are within the scope of this disclosure. Such compounds may be useful as, for example, analytical tools, probes in biological assays, or therapeutic agents.

In some embodiments, functional groups can be covalently conjugated or linked via any suitable covalent conjugation chemistry (linker) that can be used to covalently conjugate one functional group or amino acid side chain to another functional group, non-limiting examples include an amide, an ester, an ether, a thioether, an isourea, an imine, and a triazole linker. In some embodiments functional groups are covalently conjugated through click chemistry reactions as defined in the art. These include, for example, cycloaddition reactions including but not limited to 3+2 cycloadditions, strain-promoted alkyne-nitrone cycloaddition, reactions of strained alkenes, alkene and tetrazine inverse-demand Diels-Alder, Copper(I)-Catalyzed Azide-Alkyne Cycloaddition (CuAAC), Strain-promoted azide-alkyne cycloaddition, Staudinger ligation, nucleophilic ring-opening reactions, and additions to carbon-carbon multiple bonds. Some of these reactions are described for example by H. C. Kolb, M. G. Finn and K. B. Sharpless (2001); Click Chemistry: Diverse Chemical Function from a Few Good Reactions, Angewandte Chemie International Edition 40 (11): 2004-2021; Kolb and Sharpless, Drug Discovery Today 8:1128-1137, 2003; Huisgen, R. Angew. Chem. Int. Ed. Engl. 1963, 2, 565; Agard, N. J.; Baskin, J. M.; Prescher, J. A.; Lo, A.; Bertozzi, C. R. ACS Chem. Biol. 2006, 1, 644. One skilled in the art will be capable of selecting suitable buffers, pH and reaction conditions for such click reactions. In some embodiments, covalent conjugation is the result of a “bioorthogonal reaction” as defined in the art. Such reactions are, for example, described by Sletten, Ellen M.; Bertozzi, Carolyn R. (2009). Bioorthogonal Chemistry: Fishing for Selectivity in a Sea of Functionality, Angewandte Chemie International Edition 48 (38): 6974-98.; Prescher, Jennifer A; Bertozzi, Carolyn R (2005). Chemistry in living systems, Nature Chemical Biology 1 (1): 13-21.

In some embodiments, functional groups may be linked using native chemical ligation as described for example by Dawson, P. E.; Muir, T. W.; Clark-Lewis, I.; Kent, S. B. (1994) Synthesis of proteins by native chemical ligation, Science 266 (5186): 776-778. As used herein, terms such as “linkage,” “covalent conjugation,” etc. may refer to any of the chemistries described above in some embodiments. The terms “amine,” “amino group,” and/or “amine group,” when used to describe part of a covalent bond or connectivity, may be interchangeably used to indicate an amino group or an amine group to which the described element is covalently linked. In some embodiments, the amino group or amine group may be a primary amine, a secondary amine, or a fragment such as NH—⊕ to which a conjugation is made and described.

In some embodiments, the amino group or amine group may be the NH₂ group at the N-terminus of a peptide or peptide chain, or the NH₂ group of a lysine side chain, but embodiments of the present disclosure are not limited thereto. In some embodiments, the connectivity of a first group to a second group is described by reference to an amine or amino group, originating from the second group, that is part of a covalent linkage between the first group and the second group. For example, an amine of a lysine side chain on X1 may be referred to as an amine, and furthermore may be described as being conjugated through an amide bond in order to specify the structure and connectivity of the functional groups that constitute the covalent bond. If a covalent linkage is via an amine bond or amine linkage, then it is referred to as an amine linkage. It is to be understood that a carbonyl connected to an amine (e.g., a (C═O)—NH moiety) constitutes an amide bond, and thus by definition, an amine linkage is not directly connected to a carbonyl group. Stated another way, the terms “amide bond” and/or “amide linkage,” when used to describe a covalent bond or connectivity, may be interchangeably used to indicate a carbonyl connected to an amine (e.g., a (C═O)—NH moiety).

In some embodiments further modifications include attachment of a chemical entity (e.g., moiety or functional group) such as a carbohydrate group, one or more cis-diol containing groups, one or more phosphate groups, one or more catechol groups, a farnesyl group, an isofarnesyl group, a fatty acid group, or a linker for conjugation, functionalization, or other modifications meant to impact the pharmacokinetics, pharmacodynamics, and/or biophysical solution characteristics of insulin.

In some embodiments, a compound, such as a molecular conjugate, includes a human peptide hormone (e.g., as X1). In some embodiments, the peptide hormone is a polypeptide hormone of the human pancreas. In some embodiments, X1 in Formula I is NH₂. In some embodiments, a compound of Formula I is conjugated to a drug substance via an optional covalent-spacer. In some embodiments, a compound, such as a molecular conjugate, includes a human insulin or a human insulin analogue. In some embodiments, two different amine groups in insulin are covalently conjugated to as described by Formula I.

It will be understood that “human peptide hormone,” “polypeptide hormone of the human pancreas,” “Insulin,” “human insulin,” “modified insulin,” and “human insulin analogue” may be used interchangeably in some of the described embodiments; that is, for example, in certain embodiments “human insulin analogue” may instead be used in embodiments described as using human insulin. In some embodiments a compound, such as a molecular conjugate, includes a human insulin or a human insulin analogue. In some embodiments, a molecular conjugate includes a human insulin or a human insulin analogue as described by Formula I for p′=1 wherein a single amino group in insulin is conjugated to as described by Formula I. In some embodiments, the amino group is the N-terminus of the B-chain of insulin or an amino group of the side chain of a lysine. In some embodiments, two or more different amine groups in insulin are each independently covalently conjugated to as described by Formula I. In some embodiments, at least one amine groups is the N-terminus of the B-chain of insulin. In some embodiments, amino groups comprise amino groups of side chains of lysine residues in insulin.

Various suitable modifications of the peptide hormone (e.g., human polypeptide hormone, for example, Insulin), known to those having ordinary skill in the art, are included in the scope of the disclosure. In some embodiments, the polypeptide of Z1a or the optionally extended polypeptide at the N-terminus of B-chain or C-terminus of A-chain of insulin contain sequences with up to 70% sequence homology to a human polypeptide sequence. In some embodiments, the polypeptide of Z1a or the optionally extended polypeptide at the N-terminus of B-chain or C-terminus of A-chain of insulin contain one or more lysine residues that are optionally next to a proline residue, such that the proline is C-terminal to lysine. In some embodiments, the amino group of lysine residues is each independently conjugated as described by Formula I.

In some embodiments, insulin is further modified through conjugation to a sugar- or diol-containing molecule. In some embodiments, the human polypeptide hormone is a dual or triple hybrid peptide comprising sequences of two or more human peptide hormones and which can act through multiple receptors; for example, a glucose-dependent insulinotropic polypeptide (GIP) and GLP-1 receptor agonist or GLP-1/GIP/glucagon triple agonist. In some embodiments, the human polypeptide hormone is a gut hormone. In some embodiments, the human polypeptide hormone is chosen from c-peptide, adrenocorticotropic hormone (ACTH), amylin, angiotensin, atrial natriuretic peptide (ANP), calcitonin, cholecystokinin (CCK), gastrin, ghrelin, glucagon, growth hormone, follicle-stimulating hormone (FSH), insulin, leptin, melanocyte-stimulating hormone (MSH), oxytocin, parathyroid hormone (PTH), prolactin, renin, somatostatin, thyroid-stimulating hormone (TSH), thyrotropin-releasing hormone (TRH), vasopressin, vasoactive intestinal peptide, a neuropeptide, a peptide hormone that impacts cardiovascular health or appetite, a hybrid of one or more of these peptides, and an analogue of one of these peptides. In some embodiments, compounds comprise a human polypeptide hormone further modified, for example, through the covalent conjugation to polymers, XTEN protein sequences or aliphatic chains. In some embodiments, polymer modified compounds have a longer circulation time in the blood. In some embodiments, polymer modified compounds, such long-acting variants require once a day injection, or one a week injection or once a month injection. In some embodiments, a human polypeptide hormone or the analogue thereof includes one or more L- or D-amino acids that are each independently one of the twenty canonical amino acids or a non-canonical amino acid.

In some embodiments, a human polypeptide hormone or the analogue thereof includes one or more residues that are 2-aminoisobutyric acid. In some embodiments, the C-terminus of the B-chain of insulin is covalently conjugated to the N-terminus of the A-chain. In some embodiments, the C-terminus of the B-chain of insulin is covalently conjugated to the N-terminus of the A-chain and the connecting peptide is a C-peptide. In some embodiments, the C-terminus of the B-chain of insulin is covalently conjugated to the N-terminus of the A-chain and the connecting peptide is a C-peptide and further includes any intermediate compounds that comprise a conjugate of Formula I. In some embodiments, insulin includes insulin lispro, or a glargine-type of modification, or any suitable modification to human insulin analogue that impacts the pharmacokinetics or half-life of insulin in the blood.

In some embodiments, the polypeptide hormone is glucagon. In some embodiments, glucagon has additional mutations and modifications that are known to impact solubility and solution stability of glucagon. In some embodiments, a compound, such as a molecular conjugate, comprises a conjugation of a Z1a to the N-terminus of the B-chain of insulin through a peptide bond, and at least one additional conjugation described by Formula I to insulin. In some embodiments, the additional conjugation is to a lysine residue in insulin. In some embodiments, at least one such lysine is a residue between position 15 and the C-terminus of the B-chain of insulin. In some embodiments, the lysine residue is optionally next to a proline, glycine, arginine, threonine or serine. In some embodiments, one or more amino acids in Formula I is a D-amino acid. In some embodiments, any secondary or primary amine in a compound, such as a molecular conjugate described by Formula I, is each independently optionally acetylated. In some embodiments, a compound, such as a molecular conjugate has a polypeptide hormone X1 further conjugated to a drug molecule, an imaging agent, a chelator, a contrast agent, a radioactive isotope or a molecule that engages immune cells. In some embodiments, X1 is a polypeptide hormone comprising a peptide ligand that binds to an extracellular protein receptor. In some embodiments, X1 comprises a polypeptide analogue of a human polypeptide hormone that has at least 50% homology to a natural human polypeptide hormone. In some embodiments, X1 is an analogue of human insulin with up to 10 additional residues added to the A-chain or the B-chain of insulin.

In some embodiments, the term “glucose responsiveness” refers to the change in activity in the presence and absence of glucose or in a difference of lower levels and higher levels of glucose (e.g, 3 mM glucose vs 20 mM glucose). In some embodiments the activity of a conjugated insulin is assessed by the concentration of insulin (in nanomolar units (nM) of insulin) required to induce the half maximum response (EC50) in a cell-based assay. Lower EC50 concentrations of conjugated insulins have a higher activity than insulins with higher EC50 concentrations (e.g., an insulin with an EC50 of 3 nM is more active than an insulin with an EC50 of 50 nM). A “glucose response” is observed when the insulin changes from a less active EC50 (higher nM) to a more active EC50 (lower nM) in the absence and presence of glucose or in lower and higher levels of glucose, respectively.

In some embodiments, the compound, such as a molecular conjugate, comprises one or more L- or D-artificial amino acids which are not one of the twenty naturally occurring amino acids. In some embodiments, the side chains of such artificial amino acids can be covalently conjugated through a number of reactions, including bio-orthogonal reactions such as, for example, described by: Rostovtsev, V. V., Green, L. G., Fokin, V. V. & Sharpless, K. B. A stepwise huisgen cycloaddition process: copper(I)-catalyzed regioselective “ligation” of azides and terminal alkynes. Angew. Chem. Int. Ed. 41, 2596-2599 (2002), or by: Liang, Y., Mackey, J. L., Lopez, S. A., Liu, F. & Houk, K. N. Control and design of mutual orthogonality in bioorthogonal cycloadditions. J. Am. Chem. Soc. 134, 17904-17907 (2012). In some embodiments, Z1a contains one or more L- or D-artificial amino acids that are not one of the twenty naturally occurring amino acids. In some embodiments, the side chains of two amino acids in Z1a are covalently conjugated together through a triazole bond.

Insulin hormone is an important regulator of blood glucose (sugar) levels. In a normal individual, insulin is present and, when released by the pancreas, it acts to reduce blood sugar levels, for example, by binding to and activating the insulin receptor, triggering glucose absorption by liver, fat, and skeletal muscle cells. Diabetes mellitus (DM), commonly referred to as diabetes, is a group of metabolic diseases characterized by the persistence of high blood sugar levels over a prolonged period.

As used herein, “insulin” encompasses both wild-type and altered forms of insulin capable of binding to and activating the insulin receptor, or capable of causing a measurable reduction in blood glucose when administered in vivo and encompasses both wild-type and altered forms of human insulin capable of binding to and activating the human insulin receptor, or capable of causing a measurable reduction in blood glucose when administered in vivo to a human.

In some embodiments, insulin includes insulin from any species whether in purified, synthetic, or recombinant form and includes human insulin, porcine insulin, bovine insulin, sheep insulin and rabbit insulin. In some embodiments, insulin has two chains: a B- and an A-chain. In some embodiments, the chains are connected together through peptides such as, for example, c-peptide as is known in the art, or a shortened version of the c-peptide, and in other embodiments the insulin may be provided as a proinsulin (insulin precursor) that can be further processed into mature insulin. A variety of altered forms of insulin are known in the art and may be chemically altered such as by addition of a chemical moiety such as a PEG group or a fatty acyl chain. Altered insulins may be mutated including additions, deletions or substitutions of amino acids. In some embodiments the term “desB30” refers to an insulin lacking the B30 amino acid residue.

In some embodiments, insulin analogues include insulin that is chemically altered as compared to wild type human insulin, such as, but not limited to, by addition of a chemical moiety such as a PEG group or a fatty acyl chain. In some embodiments, altered insulins or insulin analogues may be mutated including additions, deletions or substitutions of amino acids. Different protomers of insulin may result from these changes and be incorporated into some embodiments. In some embodiments, active forms of insulins have fewer than 11 such modifications (e.g., 1-4, 1-3, 1-9, 1-8, 1-7, 1-6, 2-6, 2-5, 2-4, 1-5, 1-2, 2-9, 2-8, 2-7, 2-3, 3-9, 3-8, 3-7, 3-6, 3-5, 3-4, 4-9, 4-8, 4-7, 4-6, 4-5, 5-9, 5-8, 5-7, 5-6, 6-9, 6-8, 6-7, 7-9, 7-8, 8-9, 9, 8, 7, 6, 5, 4, 3, 2 or 1). As used herein, the wild-type sequence of human insulin (A-chain and B-chain), has an A-chain with the amino acid sequence GIVEQCCTSICSLYQLENYCN (SEQ ID NO:1), and a B-chain having the amino acid sequence FVNQHLCGSHLVEALYLVCGERGFFYTPKT (SEQ ID NO:2).

Human insulin differs from rabbit, porcine, bovine, and sheep insulin in amino acids A8, A9, A10, and B30, which are in order the following: Thr, Ser, Ile, Thr for human; Thr, Ser, Ile, Ser for rabbit; Thr, Ser, Ile, Ala for porcine; Ala, Gly, Val, Ala for sheep; and Ala, Ser, Val, Ala for bovine. In some embodiments, a modified insulin may be mutated at position B1, B2, B28 or B29, or at positions B28 and B29 of the B-chain. In some embodiments, a modified insulin may be mutated at A1, A2, A21 or other positions of the A-chain. For example, insulin lispro is a fast-acting modified insulin in which the lysine and proline residues on the C-terminal end of the B-chain have been reversed. Insulin aspart is a fast-acting modified insulin in which proline has been substituted with aspartic acid at position B28. It is contemplated in some embodiments of the present disclosure that insulins mutated at B28 and B29 may further include additional mutations. For example, insulin glulisine is a fast-acting modified insulin in which aspartic acid has been replaced by a lysine residue at position B3, and lysine has been replaced by a glutamic acid residue at position B29. In some embodiments, longer acting and higher stability insulin analogs are covalently modified as described by Formula I, and may contain mutations such as tyrosine at A14 replaced with glutamic acid, the tyrosine at B16 replaced with histidine, and the phenylalanine at B25 replaced with a histidine.

In some embodiments, the isoelectric point of insulins herein may be shifted relative to wild-type human insulin using any suitable method, for example by addition or substitution of suitable amino acids. In some embodiments, the isoelectric point of the modified insulins may be modulated by glucose (e.g., by interaction with glucose). For example, insulin glargine is a basal insulin in which two arginine residues have been added to the C-terminus of the B-peptide, and A21 has been replaced by glycine. In some embodiments, the insulin may not have one or more of the residues B1, B2, B3, B26, B27, B28, B29, and B30 (e.g., the insulin may be a deletion mutant at one or more of the listed residues). In some embodiments, the insulin molecule contains up to five additional amino acid residues on the N- or C-terminus of the A-chain or B-chain. In some embodiments, one or more amino acid residues are located at positions A1, A21, B1, B29, B30 and/or B31 or are missing. In some embodiments, an insulin molecule of the present disclosure is mutated such that one or more amino acids are replaced (substituted) with their acidic forms. In some embodiments, an asparagine is replaced with aspartic acid or glutamic acid. In some embodiments, glutamine is replaced with aspartic acid or glutamic acid. In some embodiments, A21 may be an aspartic acid, B3 may be an aspartic acid, or both positions may contain an aspartic acid. One skilled in the art will recognize that it is possible to make any previously reported, or widely accepted mutations or modifications to insulin that retains biological activity, and that such an insulin analogue can be used in embodiments of the present disclosure. In some embodiments, an insulin may be linked at any position to a fatty acid, or acylated with a fatty acid at any amino group, including those on lysine side chains and the alpha-amino group on the N-terminus of insulin, and the fatty acid may include a C8, C9, C10, C11, C12, C14, C15, C16, C17, or C18 chain. In some embodiments, the fatty acid chain is 8-20 carbons long. In some embodiments, is an insulin detemir, in which a myristic acid is covalently conjugated to lysine at B29, and B30 is deleted or absent. In some embodiments, position B28 of the insulin molecule is lysine and the epsilon(s)-amino group of this lysine is conjugated to a fatty acid.

In some embodiments, the N- or C-terminal end of the A- or B-chain of the modified insulin is ligated using a peptide ligase. In some embodiments, a polypeptide is added to the C-terminus of the insulin A- and/or B-chain or to the N-terminus of insulin A- and/or B-chain using a protein ligase, and in some embodiments thereof the ligase is chosen from sortases, butelases, Trypsiligases, Subtilisins, Peptiligases or enzymes having at least 75% homology to these ligases. In some embodiments, ligation is achieved through expressed protein ligation as described in: Muir T W, Sondhi D, Cole P A. “Expressed protein ligation: a general method for protein engineering.” Proc Nat Acad Sci USA. 1998; 95(12):6705-6710. In some embodiments, the polypeptide is linked to the modified insulin using Staudinger ligation, utilizing the Staudinger reaction and as described for example in Nilsson, B. L.; Kiessling, L. L.; Raines, R. T. (2000). “Staudinger ligation: A peptide from a thioester and azide”. Org. Lett. 2 (13): 1939-1941. In some embodiments, a polypeptide is conjugated to the modified insulin using Ser/Thr ligation as, for example, described in: Zhang Y, Xu C, Kam H Y, Lee C L, Li X. 2013, “Protein chemical synthesis by serine/threonine ligation.” Proc. Natl. Acad. Sci. USA. 17:6657-6662. In some embodiments, the B-chain itself has less than 32 amino acids or 34 amino acids, and in some embodiments the insulin has 4 disulfide bonds instead of 3. There are disulfide bonds present in the A and B chains of insulin. For example, a disulfide bond exists between the cysteine at position 6 of SEQ ID NO:1 and the cysteine at position 11 of SEQ ID NO:1, a disulfide bond exists between the cysteine at position 7 of SEQ ID NO:1 and the cysteine at position 7 of SEQ ID NO:2, and a disulfide bond exists between the cysteine at position 20 of SEQ ID NO:1 and the cysteine at position 19 of SEQ ID NO:2.

In some embodiments, a modified insulin of the present disclosure comprises one or more mutations and/or chemical modifications including, but not limited to one of the following insulin molecules: N^{εB29}-octanoyl-Arg^{B0}Gly^{A21}Asp^{B3}Arg^{B31}Arg^{B32}-HI, N^{εB29}-octanoyl-ArgB³¹ArgB³²—HI, N_{εB29}-octanoyl-Arg^{A0}Arg^{B31}Arg^{B32}-HI, N^{εB28}-myristoyl-Gly^{A21}Lys^{B28}Pro^{B29}Arg^{B31}Arg^{B32}-HI, N^{εB28}-myristoyl-Gly^{A21}Gln^{B3}Lys^{B28}PrO^{B30}Arg^{B31}Arg^{B32}-HI, N^{εB28}-myristoyl-Arg^{A0}Gly^{A21}Lys^{B28}Pro^{B29}Arg^{B31}Arg^{B32}-HI, N^{εB28}-myristoyl-Arg^{A0}Gly^{A21}Gln^{B3}Lys^{B28}Pro^{B29}Arg^{B31}Arg^{B32}-HI, N^{εB28}-myristoyl-Arg^{A0}Gly^{A21}Asp^{B3}Lys^{B28}Pro^{B29}Arg^{B31}Arg^{B32}-HI, N^{εB28}-myristoyl-Lys^{B28}Pro^{B29}Arg^{B31}Arg^{B32}-HI, N^{εB28}-myristoyl-Arg^{A0}Lys^{B28}Pro^{B29}Arg^{B31}Arg^{B32}-HI, N^{εB28}-octanoyl-Gly^{A21}Lys^{B28}Pro^{B29}Arg^{B31}Arg^{B32}-HI, N^{εB28}-octanoyl-Gly^{A21}Gln^{B3}Lys^{B28}Pro^{B29}Arg^{B31}Arg^{B32}-HI, N^{εB28}-octanoyl-Arg^{A0}Gly^{A21}Lys^{B28}Pro^{B29}Arg^{B31}Arg^{B32}-HI, N^{εB29}-palmitoyl-HI, N^{εB29}-myristoyl-HI, N^{εB28}-palmitoyl-Lys^{B28}Pro^{B29}-HI, N^{εB28}-myristoyl-Lys^{B28}Pro^{B29}-HI, N^{εB29}-palmitoyl-des(B30)-HI, N^{εB30}-myristoyl-Thr^{B29}Lys^{B30}-HI, N^{εB30}-palmitoyl-Thr^{B29}Lys^{B30}-HI, N^{εB29}-(N-palmitoyl-γ-glutamyl)-des(B30)-HI, N^{εB29}-(N-lithocolyl-γ-glutamyl)-des(B30)-HI, N^{εB29}-(ω-carboxyheptadecanoyl)-des(B30)-HI, N^{εB29}-(ω-carboxyheptadecanoyl)-HI, N^{εB29}-octanoyl-HI, N^{εB29}-myristoyl-Gly^{A21}Arg^{B31}Arg^{B31}-HI, N^{εB29}-myristoyl-Gly^{A21}Gln^{B3}Arg^{B31}Arg^{B32}-HI, N_{εB29}-myristoyl-Arg^{A0}Gly^{A21}Arg^{B31}Arg^{B32}-HI, N^{εB29}-Arg^{A0}Gly^{A21}Gln^{B3}Arg^{B31}Arg^{B32}-HI, N^{εB29}-myristoyl-Arg^{A0}Gly^{A21}Asp^{B3}Arg^{B31}Arg^{B32}-HI, N^{εB29}-myristoyl-Arg^{B31}Arg^{B32}-HI, N^{εB29}-myristoyl-Arg^{A0}Arg^{B31}Arg^{B32}-HI, N^{εB29}-octanoyl-Gly^{A21}Arg^{B31}Arg^{B32}-HI, N^{εB29}-octanoyl-Gly^{A21}Gln^{B3}Arg^{B31}Arg^{B32}-HI, N^{εB29}-octanoyl-Arg^{A0}Gly^{A21}Arg^{B31}Arg^{B32}-HI, N^{εB29}-octanoyl-Arg^{A0}Gly^{A21}Gln^{B3}Arg^{B31}Arg^{B32}-HI, N^{εB28}-octanoyl-Arg^{A0}Gly^{A21}Gln^{B3}Lys^{B28}Pro^{B29}Arg^{B31}Arg^{B32}-HI, N^{εB28}-octanoyl-Arg^{A0}Gly^{A21}Asp^{B3}Lys^{B28}Pro^{B29}Arg^{B31}Arg^{B32}-HI, N^{εB28}-octanoyl-Lys^{B28}Pro^{B29}Arg^{B31}Arg^{B32}-HI, N^{εB28}-octanoyl-Arg^{A0}Lys^{B28}Pro^{B29}Arg^{B31}Arg^{B32}-HI. N^{εB29}-pentanoyl-Gly^{A21}Arg^{B31}Arg^{B32}-HI, N^{αB1}-hexanoyl-Gly^{A21}Arg^{B31}Arg^{B32}-HI, N^{αA1}-heptanoyl-Gly^{A21}Arg^{B31}Arg^{B32}-HI, N^{εB29}-octanoyl-N^{αB1}-octanoyl-Gly^{A21}Arg^{B31}Arg^{B32}-HI, N^{εB29}-propionyl-N^{αA1}-propionyl-Gly^{A21}Arg^{B31}Arg^{B32}-HI, N^{αA1}-acetyl-N^{αB1}-acetyl-Gly^{A21}Arg^{B31}Arg^{B32}-HI, N^{εB29}-formyl-N^{αA1}-formyl-N^{αB1}-formyl-Gly^{A21}Arg^{B31}Arg^{B32}-HI, N^{εB29}-formyl-des(B26)-HI, N^{αB1}-acetyl-Asp^{B28}-HI, N^{εB29}-propionyl-N^{αA1}-propionyl-N^{αB1}-propionyl-Asp^{B1}Asp^{B3}Asp^{B21}-HI, N^{εB29}-pentanoyl-Gly^{A21}-HI, N^{αB1}-hexanoyl-Gly^{A21}-HI, N^{αA1}-heptanoyl-Gly^{A21}-HI, N^{εB29}-octanoyl-N^{αB1}-octanoyl-Gly^{A21}-HI, N^{εB29}-propionyl-N^{αA1}-propionyl-Gly^{A21}-HI, N^{αA1}-acetyl-N^{αB1}-acetyl-Gly^{A21}-HI, N^{εB29}-formyl-N^{αA1}-formyl-N^{αB1}-formyl-Gly^{A21}-HI, N^{εB29}-butyryl-des(B30)-HI, N^{αB31}-butyryl-des(B30)-HI, N^{αA1}-butyryl-des(B30)-HI, N^{εB29}-butyryl-N^{αB31}-butyryl-des(B30)-HI, N^{εB29}-butyryl-N^{αA1}-butyryl-des(B30)-HI, N^{αA1}-butyryl-N^{αB31}-butyryl-des(B30)-HI, N^{εB29}-butyryl-N^{αA1}-butyryl-N^{αB31}-butyryl-des(B30)-HI, Lys^{B28}Pro^{B29}-HI (insulin lispro), Asp^{B28}-HI (insulin aspart), Lys^{B3}Glu^{B29}-HI (insulin glulisine), Arg^{B31}Arg^{B32}-HI (insulin glargine), N^{εB29}-myristoyl-des(B30)-HI (insulin detemir), Ala^{B26}-HI, Asp^{B1}-HI, Arg^{A0}-HI, Asp^{B1}Glu^{B13}-HI, Gly^{A21}-HI, Gly^{A21}Arg^{B31}Arg^{B32}-HI, Arg^{A0}Arg^{B31}Arg^{B32}-HI, Arg^{A0}Gly^{A21}Arg^{B31}Arg^{B32}-HI, des(B30)-HI, des(B27)-HI, des(B28-B30)-HI, des(B1)-HI, des(B1-B3)-HIN^{εB29}-tridecanoyl-des(B30)-HI, N^{εB29}-tetradecanoyl-des(B30)-HI, N^{εB29}-decanoyl-des(B30)-HI, N^{εB29}-dodecanoyl-des(B30)-HI, N^{εB29}-tridecanoyl-Gly^{A21}-des(B30)-HI, N^{εB29}-tetradecanoyl-Gly^{A21}-des(B30)-HI, N^{εB29}-decanoyl-Gly^{A21}-des(B30)-HI, N^{εB29}-dodecanoyl-Gly^{A21}-des(B30)-HI, N^{εB29}-tridecanoyl-Gly^{A21}Gln^{B3}-des(B30)-HI, N^{εB29}-tetradecanoyl-Gly^{A21}Gln^{B3}-des(B30)-HI, N^{εB29}-decanoyl-Gly^{A21}-Gln^{B3}-des(B30)-HI, N^{εB29}-dodecanoyl-Gly^{A21}-Gln^{B3}-des(B30)-HI, N^{εB29}-tridecanoyl-Ala^{A21}-des(B30)-HI, N^{εB29}-tetradecanoyl-Ala^{A21}-des(B30)-HI, N^{εB29}-decanoyl-Ala^{A21}-des(B30)-HI, N^{εB29}-dodecanoyl-Ala^{A21}-des(B30)-HI, N^{εB29}-tridecanoyl-Ala^{A21}-Gln^{B3}-des(B30)-HI, N^{εB29}-tetradecanoyl-Ala^{A21}Gln^{B3}-des(B30)-HI, N^{εB29}-decanoyl-Ala^{A21}Gln^{B3}-des(B30)-HI, N^{εB29}-dodecanoyl-Ala^{A21}Gln^{B3}-des(B30)-HI, N^{εB29}-tridecanoyl-Gln^{B3}-des(B30)-HI, N^{εB29}-tetradecanoyl-Gln^{B3}-des(B30)-HI, N^{εB29}-decanoyl-Gln^{B3}-des(B30)-HI, N^{εB29}-dodecanoyl-Gln^{B3}-des(B30)-HI, N^{εB29}-Z1-Gly^{A21}-HI, N^{εB29}-Z2-Gly^{A21}-HI, N^{εB29}-Z4-Gly^{A21}-HI, N^{εB29}-Z3-Gly^{A21}-HI, N^{εB29}-Z1-Ala^{A21}-HI, N^{εB29}-Z2-Ala^{A21}-HI, N^{εB29}-Z4-Ala^{A21}-HI, N^{εB29}-Z3-Ala^{A21}-HI, N^{εB29}-Z1-Gly^{A21}Gln^{B3}-HI, N^{εB29}-Z2-Gly^{A21}Gln^{B3}-HI, N^{εB29}-Z4-Gly^{A21}Gln^{B3}-HI, N^{εB29}-Z3-Gly^{A21}Gln^{B3}-HI, N^{εB29}-Z1-Ala^{A21}Gln^{B3}-HI, N^{εB29}-Z2-Ala^{A21}Gln^{B3}-HI, N^{εB29}-Z4-Ala^{A21}Gln^{B3}-HI, N^{εB29}-Z3-Ala^{A21}Gln^{B3}-HI, N^{εB29}-Z1-Gln^{B3}-HI, N^{εB29}-Z2-Gln^{B3}-HI, N^{εB29}-Z4-Gln^{B3}-HI, N^{εB29}-Z3-Gln^{B3}-HI, N^{εB29}-Z1-Glu^{B30}-HI, N^{εB29}-Z2-Glu^{B30}-HI, N^{εB29}-Z4-Glu^{B30}-HI, N^{εB29}-Z3-Glu^{B30}-HI, N^{εB29}-Z1-Gly^{A21}Glu^{B30}-HI, N^{εB29}-Z2-Gly^{A21}Glu^{B30}-HI, N^{εB29}-Z4-Gly^{A21}Glu^{B30}-HI, N^{εB29}-Z3-Gly^{A21}Glu^{B30}-HI, N^{εB29}-Z1-Gly^{A21}Gln^{B3}Glu^{B30}-HI, N^{εB29}-Z2-Gly^{A21}Gln^{B3}Glu^{B30}-HI, N^{εB29}-Z4-Gly^{A21}Gln^{B3}Glu^{B30}-HI, N^{εB29}-Z3-Gly^{A21}Gln^{B3}Glu^{B30}-HI, N^{εB29}-Z1-Ala^{A21}Glu^{B30}-HI, N^{εB29}-Z2-Ala^{A21}Glu^{B30}-HI, N^{εB29}-Z4-Ala^{A21}Gln^{B30}-HI, N^{εB29}-Z3-Ala^{A21}Glu^{B30}-HI, N^{εB29}-Z1-Ala^{A21}Gln^{B3}Glu^{B30}-HI, N^{εB29}-Z2-Ala^{A21}Gln^{B3}Glu^{B30}-HI, N^{εB29}-Z4-Ala^{A21}Gln^{B3}Glu^{B30}-HI, N^{εB29}-Z3-Ala^{A21}Gln^{B3}Glu^{B30}-HI, N^{εB29}-Z1-Gln^{B3}Glu^{B30}-HI, N^{εB29}-Z2-Gln^{B3}Glu^{B30}-HI, N^{εB29}-Z4-Gln^{B3}Glu^{B30}-HI, N^{εB29}-Z3-Gln^{B3}Glu^{B30}-HI and where Z1 is tridecanoyl, Z2 is tetradecanoyl, Z3 is dodecanoyl, Z4 is decanoyl, and HI is human insulin.

In some embodiments, insulin includes one or more of the following mutations and/or chemical modifications: N^{εB28}-XXXXX-Lys^{B28}Pro^{B29}-HI, N^{αB1}-XXXXX-Lys^{B28}Pro^{B29}-HI, N^{αA1}-XXXXX-Lys^{B28}Pro^{B29}-HI, N^{εB28}-XXXXX-N^{αB1}-XXXXX-Lys^{B28}Pro^{B29}-HI, N^{εB28}-XXXXX-N^{αA1}-XXXXX-Lys^{B28}Pro^{B29}-HI, N^{αA1}-XXXXX-N^{αB1}-XXXXX-Lys^{B28}Pro^{B29}-HI, N^{εB28}-XXXXX-N^{αA1}-XXXXX-N^{αB1}-XXXXX-Lys^{B28}Pro^{B29}-HI, N^{εB29}-XXXXX-HI, N^{αB1}-XXXXX-HI, N^{αA1}-XXXXX-HI, N^{εB29}-XXXXX-N^{αB1}-XXXXX-HI, N^{εB29}-XXXXX-N^{αA1}-XXXXX-HI, N^{αA1}-XXXXX-N^{αB1}-XXXXX-HI, N^{εB29}-XXXXX-N^{αA1}-XXXXX-N^{αB1}-XXXXX-HI, N^{εB29}-YYYYY-HI, N^{αB1}-YYYYY-HI, N^{αA1}-YYYYY-HI, N^{εB29}-YYYYY-N^{αB1}-YYYYY-HI, N^{εB29}-YYYYY-N^{αA1}-YYYYY-HI, N^{αA1}-YYYYY-N^{αB1}-YYYYY-HI, N^{εB29}-YYYYY-N^{αA1}-YYYYY-N^{αB1}-YYYYY-HI, N^{εB28}-YYYYY-Lys^{B28}Pro^{B29}-HI, N^{εB21}-YYYYY-Lys^{B28}Pro^{B29}-HI, N^{αA1}-YYYYY-Lys^{B28}Pro^{B29}-HI, N^{εB28}-YYYYY-N^{αB1}-YYYYY-Lys^{B28}Pro^{B29}-HI, N^{εB28}-YYYYY-N_{αA1}-YYYYY-Lys^{B28}Pro^{B29}-HI, N^{αB1}-YYYYY-N^{αB1}-YYYYY-Lys^{B28}Pro^{B29}-HI, N^{εB28}-YYYYY-N^{αA1}-YYYYY-N^{αB1}-YYYYY-Lys^{B28}Pro^{B29}-HI, and where YYYYY is one of acetyl or formyl and where XXXXX is one of: propionyl, butyryl, pentanoyl, hexanoyl, heptanoyl, octanoyl, nonanoyl or decanoyl and HI is human insulin.

In some embodiments, insulin may be conjugated through a reactive moiety that is naturally present within the insulin structure or is added prior to conjugation, including, for example, carboxyl or reactive ester, amine, hydroxyl, aldehyde, sulfhydryl, maleimidyl, alkynyl, azido, etc. moieties. Insulin naturally includes reactive alpha-terminal amine and epsilon-amine lysine groups to which NHS-ester, isocyanates or isothiocyanates can be covalently conjugated. In some embodiments, a modified insulin may be employed in which a suitable amino acid (e.g., a lysine or a non-natural amino acid) has been added or substituted into the amino acid sequence in order to provide an alternative point of conjugation in addition to the modified amino acids of the embodiments described herein. In some embodiments, the conjugation process may be controlled by selectively blocking certain reactive moieties prior to conjugation. In some embodiments, insulin may include any combination of modifications and the present disclosure also encompasses modified forms of non-human insulins (e.g., porcine insulin, bovine insulin, rabbit insulin, sheep insulin, etc.) that comprise any one of the aforementioned modifications. It is understood that some embodiments include these and other previously described modified insulins such as those described in U.S. Pat. Nos. 5,474,978; 5,461,031; 4,421,685; 7,387,996; 6,869,930; 6,174,856; 6,011,007; 5,866,538; 5,750,497; 6,906,028; 6,551,992; 6,465,426; 6,444,641; 6,335,316; 6,268,335; 6,051,551; 6,034,054; 5,952,297; 5,922,675; 5,747,642; 5,693,609; 5,650,486; 5,547,929; 5,504,188; US 2015/0353619, including non-natural amino acids described or referenced herein and including such modifications to the non-human insulins described herein. It is also to be understood that in some embodiments the insulin may be covalently conjugated to polyethylene glycol polymers, such as polyethylene glycol polymers of no more than Mn 60,000, or covalently conjugated either through permanent or reversible bonds to albumin.

In some embodiments, a compound, such as a molecular conjugate, is conjugated to a chelator, and in some embodiments the chelator can be used to capture a radioactive payload, such as gallium 68, copper 64, lutetium 177, or actinium 225. In some embodiments, the chelator is based on DOTA, NOTA, TETA, or 4-arm DOTA, and in some embodiments, the chelator can be linked to the peptide using a PEG linker through amide bonds to the chelator and to the peptide.

In some embodiments, the activity, bioavailability, solubility, isoelectric point, charge and/or hydrophobicity of the modified insulins can be controlled through chemical modifications and/or as result of interaction of a small molecule such as a sugar with the compounds, such as a molecular conjugates, described herein which are either covalently linked or mixed with insulin.

In some embodiments one or more elements, functional groups, or atoms may be specifically omitted or excluded from a depicted structure (e.g., a terminal functional group may be replaced by a hydrogen atom, or a linking group may be replaced by a bond), for example in Formulae FF1-FF224, and it will be understood that such omitted or excluded elements make these groups (structures) distinct and non-equivalent. For example, if an alternative version (variation) of a formula structure does not have a nitro group in R1 for B1 or B2, that variation is not equivalent (e.g., is structurally and chemically inequivalent) to a structure that includes the nitro group, at least because the nitro group changes the pKa of B1 and B2 in physiological conditions and hence the overall affinity of Z1c for glucose.

### Rotational Constrained Tethered Boron Conjugates.

In some embodiments, aromatic boron-containing compounds and/or aromatic boron-containing groups are rotationally constrained tether boron conjugates. In some embodiments, rotationally constrained tether boron conjugates presented in this disclosure contain scaffolds that are rotationally hindered by disfavored steric interactions (e.g. gauche vs anti interactions of substituents), hindered rotation due to bond hybridization (e.g., cis- vs trans-amide rotations), or through rigid covalent bonds (e.g., (E) vs (Z) configurations for alkene moieties). For example, formulae FF50-FF62, FF116, and FF121-134 contain alkyl functionalities geminal (e.g., attached to the same atom) to the amine groups that are covalently conjugated to the boronic acid functionalized moieties. Alkyl functionalities may limit the accessible dihedral angles and the rotation freedom around the C—C or C—X bond (commonly referred to as χ (chi) dihedral angles in amino acids). For example the hydroxyl sidechain on a serine residue can access dihedral angles of 60°, 180°, or 240° (−60°) with near equal distribution while the hydroxyl sidechain of threonine may only adopt dihedral angles of 180° or 240° (−60°). The presence of a methyl group geminal to the hydroxy on threonine may provide steric bulk, creating unfavorable interactions when other bulky substituents are in a gauche conformation relative to the methyl. Formulae FF50-FF62, FF116, and FF121-134 contain geminal alkyl substituents which may limit the accessible dihedral angles that the boron conjugated amines adopt, influencing adopted dihedral angles and placing the boronic functionalized groups closer together and allowing for increased binding of the conjugates to target molecules such as proteins or sugars.

In some embodiments, the stereochemistry of isomeric structures (e.g., the stereochemistry of a compound, such as a molecular conjugate, for example within the Z1c moiety) may selectively increase the affinity of the conjugate (e.g., the Z1c moiety) for a specific target diol, such as glucose. For example, in some embodiments one or more stereoisomers (e.g., cis- or trans-, (R) or (S), and (E) or (Z)) of Z1c may be selected to increase or decreases the affinity of Z1c (and the molecular architecture or conjugate as a whole) for glucose. In some embodiments, the cis form of Formulae FF1-FF224 is used when applicable (e.g., Z1c includes a structure having cis stereochemistry). In some embodiments, the trans form of Formulae FF1-FF224 is used when applicable, e.g., when Formulae FF1-FF224 includes two stereocenters linked by a bond, (e.g., Z1c includes a structure having trans stereochemistry). In some embodiments, the R form of Formulae FF1-FF224 is used when applicable, e.g., when Formulae FF1-FF224 includes at least one stereocenter, (e.g., Z1c includes a structure having R stereochemistry). In some embodiments, the S form of Formulae FF1-FF224 is used when applicable, e.g., when Formulae FF1-FF224 includes at least one stereocenter, (e.g., Z1c includes a structure having S stereochemistry). In some embodiments, the S,S form of Formulae FF1-FF224 is used when applicable, e.g., when Formulae FF1-FF224 includes two stereocenters linked by a bond, (e.g., Z1c includes a structure having S,S stereochemistry). In some embodiments, the S,R form of Formulae FF1-FF224 is used when applicable, e.g., when Formulae FF1-FF224 includes two stereocenters linked by a bond, (e.g., Z1c includes a structure having S,R stereochemistry). In some embodiments, the R,R form of Formulae FF1-FF224 is used when applicable, e.g., when Formulae FF1-FF224 includes two stereocenters linked by a bond, (e.g., Z1c includes a structure having R,R stereochemistry). In some embodiments, the R,S form of Formulae FF1-FF224 is used when applicable, e.g., when Formulae FF1-FF224 includes two stereocenters linked by a bond, (e.g., Z1c includes a structure having R,S stereochemistry). In some embodiments, a compound, such as a molecular conjugate, includes one or more tautomers of a compound, such as a molecular conjugate, disclosed herein. In some embodiments, a compound, such as a molecular conjugate, includes one or more stereoisomer or a mixture of stereoisomers of a compound, such as a molecular conjugate, disclosed herein.

In some embodiments, a compound, such as a molecular conjugate, is covalently conjugated to glucagon, GLP-1, GLP-2 or a variation of any of these (e.g., any variation with deletions, insertions and/or replacements of one or more amino acids). In some embodiments any suitable chemical modifications made to insulin discussed herein can be made to glucagon. In some embodiments the conjugate is mixed with a second or drug substance or one or more compounds chosen from: aminoethylglucose, aminoethylbimannose, aminoethyltrimannose, D-glucose, D-galactose, D-Allose, D-Mannose, D-Gulose, D-Idose, D-Talose, N-Azidomannosamine (ManNAz) or N-Azidogalactoseamine (GalNAz) or N-azidoglucoseamine (GlcNAz), 2′-fluororibose, 2′-deoxyribose, glucose, sucrose, maltose, mannose, derivatives of these (e.g., glucosamine, mannosamine, methylglucose, methylmannose, ethylglucose, ethylmannose, etc.), sorbitol, inositol, galactitol, dulcitol, xylitol, arabitol and/or higher order combinations of these (such as linear and/or branched bimannose, linear and/or branched trimannose), molecules containing cis-diols, catechols, tris, and DOPA molecules such as L-DOPA or L-3,4-dihydroxyphenylalanine.

Moreover, one skilled in the art will recognize that in some embodiments, one or more of suitable proteinogenic artificial amino acids can be used (included) in Z1a. For example, in some embodiments one or more of the following artificial amino acids can be used based on the methods described in and referenced through, and the list of amino acids provided in: Liu, C. C.; Schultz, P. G. (2010). “Adding new chemistries to the genetic code.” Annual Review of Biochemistry 79: 413-44. One skilled in the art will recognize that, in some embodiments, artificial amino acids can be incorporated by peptide synthesis in Z1a which is then covalently conjugated to the drug or insulin, and these include the amino acids referenced herein as well as previously reported non-proteinogenic amino acids. In some embodiments, artificial amino acids exist (e.g., may be included) in the insulin, and in some embodiments thereof, proteinogenic artificial amino acids can be incorporated through recombinant protein expression using suitable methods and approaches, including those described in United States patent and patent applications including: US 2008/0044854, U.S. Pat. Nos. 8,518,666, 8,980,581, US 2008/0044854, US 20140045261, US 2004/0053390, U.S. Pat. Nos. 7,229,634, 8,236,344, US 2005/0196427, US 2010/0247433, U.S. Pat. Nos. 7,198,915, 7,723,070, US 2002/0042097, US 2004/0058415, US 2008/0026422, US 2008/0160609, US 2010/0184193, US 2012/0077228, US 2014/025599, U.S. Pat. Nos. 7,198,915, 7,632,492, 7,723,070, and other proteinogenic artificial amino acids may be introduced recombinantly using methods and approaches described in: U.S. Pat. Nos. 7,736,872, 7,816,320, 7,829,310, 7,829,659, 7,883,866, 8,097,702, 8,946,148.

In some embodiments cyclic amino acids such as 3-hydroxyproline, 4-hydroxyproline, aziridine-2-earboxylic acid, azetidine-2-carboxylic acid, piperidine-2-carboxylic acid, 3-carboxy-morpholine, 3-carboxy-thiamorpholine, 4-oxaproline, pyroglutamic acid, 1,3-oxazolidine-4-carboxylic acid, 1,3-thiazolidine-4-carboxylic acid, 3-thiaproline, 4-thiaproline, 3-selenoproline, 4-selenoproline, 4-ketoproline, 3,4-dehydroproline, 4-aminoproline, 4-fluoroproline, 4,4-difluoroproline, 4-chloroproline, 4,4-dichloroproline, 4-bromoproline, 4,4-dibromoproline, 4-methylproline, 4-ethylproline, 4-cyclohexyl-proline, 3-phenylproline, 4-phenylproline, 3,4-phenylproline, 4-azidoproline, 4-carboxyproline, a-methylproline, a-ethylproline, a-propylproline, a-allylproline, a-benzylproline, a-(4-fluorobenzyl)-proline, a-(2-chlorobenzyl)-proline, a-(3-chlorobenzyl)-proline, a-(2-bromobenzyl)-proline, a-(4-bromobenzyl)-proline, a-(4-methylbenzyl)-proline, a-(diphenylmethyl)-proline, a-(naphthylmethyl)-proline, D-proline, or S-homoproline, (2S,4S)-4-fluoro-L-proline, (2S,4R)-4-fluoro-L-proline, (2S)-3,4-dehydro-L-proline, (2S,4S)-4-hydroxy-L-proline, (2S,4R)-4-hydroxy-L-proline, (2S,4S)-4-azido-L-proline, (2S)-4,4-difluoro-L-proline, (2S)-azetidine-2-carboxylic acid, (2S)-piperidine-2-carboxylic acid, or (4R)-1,3-thiazolidine-4-carboxylic acid can be used in the molecular architecture that is conjugated to insulin.

It is to be understood that in some embodiments, a specific orientation of amino acids is achieved using, for example, methods of Albericio, F. (2000). Solid-Phase Synthesis: A Practical Guide (1 ed.). Boca Raton: CRC Press. p. 848. In some embodiments a compound, such as a molecular conjugate, can bind to a diol, a catechol, a hexose sugar, glucose, xylose, fucose, galactosamine, glucosamine, mannosamine, galactose, mannose, fructose, galacturonic acid, glucuronic acid, iduronic acid, mannuronic acid, acetyl galactosamine, acetyl glucosamine, acetyl mannosamine, acetyl muramic acid, 2-keto-3-deoxy-glycero-galacto-nononic acid, acetyl neuraminic acid, glycolyl neuraminic acid, a neurotransmitter, dopamine, or a disaccharide or polymer of saccharides or diols.

In some embodiments modifications or intermediates may include the use of an N-methyliminodiacetic acid (MIDA) group to make a MIDA conjugated boronate or a MIDA boronate; such modifications can be used during preparation of boronates towards the final structures of use (e.g., in embodiments of methods for preparing the conjugates described herein). In some embodiments boronic acid pinacol esters are used towards the final structures wherein the pinacol group can be readily removed using standard techniques by one skilled in the art. The MIDA-protected boronate esters are easily handled, stable under air, compatible with chromatography, and unreactive under standard anhydrous cross-coupling conditions and easily deprotected at room temperature under mild aqueous basic conditions such as 1M NaOH, or even NaHCO₃, or as described by Lee, S. J. et al. (2008). J. Am. Chem. Soc. 130:466.

The biological mechanism by which wild type insulin binds to the insulin receptor is previously reported in Menting, J. G. et al. (2013). Nature 493, 241-245; and Menting, J. G. et al. (2014). “Protective hinge in insulin opens to enable its receptor engagement.” Proc. Natl. Acad. Sci. U.S.A. 111, E3395-3404. The activity of such insulin can be measured using any suitable technique, for example, by using in vitro insulin receptor binding with TyrA14-¹²⁵I human insulin as tracer and utilizing antibody binding beads with an insulin receptor monoclonal antibody. In some embodiments, animal models can be used for in vivo assessment of insulin activity during glucose challenge using methods that are known to one skilled in the art. In some embodiments, a compound, such as a molecular conjugate, is partially or fully expressed along with a recombinant protein of interest such as insulin. The processes for expression of insulin in E. coli are known and can be easily performed by one skilled in the art e.g., by using the procedures outlined in Jonasson (1996). Eur. J. Biochem. 236:656-661; Cowley (1997). FEBS Lett. 402:124-130; Cho (2001). Biotechnol. Bioprocess Eng. 6: 144-149; Tikhonov (2001). Protein Exp. Pur. 21: 176-182; Malik (2007). Protein Exp. Pur. 55: 100-111; and Min (2011). J. Biotech. 151:350-356. In the most common process, the protein is expressed as a single-chain proinsulin construct with a fission protein or affinity tag. The compound, such as a molecular conjugate, that includes Z1a when Z1a is present can be expressed as part of proinsulin, then modified chemically to conjugate, through amide linkages, to structures of interest. This approach provides good yield and reduces experimental complexity by decreasing the number of processing steps and allows refolding in a native-like insulin, see for example, Jonasson, Eur. J. Biochem. 236:656-661 (1996); Cho, Biotechnol Bioprocess Eng. 6: 144-149 (2001); Tikhonov, Protein Exp. Pur. 21:176-182 (2001); Min, J. Biotech. 151:350-356 (2011)). When expressed in E. coli, proinsulin is usually found in inclusion bodies and can be easily purified by one skilled in the art.

In some embodiments, a compound, such as a molecular conjugate, may be formulated for injection. For example, it may be formulated for injection into a subject, such as a human. In some embodiments, the composition may be a pharmaceutical composition, such as a sterile, injectable pharmaceutical composition. In some embodiments, the composition may be formulated for subcutaneous injection. In some embodiments, the composition is formulated for transdermal, intradermal, transmucosal, nasal, inhalable or intramuscular administration. In some embodiments, the composition may be formulated in an oral dosage form or a pulmonary dosage form. Pharmaceutical compositions suitable for injection may include sterile aqueous solutions containing, for example, sugars, polyalcohols such as mannitol and sorbitol, phenol, meta-cresol, and sodium chloride and dispersions may be prepared in glycerol, liquid polyethylene glycols, and mixtures thereof and in oils and the carrier can, for example, be a solvent or dispersion medium containing, for example, water, saccharides, ethanol, polyol (for example, glycerol, propylene glycol, and liquid polyethylene glycol, and the like), and suitable mixtures thereof. One skilled in the art recognizes that specific formulations can be developed to best suit the application and method of use of the molecular architectures of the invention. General considerations in the formulation and manufacture of pharmaceutical compositions, routes of administrating and including suitable pharmaceutically acceptable carriers may be found, for example, in Remington's Pharmaceutical Sciences, 19th ed., Mack Publishing Co., Easton, Pa., 1995. In some embodiments, the pharmaceutical composition may include zinc, e.g., Zn²⁺ along with insulin if the compound, such as a molecular conjugate, comprises insulin. Such zinc formulations are, for example, described in U.S. Pat. No. 9,034,818. For example, the pharmaceutical composition may comprise zinc at a molar ratio to the modified insulin of about M:N where M is 1-11 and N is 6-1. In some embodiments, such modified insulins may be stored in a pump, and that pump being either external or internal to the body releases the modified insulins. In some embodiments, a pump may be used to release a constant amount of modified insulin wherein the insulin is glucose responsive and can automatically adjust activity based on the levels of glucose in the blood and/or the release rate from the injection site. In some embodiments, the compositions may be formulated in dosage unit form for ease of administration and uniformity of dosage. In some embodiments, the pharmaceutical composition may further include a second insulin type to provide fast-acting or basal-insulin in addition to the effect afforded by the molecular architecture. In some embodiments, a compound, such as a molecular conjugate, is injected separately from insulin but modulates the activity of insulin by binding to insulin, and in some embodiments this activity change is dependent on glucose.

In some embodiments, the pharmaceutical composition comprises one or more of the compounds disclosed herein and at least one additional component selected from pharmaceutically acceptable carriers, pharmaceutically acceptable vehicles, and pharmaceutically acceptable excipients. In some embodiments, the pharmaceutical composition comprises a compound of Formula I and at least one additional component selected from pharmaceutically acceptable carriers, pharmaceutically acceptable vehicles, and pharmaceutically acceptable excipients.

In some embodiments the present disclosure includes compounds that can be part of a kit, wherein the kit includes a compound, such as a molecular conjugate, such as a compound comprising modified insulin, as well as a pharmaceutically acceptable carrier, and for injections may include a syringe or pen. In some embodiments, a kit may include a syringe or pen that is pre-filled with a pharmaceutical composition that includes the compound, such as a molecular conjugate, with a liquid carrier. Alternatively, a kit may include a separate container such as a vial with a pharmaceutical composition that includes the compound, such as a molecular conjugate, with a dry carrier and an empty syringe or pen. In some embodiments, such a kit may include a separate container that has a liquid carrier, which can be used to reconstitute a given composition that can then be taken up into the syringe or pen. In some embodiments, a kit may include instructions. In some embodiments, the kit may include blood glucose measuring devices that either locally or remotely calculate an appropriate dose of the modified insulin that is to be injected at a given point in time, or at regular intervals. Such a dosing regimen is unique to the patient and may, for example, be provided as instruction to program a pump either by a person or by a computer. The kit may include an electronic device which transfers blood glucose measurements to a second computer, either locally or elsewhere (for example, in the cloud) which then calculate the correct amount of compound, such as a molecular conjugate, comprising, e.g., a modified insulin that needs to be used by the patient at a certain time.

In some embodiments, the invention relates to a method for treating a disease or condition in a subject, comprising administering to the subject a composition including a compound, such as a molecular conjugate, described herein. In some embodiments, the disease or condition may be hyperglycemia, type 2 diabetes, impaired glucose tolerance, type 1 diabetes, obesity, metabolic syndrome X, or dyslipidemia, diabetes during pregnancy, pre-diabetes, Alzheimer's disease, MODY 1, MODY 2 or MODY 3 diabetes, mood disorders and psychiatric disorders. It will be appreciated that this combination approach may also be used with insulin resistant patients who are receiving an insulin sensitizer or a secondary drug for diabetes (such as, for example, a biguanide such as metformin, a glitazone) or/and an insulin secretagogue (such as, for example, a sulfonylurea, GLP-1, exendin-4 etc.) or amylin.

In some embodiments, a compound, such as a molecular conjugate, of the present disclosure may be administered to a patient who is receiving at least one additional therapy or taking at least one additional drug or therapeutic protein. In some embodiments, the at least one additional therapy is intended to treat the same disease or disorder as the administered compound, such as a molecular conjugate. In some embodiments, the at least one additional therapy is intended to treat a side-effect of the compound, such as a molecular conjugate, or as an adjuvant. The timeframe of the two therapies may differ or be the same; they may be administered on the same or different schedules as long as there is a period when the patient is receiving a benefit from both therapies. The two or more therapies may be administered within the same or different formulations as long as there is a period when the patient is receiving a benefit from both therapies. Any of these approaches may be used to administer more than one anti-diabetic drug to a subject.

In some embodiments a therapeutically effective amount of the compound, such as a molecular conjugate, which is sufficient amount to treat (meaning for example to ameliorate the symptoms of, delay progression of, prevent recurrence of, or delay onset of) the disease or condition at a reasonable benefit to risk ratio will be used. In some embodiments, this may involve balancing of the efficacy and additional safety to toxicity. By additional safety, it is meant that, for example, the compound, such as a molecular conjugate, can be responsive to changes in blood glucose levels or level of other molecules, even when the patient is not actively monitoring the levels of that molecule, such as blood glucose levels at a given timeframe, for example during sleep. In some embodiments, therapeutic efficacy and toxicity may be determined by standard pharmacological procedures in cell cultures or in vivo with experimental animals, and for example measuring ED₅₀ and LD₅₀ for therapeutic index of the drug. In some embodiments, the average daily dose of insulin with the molecular architecture is in the range of 5 to 400 U, (for example 30-150 U where 1 Unit of insulin is about 0.04 mg). In some embodiments, an amount of compound, such as a molecular conjugate, with these insulin doses is administered on a daily basis or bi-daily basis or by every three days or by every 4 days. In some embodiments the basis is determined by an algorithm, which can be computed by a computer. In some embodiments, an amount of compound, such as a molecular conjugate, with 5 to 10 times these doses is administered on a weekly basis or at regular intervals. In some embodiments, an amount of conjugate with 10 to 20 times these doses is administered on a bi-weekly basis or at regular intervals. In some embodiments, an amount of compound, such as a molecular conjugate, with 20 to 40 times these doses is administered on a monthly basis or at regular intervals. In some embodiments, the C-terminus of the A-chain of insulin may be further extended with a peptide (amino acid sequence) including 1-20 amino acid residues. In some embodiments the insulin analogue is a desB30 insulin.

In some embodiments, Z1a is an amino acid or a peptide. In at least some embodiments, the Z1a includes (is composed of) 1-50 amino acid residues, for example, 1 residue, 50 residues, or any intermediate number of residues (such as e.g., 10, 15, 25, 30, 42 residues, etc.). In some embodiments, the Z1a includes 1-15 amino acids. In at least some embodiments, the peptide Z1a includes 1-8 amino acids. In some embodiments, Z1a includes 5 to 6 amino acids. In some embodiments, Z1a comprises at least one amino acid independently selected from the: Alanine (A), Asparagine (N), Glutamine (Q), Threonine (T), Methionine (M), Histidine (H), Cysteine (C), Valine (V), Isoleucine (I), Lysine (K), and Leucine (L), and the rest of the amino acids each independent selected from any of the twenty naturally occurring amino acids. In some embodiments, Z1a may include diaminopropionic acid, diaminobutyric acid, or ornithine. In some embodiments, Z1a includes 1 to 5 lysines (K). In some embodiments, Z1a includes 1 to 3 K amino acids. In some embodiments Z1a includes 5 to 6 amino acids and at least one or more amino acids are K. In some embodiments, Z1a includes 5 to 6 amino acids and 1 to 3 amino acids are K. In some embodiments, Z1a is selected from any of KA, KD, KE, KF, KG, KH, KI, KL, KN, KP, KQ, KR, KS, KT, KY, KAA, KAD, KAE, KAF, KAG, KAH, KAI, KAL, KAN, KAQ, KAR, KAS, KAT, KAY, KDA, KDD, KDE, KDF, KDG, KDH, KDI, KDL, KDN, KDQ, KDR, KDS, KDT, KDY, KEA, KED, KEE, KEF, KEG, KEH, KEI, KEL, KEN, KEQ, KER, KES, KET, KEY, KFA, KFD, KFE, KFF, KFG, KFH, KFI, KFL, KFN, KFQ, KFR, KFS, KFT, KFY, KGA, KGD, KGE, KGF, KGG, KGH, KGI, KGL, KGN, KGQ, KGR, KGS, KGT, KGY, KHA, KHD, KHE, KHF, KHG, KHH, KHI, KHL, KHN, KHQ, KHR, KHS, KHT, KHY, KIA, KID, KIE, KIF, KIG, KIH, KII, KIL, KIN, KIQ, KIR, KIS, KIT, KIY, KLA, KLD, KLE, KLF, KLG, KLH, KLI, KLL, KLN, KLQ, KLR, KLS, KLT, KLY, KNA, KND, KNE, KNF, KNG, KNH, KNI, KNL, KNN, KNQ, KNR, KNS, KNT, KNY, KPA, KPD, KPE, KPF, KPG, KPH, KPI, KPL, KPN, KPQ, KPR, KPS, KPT, KPY, KQA, KQD, KQE, KQF, KQG, KQH, KQI, KQL, KQN, KQQ, KQR, KQS, KQT, KQY, KRA, KRD, KRE, KRF, KRG, KRH, KRI, KRL, KRN, KRQ, KRR, KRS, KRT, KRY, KSA, KSD, KSE, KSF, KSG, KSH, KSI, KSL, KSN, KSQ, KSR, KSS, KST, KSY, KTA, KTD, KTE, KTF, KTG, KTH, KTI, KTL, KTN, KTQ, KTR, KTS, KTT, KTY, KYA, KYD, KYE, KYF, KYG, KYH, KYI, KYL, KYN, KYQ, KYR, KYS, KYT, KYY, SEQ ID NOs 75-24014, 24037-24046. In some embodiments, Z1a is selected from KSNAPQK (SEQ ID NO:24037), KNASPQK (SEQ ID NO:24038), KLWAVK (SEQ ID NO:24039), KGARLK (SEQ ID NO:24040), ADKKTLN (SEQ ID NO:24041), KGSHK (SEQ ID NO:4238), KNSTK (SEQ ID NO:5085), GKNSTK (SEQ ID NO:13989), GKGSHK (SEQ ID NO:13198), GSHKGSHK (SEQ ID NO:24042), GKPSHKP (SEQ ID NO:24043), GKGPSK (SEQ ID NO:24044), GKGSKK (SEQ ID NO:24045), and GKKPGKK (SEQ ID NO:24046).

In some embodiments Z1a is appended to the N-terminus and/or C-terminus, and/or inserted into the sequence of the A-chain or B-chain of insulin.

### Compounds

In some embodiments, the present disclosure provides a compound comprising X1 and one or more Z1c, or a tautomer, stereoisomer or a mixture of stereoisomers, or a pharmaceutically acceptable salt, or hydrate, or isotopic derivative thereof, wherein: X1 comprises:
(i) NH₂ or OH (for example, X1 is NH₂ or OH);
(ii) a drug substance comprising an amine;
(iii) a drug substance that is covalently conjugated to an amine containing linker; or
(iv) an amine configured to be covalently conjugated to a drug substance;
wherein each Z1c is independently selected from Formulae FF1-FF224; and wherein each Z1c is covalently conjugated, directly or indirectly, to an amine in X1 or to OH when X1 is OH.

In some embodiments, Z1c is independently selected from Formulae FF1-FF48, Formulae FF49-FF88, FF89-FF112, FF113-FF136, FF137-FF160, FF161-FF164, FF165-FF166, FF167-FF192, FF193-FF209, and FF210-FF224.

Formulae FF1-FF48 are:

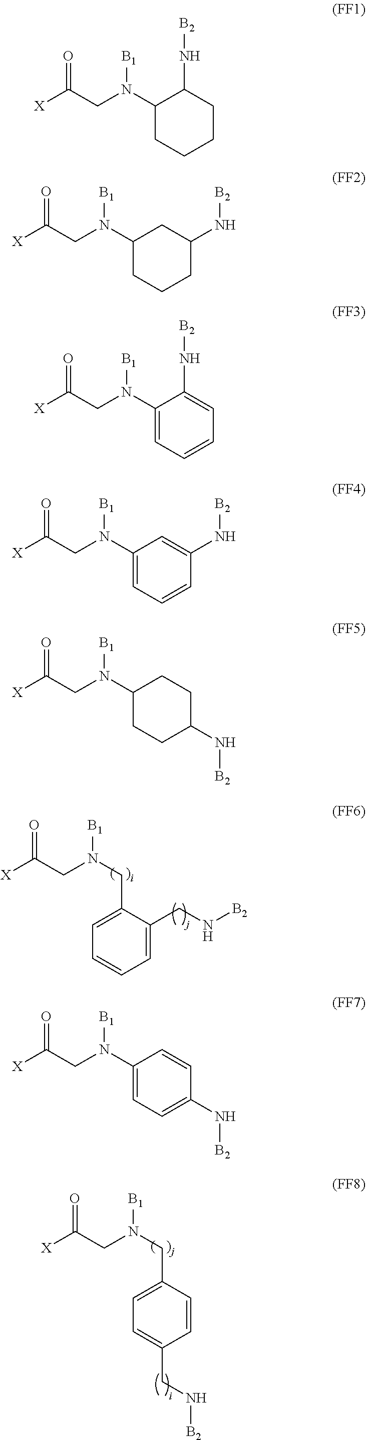

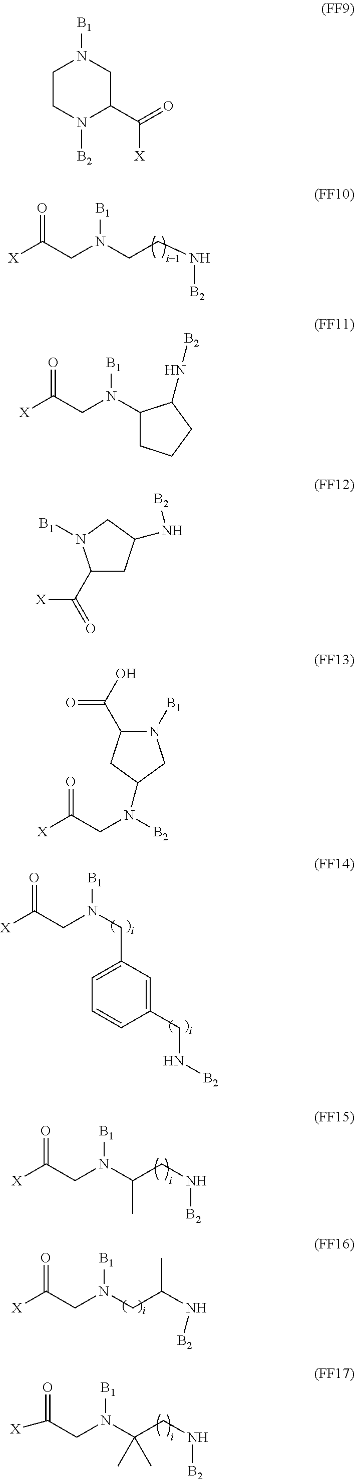

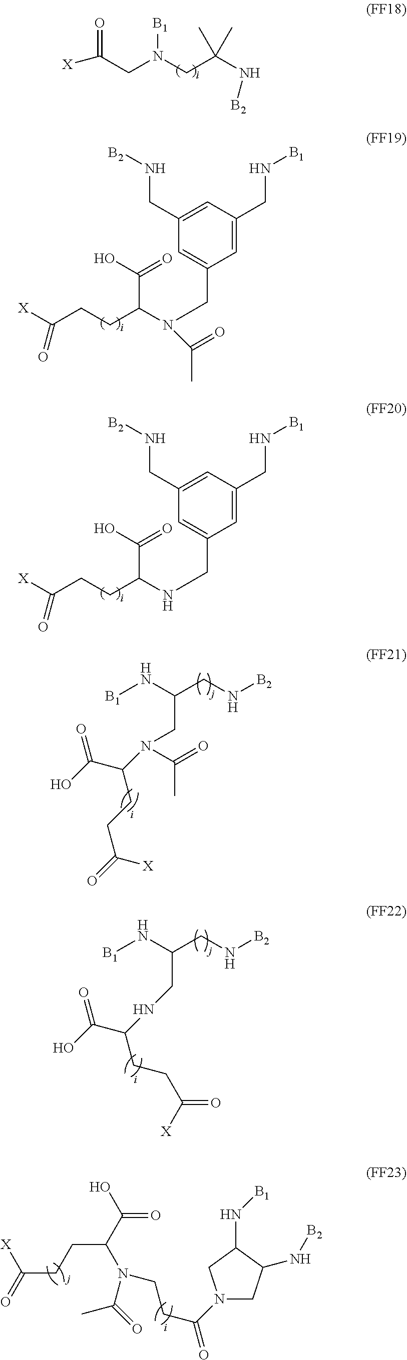

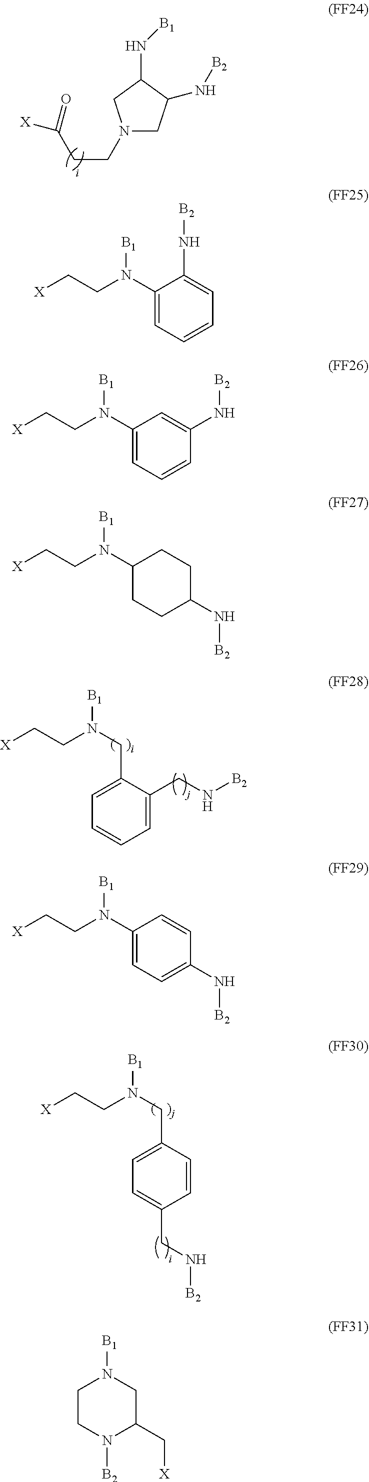

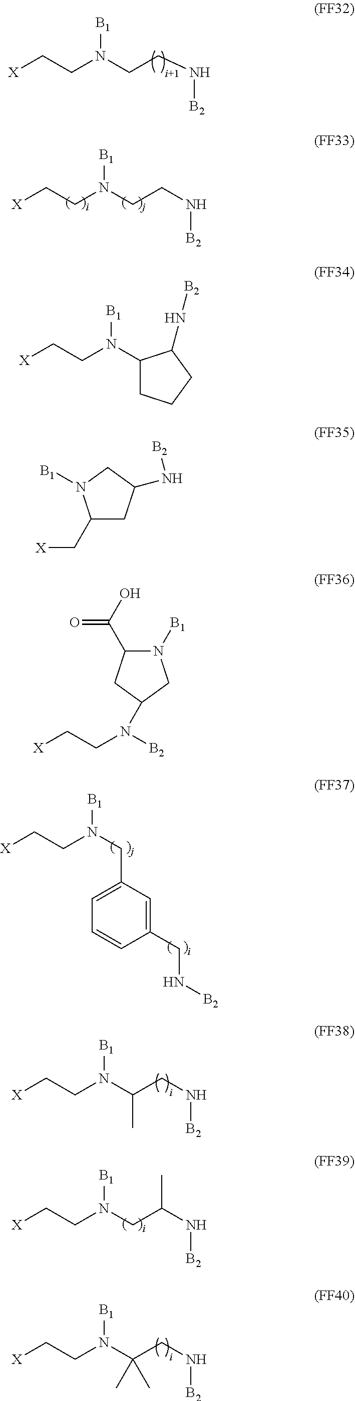

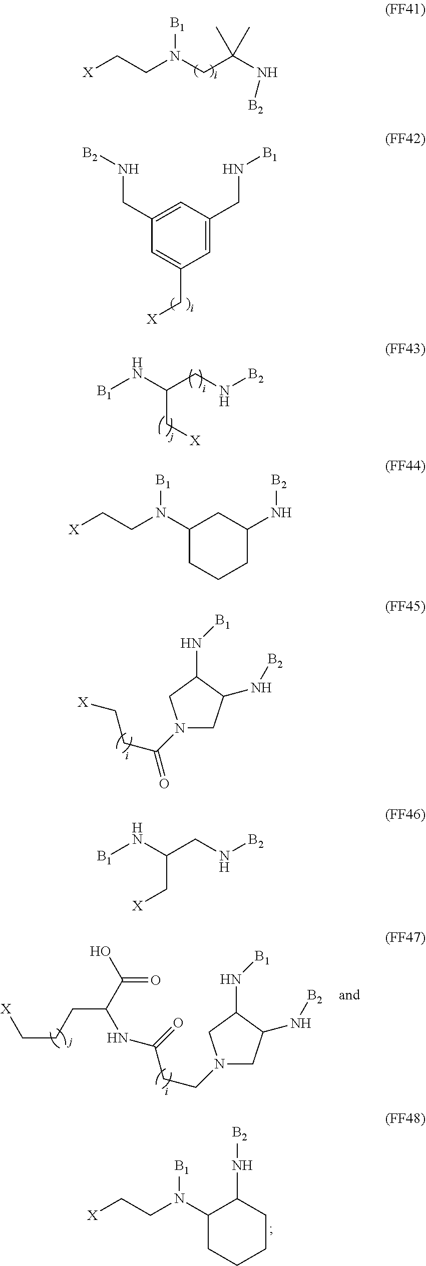

wherein X represents a point of covalent attachment either directly to an amine in X1 or to an amine that is covalently conjugated directly or indirectly to X1, or to OH when X1 is OH;
i is 1, 2, 3, 4, 5, 6, or 7;
j is 1, 2, 3, 4, 5, 6, or 7; and B₁ and B₂, which may be identical or different, each independently represents an aromatic boron-containing group.

Formulae FF49-FF88 are:

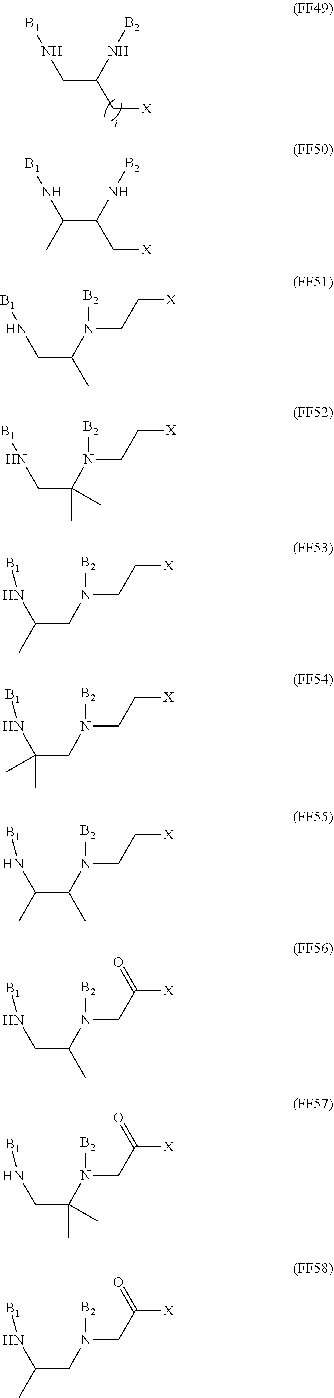

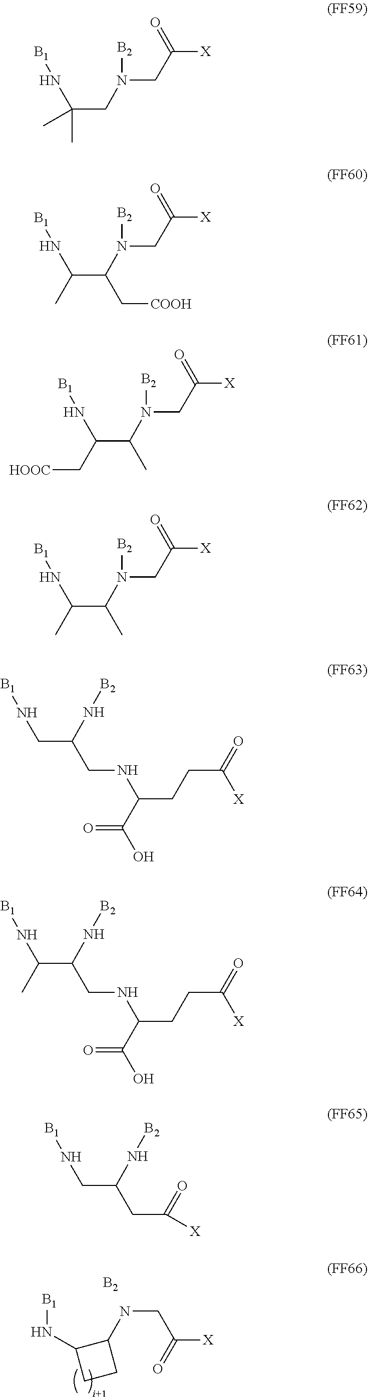

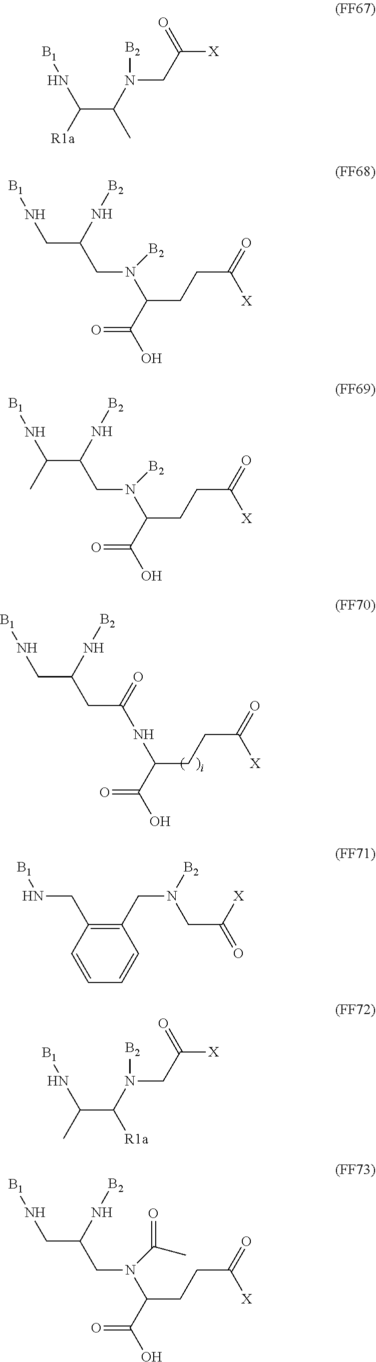

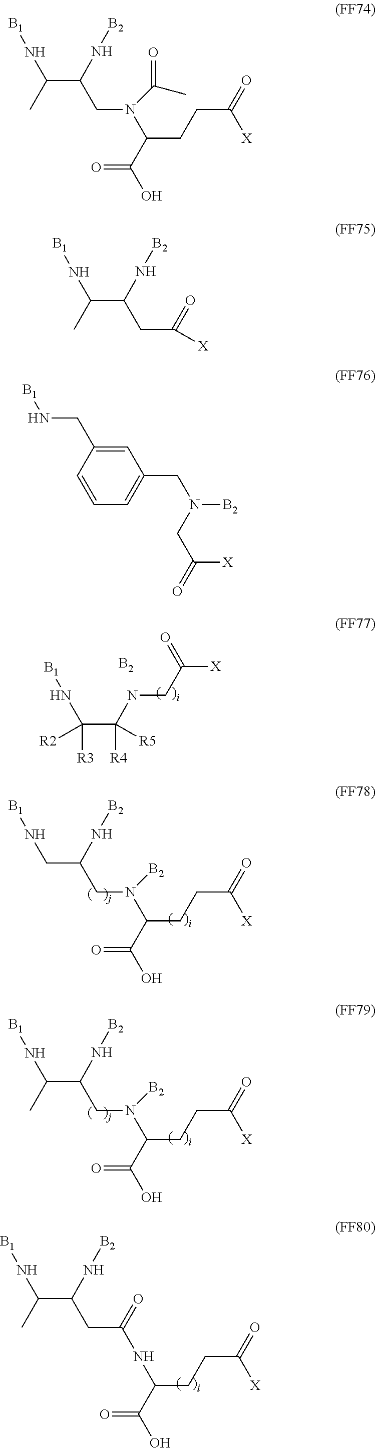

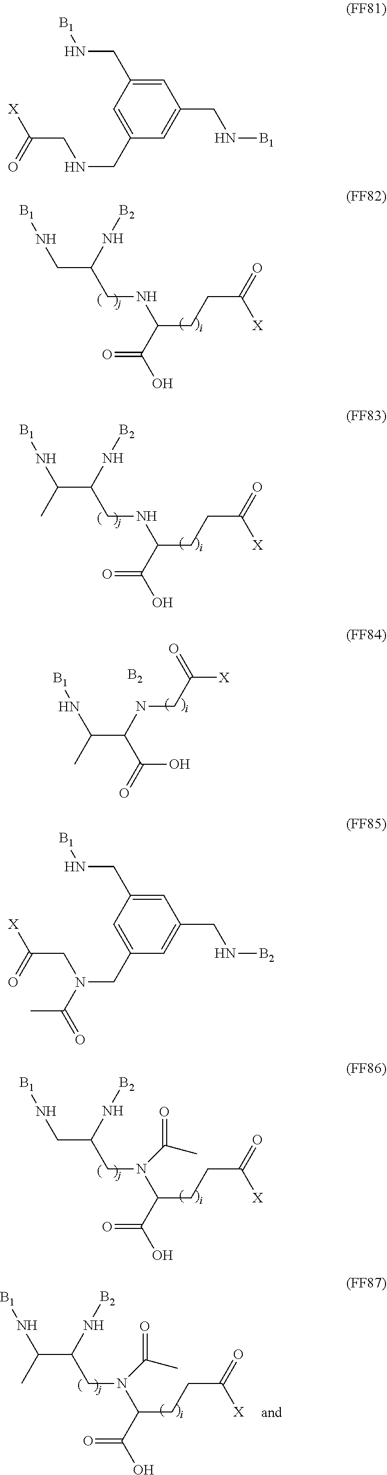

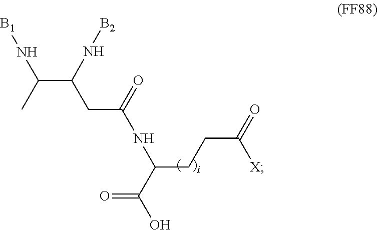

wherein X represents a point of covalent attachment either directly to an amine in X1 or to an amine that is covalently conjugated directly or indirectly to X1, or to OH when X1 is OH;
i is 1, 2, 3, 4, 5, 6, or 7;
j is 1, 2, 3, 4, 5, 6, or 7; R1a is selected from COOH, CH₃, H, and OH; R2, R3, R4 and R5 are each independently selected from CH₃, H, OH, and COOH, and at least one of R2, R3, R4 and R5 is CH₃ or OH; and B₁ and B₂, which may be identical or different, are each independently an aromatic boron-containing group.

Formulae FF89-FF112 are:

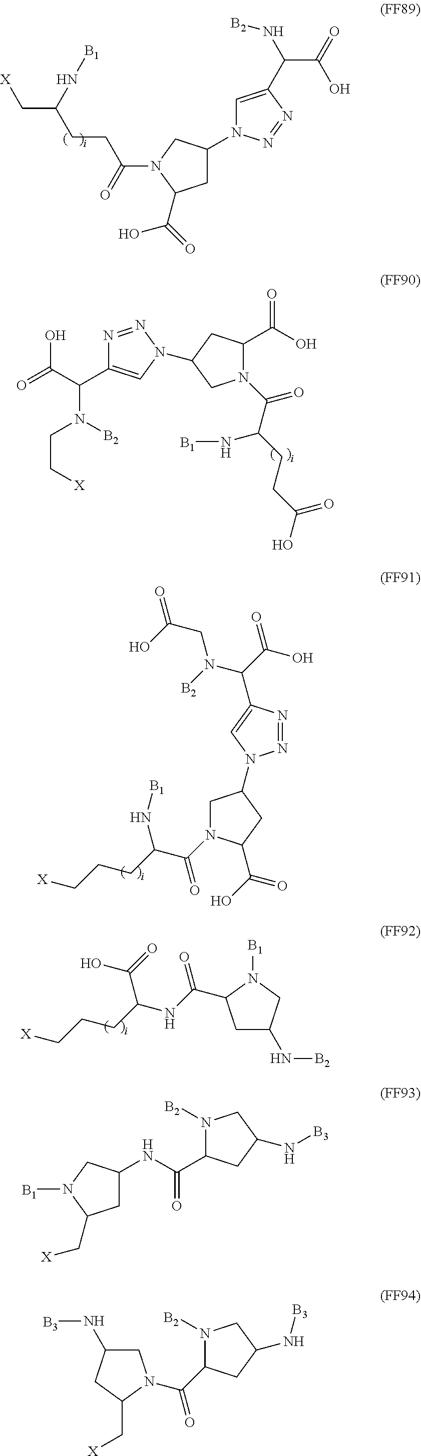

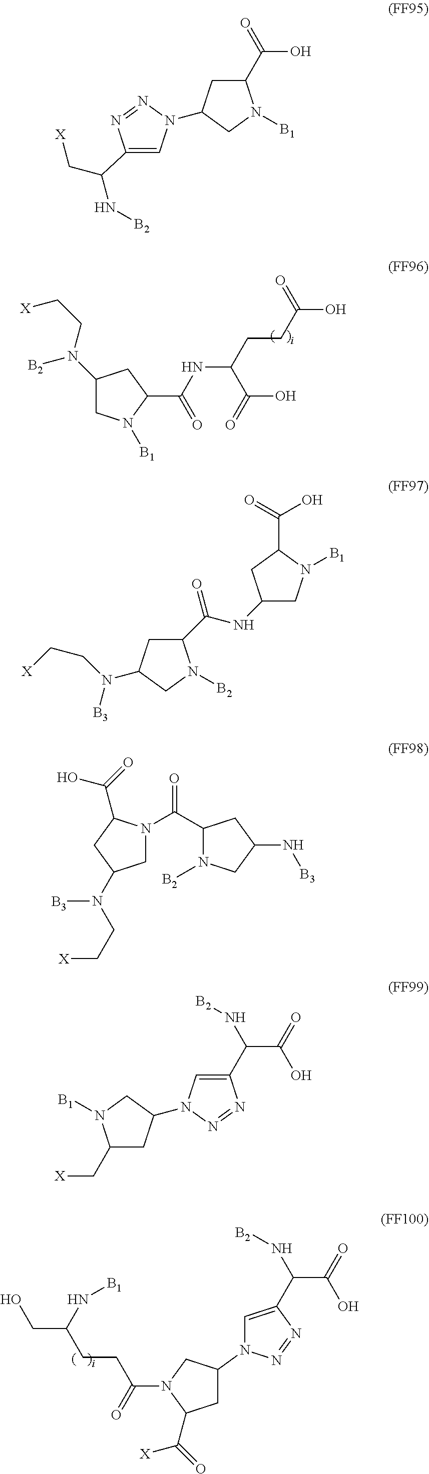

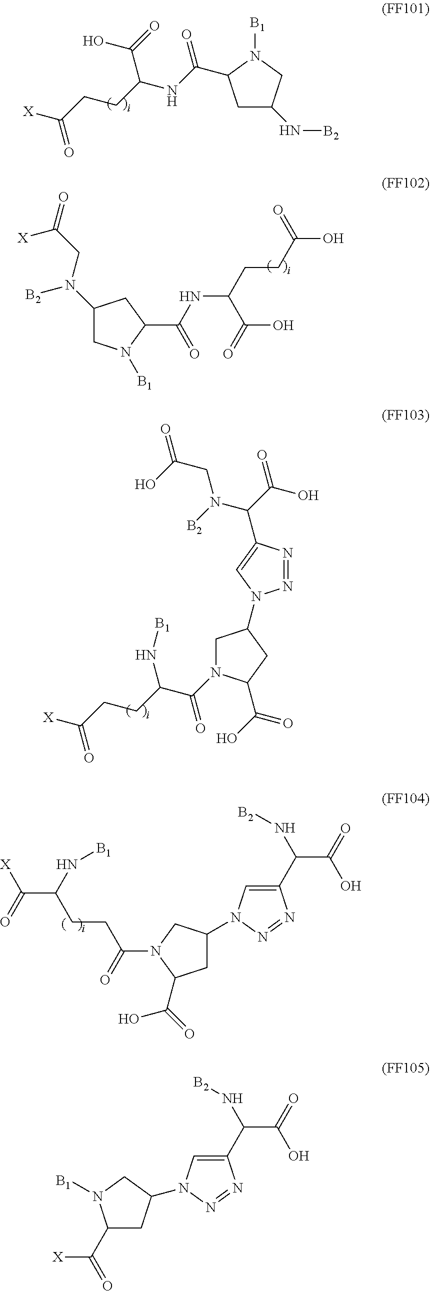

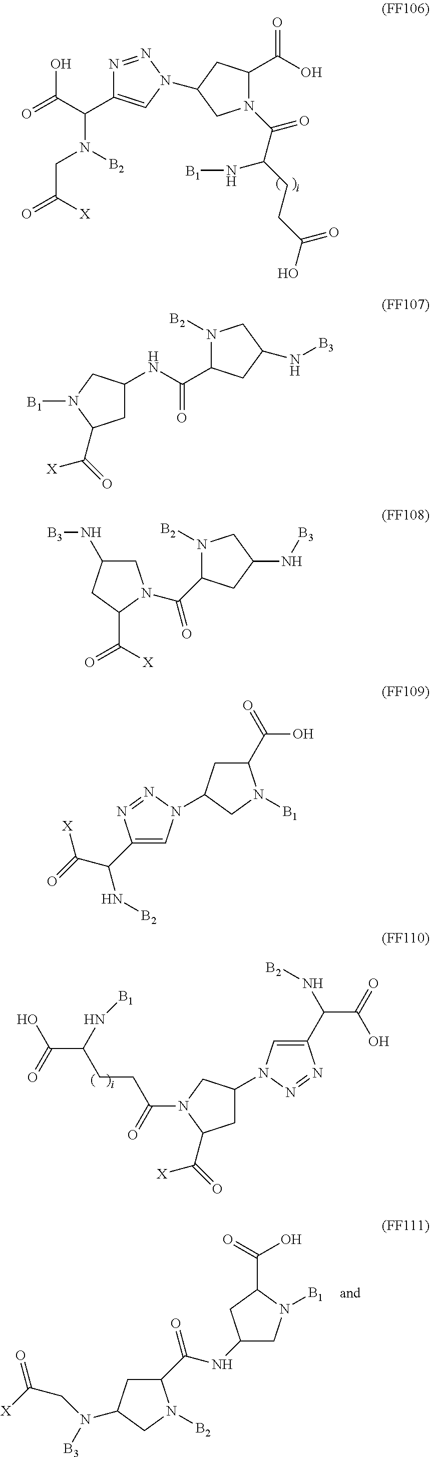

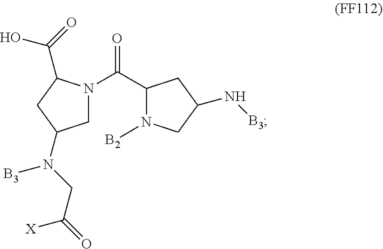

wherein X represents a point of covalent attachment either directly to an amine in X1 or to an amine that is covalently conjugated directly or indirectly to X1, or to OH when X1 is OH;
i is 1, 2, 3,4, 5, 6, or 7; and
B₁, B₂ and B₃, which may be identical or different, are each independently an aromatic boron-containing group, a carboxylic acid derivative, or a H, wherein in each FF89-FF112 structure containing B1, B2 and B3 groups, at least two of the B1, B2 and B3 groups are independently an aromatic boron-containing group.

Formulae FF113-FF136 are:

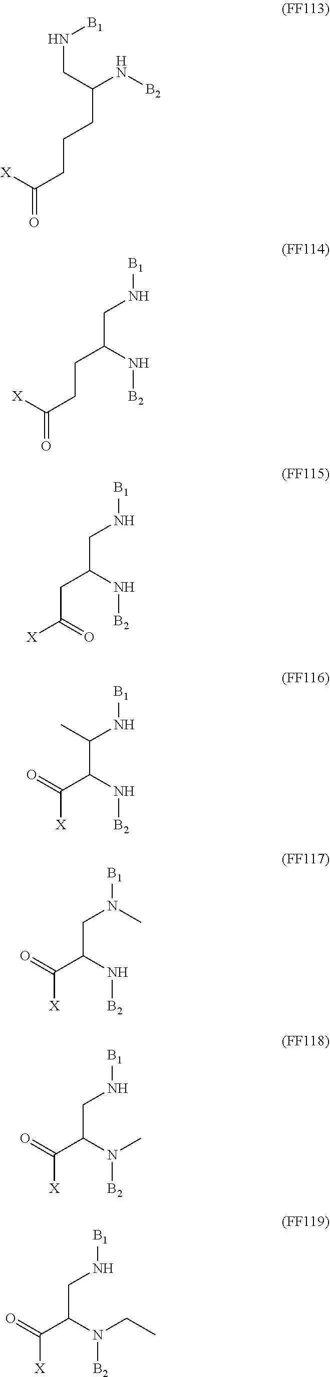

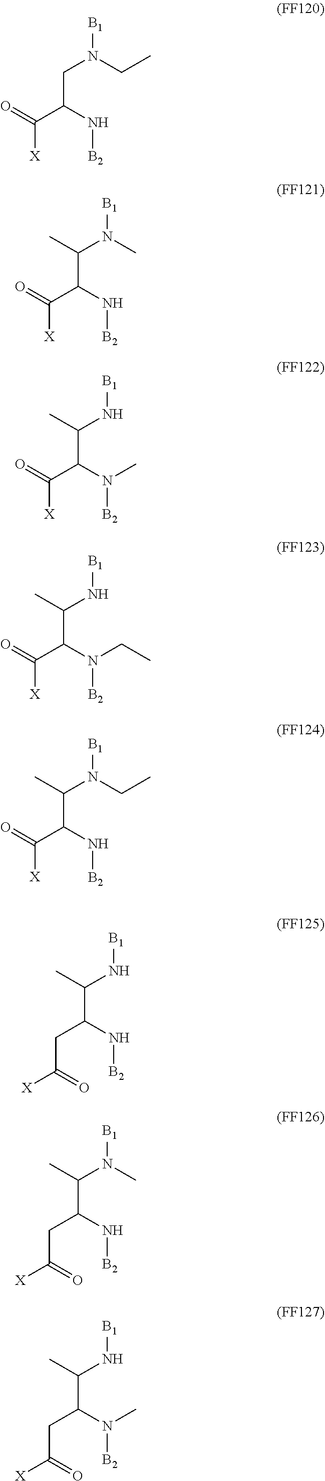

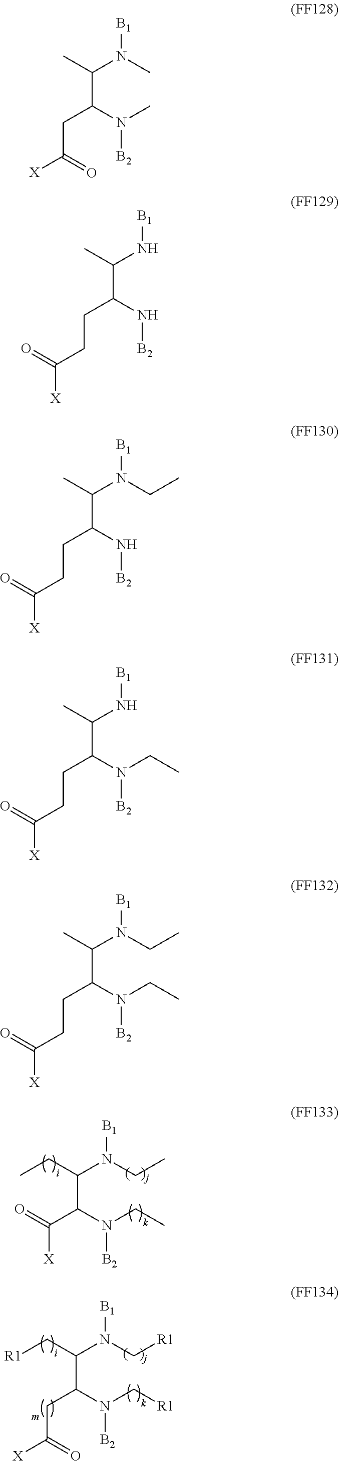

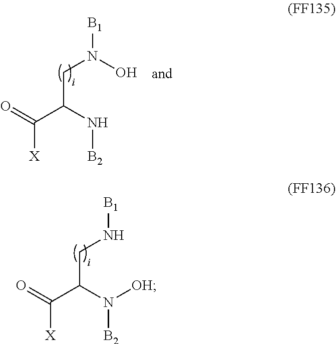

wherein X represents a point of covalent attachment either directly to an amine in X1 or to an amine that is covalently conjugated directly or indirectly to X1, or to OH when X1 is OH;
i is 0, 1, 2, 3, 4, 5, 6, or 7;
j is 0, 1, 2, 3, 4, 5, 6, or 7;
k is 0, 1, 2, 3, 4, 5, 6, or 7;
m is 0, 1, 2, 3, 4, 5, 6, or 7;
wherein i+j+k+m is greater than 0;
each R1 is independently selected from H, an alkyl group, an acyl group, a cycloalkyl group, a haloalkyl group, an aryl group, and a heteroaryl group, each R1 optionally comprises one or more alkyl-halide, halide, sulfhydryl, aldehyde, amine, acid, hydroxyl, alkyl or aryl groups; and
B₁ and B₂, which may be identical or different, each independently represents an aromatic boron-containing group.

Formulae FF137-FF160 are:

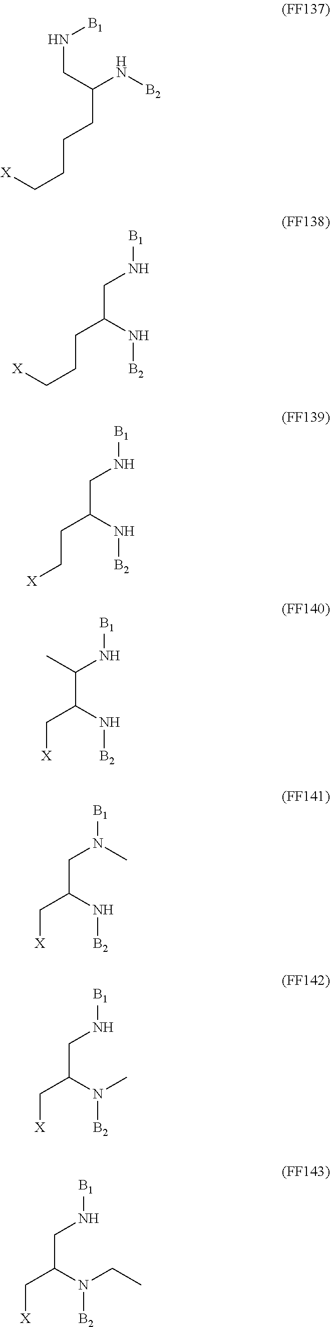

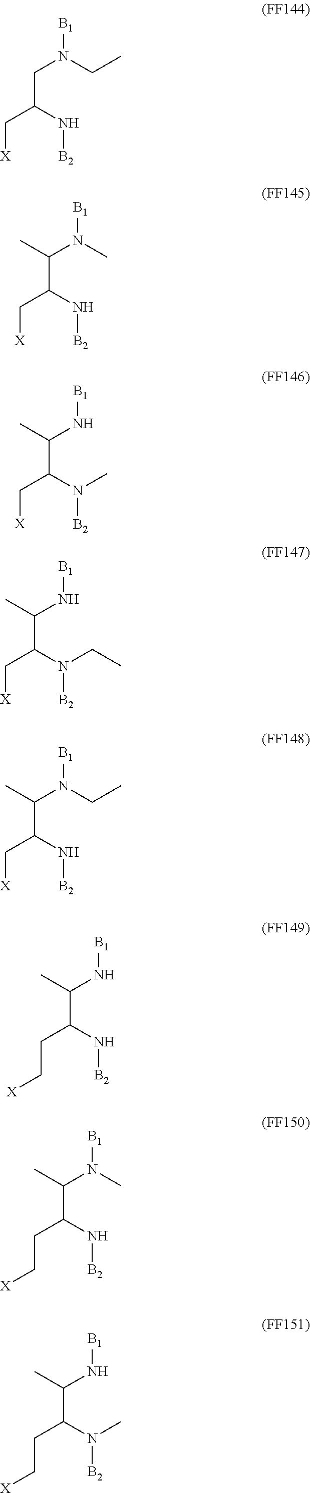

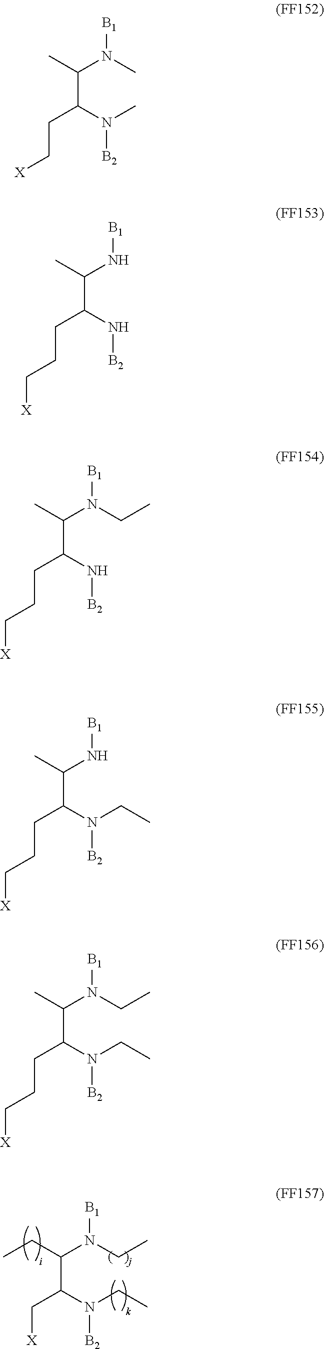

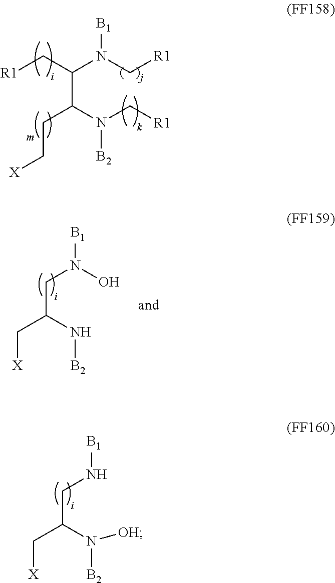

wherein X represents a point of covalent attachment either directly to an amine in X1 or to an amine that is covalently conjugated directly or indirectly to X1, or to OH when X1 is OH;
i is 0, 1, 2, 3, 4, 5, 6, or 7;
j is 0, 1, 2, 3, 4, 5, 6, or 7;
k is 0, 1, 2, 3, 4, 5, 6, or 7;
m is 0, 1, 2, 3, 4, 5, 6, or 7;
wherein i+j+k+m is greater than 0;
each R1 is independently selected from H, an alkyl group, an acyl group, a cycloalkyl group, a haloalkyl group, an aryl group, and a heteroaryl group, each R1 optionally comprises one or more alkyl-halide, halide, sulfhydryl, aldehyde, amine, acid, hydroxyl, alkyl or aryl groups; and
B₁ and B₂, which may be identical or different, each independently represents an aromatic boron-containing group. In one embodiment, B1 and B₂ may be identical or different.

Formulae FF161-FF164 are:

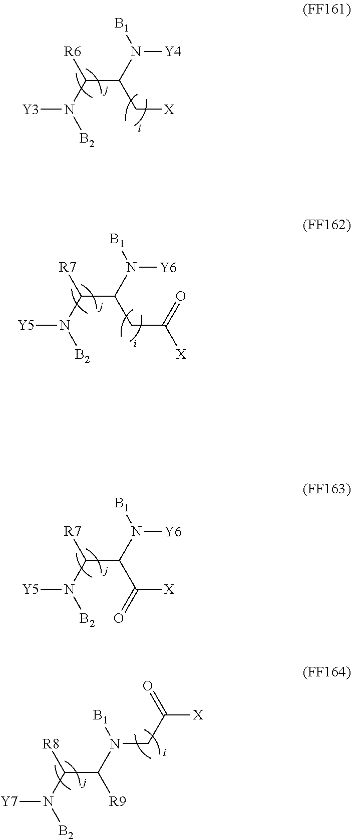

wherein X represents a point of covalent attachment either directly to an amine in X1 or to an amine that is covalently conjugated directly or indirectly to X1, or to OH when X1 is OH;
i is 1, 2, 3, 4, or 5 (e.g., 1, 2, 3, or 5);
j is 1, 2, 3, 4, or 5 (e.g., 1, 2, 3, or 5);
each R6, R7, R8, and R9 for different values of j is independently selected from H, CF₃, CH₃, CHF₂, and (CH₂)ₘCH₃, wherein m is 1, 2, 3, 4, or 5;
Y3, Y4, Y5, Y6 and Y7 are each independently selected from H, CH₂—X4, and Formulae IV-1 to IV-135;
wherein X4 is selected from —COOH, —(CH₂)ₘCOOH, an alkyl group, an acyl group, a cycloalkyl group, a haloalkyl group, an aryl group, and a heteroaryl group, each X4 optionally comprises one or more alkyl-halide, halide, sulfhydryl, aldehyde, amine, acid, hydroxyl, alkyl or aryl groups; wherein m is 1, 2, 3, 4, or 5;
wherein at least one of Y5, Y6 and Y7 in Formulae FF162 and FF163 is not H and at least one of Y7, R8 and R9 in FF164 is not H; and
wherein Formulae IV-1 to IV-135 are:

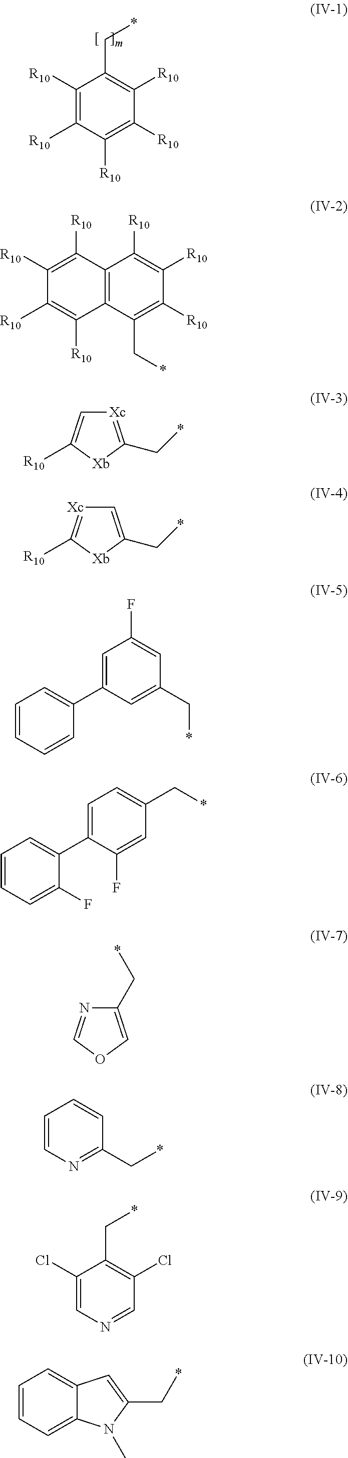

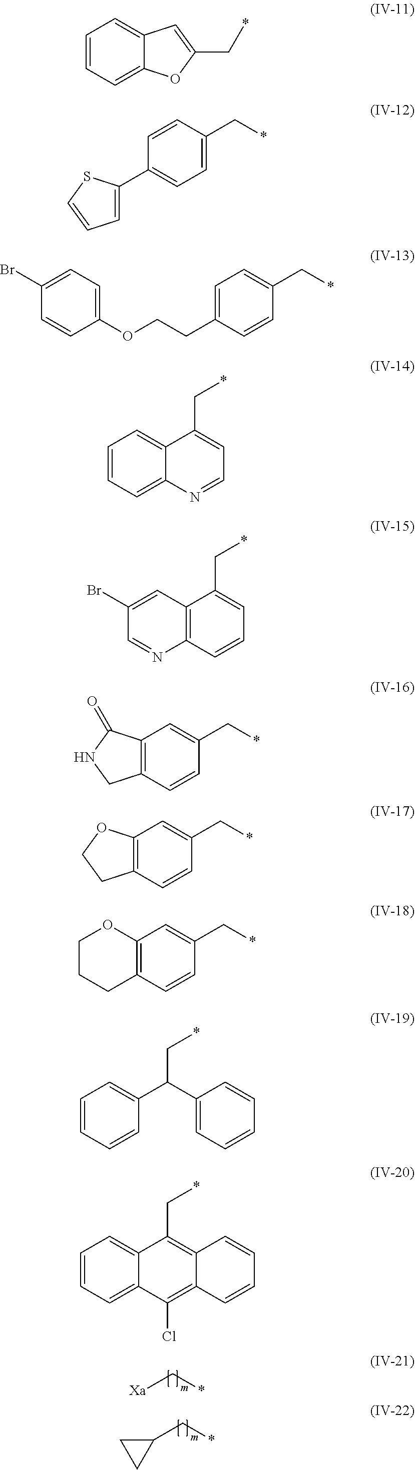

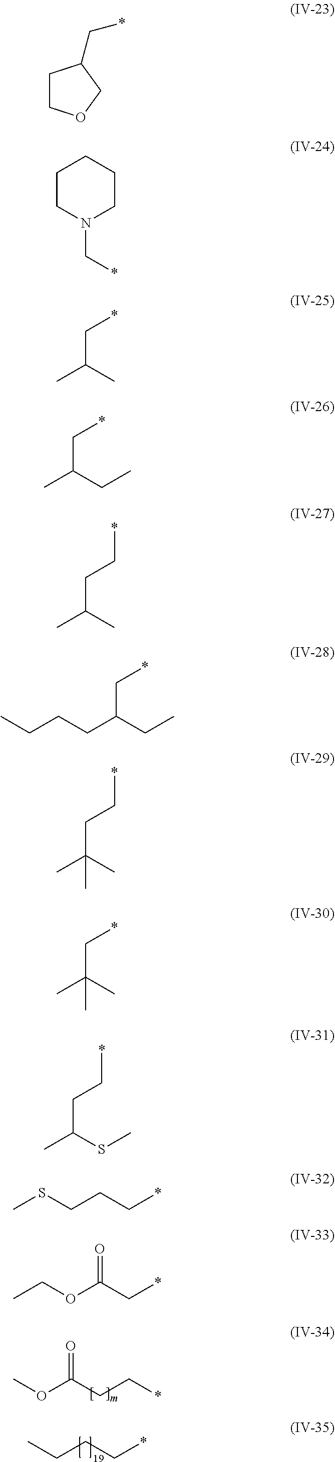

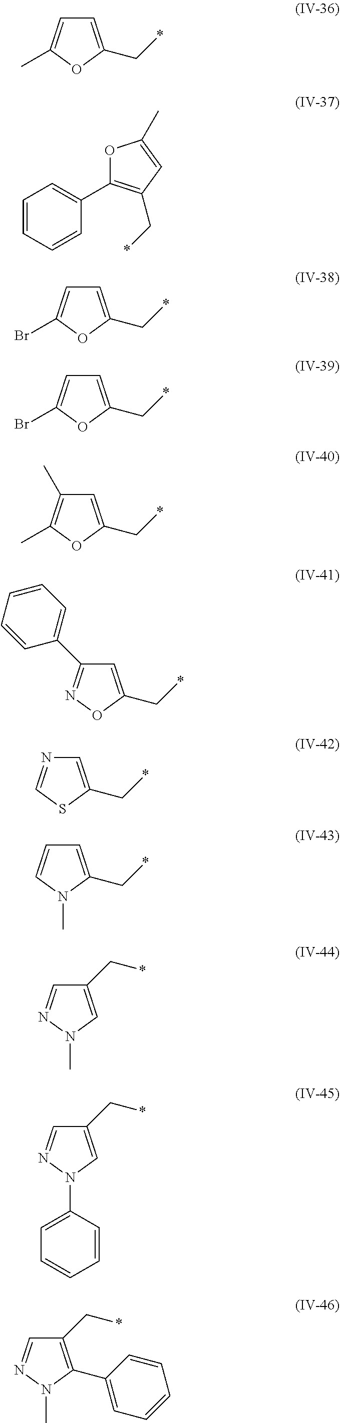

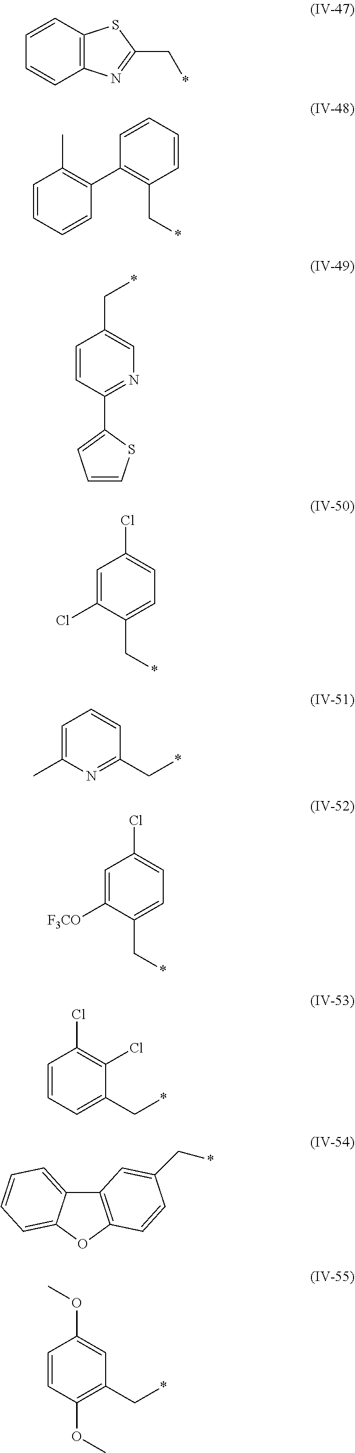

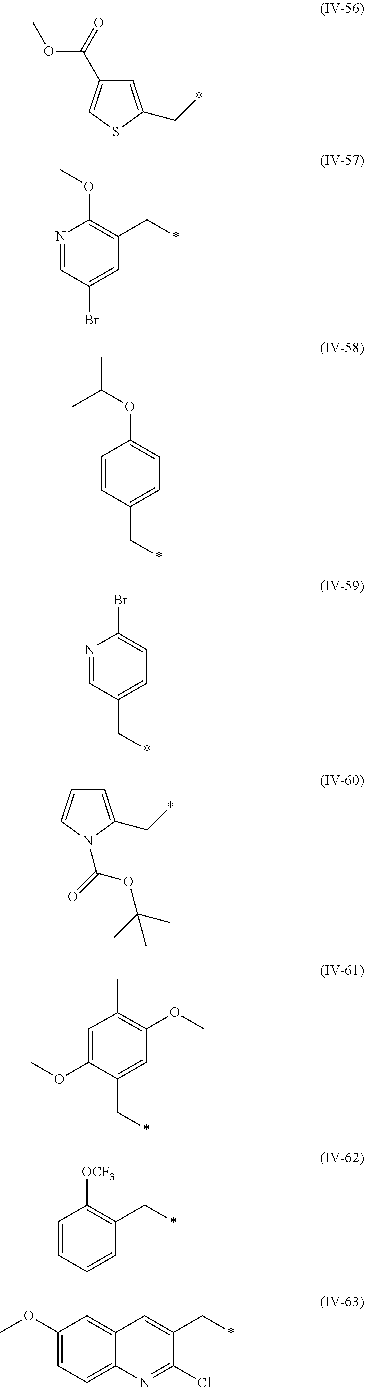

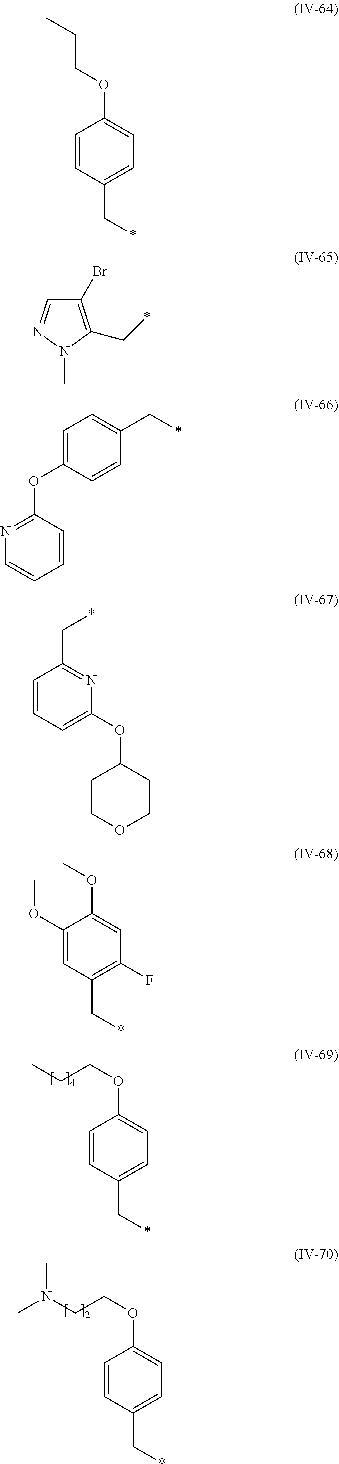

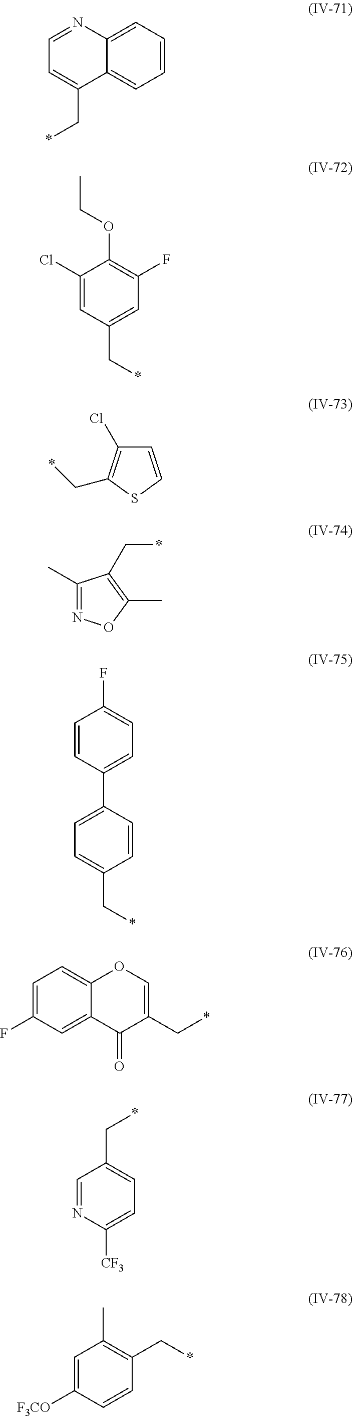

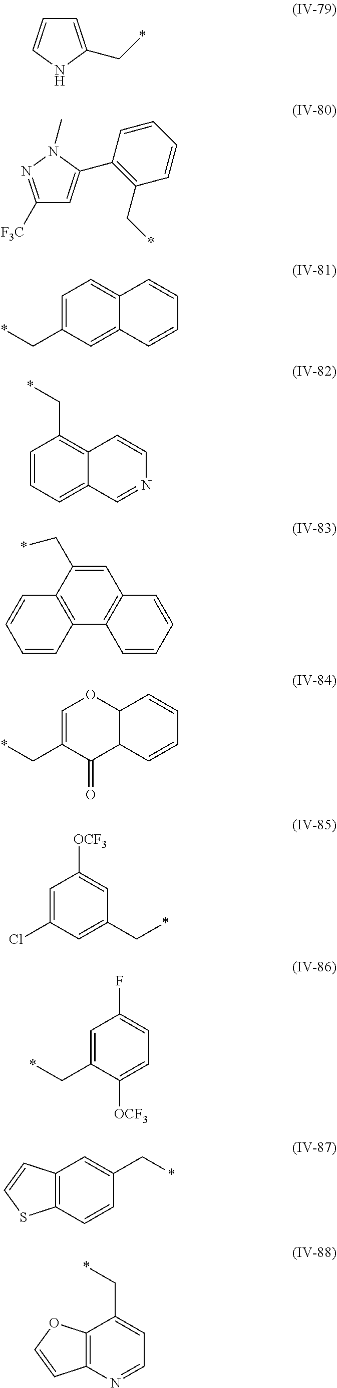

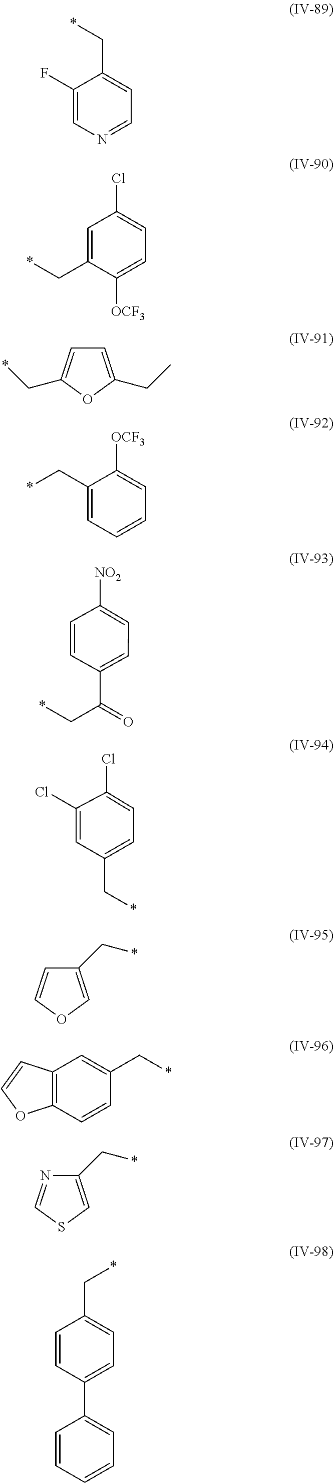

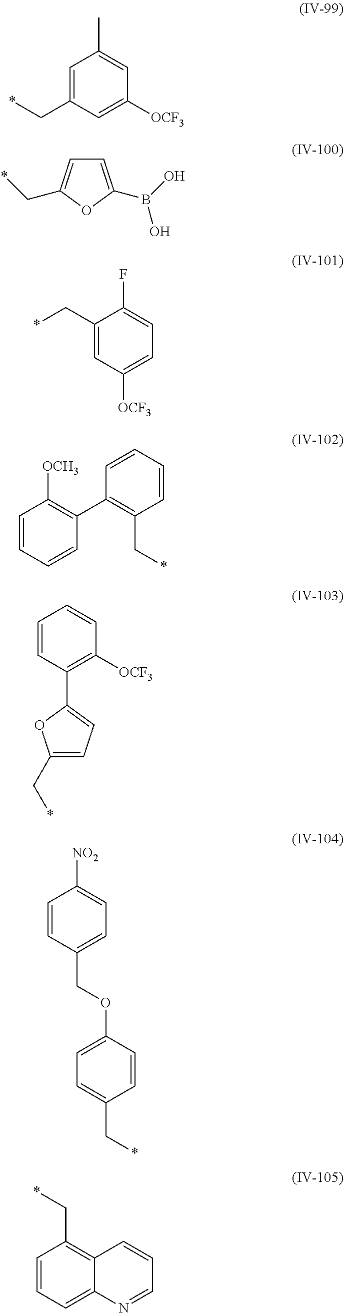

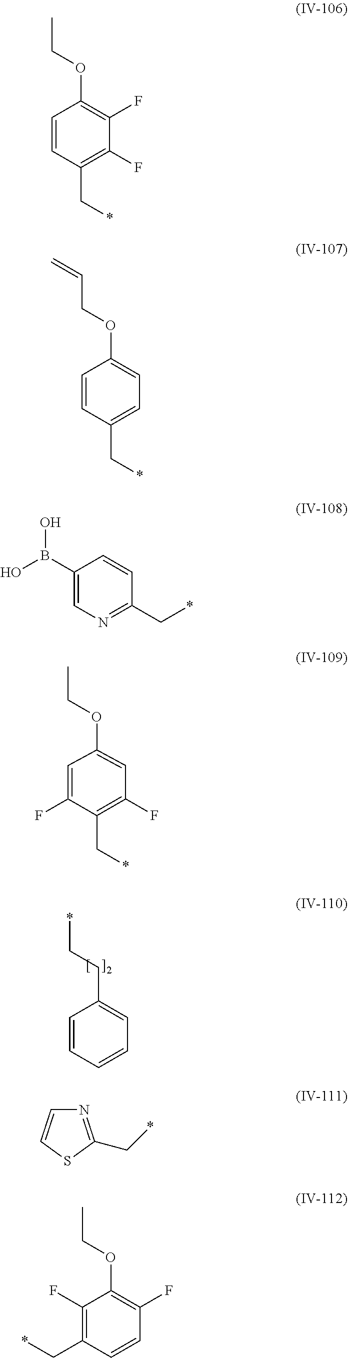

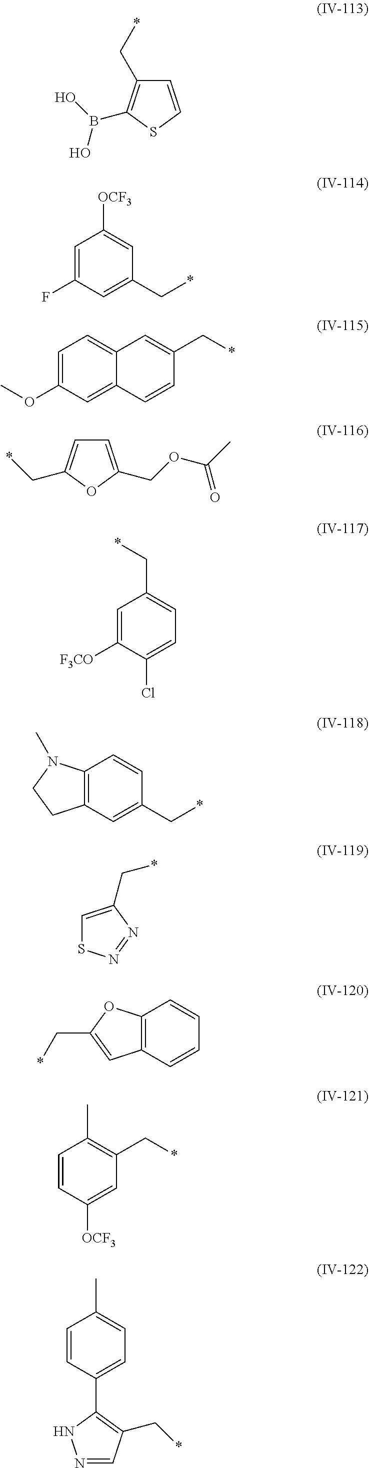

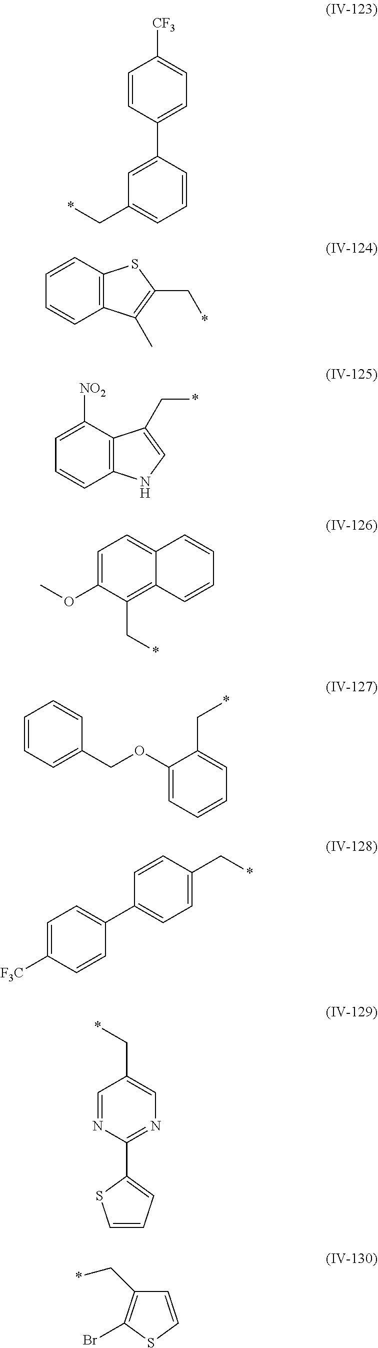

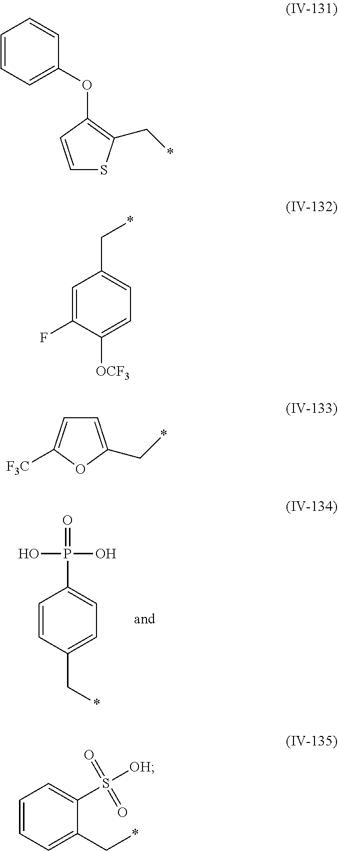

wherein Xa represents CH═O, CHF₂, CF₃, CH₂SH, COOH, CH₂OH, CH₂NO₂, CH₂NH₂, CH₃, C(CH₃)₃, CH(CH₃)₂, CH((CH₂)₃CH₃)₂, or CH(CH₂CH₃)₂;
Xb represents O, NH, CH₂, or S;
Xc represents CH or N;
each R₁₀ is independently selected from H, F, Cl, Br, CH₃, CF₃, CH═O, OH, COOH, and (CH₂)ₙCH₃,
m is 1, 2, 3, 4, or 5; and n is 1, 2, 3, 4, or 5;
B₁ and B₂, which may be identical or different, each independently represents an aromatic boron-containing group; and
* in Formulae IV-1 to IV-135 represents the point of attachment to corresponding Formulae FF161-164.

In some embodiments, when j is 4, X is not NH₂ for FF163.

Formulae FF165-FF166 are:

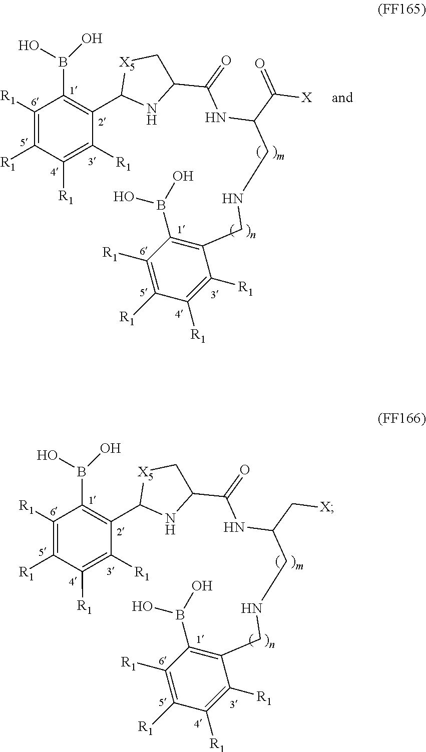

wherein X represents a point of covalent attachment either directly to an amine in X1 or to an amine that is covalently conjugated directly or indirectly to X1, or to OH when X1 is OH;
m is 1, 2, 3, 4, 5, 6, or 7;
n is 1, 2, 3, 4, 5, 6, or 7;
X5 is S, O, or NH; and
each R₁ is independently selected from H, F, Cl, Br, OH, CH₂—NH₂, NH₂, (C═O)—NH₂, CH═O, SO₂CH₃, SO₂CF₃, CF₃, CHF₂, NO₂, CH₃, OCH₃, O(CH₂)ₘCH₃, —(SO₂)NH CH₃, —(SO₂)NH(CH₂)ₘCH₃, and OCF₃, wherein m is 1, 2, 3, 4, 5, 6, or 7.

Formulae FF167-FF192 are:

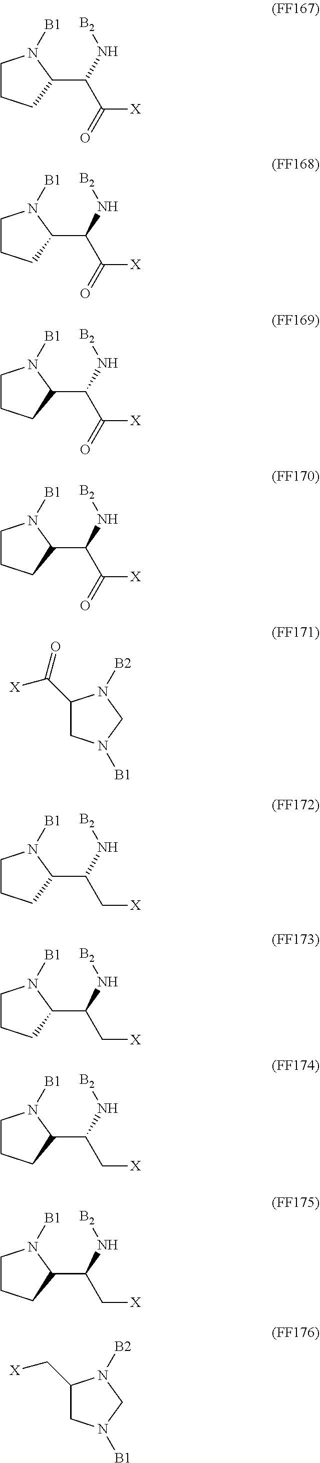

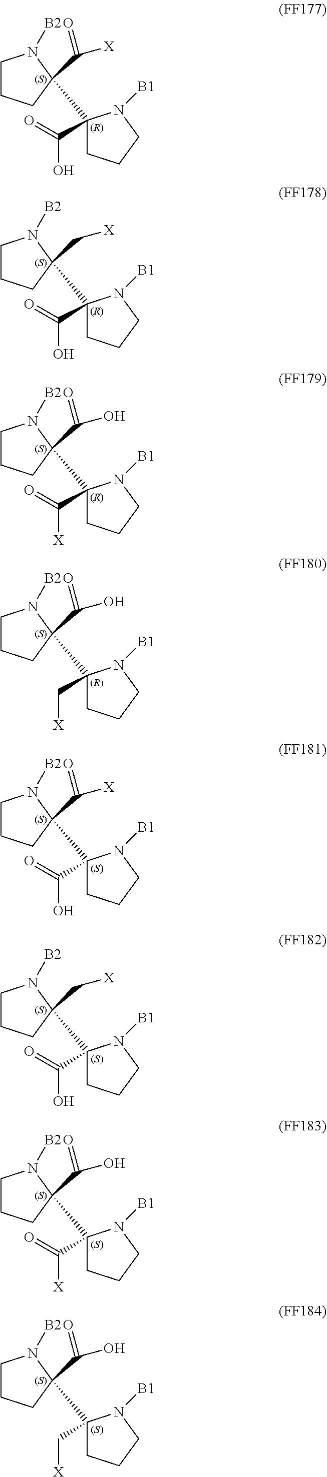

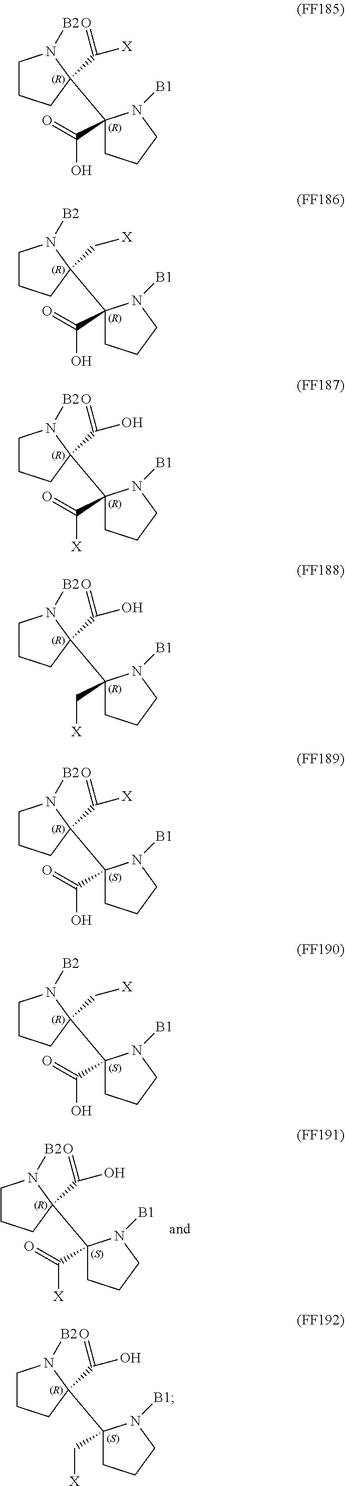

wherein X represents a point of covalent attachment either directly to an amine in X1 or to an amine that is covalently conjugated directly or indirectly to X1, or to OH when X1 is OH; and
B₁ and B₂, which may be identical or different, each independently represents an aromatic boron-containing group.

Formulae FF193-FF209 are:

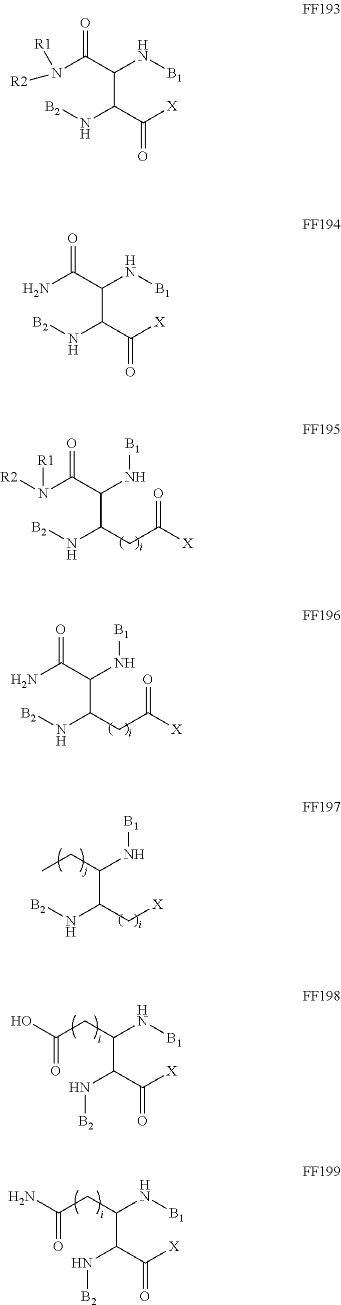

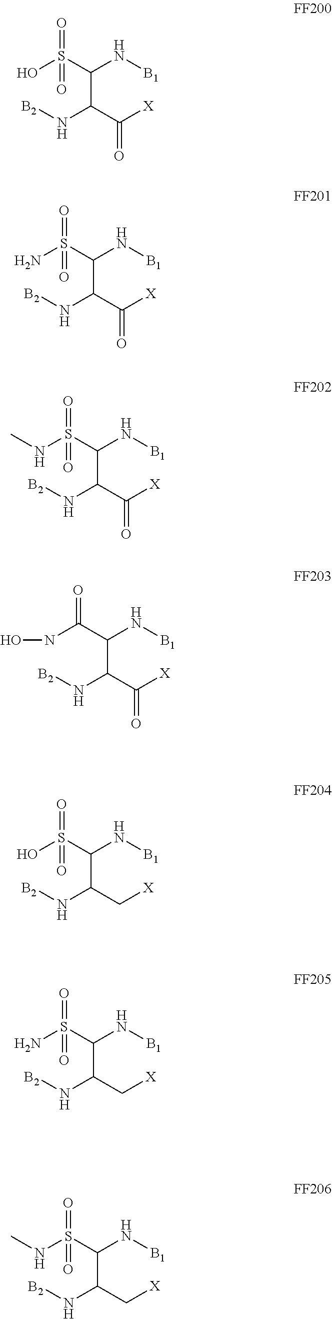

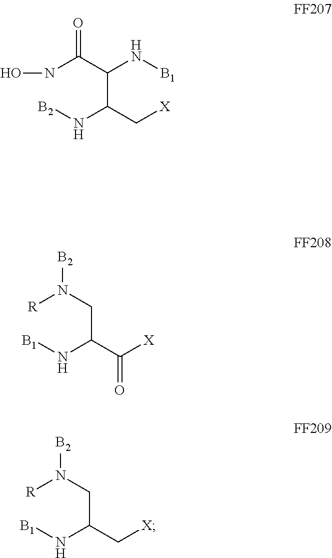

wherein R in FF208 and FF209 is an alkyl, aryl or halide that is covalently conjugated through at least one CH₂ group to the amino group in the side chain of FF208 or FF209,
R1 and R2 are independently selected from H, CH3, alkyl, and formulae IV-1 to IV-135;
i is 1, 2, 3,4, or 5;
j is 1, 2, 3, 4, or 5; and
wherein X represents a point of covalent attachment either directly to an amine in X1 or to an amine that is covalently conjugated directly or indirectly to X1, or to OH when X1 is OH; and
B₁ and B₂, which may be identical or different, each independently represents an aromatic boron-containing group.

Formulae FF210-FF224 are:

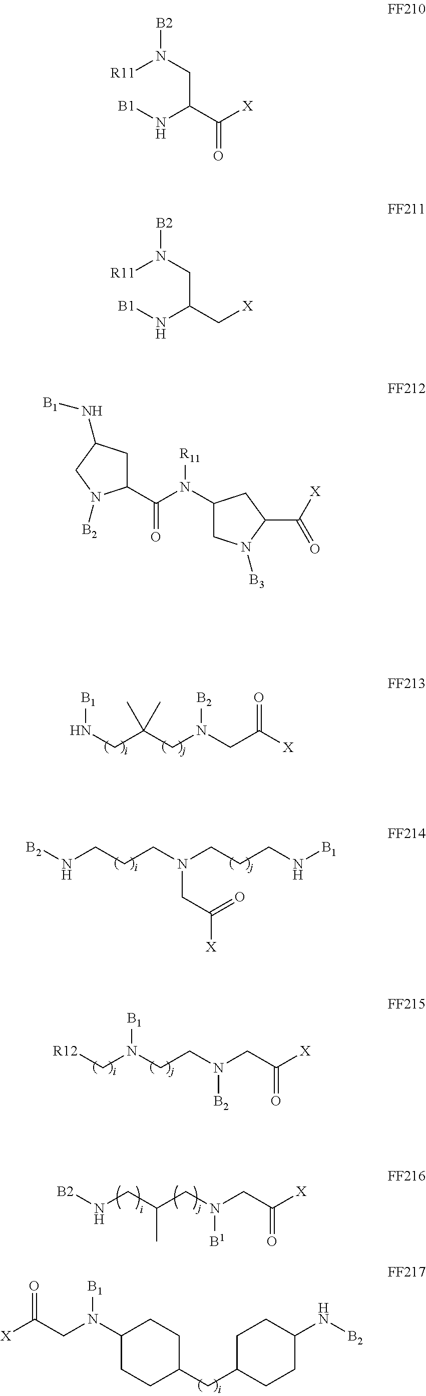

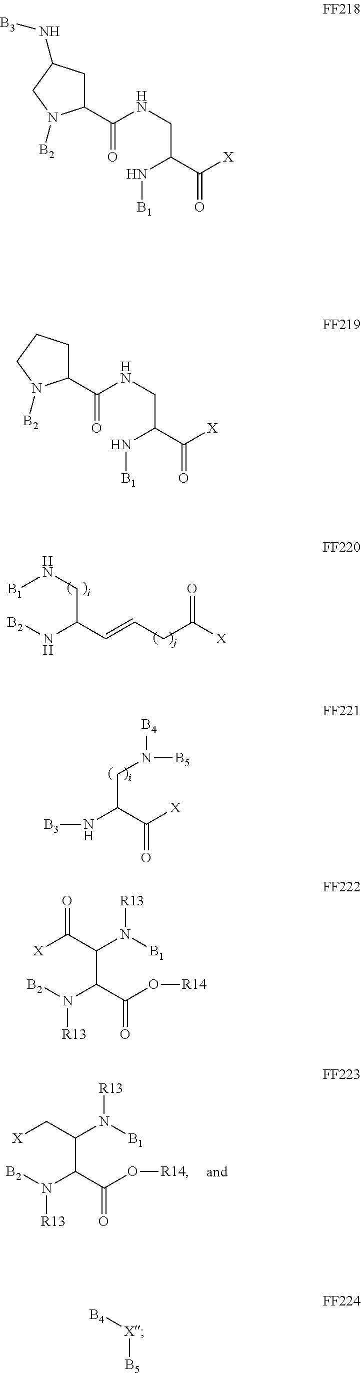

wherein R11 in FF210 to FF212 is selected from Formulae IV-1 to IV-135 and R12 is selected from an amine, a hydroxyl, an alkyl, and a halide group;
wherein each R13 is independently selected from H, CH₃, alkyl, aryl and Formulae IV-1 to IV-135; R14 is selected from H, CH₃, alkyl, aryl and heteroaryl;
wherein X represents a point of covalent attachment either directly to an amine in X1 or to an amine that is covalently conjugated directly or indirectly to X1, or to OH when X1 is OH;
X″ represents a point of covalent attachment to an amine —N in the compound, wherein — represents a single covalent bond to a CH₂ or CH group in the compound;
i is 1, 2, 3, 4, or 5;
j is 1, 2, 3, 4, or 5; and
B₁, B₂, B₃, B₄, B₅, and B₆ each independently represents an aromatic boron-containing group, wherein in each FF structure containing B₁, B₂ and B₃ groups, at least two of the B1, B2 and B3 groups are independently an aromatic boron-containing group; and
wherein at least one primary or secondary amine in FF1-FF223 is optionally covalently conjugated to B6.

In some embodiments, the present disclosure provides a compound of Formula (I) or a molecular conjugate represented by Formula I, or a stereoisomer or a mixture of stereoisomers, or pharmaceutically acceptable salt thereof:

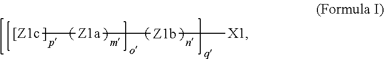

wherein X1 comprises:
   (i) NH₂ or OH (for example, X1 is NH₂ or OH),
   (ii) a polypeptide drug substance comprising an amine,
   (iii) a polypeptide drug substance that is covalently conjugated to an amine containing linker, or
   (iv) an amine configured to be covalently conjugated to a polypeptide drug substance;
each Z1c is independently selected from Formulae FF1-FF224 and covalently conjugated either directly, or via Z1a and/or Z1b, to X1; each Z1a independently comprises 1 to 50 amino acids connected together using amide or peptide bonds; each Z1b is independently a small-molecule linker; each m′ is independently 0 or 1; each n′ is independently 0 or a positive integer; each o′ is independently an integer of 1 or greater; each p′ is a positive integer; and q′ is a positive integer of at least 1 and not more than two times the total number of amine groups in X1, wherein when any of n′, o′, p′, or q′ is 2 or more, the corresponding groups Z1a, Z1b, and Z1c are independently selected and may be the same or different; wherein each Z1c is independently covalently conjugated, directly or indirectly, to an amine of Z1a, to an amine of Z1b, or to X1; and wherein optionally the molecular conjugate may comprise one or more isotopes at any position of the molecular conjugate of Formula I.

In at least some embodiments, X1 comprises one of:
(i) NH₂ or OH (for example, X1 is NH₂ or OH),
(ii) a polypeptide drug substance comprising an amine,
(iii) a polypeptide drug substance that is covalently conjugated to an amine containing linker, or
(iv) an amine configured to be covalently conjugated to a polypeptide drug substance.

In at least some embodiments, the compound is a molecular conjugate represented by Formula I, or a stereoisomer or a mixture of stereoisomers, or pharmaceutically acceptable salts:

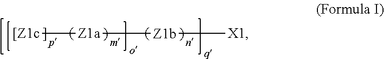

wherein:
X1 is NH₂ or OH; or
X1 comprises:
   i. a polypeptide drug substance comprising an amine;
   ii. a polypeptide drug substance that is covalently conjugated to an amine containing linker; or
   iii. an amine configured to be covalently conjugated to a polypeptide drug substance;
each Z1c is independently selected from Formulae FF1-FF224 and covalently conjugated either directly, or via Z1a and/or Z1b, to X1;
each Z1a independently comprises 1 to 50 amino acids connected together using amide or peptide bonds; each Z1b is independently a small-molecule linker;
each m′ is independently 0 or 1;
each n′ is independently 0 or a positive integer;
each o′ is independently an integer greater than or equal to 1;
each p′ is a positive integer; and
q′ is a positive integer of at least 1 and not more than two times the total number of amine groups in X1, wherein when any of n′, o′, p′, or q′ is 2 or more, the corresponding groups Z1a, Z1b, and Z1c are independently selected and may be the same or different; wherein each Z1c is independently covalently conjugated, directly or indirectly, to an amine of Z1a, to an amine of Z1b, or to X1; and wherein optionally the molecular conjugate may comprise one or more isotopes at any position of the molecular conjugate of Formula I. In some embodiments, the compound is additionally covalently conjugated as described by Formula I, and/or wherein one or more amines are each independently acetylated and/or independently alkylated.

In some embodiments, the compound of Formula I is covalently conjugated to B₁ using a covalent linkage X—B₁, wherein X is an amino group in Formula I.

In some embodiments, X1 comprises a polypeptide drug substance and the covalent conjugation to X1 is to amino group(s) in one or more lysine residues and/or to the N-terminal amino groups in X1.

In some embodiments, the compound comprises at least one of B₁, B₂ and B₃ independently selected from Formulae F1-F12 or wherein the compound comprises at least one of B₄, B₅ and B₆ independently selected from Formulae F1-F10,
wherein Formulae F1-F10 are:

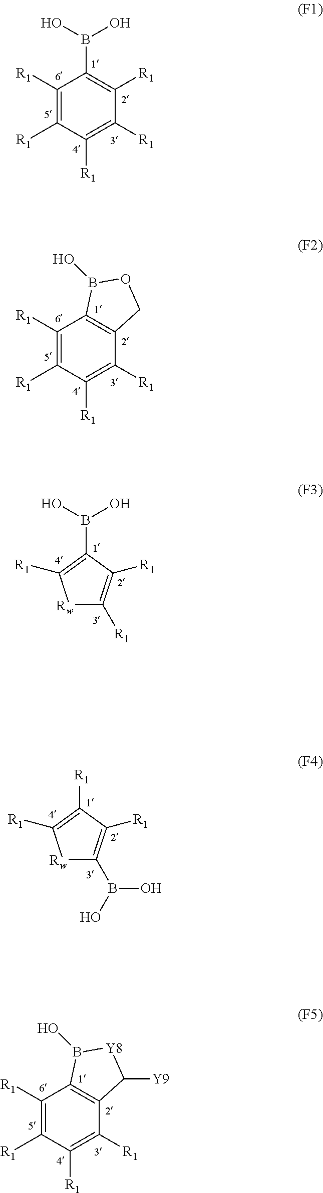

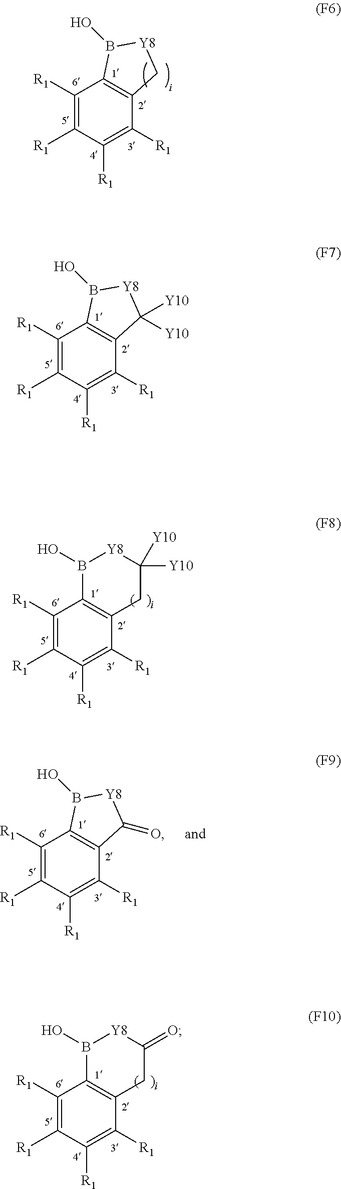

wherein for B₁, B₂, and B₃:
one R₁ represents (C=O)—*, S(═O)(═O)—*, (CH₂)ₘ(C═O)—*, or (CH₂)ₘ—*, wherein —* represents an attachment point to the rest of Z1c, and m is 1, 2, 3, 4, 5, 6, or 7; and
each remaining R₁ is independently selected from H, F, Cl, Br, OH, CH₂—NH₂, NH₂, (C═O)—NH₂, CH=O, SO₂CH₃, SO₂CF₃, CF₃, CHF₂, NO₂, CH₃, OCH₃, O(CH₂)ₘCH₃, —(SO₂)NH CH₃, —(SO₂)NH(CH₂)ₘCH₃, and OCF₃, wherein m is 1, 2, 3, 4, 5, 6, or 7;
wherein for B₄ and B₅:
one R1 for B₄ represents (CH₂)ₘ—Ø, wherein —Ø represents an attachment point to the rest of Z1c and one R₁ for B₅ represents (C═O)—*, S(═O)(═O)—*, (CH₂)ₘ(C═O)—*, or (CH₂)ₘ—*, wherein —* represents an attachment point to the rest of Z1c, and m is 1, 2, 3, 4, 5, 6, or 7; and
each remaining R₁ is independently selected from H, F, Cl, Br, OH, CH₂—NH₂, NH₂, (C═O)—NH₂, CH═O, SO₂CH₃, SO₂CF₃, CF₃, CHF₂, NO₂, CH₃, OCH₃, O(CH₂)ₘCH₃, —(SO₂)NH CH₃, —(SO₂)NH(CH₂)ₘCH₃, and OCF₃, wherein m is 1, 2, 3,4, 5, 6, or 7;
wherein for B₆:
one R₁ for B₆ represents (CH₂)ₘ—Ø, wherein —Ø represents an attachment point to the rest of the compound, and m is 1, 2, 3, 4, 5, 6, or 7; and
each remaining R₁ is independently selected from H, F, Cl, Br, OH, CH₂—NH₂, NH₂, (C═O)—NH₂, CH═O, SO₂CH₃, SO₂CF₃, CF₃, CHF₂, NO₂, CH₃, OCH₃, O(CH₂)ₘCH₃, —(SO₂)NH CH₃, —(SO₂)NH(CH₂)ₘCH₃, and OCF₃, wherein m is 1, 2, 3, 4, 5, 6, or 7;
wherein,
for Formulae F3-F4:
   R_{w} is O or S;
for Formulae F5-F10:
Y8 is selected from O, N, and NR, wherein R is an alkyl group or H;
Y9 is H, CH₃, or an alkyl group, provided that when Y8 is O, Y9 is a CH₃ or an alkyl group;
each Y10 is independently selected from H, CH₃, F, CF₃, and OCH₃; and
i is 1, 2, or 3; and
   wherein Formulae F11-F12 are:

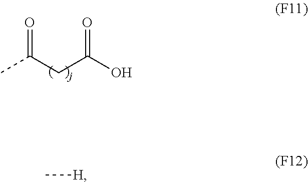

j is 1, 2, 3, 4, 5, 6, or 7; and
   — represents an attachment point to the rest of Z1c.

In at least some embodiments, B₁, B₂ and B₃ may be identical or different. If B₁, B₂ and B₃ are all present in a compound of the present disclosure, then each is independently an aromatic boron-containing group, a carboxylic acid derivative, or a H, wherein in each FF structure (i.e., FF1 to FF224) containing B₁, B₂ and B₃ groups, at least two of the B1, B2 and B3 are independently an aromatic boron-containing group.

In some embodiments, the compound comprises at least one Z1b selected from Formulae IIa-IIai or Formulae IIIa-IIIai,
wherein Formulae IIa-IIai are:

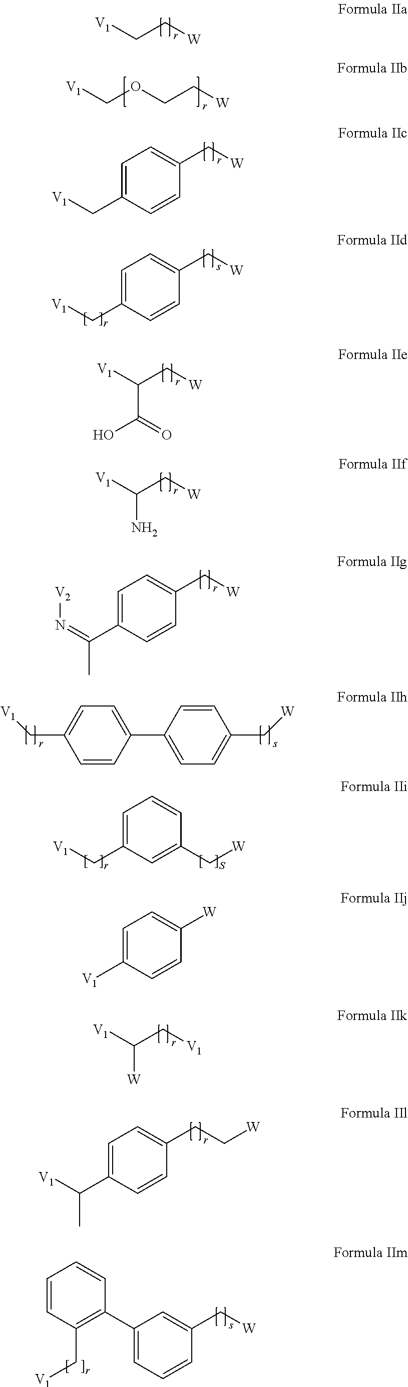

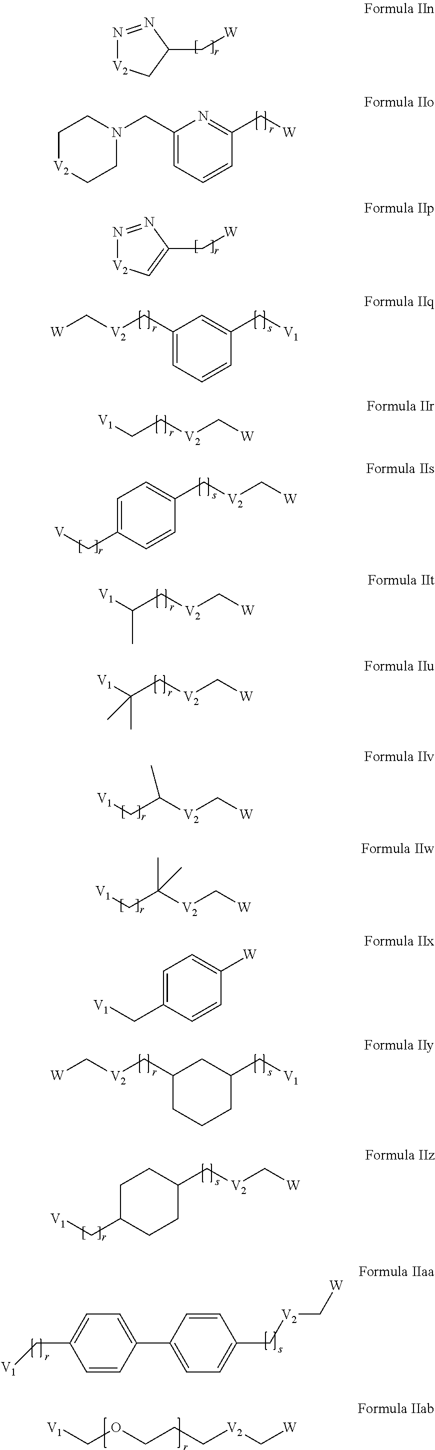

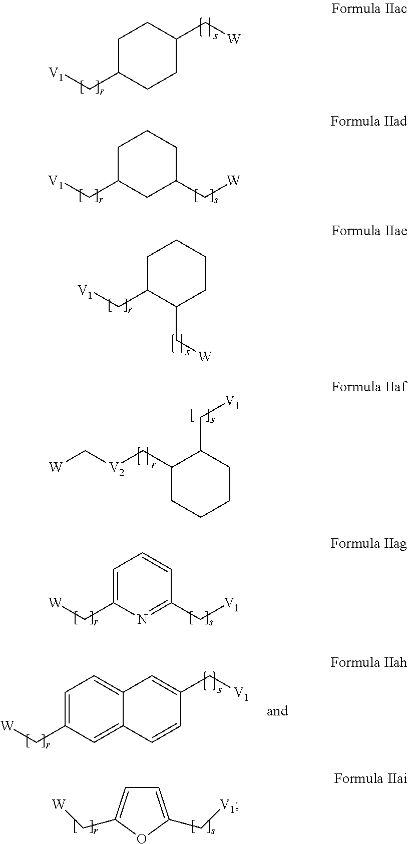

wherein:
r is 0, 1, 2,3, 4, or 5;
s is 0, 1, 2, 3, 4, or 5;
W represents CH₂—˜ or (C═O)—˜, wherein —˜ is a covalent linkage to X1; and
each V₁ is independently selected from NH—†, CH₂—†, and (C═O)—† and each V₂ is N—†, wherein —† is a covalent linkage towards successive Z1b, Z1a or Z1c, provided that V₁ is NH—† when connected to Z1c; and the covalent linkages between Z1a and Z1b units each independently comprise an amine linkage or an amide linkage; and when n′=0 and m′=1, Z1a is directly conjugated to X1 by an amine linkage or amide linkage, and
wherein Formulae IIIa-IIIai are:

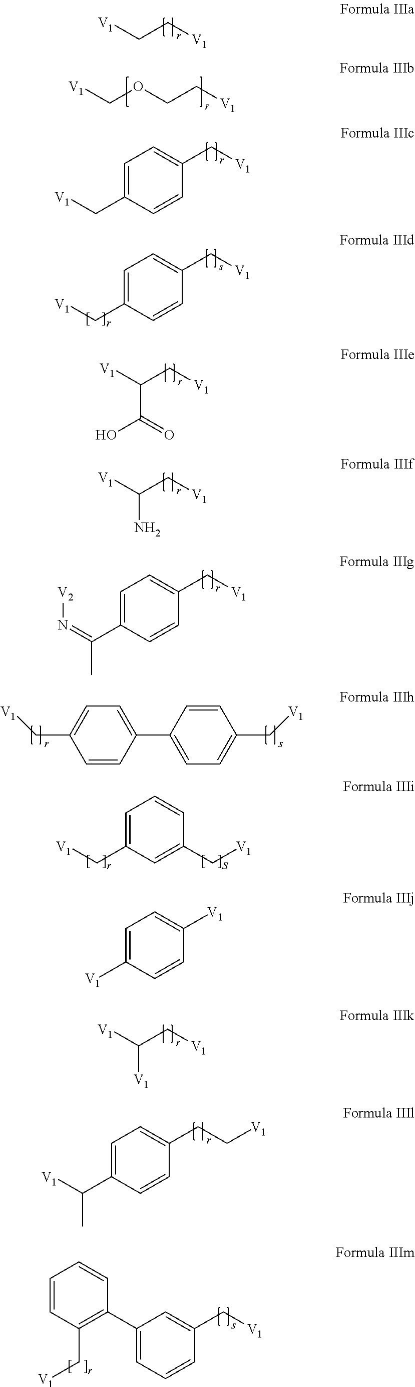

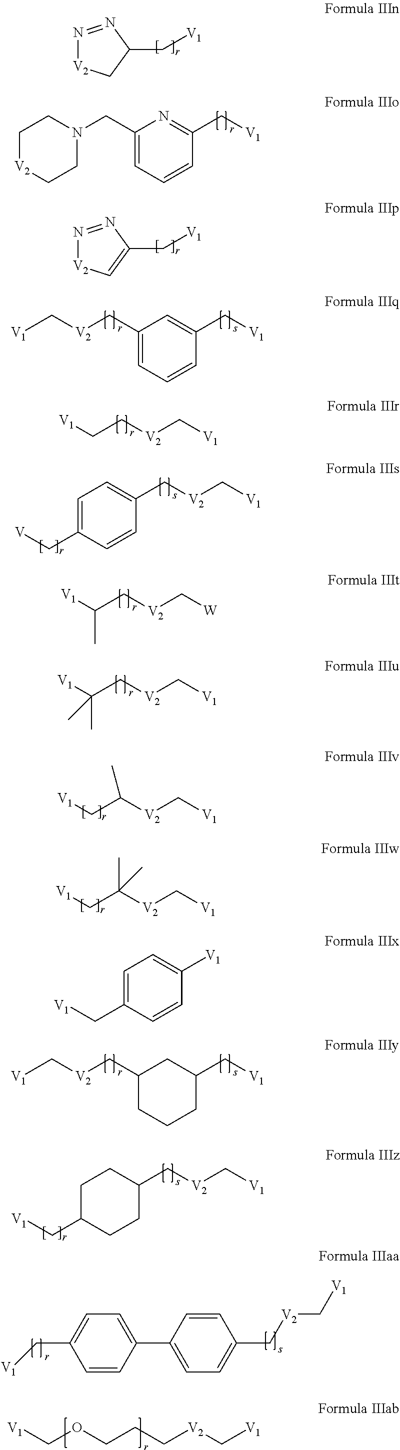

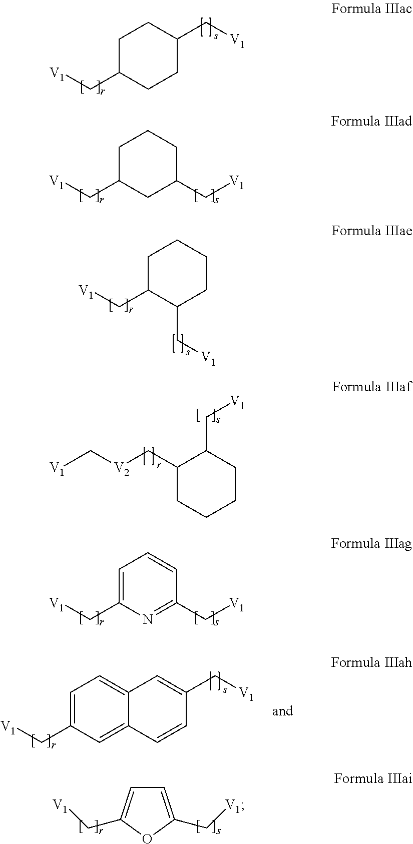

wherein:
r is 1, 2, 3, 4, or 5;
s is 1, 2, 3, 4, or 5; and
each V₁ is independently selected from NH—†, CH₂—†, and (C═O)—† and each V₂ is N—†, wherein —† is a covalent linkage towards successive Z1b, Z1a or Z1c, provided that V₁ is NH—† when connected to Z1c; and the covalent linkages between Z1a and Z1b units each independently comprise an amine linkage or an amide linkage; and when n′=0 and m′=1, Z1a is directly conjugated to X1 by an amine linkage or amide linkage.

In some embodiments, at least one Z1c is covalently conjugated indirectly via a linker (indirect linker) to the compound (e.g., the compound of Formula I). In some embodiments, the linker is selected from (i) Formulae FL1-FL19:

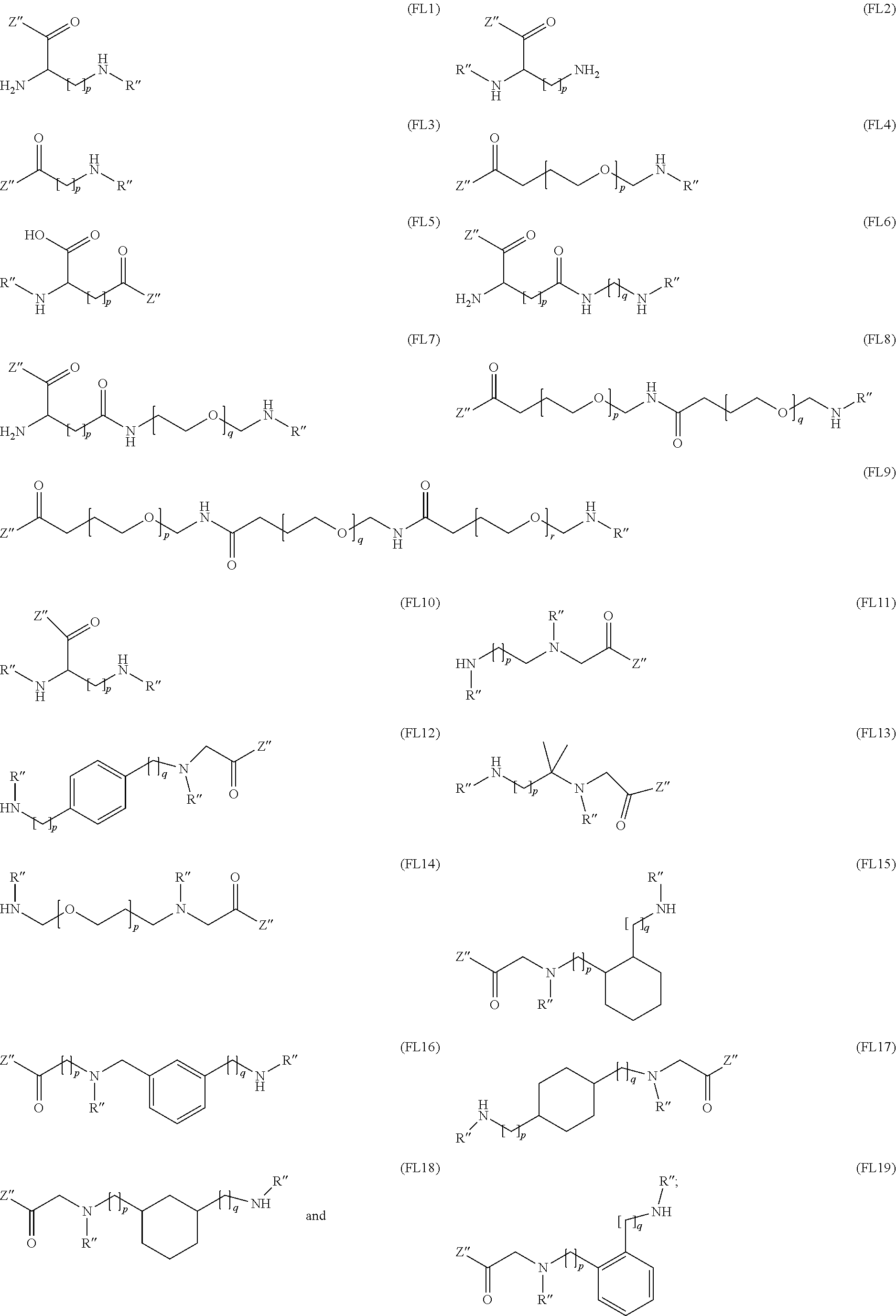

wherein, in Formulae FL1 to FL19:
Z″ represents an attachment point toward X1;
R″ represents an attachment point toward Z1c;
p is 1, 2, 3, 4, or 5,
q is 1, 2,3, 4, or 5,
r is 1, 2, 3, 4, or 5; and
any primary amine is optionally acetylated or alkylated; and
(ii) an L- or D-amino acid comprising at least one amine group directly conjugated to Z1c, wherein an acid functional group of the amino acid is conjugated toward X1 in Formula I.

In some embodiments, n′ is 1 and each of the Z1b is independently selected from (i) Formulae FL1-FL19:

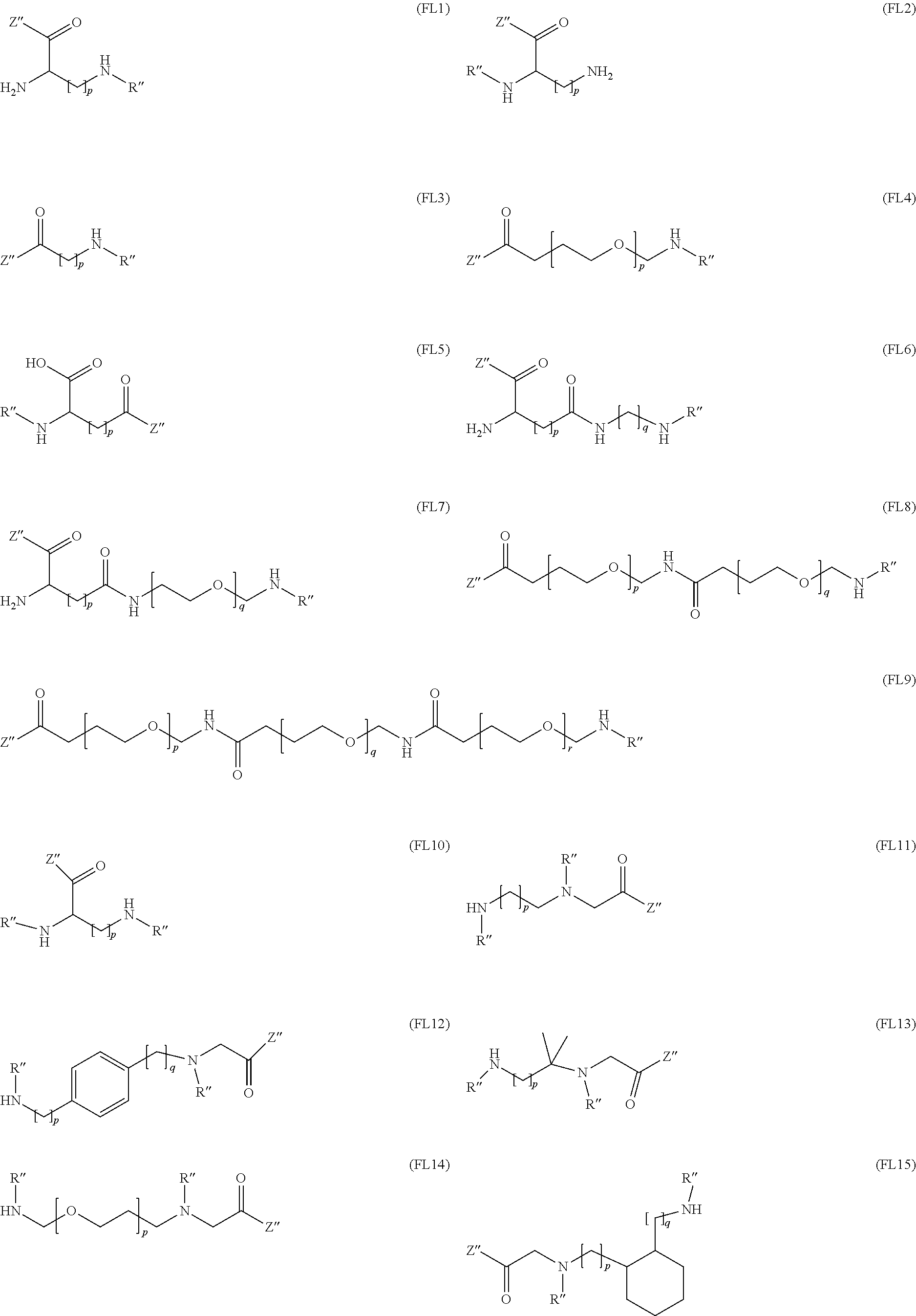

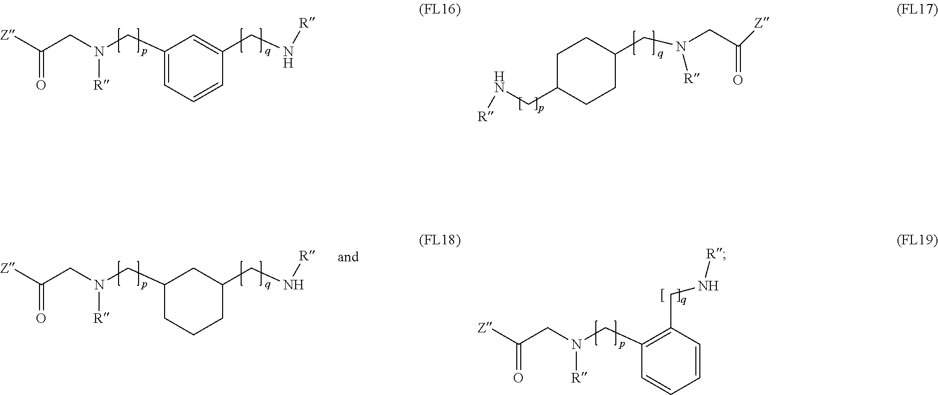

wherein, in Formulae FL1 to FL19:
Z″ represents an attachment point toward X1;
R″ represents an attachment point toward Z1c;
p is 1, 2, 3, 4, or 5,
q is 1, 2, 3, 4, or 5,
r is 1, 2, 3, 4, or 5; and
any primary amine is optionally acetylated or alkylated; and
(ii) an L- or D-amino acid comprising at least one amine group directly conjugated to Z1c, wherein an acid functional group of the amino acid is conjugated toward X1 in Formula I.

In some embodiments, the compound of Formula (I) is selected from:

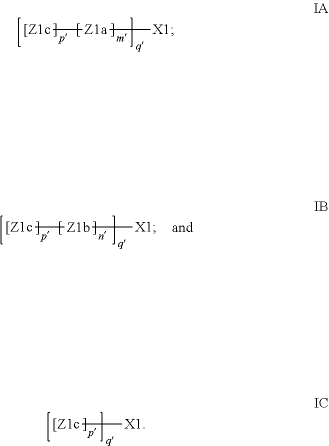

In some embodiments, the compound of Formula I is selected from:

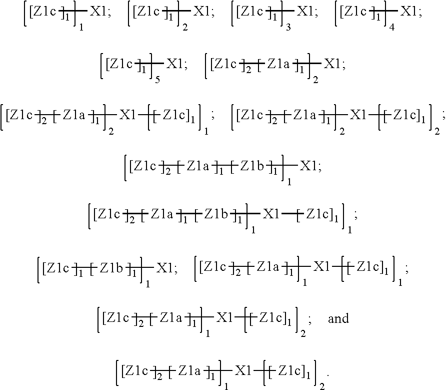

In some embodiments, the compound of Formula I is selected from:

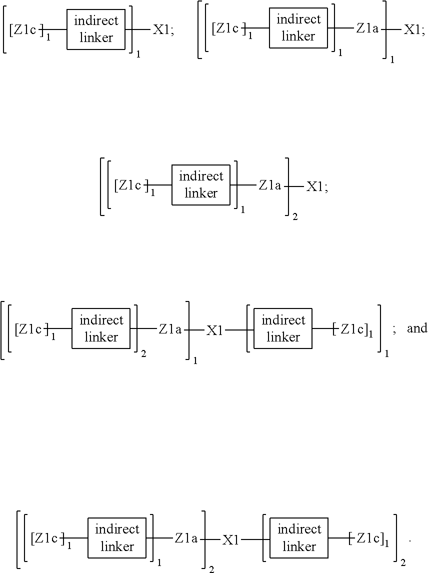

In some embodiments, the compound is

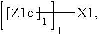

when p′=1, m′=0, o′=1, n′=0, and q′=1.

In some embodiments, the compound is

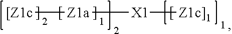

when p′=1 and 2, m′=0 and 1, o′=1 and 1, n′=0, and q′=2 and 1.

In some embodiments, in Formula I, Z1c is directly connected to X1 through an optional covalent-spacer, and the optional covalent-spacer is independently selected from gamma-glutamic acid, beta-alanine, and

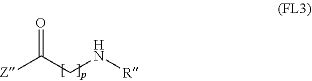

Formula FL3, wherein p is 1 or 2; and

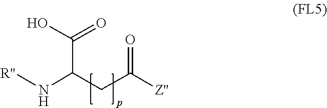

Formula FL5, wherein p is 2, 3, or 4;

In some embodiments, X1 is OH or NH₂, and X1 further comprises a drug substance covalently conjugated directly or indirectly to the compound.

In at least some embodiments, the compound of the present disclosure comprises a drug substance comprising a polypeptide hormone, a human polypeptide hormone and/or insulin, or an analogue thereof, or a hybrid polypeptide comprising one or more combinations thereof.

In at least some embodiments, the compound of the present disclosure comprises an amine in the compound that is conjugated via an amide linkage to an aromatic boron-containing compound (e.g., group). In some embodiments, the aromatic boron-containing group is selected from a phenylboronic acid, boroxole, and phenylboronate.

In at least some embodiments, the compound of the present disclosure is dehydrated (loses) by 1, 2, 3, 4, 5, 6, 7, 8, or more water molecules.

In at least some embodiments, the compound of the present disclosure is formulated in a solution comprising one or more of a buffer, stabilizer, vasodilator, preservative, surfactant, salt, sugar, or compounds containing one or more hydroxyls, alcohols, diols, or phenols. For example, the solution could comprise one or more of citrate, zinc, and/or cresol.

In at least some embodiments, X1 comprises a human polypeptide hormone of the human pancreas, insulin, glucagon, GLP-1, a somatostatin, a gastric inhibitory polypeptide, a glucose-dependent insulinotropic polypeptide, a hybrid peptide comprising sequences from two or more human polypeptide hormones, or an analogue thereof.

In some embodiments, X1 comprises human insulin or a human insulin analogue comprising an A-chain and a B-chain, wherein the A-chain comprises a sequence selected from SEQ ID NOs 1 and 3 to 33, and the B-chain comprises a sequence selected from SEQ ID NOs 2 and 34 to 74, 24047, and 24048;
each Z1c is independently selected from FF1, FF10, FF12, FF14, FF15, FF114, FF115, FF116, FF163, FF193, FF194, FF203, and FF221-FF224 and covalently conjugated either directly, or indirectly via the linker, to Z1a and/or Z1b, or to X1;
each Z1a is independently absent or independently comprises a sequence selected from K, GK, KGSH (SEQ ID NO:24049), KGSHK (SEQ ID NO:4238), KNSTK (SEQ ID NO:5085), GKASHK (SEQ ID NO:12414), GKEEEK (SEQ ID NO:12677), GKEEHK (SEQ ID NO:12680), GKGHSK (SEQ ID NO:13120), GKGSH (SEQ ID NO:24050), GKGSHK (SEQ ID NO:13198), GKGSTK (SEQ ID NO:13205), GKHENK (SEQ ID NO:13271), GKNSHK (SEQ ID NO:13982), GKNSTK (SEQ ID NO:13989), GKQSSK (SEQ ID NO:14380), GKYQFK (SEQ ID NO:15128), GKGSKK (SEQ ID NO:24045), GKKPGKK (SEQ ID NO:24046), GKGPSK (SEQ ID NO:24044), GKPSHKP (SEQ ID NO:24043), and GSHKGSHK (SEQ ID NO:24042);
each linker is selected from FL1, FL3, FL4, and FL5;
each m′ is independently 0 or 1;
each n′ is independently 0, 1, 2, or 3;
each o′ is independently 1, 2, 3, 4, or 5;
each p′ is 1, 2, 3, 4, or 5; and
q′ is 1, 2, 3, or 4, wherein when any of n′, o′, p′, or q′ is 2 or more, the corresponding groups Z1a, Z1b, and Z1c are independently selected and may be the same or different; and
wherein each Z1c is independently covalently conjugated, directly or indirectly, to an amine of Z1a, to an amine of Z1b, or to X1.

In some embodiments, X1 comprises the human insulin or human insulin analogue comprising an A-chain and a B-chain, wherein the A-chain comprises SEQ ID NO:1; and the B-chain is selected from SEQ ID NOs 2, 36, 24047, and 24048;
each Z1c is independently selected from FF1, FF10, FF12, FF14, FF15, FF114, FF115, FF116, FF193, FF194, FF203, and FF221-FF224 and covalently conjugated either directly, or indirectly via the linker, to Z1a and/or Z1b, or to X1;
each Z1a independently comprises a sequence selected from K, GK, KGSH (SEQ ID NO:24049), KGSHK (SEQ ID NO:4238), KNSTK (SEQ ID NO:5085), GKASHK (SEQ ID NO:12414), GKEEEK (SEQ ID NO:12677), GKEEHK (SEQ ID NO:12680), GKGHSK (SEQ ID NO:13120), GKGSH (SEQ ID NO:24050), GKGSHK (SEQ ID NO:13198), GKGSTK (SEQ ID NO:13205), GKHENK (SEQ ID NO:13271), GKNSHK (SEQ ID NO:13982), GKNSTK (SEQ ID NO:13989), GKQSSK (SEQ ID NO:14380), GKYQFK (SEQ ID NO:15128), GKGSKK (SEQ ID NO:24045), GKKPGKK (SEQ ID NO:24046), GKGPSK (SEQ ID NO:24044), GKPSHKP (SEQ ID NO:24043), and GSHKGSHK (SEQ ID NO:24042);
each linker is independently absent or independently selected from FL3 and FL5;
each m′ is independently 0 or 1;
each n′ is independently 0 or 2;
each o′ is independently 1, 2, or 3;
each p′ is 1, 2, or 3; and
q′ is 1, 2, or 3, wherein when any of n′, o′, p′, or q′ is 2 or more, the corresponding groups Z1a, Z1b, and Z1c are independently selected and may be the same or different;
wherein each Z1c is independently covalently conjugated, directly or indirectly, to an amine of Z1a, to an amine of Z1b, or to X1.

In some embodiments, each of the Z1a is independently absent or independently comprises a sequence selected from K, GK, KGSH (SEQ ID NO:24049), GKGSH (SEQ ID NO:24050), KGSHK (SEQ ID NO:4238), and GKGSHK (SEQ ID NO:13198).

In some embodiments, each of the Z1c is independently selected from FF1, FF10, FF12, FF14, FF15, FF114, FF115, FF116, and FF221-FF224. In some embodiments, B₁ and B₂ are independently selected from Formulae F1 and F2. In some embodiments, the B₁ and the B₂ are F2. In some embodiments, at least one R1 in B₁ or B₂ is F or CF₃. In some embodiments, Z1b is independently absent, FL3, or FL5. In some embodiments, each of the Z1c is independently selected from FF10, FF12, FF116, FF221, FF222, and FF224. In some embodiments, each B₁ and B₂ is F2 and is covalently conjugated to Z1c using an amide linkage,
each Z1b is independently absent; FL3 wherein p is 1, 2, or 3; or FL5 wherein p is 2, 3, or 4;
each FF is independently selected from FF10, FF12, FF116, FF134, FF163, FF193, FF203, FF221, FF222 and FF224; wherein FF12 and FF222 has either (S,R) or (S,S) stereochemistry;
each Z1c is conjugated either directly or indirectly through FL3 or FL5 to the amine group in one or more lysine side chain in X1 or the N-terminus in X1; and
X1 is a polypeptide drug substance and/or an insulin optionally having from 0 to 4 residues replaced, inserted, or mutated to lysines, and wherein the lysines are each conjugated directly or indirectly to a Z1c.

In some embodiments, Z1c is FF224, n′ is 0, and Z1a is an amine containing amino acid.

In some embodiments, the compound is selected from

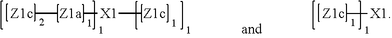

In some embodiments, Z1c is covalently conjugated directly to X1 via a linker, and wherein the linker is independently selected from gamma-glutamic acid, beta-alanine, and

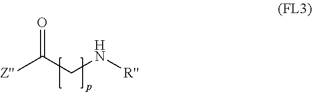

Formula FL3,
wherein p is 1, 2, or 3; and

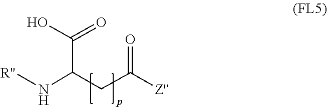

Formula FL5,
wherein p is 2, 3, or 4.

In some embodiments, the compound further comprises a drug substance covalently conjugated directly or indirectly to the compound.

In some embodiments, the compound of Formula I is selected from Examples 315, 318, 320, 605-608, 610-612, 589-595, 562-574, and 803-876.

In some embodiments, X1 is a polypeptide drug substance and/or an insulin optionally having from 0 to 4 residues replaced, inserted, or mutated to lysines, and wherein the lysines are each conjugated to a Z1c.

In some embodiments, one or more amines are each independently acetylated and/or independently alkylated.

In some embodiments, X1 comprises a polypeptide drug substance and the covalent conjugation to X1 is to amino group(s) in one or more lysine residues and/or to the N-terminal amino groups in X1.

In some embodiments, each R1 in FF1-FF224 is independently selected from a C₁-C₂₂ alkyl group, a C₁-C₂₂ acyl group, a (C₃-C₈)cycloalkyl group, a C₁-C₂₂ haloalkyl group, an aryl group, and a heteroaryl group, each R1 optionally comprises one or more C₁-C₂₂ alkyl-halide, halide, sulfhydryl, aldehyde, amine, acid, hydroxyl, C₁-C₂₂ alkyl, or aryl groups.

In some embodiments, X4 is selected from —COOH, —(CH₂)ₘCOOH, a C₁-C₂₂ alkyl group, a C₁-C₂₂acyl group, a (C₃-C₈)cycloalkyl group, a C₁-C₂₂ haloalkyl group, an aryl group, and a heteroaryl group, each X4 optionally comprises one or more C₁-C₂₂ alkyl-halide, halide, sulfhydryl, aldehyde, amine, acid, hydroxyl, C₁-C₂₂ alkyl, or aryl groups; wherein m is 1, 2, 3, 4, or 5.

In some embodiments, the alkyl group of Y9 is a C₁-C₂₂ alkyl. In some embodiments, Y9 is CH₃.

In some embodiments, at least one primary or secondary amine in FF1-FF223 is covalently conjugated to B6.

In some embodiments, an amine in the compound is conjugated via an amide linkage to an aromatic boron-containing group.

In some embodiments, the aromatic boron-containing group is selected from a phenylboronic acid, boroxole, and phenylboronate.

In some embodiments, the compound is formulated in a solution comprising one or more of a buffer, stabilizer, vasodilator, preservative, surfactant, salt, sugar, or compounds containing one or more hydroxyls, alcohols, diols, or phenols. In some embodiments, the solution comprises one or more of citrate, zinc, and/or cresol.

In some embodiments, Z1c is conjugated to a cysteine.

In some embodiments, the compound (e.g., the compound of Formula I) is covalently conjugated either directly or through a linker to a diol, sugar, carbohydrate or a diol containing molecule.

In some embodiments, the compound (e.g., the compound of Formula I) is covalently conjugated to an antibody, albumin or a fragment thereof, or covalently conjugated either directly or through a linker to a molecule that can bind to at least one protein present in human plasma. In at least one embodiment, the present disclosure provides a method to administer the compounds disclosed herein to a human subject as a therapeutic or prophylactic agent.

In some embodiments, the compounds disclosed herein are used as intermediate compounds for the manufacture of any compounds disclosed herein.

In some embodiments, the compounds disclosed herein comprise at least one Z1c. In at least some embodiments, the Z1c is a boron containing compound. In some embodiments, a subset of boron containing compounds is selected from a non-aromatic and/or an aromatic boron-containing group. In some embodiments, Z1c is an aromatic boron-containing group. In at least one embodiment, the compound of the present disclosure comprises at least one Z1c selected from:

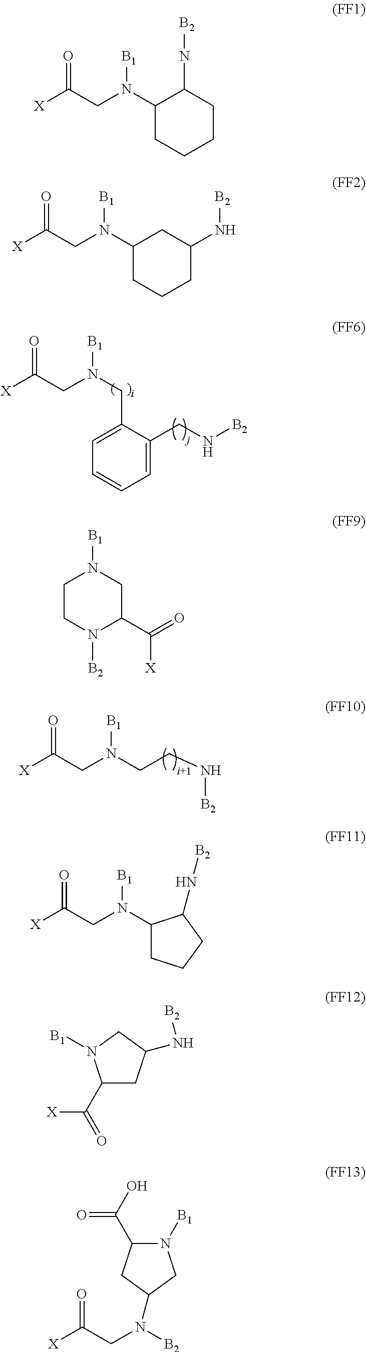

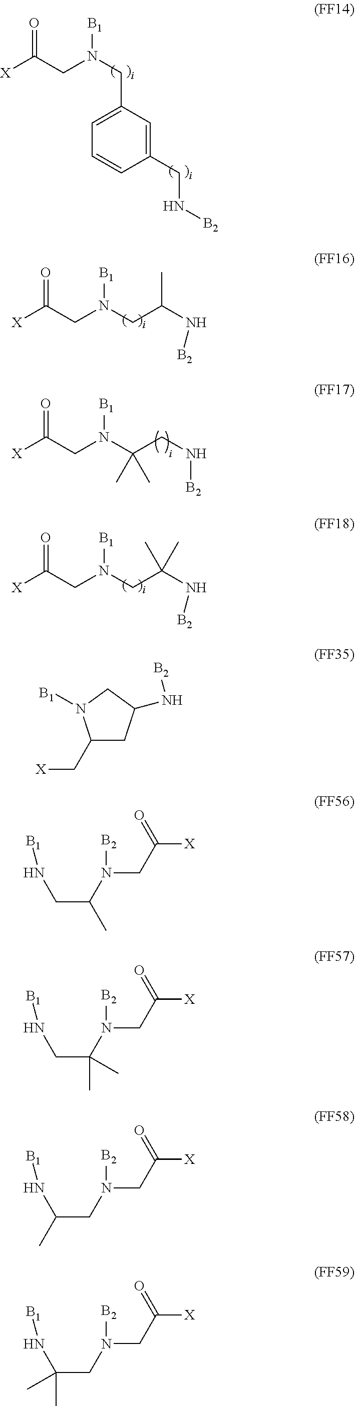

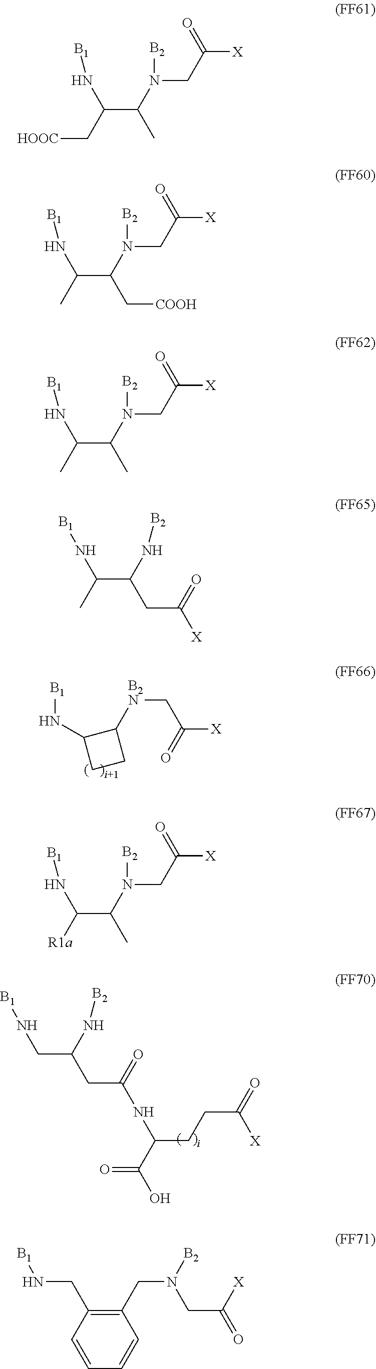

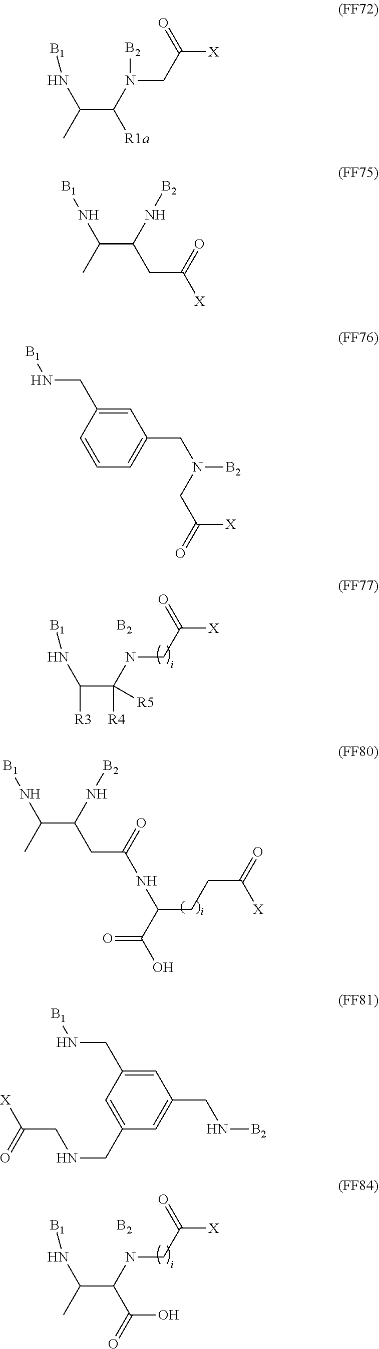

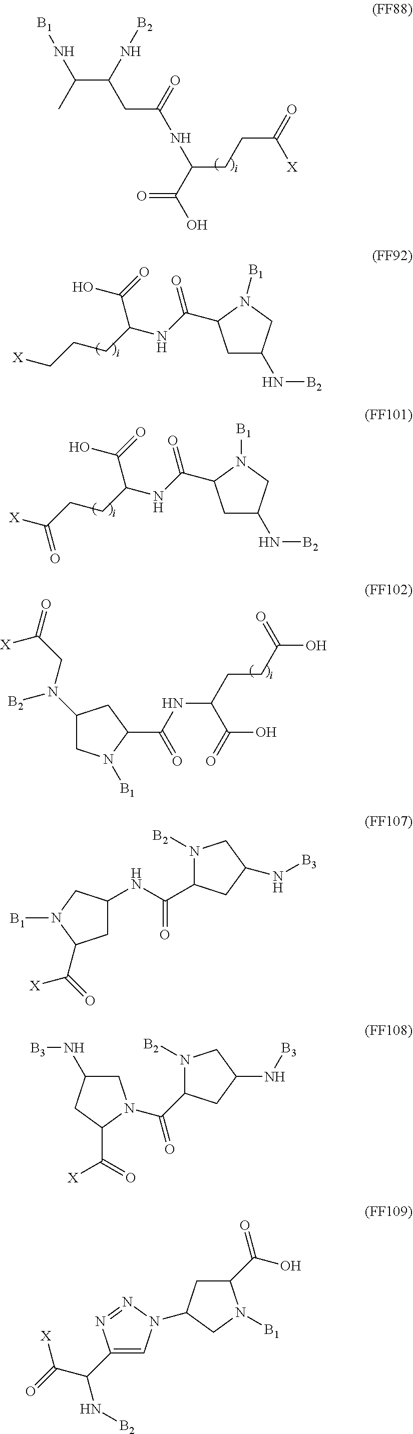

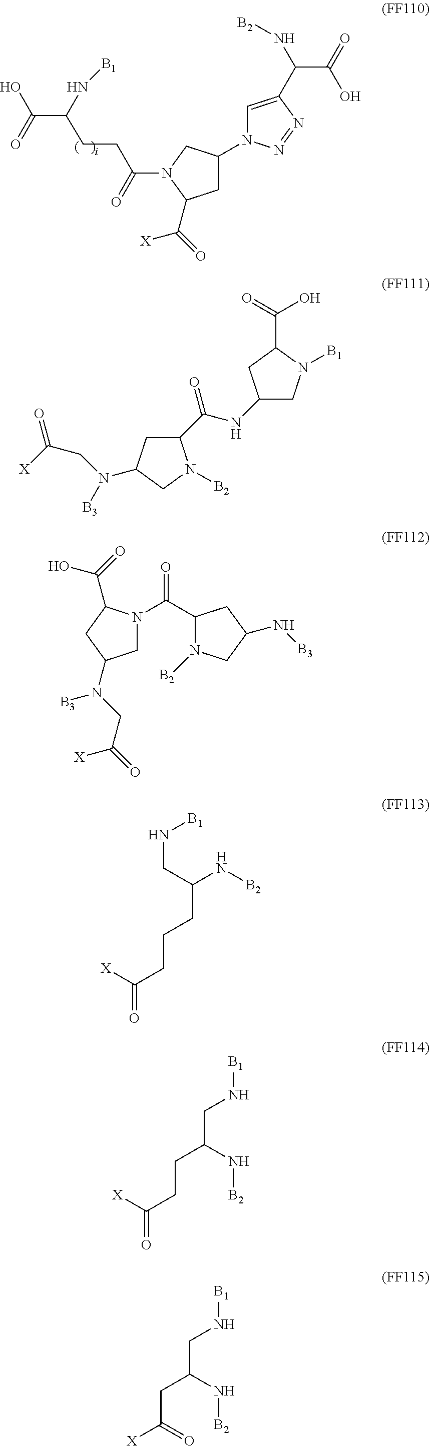

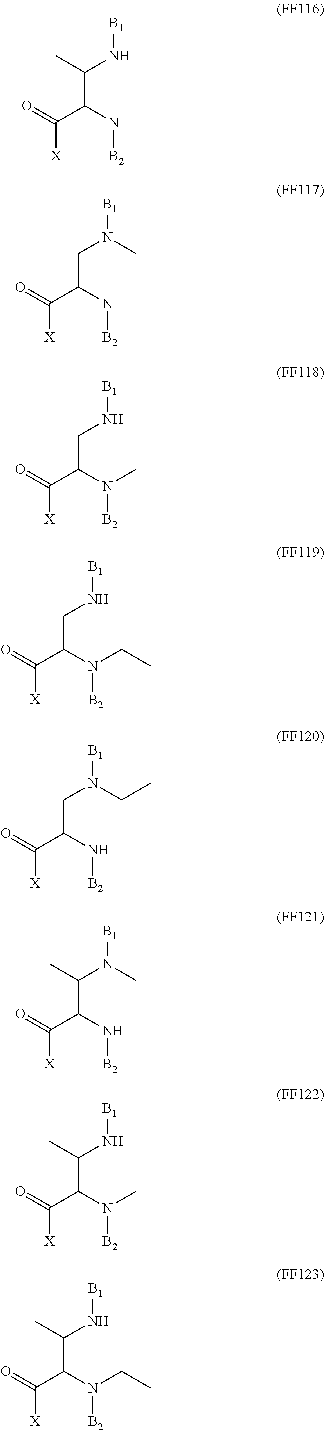

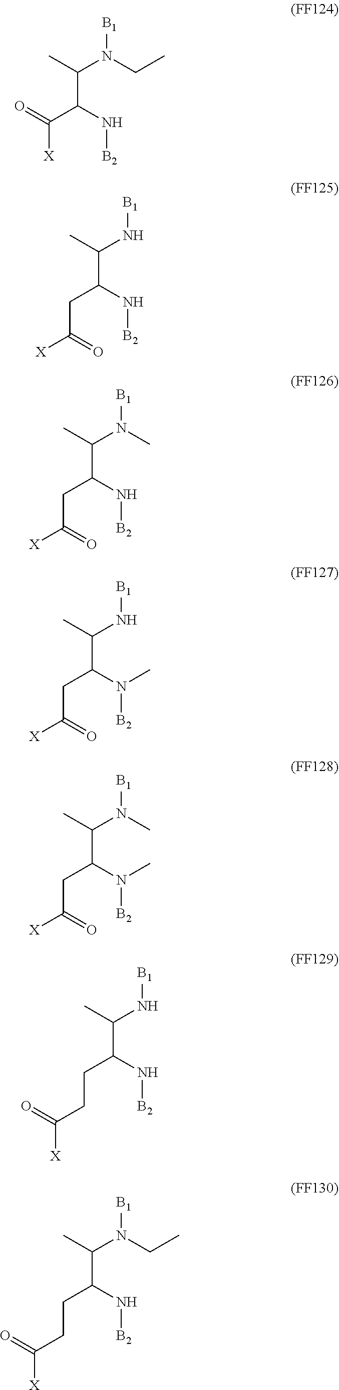

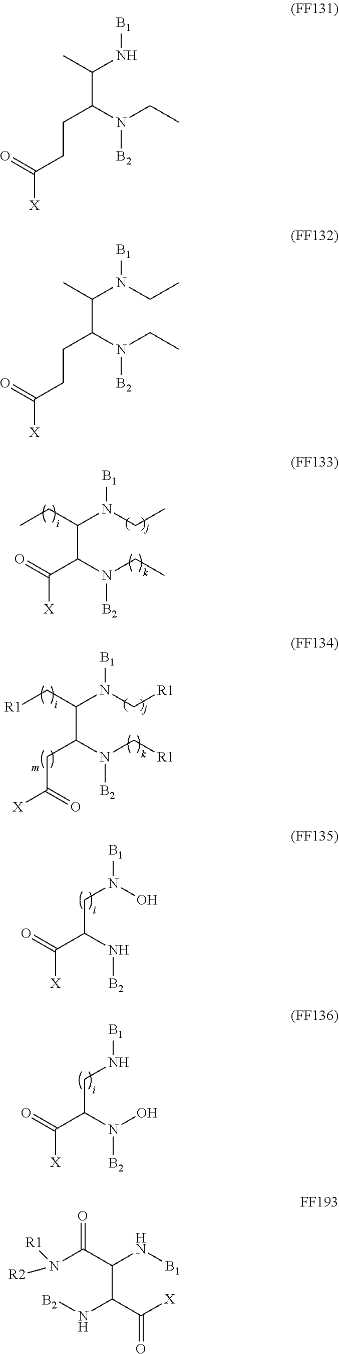

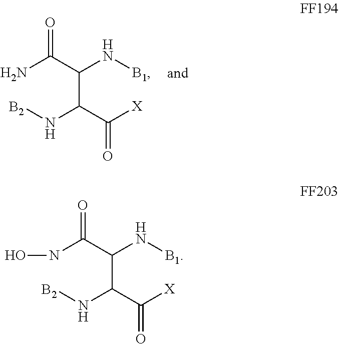

In at least some embodiments, the Z1c is selected from FF1-FF224. In some embodiments, the compound comprises at least one Z1c having at least one chiral center and selected from FF1, FF2, FF5, FF9, FF11-FF13, FF15-FF24, FF27, FF31, FF34-FF36, FF38, FF39, FF43-FF58, FF60-FF70, FF72-FF75, FF77-FF80, FF82-FF84, FF86-FF212, FF216-FF220, FF222, FF223, and combinations thereof.

In some embodiments, the compound comprises at least one FF12 and/or FF116. In some embodiments, the stereochemistry of FF12 and FF116 is independently selected from (S,S); (S,R); (R,R); and (R,S).

In some embodiments, X1 comprises human insulin or a human insulin analogue comprising an A-chain and a B-chain, wherein the C-terminus of the A-chain of the human insulin analogue is optionally extended with a polypeptide of up to 20 residues, and/or the N-terminus of the B-chain of the human insulin analogue is optionally extended with a polypeptide of up to 10 residues. In some embodiments, one to six residues of the insulin A-chain and/or the insulin B-chain are deleted or mutated.

In some embodiments, X1 comprises at least one lysine having an amine side chain, and Z1c is covalently conjugated directly to the amine side chain. In some embodiments, the compound of the present disclosure comprises at least one Z1a comprising one or more amino acids having an amine side chain, and wherein the one or more amino acids are selected from lysine, diaminopropionic acid, diaminobutyric acid, and ornithine; and wherein Z1c is covalently conjugated, directly or indirectly, to the amine side chain.

In some embodiments, the compound of the present disclosure may include one or more isotopes selected from deuterium, tritium, carbon-13, carbon-14, and iodine-124. In at least one embodiment, the compound comprises deuterium.

In some embodiments, X1 comprises a drug substance covalently conjugated to at least one Z1c through an acid containing linker. In some embodiments, a composition of the present disclosure comprises at least one compound as disclosed herein (e.g., a compound comprising X1 and one or more Z1c, Formula I), or a tautomer, stereoisomer or a mixture of stereoisomers, or a pharmaceutically acceptable salt, or hydrate, or isotopic derivative thereof formulated together with one or more pharmaceutically acceptable carriers.

In some embodiments, the present disclosure also provides a composition or a mixture comprising at least one compound as disclosed herein, for use as a medicament for the treatment of diabetes, for control of blood sugar levels, or to control the release of a drug based on physiological levels of diol containing small molecules or sugars.

In some disclosed embodiments are a method of administering a compound as disclosed herein to a human subject as a therapeutic or prophylactic agent.

In some embodiments, the disclosure provides a method of making a compound as disclosed herein comprising at least one alkylation and/or amidation step.

In some embodiments, the disclosure provides a method of treating a subject by administering a device or formulation comprising a compound as disclosed herein, such as Examples 1-880. For example, the device can be a fixed dose injector, microdosing injector, an internal or external patch.

In some embodiments, the disclosure provides a method of treating or preventing diabetes, impaired glucose tolerance, hyperglycemia, or metabolic syndrome (metabolic syndrome X, insulin resistance syndrome) comprising administering to a subject in need thereof a therapeutically effective amount of a compound disclosed herein.

In at least some embodiments, the present disclosure is directed to a compound of Formulae FF1-FF224, or a tautomer, stereoisomer or a mixture of stereoisomers, or a pharmaceutically acceptable salt, or hydrate, or isotopic derivative thereof. In at least some embodiments, the present disclosure is directed to a compound selected from Formulae FF1-FF48, Formulae FF49-FF88, FF89-FF112, FF113-FF136, FF137-FF160, FF161-FF164, FF165-FF166, FF167-FF192, FF193-FF209, and FF210-FF224.

In some embodiments, the present disclosure is directed to a compound selected from Formulae FF1-FF48,
wherein X is selected from an amine, OH, and halogen; and
i is 1, 2, 3, 4, 5, 6, or 7;
j is 1, 2, 3, 4, 5, 6, or 7; and
B₁ and B₂, which may be identical or different, each independently represents an aromatic boron-containing group.

In some embodiments, the present disclosure is directed to a compound selected from Formulae FF49-FF88,
wherein X is selected from an amine, OH, and halogen;
i is 1, 2, 3, 4, 5, 6, or 7;
j is 1, 2, 3, 4, 5, 6, or 7;
R1a is selected from COOH, CH₃, H, and OH;
R2, R3, R4 and R5 is each independently selected from CH₃, H, OH, and COOH, and at least one of R2, R3, R4 and R5 is CH₃ or OH; and
B₁ and B₂, which may be identical or different, are each independently an aromatic boron-containing group.

In some embodiments, the present disclosure is directed to a compound selected from Formulae FF89-FF112,
wherein X is selected from an amine, OH, and halogen;
i is 1, 2, 3, 4, 5, 6, or 7; and
B₁, B₂ and B₃, which may be identical or different, each independently represents an aromatic boron-containing group, a carboxylic acid derivative, or a H, wherein at least two of B1, B2 and B3 in each FF structure are independently an aromatic boron-containing group.

In some embodiments, the present disclosure is directed to a compound selected from Formulae FF113-FF136,
wherein X is selected from an amine, OH, and halogen;
i is 0, 1, 2, 3, 4, 5, 6, or 7;
j is 0, 1, 2, 3, 4, 5, 6, or 7;
k is 0, 1, 2, 3, 4, 5, 6, or 7;
m is 0, 1, 2, 3, 4, 5, 6, or 7;
wherein i+j+k+m is greater than 0
each R1 is independently selected from H, an alkyl group, an acyl group, a cycloalkyl group, a haloalkyl group, an aryl group, and a heteroaryl group, each R1 optionally comprises one or more alkyl-halide, halide, sulfhydryl, aldehyde, amine, acid, hydroxyl, alkyl or aryl groups; and
B₁ and B₂, which may be identical or different, each independently represents an aromatic boron-containing group.

In some embodiments, the present disclosure is directed to a compound selected from Formulae FF137-FF160,
wherein X is selected from an amine, OH, and halogen;
i is 0, 1, 2, 3, 4, 5, 6, or 7;
j is 0, 1, 2, 3, 4, 5, 6, or 7;
k is 0, 1, 2, 3, 4, 5, 6, or 7;
m is 0, 1, 2, 3, 4, 5, 6, or 7;
wherein i+j+k+m is greater than 0;
each R1 is independently selected from H, an alkyl group, an acyl group, a cycloalkyl group, a haloalkyl group, an aryl group, and a heteroaryl group, each R1 optionally comprises one or more alkyl-halide, halide, sulfhydryl, aldehyde, amine, acid, hydroxyl, alkyl or aryl groups; and
B₁ and B₂, which may be identical or different, each independently represents an aromatic boron-containing group.

In some embodiments, the present disclosure is directed to a compound selected from Formulae FF161-FF164,
wherein X is selected from an amine, OH, and halogen;
i is 1, 2, 3, 4, or 5 (e.g., 1, 2, 3, or 5);
j is 1, 2, 3, 4, or 5 (e.g., 1, 2, 3, or 5);
each R6, R7, R8, and R9 for different values of j is independently selected from H, CF₃, CH₃, CHF₂, and (CH₂)ₘCH₃, wherein m is 1, 2, 3, 4, or 5;
Y3, Y4, Y5, Y6 and Y7 are each independently selected from H, CH₂—X4, and Formulae IV-1 to IV-135 (as previously defined);
wherein X4 is selected from —COOH, —(CH₂)ₘCOOH, an alkyl group, an acyl group, a cycloalkyl group, a haloalkyl group, an aryl group, and a heteroaryl group, each optionally comprising one or more alkyl-halide, halide, sulfhydryl, aldehyde, amine, acid, hydroxyl, alkyl or aryl groups; wherein m is 1, 2, 3, 4, or 5;
wherein at least one of Y5, Y6, and Y7 in Formulae FF162 and FF163 is not H; and
at least one of Y7, R8 and R9 in FF164 is not H; and
Xa represents CH═O, CHF₂, CF₃, CH₂SH, COOH, CH₂OH, CH₂NO₂, CH₂NH₂, CH₃, C(CH₃)₃, CH(CH₃)₂, CH((CH₂)₃CH₃)₂, or CH(CH₂CH₃)₂;
Xb represents O, NH, CH₂, or S;
Xc represents CH or N;
each R₁₀ is independently selected from H, F, Cl, Br, CH₃, CF₃, CH═O, OH, COOH, and (CH₂)·CH₃,
m is 1, 2, 3, 4, or 5; and n is 1, 2, 3, 4, or 5; and
B₁ and B₂, which may be identical or different, each independently represents an aromatic boron-containing group. In some embodiments, when j is 4, X is not NH₂ for FF163.

In some embodiments, the present disclosure is directed to a compound selected from Formulae FF165-FF166,
wherein X is selected from an amine, OH, and halogen;
m is 1, 2, 3, 4, 5, 6, or 7;
n is 1, 2, 3, 4, 5, 6, or 7;
X5 is S, O, or NH; and
each R₁ is independently selected from H, F, Cl, Br, OH, CH₂—NH₂, NH₂, (C═O)—NH₂, CH═O, SO₂CH₃, SO₂CF₃, CF₃, CHF₂, NO₂, CH₃, OCH₃, O(CH₂)ₘCH₃, —(SO₂)NH CH₃, —(SO₂)NH(CH₂)ₘCH₃, and OCF₃, wherein m is 1, 2, 3, 4, 5, 6, or 7.

In some embodiments, the present disclosure is directed to a compound selected from Formulae FF167-FF192,
wherein X is selected from an amine, OH, and halogen; and
B₁ and B₂, which may be identical or different, each independently represents an aromatic boron-containing group.

In some embodiments, the present disclosure is directed to a compound selected from Formulae FF193-FF209,
wherein R in FF208 and FF209 is an alkyl, aryl or halide that is covalently conjugated through at least one CH₂ group to the amino group in the side chain of FF208 or FF209;
R1 and R2 are independently selected from H, CH₃, alkyl, and formulae IV-1 to IV-135;
i is 1, 2, 3, 4, or 5;
j is 1, 2, 3, 4, or 5; and
wherein X is selected from an amine, OH, and halogen; and
B₁ and B₂, which may be identical or different, each independently represents an aromatic boron-containing group.

In some embodiments, the present disclosure is directed to a compound selected from Formulae FF210-FF224,
wherein R11 in FF210 to FF212 is independently selected from Formulae IV-1 to IV-135 and R12 is selected from an amine, a hydroxyl, an alkyl, and a halide group;
wherein each R13 is independently selected from H, CH₃, alkyl, aryl, and formulae IV-1 to IV-135; R14 is selected from H, CH₃, alkyl, aryl, and heteroaryl; wherein X is independently selected from an amine, OH, and halogen;
X″ is an amine;
i is 1, 2, 3, 4, or 5;
j is 1, 2, 3, 4, or 5; and
B₁, B₂, B₃, B₄, B₅, and B₆, each independently represents an aromatic boron-containing group, wherein in any compound containing B₁, B₂ and B₃ groups, at least two groups are independently an aromatic boron-containing group.

In at least some embodiments, when X is an amine in any one of Formulae FF1 to FF223, X is optionally acetylated or alkylated.

In some embodiments, the compound comprises at least one of B₁, B₂ and B₃ independently selected from Formulae F1-F12 or wherein the compound comprises at least one of B₄, B₅ and B₆ independently selected from Formulae F1-F10. In at least some embodiments, B1, B₂ and B₃ may be identical or different. If B1, B₂ and B₃ are all present in a compound of the present disclosure, then each is independently an aromatic boron-containing group, a carboxylic acid derivative, or a H, with the proviso that in each FF structure (i.e., FF1 to FF224) containing B1, B₂ and B₃ groups, at least two groups are independently an aromatic boron-containing group.

In some embodiments, for B1, B₂, B₃:
one R1 represents (C═O)—*, S(═O)(═O)—*, (CH₂)ₘ(C═O)—*, or (CH₂)ₘ—*, wherein —* represents an attachment point to the rest of Z1c, and m is 1, 2, 3, 4, 5, 6, or 7;
each remaining R₁ is independently selected from H, F, Cl, Br, OH, CH₂—NH₂, NH₂, (C═O)—NH₂, CH═O, SO₂CH₃, SO₂CF₃, CF₃, CHF₂, NO₂, CH₃, OCH₃, O(CH₂)ₘCH₃, —(SO₂)NH CH₃, —(SO₂)NH(CH₂)ₘCH₃, and OCF₃, wherein m is 1, 2, 3, 4, 5, 6, or 7;

In some embodiments, for B₄, B₅:
one R₁ for B₄ represents (CH₂)ₘ—Ø, wherein —Ø represents the attachment point (representing a covalent bond) to an amine in X1 and one R₁ for B₅ represents (C═O)—*, S(═O)(═O)—*, (CH₂)ₘ(C═O)—*, or (CH₂)ₘ—*, wherein —* represents the attachment point to the same amine in X1, and m is 1, 2, 3, 4, 5, 6, or 7;
each remaining R₁ is independently selected from H, F, Cl, Br, OH, CH₂—NH₂, NH₂, (C═O)—NH₂, CH═O, SO₂CH₃, SO₂CF₃, CF₃, CHF₂, NO₂, CH₃, OCH₃, O(CH₂)ₘCH₃, —(SO₂)NH CH₃, —(SO₂)NH(CH₂)ₘCH₃, and OCF₃, wherein m is 1, 2, 3, 4, 5, 6, or 7.

In some embodiments, for B₆:
one R1 for B₆ represents (CH₂)ₘ—Ø, wherein —Ø represents the attachment point (representing a covalent bond) to the rest of the compound, and m is 1, 2, 3, 4, 5, 6, or 7;
each remaining R₁ is independently selected from H, F, Cl, Br, OH, CH₂—NH₂, NH₂, (C═O)—NH₂, CH═O, SO₂CH₃, SO₂CF₃, CF₃, CHF₂, NO₂, CH₃, OCH₃, O(CH₂)ₘCH₃, —(SO₂)NH CH₃, —(SO₂)NH(CH₂)ₘCH₃, and OCF₃, wherein m is 1, 2, 3,4, 5, 6, or 7.

In some embodiments,

### for Formulae F3-F4:

R_{w} is O or S;
for Formulae F5-F10:
Y8 is selected from O, N, and NR, wherein R is an alkyl group or H;
Y9 is H, CH₃, or an alkyl group, provided that when Y8 is O, Y9 is a CH₃ or higher alkyl group;
each Y10 is independently selected from H, CH₃, F, CF₃, and OCH₃; and
i is 1, 2, or 3.

In some embodiments, the compound is selected from:
N-(3-(3-borono-5-nitrobenzamido)propyl)-N-(3-borono-5-nitrobenzoyl)glycine (DS01);
N-(4-((4-(3-borono-5-nitrobenzamido)cyclohexyl)methyl)cyclohexyl)-N-(3-borono-5-nitrobenzoyl)glycine (DS02);
N-(4-((3-borono-5-nitrobenzamido)methyl)benzyl)-N-(3-borono-5-nitrobenzoyl)glycine (DS03);
N-(3-((3-borono-5-nitrobenzamido)methyl)benzyl)-N-(3-borono-5-nitrobenzoyl)glycine (DS04);
N-(4-(3-borono-5-nitrobenzamido)butyl)-N-(3-borono-5-nitrobenzoyl)glycine (DS05);
N-(3-(3-borono-5-fluorobenzamido)propyl)-N-(3-borono-5-fluorobenzoyl)glycine (DS06);
N-(3-(3-borono-5-fluorobenzamido)-2,2-dimethylpropyl)-N-(3-borono-5-fluorobenzoyl)glycine (DS07);
bis(3-(3-borono-5-fluorobenzamido)propyl)glycine (DS08);
N-(4-((3-borono-5-fluorobenzamido)methyl)benzyl)-N-(3-borono-5-fluorobenzoyl)glycine (DS09);
N-(3-((3-borono-5-fluorobenzamido)methyl)benzyl)-N-(3-borono-5-fluorobenzoyl)glycine (DS10);
N-(2-(3-borono-5-fluorobenzamido)cyclohexyl)-N-(3-borono-5-fluorobenzoyl)glycine (DS11);
N-(3-(3-borono-4-fluorobenzamido)propyl)-N-(3-borono-4-fluorobenzoyl)glycine (DS12);
N-(4-((4-(3-borono-4-fluorobenzamido)cyclohexyl)methyl)cyclohexyl)-N-(3-borono-4-fluorobenzoyl)glycine (DS13);
N-(3-(3-borono-4-fluorobenzamido)-2,2-dimethylpropyl)-N-(3-borono-4-fluorobenzoyl)glycine (DS14);
N-(4-((3-borono-4-fluorobenzamido)methyl)benzyl)-N-(3-borono-4-fluorobenzoyl)glycine (DS15);
N-(3-((3-borono-4-fluorobenzamido)methyl)benzyl)-N-(3-borono-4-fluorobenzoyl)glycine (DS16);
N-((1S,2R)-2-(3-borono-4-fluorobenzamido)cyclohexyl)-N-(3-borono-4-fluorobenzoyl)glycine (DS17);
N-((1S,2S)-2-(3-borono-4-fluorobenzamido)cyclohexyl)-N-(3-borono-4-fluorobenzoyl)glycine (DS18);
N-(3-(3-borono-5-bromobenzamido)propyl)-N-(3-borono-5-bromobenzoyl)glycine (DS19);
N-(4-((4-(3-borono-5-bromobenzamido)cyclohexyl)methyl)cyclohexyl)-N-(3-borono-5-bromobenzoyl)glycine (DS20);
bis(3-(3-borono-5-bromobenzamido)propyl)glycine (DS21);
N-(4-((3-borono-5-bromobenzamido)methyl)benzyl)-N-(3-borono-5-bromobenzoyl)glycine (DS22);
N-(3-((3-borono-5-bromobenzamido)methyl)benzyl)-N-(3-borono-5-bromobenzoyl)glycine (DS23);
N-(2-(3-borono-5-bromobenzamido)cyclohexyl)-N-(3-borono-5-bromobenzoyl)glycine (DS24);
N-(3-(4-borono-3-fluorobenzamido)propyl)-N-(4-borono-3-fluorobenzoyl)glycine (DS25);
N-(4-((4-(4-borono-3-fluorobenzamido)cyclohexyl)methyl)cyclohexyl)-N-(4-borono-3-fluorobenzoyl)glycine (DS26);
N-(3-(4-borono-3-fluorobenzamido)-2,2-dimethylpropyl)-N-(4-borono-3-fluorobenzoyl)glycine (DS27);
bis(3-(4-borono-3-fluorobenzamido)propyl)glycine (DS28);
N-(4-((4-borono-3-fluorobenzamido)methyl)benzyl)-N-(4-borono-3-fluorobenzoyl)glycine (DS29);
N-(3-((4-borono-3-fluorobenzamido)methyl)benzyl)-N-(4-borono-3-fluorobenzoyl)glycine (DS30);
N-((1S,2R)-2-(4-borono-3-fluorobenzamido)cyclohexyl)-N-(4-borono-3-fluorobenzoyl)glycine (DS31);
N-(1-hydroxy-1,3-dihydrobenzo[c][1,2]oxaborole-6-carbonyl)-N-(3-(1-hydroxy-1,3-dihydrobenzo[c][1,2]oxaborole-6-carboxamido)propyl)glycine (DS32);
N-(1-hydroxy-1,3-dihydrobenzo[c][1,2]oxaborole-6-carbonyl)-N-(5-(1-hydroxy-1,3-dihydrobenzo[c][1,2]oxaborole-6-carboxamido)pentyl)glycine (DS33);
N-(1-hydroxy-1,3-dihydrobenzo[c][1,2]oxaborole-6-carbonyl)-N-(3-(1-hydroxy-1,3-dihydrobenzo[c][1,2]oxaborole-6-carboxamido)-2,2-dimethylpropyl)glycine (DS34);
bis(3-(1-hydroxy-1,3-dihydrobenzo[c][1,2]oxaborole-6-carboxamido)propyl)glycine (DS35);
N-(1-hydroxy-1,3-dihydrobenzo[c][1,2]oxaborole-6-carbonyl)-N-(3-((1-hydroxy-1,3-dihydrobenzo[c][1,2]oxaborole-6-carboxamido)methyl)benzyl)glycine (DS36);
N-(1-hydroxy-1,3-dihydrobenzo[c][1,2]oxaborole-6-carbonyl)-N-((1S,2R)-2-(1-hydroxy-1,3-dihydrobenzo[c][1,2]oxaborole-6-carboxamido)cyclohexyl)glycine (DS37);
N-(1-hydroxy-1,3-dihydrobenzo[c][1,2]oxaborole-6-carbonyl)-N-(4-(1-hydroxy-1,3-dihydrobenzo[c][1,2]oxaborole-6-carboxamido)butyl)glycine (DS38);
N-(1-hydroxy-1,3-dihydrobenzo[c][1,2]oxaborole-6-carbonyl)-N-((1S,2S)-2-(1-hydroxy-1,3-dihydrobenzo[c][1,2]oxaborole-6-carboxamido)cyclohexyl)glycine (DS39);
(R)—N-(1-hydroxy-1,3-dihydrobenzo[c][1,2]oxaborole-6-carbonyl)-N-(2-(1-hydroxy-1,3-dihydrobenzo[c][1,2]oxaborole-6-carboxamido)propyl)glycine (DS40);
(S)—N-(1-hydroxy-1,3-dihydrobenzo[c][1,2]oxaborole-6-carbonyl)-N-(2-(1-hydroxy-1,3-dihydrobenzo[c][1,2]oxaborole-6-carboxamido)propyl)glycine (DS41);
N-(1-hydroxy-1,3-dihydrobenzo[c][1,2]oxaborole-6-carbonyl)-N-(2-(1-hydroxy-1,3-dihydrobenzo[c][1,2]oxaborole-6-carboxamido)cyclohexyl)glycine (DS42);
N-(3-(4-borono-3,5-difluorobenzamido)propyl)-N-(4-borono-3,5-difluorobenzoyl)glycine (DS43);
N-(3-(4-borono-2-fluorobenzamido)propyl)-N-(4-borono-2-fluorobenzoyl)glycine (DS44);
N-(2-(N-ethyl-1-hydroxy-1,3-dihydrobenzo[c][1,2]oxaborole-6-carboxamido)ethyl)-N-(1-hydroxy-1,3-dihydrobenzo[c][1,2]oxaborole-6-carbonyl)glycine (DS45);
N-(1-hydroxy-1,3-dihydrobenzo[c][1,2]oxaborole-6-carbonyl)-N-(2-(1-hydroxy-N-(2-hydroxyethyl)-1,3-dihydrobenzo[c][1,2]oxaborole-6-carboxamido)ethyl)glycine (DS46);
N-(1-hydroxy-1,3-dihydrobenzo[c][1,2]oxaborole-6-carbonyl)-N-(5-(1-hydroxy-1,3-dihydrobenzo[c][1,2]oxaborole-6-carboxamido)hexyl)glycine (DS47);
N-(1-hydroxy-1,3-dihydrobenzo[c][1,2]oxaborole-6-carbonyl)-N-(4-((4-(1-hydroxy-1,3-dihydrobenzo[c][1,2]oxaborole-6-carboxamido)cyclohexyl)methyl)cyclohexyl)glycine (DS48);
((2S,4S)-1-(1-hydroxy-1,3-dihydrobenzo[c][1,2]oxaborole-6-carbonyl)-4-(1-hydroxy-1,3-dihydrobenzo[c][1,2]oxaborole-6-carboxamido)pyrrolidine-2-carbonyl)glycine (DS49);
((2S,4S)-4-(3-borono-4-fluorobenzamido)-1-(3-borono-4-fluorobenzoyl)pyrrolidine-2-carbonyl)glycine (DS50);
((2S,4S)-4-(3-borono-5-nitrobenzamido)-1-(3-borono-5-nitrobenzoyl)pyrrolidine-2-carbonyl)glycine (DS51);
((2S,4S)-4-(5-borono-2-fluorobenzamido)-1-(5-borono-2-fluorobenzoyl)pyrrolidine-2-carbonyl)glycine (DS52);
(S)-(1,4-bis(1-hydroxy-1,3-dihydrobenzo[c][1,2]oxaborole-6-carbonyl)piperazine-2-carbonyl)glycine (DS53);
(S)—N-(3-amino-2-(1-hydroxy-1,3-dihydrobenzo[c][1,2]oxaborole-6-carboxamido)-3-oxopropyl)-N-benzyl-1-hydroxy-1,3-dihydrobenzo[c][1,2]oxaborole-6-carboxamide (DS54);
(S)—N-(3-amino-2-(1-hydroxy-1,3-dihydrobenzo[c][1,2]oxaborole-6-carboxamido)-3-oxopropyl)-1-hydroxy-N-(4-(trifluoromethyl)benzyl)-1,3-dihydrobenzo[c][1,2]oxaborole-6-carboxamide (DS55);
(S)—N-(3-amino-2-(1-hydroxy-1,3-dihydrobenzo[c][1,2]oxaborole-6-carboxamido)-3-oxopropyl)-N-ethyl-1-hydroxy-1,3-dihydrobenzo[c][1,2]oxaborole-6-carboxamide (DS56);
(S)—N-(3-amino-2-(1-hydroxy-1,3-dihydrobenzo[c][1,2]oxaborole-6-carboxamido)-3-oxopropyl)-1-hydroxy-N-propyl-1,3-dihydrobenzo[c][1,2]oxaborole-6-carboxamide (DS57);
(S)—N-(3-amino-2-(1-hydroxy-1,3-dihydrobenzo[c][1,2]oxaborole-6-carboxamido)-3-oxopropyl)-1-hydroxy-N-isobutyl-1,3-dihydrobenzo[c][1,2]oxaborole-6-carboxamide (DS58);
(S)—N-(3-amino-2-(1-hydroxy-1,3-dihydrobenzo[c][1,2]oxaborole-6-carboxamido)-3-oxopropyl)-1-hydroxy-N-((5-(thiophen-2-yl)pyridin-2-yl)methyl)-1,3-dihydrobenzo[c][1,2]oxaborole-6-carboxamide (DS59);
(S)—N-(3-amino-2-(1-hydroxy-1,3-dihydrobenzo[c][1,2]oxaborole-6-carboxamido)-3-oxopropyl)-1-hydroxy-N-isopentyl-1,3-dihydrobenzo[c][1,2]oxaborole-6-carboxamide (DS60);
(S)—N-(3-amino-2-(1-hydroxy-1,3-dihydrobenzo[c][1,2]oxaborole-6-carboxamido)-3-oxopropyl)-1-hydroxy-N-(quinolin-5-ylmethyl)-1,3-dihydrobenzo[c][1,2]oxaborole-6-carboxamide (DS61);
(S)—N-(3-amino-2-(1-hydroxy-1,3-dihydrobenzo[c][1,2]oxaborole-6-carboxamido)-3-oxopropyl)-1-hydroxy-N-(2-(trifluoromethoxy)benzyl)-1,3-dihydrobenzo[c][1,2]oxaborole-6-carboxamide (DS62);
(S)—N-(3-amino-2-(1-hydroxy-1,3-dihydrobenzo[c][1,2]oxaborole-6-carboxamido)-3-oxopropyl)-1-hydroxy-N-(4-(methylsulfonyl)benzyl)-1,3-dihydrobenzo[c][1,2]oxaborole-6-carboxamide (DS63);
(3-((2S,4S)-4-(5-borono-2-(methylsulfonyl)benzamido)-2-carbamoylpyrrolidine-1-carbonyl)-4-(methylsulfonyl)phenyl)boronic acid (DS64);
(4-(((3S,5S)-1-(4-borono-2,6-difluorobenzoyl)-5-carbamoylpyrrolidin-3-yl)carbamoyl)-3,5-difluorophenyl)boronic acid (DS65);
(R,E)-4,5-bis(1-hydroxy-1,3-dihydrobenzo[c][1,2]oxaborole-6-carboxamido)pent-2-enoic acid (DS66);
(2S,4S)-1-(1-hydroxy-4-(trifluoromethyl)-1,3-dihydrobenzo[c][1,2]oxaborole-6-carbonyl)-4-(1-hydroxy-4-(trifluoromethyl)-1,3-dihydrobenzo[c][1,2]oxaborole-6-carboxamido)pyrrolidine-2-carboxamide (DS67);
N,N′-((2S,3S)-1-amino-1-oxobutane-2,3-diyl)bis(1-hydroxy-1,3-dihydrobenzo[c][1,2]oxaborole-6-carboxamide) (DS68);
(R)-3,4-bis(1-hydroxy-1,3-dihydrobenzo[c][1,2]oxaborole-6-carboxamido)butanoic acid (DS69);
3-((2S,4S)-1-(1-hydroxy-1,3-dihydrobenzo[c][1,2]oxaborole-6-carbonyl)-4-(1-hydroxy-1,3-dihydrobenzo[c][1,2]oxaborole-6-carboxamido)pyrrolidine-2-carboxamido)propanoic acid (DS70);
(S)-3-(1-hydroxy-1,3-dihydrobenzo[c][1,2]oxaborole-5-carboxamido)-4-(1-hydroxy-1,3-dihydrobenzo[c][1,2]oxaborole-6-carboxamido)butanoic acid (DS71);
(R)-4-(1-hydroxy-1,3-dihydrobenzo[c][1,2]oxaborole-5-carboxamido)-5-(1-hydroxy-1,3-dihydrobenzo[c][1,2]oxaborole-6-carboxamido)pentanoic acid (DS72);
(2S,4R)-1-(1-hydroxy-1,3-dihydrobenzo[c][1,2]oxaborole-6-carbonyl)-4-(1-hydroxy-1,3-dihydrobenzo[c][1,2]oxaborole-6-carboxamido)pyrrolidine-2-carboxylic acid (DS73);
(2S,4R)-1-(1-hydroxy-4-(trifluoromethyl)-1,3-dihydrobenzo[c][1,2]oxaborole-6-carbonyl)-4-(1-hydroxy-4-(trifluoromethyl)-1,3-dihydrobenzo[c][1,2]oxaborole-6-carboxamido)pyrrolidine-2-carboxylic acid (DS74);
(2S,3S)-3-(1-hydroxy-4-(trifluoromethyl)-1,3-dihydrobenzo[c][1,2]oxaborole-6-carboxamido)-2-(1-hydroxy-7-(trifluoromethyl)-1,3-dihydrobenzo[c][1,2]oxaborole-5-carboxamido)butanoic acid (DS75);
(R)-5-(1-hydroxy-4-(trifluoromethyl)-1,3-dihydrobenzo[c][1,2]oxaborole-6-carboxamido)-4-(1-hydroxy-7-(trifluoromethyl)-1,3-dihydrobenzo[c][1,2]oxaborole-5-carboxamido)pentanoic acid (DS76);
((2S,4S)-1-(5-borono-2-nitrobenzoyl)-4-(1-hydroxy-1,3-dihydrobenzo[c][1,2]oxaborole-6-carboxamido)pyrrolidine-2-carbonyl)glycine (DS77);
((2S,4S)-1-(5-borono-2-(methylsulfonyl)benzoyl)-4-(1-hydroxy-1,3-dihydrobenzo[c][1,2]oxaborole-6-carboxamido)pyrrolidine-2-carbonyl)glycine (DS78);
((2S,4S)-1-(3-borono-2,6-difluorobenzoyl)-4-(1-hydroxy-1,3-dihydrobenzo[c][1,2]oxaborole-6-carboxamido)pyrrolidine-2-carbonyl)glycine (DS79);
(S)-(3-((3-borono-4-fluorobenzyl)(5,6-diamino-6-oxohexyl)carbamoyl)-5-nitrophenyl)boronic acid (DS80);
(S)-(3-((4-borono-3,5-difluorobenzyl)(5,6-diamino-6-oxohexyl)carbamoyl)-5-nitrophenyl)boronic acid (DS81);
(S)-(3-((3-boronobenzyl)(5,6-diamino-6-oxohexyl)carbamoyl)-5-nitrophenyl)boronic acid (DS82);
(S)-(3-((4-borono-2-methoxybenzyl)(5,6-diamino-6-oxohexyl)carbamoyl)-5-nitrophenyl)boronic acid (DS83);
(S)-(3-((4-borono-2-(trifluoromethyl)benzyl)(5,6-diamino-6-oxohexyl)carbamoyl)-5-nitrophenyl)boronic acid (DS84);
(S)-(5-((3-borono-N-(5,6-diamino-6-oxohexyl)-4-fluorobenzamido)methyl)-2-fluorophenyl)boronic acid (DS85);
(S)-(5-((4-borono-3,5-difluorobenzyl)(5,6-diamino-6-oxohexyl)carbamoyl)-2-fluorophenyl)boronic acid (DS86);
(S)-(3-((3-borono-N-(5,6-diamino-6-oxohexyl)-4-fluorobenzamido)methyl) phenyl)boronic acid (DS87);
(S)-(5-((4-borono-2-methoxybenzyl)(5,6-diamino-6-oxohexyl)carbamoyl)-2-fluorophenyl)boronic acid (DS88);
(S)-(5-((4-borono-3-(trifluoromethyl)benzyl)(5,6-diamino-6-oxohexyl)carbamoyl)-2-fluorophenyl)boronic acid (DS89);
(S)-(4-((3-borono-4-fluorobenzyl)(5,6-diamino-6-oxohexyl)carbamoyl)-2-fluorophenyl)boronic acid (DS90);
(S)-(4-((4-borono-3,5-difluorobenzyl)(5,6-diamino-6-oxohexyl)carbamoyl)-2-fluorophenyl)boronic acid (DS91);
(S)-(4-((3-boronobenzyl)(5,6-diamino-6-oxohexyl)carbamoyl)-2-fluorophenyl)boronic acid (DS92);
(S)-(4-((4-borono-2-methoxybenzyl)(5,6-diamino-6-oxohexyl)carbamoyl)-2-fluorophenyl)boronic acid (DS93);
(S)-(4-((4-borono-2-(trifluoromethyl)benzyl)(5,6-diamino-6-oxohexyl)carbamoyl)-2-fluorophenyl)boronic acid (DS94);
(S)-(5-((3-borono-5-bromo-N-(5,6-diamino-6-oxohexyl)benzamido)methyl)-2-fluorophenyl)boronic acid (DS95);
(S)-(3-((4-borono-3,5-difluorobenzyl)(5,6-diamino-6-oxohexyl)carbamoyl)-5-bromophenyl)boronic acid (DS96);
(S)-(3-((3-borono-5-bromo-N-(5,6-diamino-6-oxohexyl)benzamido)methyl) phenyl)boronic acid (DS97);
(S)-(3-((3-borono-5-bromo-N-(5,6-diamino-6-oxohexyl)benzamido)methyl)-5-methoxyphenyl)boronic acid (DS98);
(S)-(3-((4-borono-2-(trifluoromethyl)benzyl)(5,6-diamino-6-oxohexyl)carbamoyl)-5-bromophenyl)boronic acid (DS99);
(S)-(3-((3-borono-4-fluorobenzyl)(5,6-diamino-6-oxohexyl)carbamoyl)-5-fluorophenyl)boronic acid (DS100);
(S)-(3-((4-borono-3-methoxybenzyl)(5,6-diamino-6-oxohexyl)carbamoyl)-5-fluorophenyl)boronic acid (DS101);
(S)-(3-((4-borono-2-(trifluoromethyl)benzyl)(5,6-diamino-6-oxohexyl)carbamoyl)-5-fluorophenyl)boronic acid (DS102);
(S)-(4-((N-(5,6-diamino-6-oxohexyl)-1-hydroxy-1,3-dihydrobenzo[c][1,2]oxaborole-6-carboxamido)methyl)-2-fluorophenyl)boronic acid (DS103);
(S)-(4-((N-(5,6-diamino-6-oxohexyl)-1-hydroxy-1,3-dihydrobenzo[c][1,2]oxaborole-6-carboxamido)methyl)-2,6-difluorophenyl)boronic acid (DS104);
(S)-(3-((N-(5,6-diamino-6-oxohexyl)-1-hydroxy-1,3-dihydrobenzo[c][1,2]oxaborole-6-carboxamido)methyl)phenyl)boronic acid (DS105);
(S)-(4-((N-(5,6-diamino-6-oxohexyl)-1-hydroxy-1,3-dihydrobenzo[c][1,2]oxaborole-6-carboxamido)methyl)-3-methoxyphenyl)boronic acid (DS106);
(S)—N-(5,6-diamino-6-oxohexyl)-1-hydroxy-N-((1-hydroxy-1,3-dihydrobenzo[c][1,2]oxaborol-6-yl)methyl)-1,3-dihydrobenzo[c][1,2]oxaborole-6-carboxamide (DS107);
(S)—N-(4-amino-3-(1-hydroxy-1,3-dihydrobenzo[c][1,2]oxaborole-6-carboxamido)-4-oxobutyl)-1-hydroxy-N-((1-hydroxy-1,3-dihydrobenzo[c][1,2]oxaborol-6-yl)methyl)-1,3-dihydrobenzo[c][1,2]oxaborole-6-carboxamide (DS108);
(S)—N-(6-amino-5-(1-hydroxy-1,3-dihydrobenzo[c][1,2]oxaborole-6-carboxamido)-6-oxohexyl)-1-hydroxy-N-((1-hydroxy-1,3-dihydrobenzo[c][1,2]oxaborol-6-yl)methyl)-1,3-dihydrobenzo[c][1,2]oxaborole-6-carboxamide (DS109);
(2S,4S)-1-(1-hydroxy-1,3-dihydrobenzo[c][1,2]oxaborole-6-carbonyl)-4-(1-hydroxy-1,3-dihydrobenzo[c][1,2]oxaborole-6-carboxamido)pyrrolidine-2-carboxylic acid (DS110);
(2S,3S)-2-(1-hydroxy-1,3-dihydrobenzo[c][1,2]oxaborole-5-carboxamido)-3-(1-hydroxy-1,3-dihydrobenzo[c][1,2]oxaborole-6-carboxamido)butanoic acid (DS111); and
(2S,4R)-1-(1-hydroxy-1,3-dihydrobenzo[c][1,2]oxaborole-6-carbonyl)-4-(1-hydroxy-1,3-dihydrobenzo[c][1,2]oxaborole-6-carboxamido)pyrrolidine-2-carboxylic acid (DS112).

In at least one embodiment, the compound of the present disclosure may be used, as an intermediate in the manufacture of a drug substance or a therapeutic of a prophylactic compound.

In another aspect, the disclosure provides a human insulin analog, comprising an A-chain and a B-chain, wherein the sequence of the A-chain comprises:

X_{aa′}X_{bb′}X_{cc′}X_{dd′}X_{ee′}X_{ff′}X_{gg′}VEQCCX_{hh′}X_{ii′}JCSLYQLENYCNX_{jj′}X_{kk′}X_{ll′}X_{mm′}X_{nn′}X_{oo′}X_{pp′} (SEQ ID NO:24015); and wherein the sequence of the B-chain comprises:
(i)

(SEQ ID NO: 24016)
XₐₐX_{bb}X_{cc}X_{dd}KXₑₑX_{ff}X_{gg}XₕₕXᵢᵢXⱼⱼKXₖₖXₗₗXₘₘXₙₙQHLCGSHLVEALYLVC
XₒₒXₚₚX_{qq}GFFYTXᵣᵣXₛₛXₜₜXᵤᵤXᵥᵥX_{ww},

wherein X_{aa′}, X_{bb′}, X_{cc′}, X_{dd′}, X_{ee′}, X_{ff′}, X_{gg′}, X_{hh′}, X_{ii′}, X_{jj′}, X_{kk′}, X_{ll′}, X_{mm′}, X_{nn′}, X_{oo′}, X_{pp′}, Xₐₐ, X_{bb}, X_{cc}, X_{dd}, Xₑₑ, X_{ff}, X_{gg}, Xₕₕ, Xᵢᵢ, Xⱼⱼ, Xₖₖ, Xₗₗ, Xₘₘ, Xₙₙ, Xₒₒ, Xₚₚ, X_{qq}, Xᵣᵣ, Xₛₛ, Xₜₜ, Xᵤᵤ, Xᵥᵥ, and X_{ww} are each independently either absent or selected from amino acid residues A, D, E, F, G, H, I, K, L, N, P, Q, R, S, T, V, Y and W,
(ii)

(SEQ ID NO: 24017)
XₐₐX_{bb}X_{cc}X_{dd}KPXₑₑX_{ff}X_{gg}XₕₕXᵢᵢXⱼⱼXₖₖXₗₗXₘₘXₙₙQHLCGSHLVEALYLVC
XₒₒXₚₚX_{qq}GFFYTXᵣᵣXₛₛXₜₜXᵤᵤXᵥᵥX_{ww},

wherein X_{aa′}, X_{bb′}, X_{cc′}, X_{dd′}, X_{ee′}, X_{ff′}, X_{gg′}, X_{hh′}, X_{ii′}, X_{jj′}, X_{kk′}, X_{ll′}, X_{mm′}, X_{nn′}, X_{oo′}, X_{pp′}, Xₐₐ, X_{bb}, X_{cc}, X_{dd}, X_{ff}, X_{gg}, Xₕₕ, Xᵢᵢ, Xⱼⱼ, Xₖₖ, Xₗₗ, Xₘₘ, Xₙₙ, Xₒₒ, Xₚₚ, X_{qq}, Xᵣᵣ, Xₛₛ, Xₜₜ, Xᵤᵤ, Xᵥᵥ, and X_{ww} are each independently either absent or selected from amino acid residues A, D, E, F, G, H, I, K, L, N, P, Q, R, S, T, V, Y and W, and wherein Xₑₑ is selected from amino acid residues A, E, F, H, I, K, L, N, P, Q, R, S, T, V, Y and W,
(iii)

(SEQ ID NO: 24018)
XₐₐX_{bb}X_{cc}X_{dd}KXₑₑX_{ff}X_{gg}XₕₕXᵢᵢXⱼⱼKXₖₖXₗₗXₘₘXₙₙQHLCGSHLVEALYLVC
XₒₒXₚₚX_{qq}GFFYTXᵣᵣXₛₛXₜₜXᵤᵤXᵥᵥX_{ww},

wherein X_{aa′}, X_{bb′}, X_{cc′}, X_{dd′}, X_{ee′}, X_{ff′}, X_{gg′}, X_{hh′}, X_{ii′}, X_{jj′}, X_{kk′}, X_{ll′}, X_{mm′}, X_{nn′}, X_{oo′}, X_{pp′}, Xₐₐ, X_{bb}, X_{cc}, X_{dd}, Xₑₑ, X_{ff}, X_{gg}, Xₕₕ, Xᵢᵢ, Xⱼⱼ, Xₖₖ, Xₗₗ, Xₘₘ, Xₙₙ, Xₒₒ, Xₚₚ, X_{qq}, Xᵣᵣ, Xₛₛ, Xₜₜ, Xᵤᵤ, Xᵥᵥ, and X_{ww}, are each independently either absent or selected from amino acid residues A, D, E, F, G, H, I, K, L, N, P, Q, R, S, T, V, Y, W and at least one of Xₑₑ, X_{ff}, X_{gg}, Xₕₕ, Xᵢᵢ, Xⱼⱼ is present and at least one of Xₑₑ, X_{ff}, X_{gg}, Xₕₕ, Xᵢᵢ, Xⱼⱼ is G,
(iv)

(SEQ ID NO: 24019)
XₐₐX_{bb}X_{cc}X_{dd}KXₑₑX_{ff}X_{gg}XₕₕXᵢᵢXⱼⱼKXₖₖXₗₗXₘₘXₙₙQHLCGSHLVEALYLVC
XₒₒXₚₚX_{gg}GFFYTXᵣᵣXₛₛXₜₜXᵤᵤXᵥᵥX_{ww},

wherein X_{aa′}, X_{bb′}, X_{cc′}, X_{dd′}, X_{ee′}, X_{ff′}, X_{gg′}, X_{hh′}, X_{ii′}, X_{jj′}, X_{kk′}, X_{ll′}, X_{mm′}, X_{nn′}, X_{oo′}, X_{pp′}, Xₐₐ, X_{bb}, X_{cc}, X_{dd}, Xₑₑ, X_{ff}, X_{gg}, Xₕₕ, Xᵢᵢ, Xⱼⱼ, Xₖₖ, Xₗₗ, Xₘₘ, Xₙₙ, Xₒₒ, Xₚₚ, X_{qq}, Xᵣᵣ, Xₛₛ, Xₜₜ, Xᵤᵤ, Xᵥᵥ, and X_{ww} are each independently either absent or selected from amino acid residues A, D, E, F, G, H, I, K, L, N, P, Q, R, S, T, V, Y, W and at least one of Xₑₑ, X_{ff}, X_{gg}, Xₕₕ, Xᵢᵢ, Xⱼⱼ is present and at least one of Xₑₑ, X_{ff}, X_{gg}, Xₕₕ, Xᵢᵢ, Xⱼⱼ is S, or
(v)

(SEQ ID NO: 24020)
XₐₐX_{bb}X_{cc}X_{dd}KXₑₑX_{ff}X_{gg}XₕₕXᵢᵢXⱼⱼKXₖₖXₗₗXₘₘXₙₙQHLCGSHLVEALYLVC
XₒₒXₚₚX_{qq}GFFYTXᵣᵣXₛₛXₜₜXᵤᵤXᵥᵥX_{ww},

wherein X_{aa′}, X_{bb′}, X_{cc′}, X_{dd′}, X_{ee′}, X_{ff′}, X_{gg′}, X_{hh′}, X_{ii′}, X_{jj′}, X_{kk′}, X_{ll′}, X_{mm′}, X_{nn′}, X_{oo′}, X_{pp′}, Xₐₐ, X_{bb}, X_{cc}, X_{dd}, Xₑₑ, X_{ff}, X_{gg}, Xₕₕ, Xᵢᵢ, Xⱼⱼ, Xₖₖ, Xₗₗ, Xₘₘ, Xₙₙ, Xₒₒ, Xₚₚ, X_{qq}, Xᵣᵣ, Xₛₛ, Xₜₜ, Xᵤᵤ, Xᵥᵥ, and X_{ww} are each independently either absent or selected from amino acid residues A, D, E, F, G, H, I, K, L, N, P, Q, R, S, T, V, Y, W and at least two of Xₑₑ, X_{ff}, X_{gg}, Xₕₕ, Xᵢᵢ, Xⱼⱼ are present and at least one of Xₑₑ, X_{ff}, X_{gg}, Xₕₕ, Xᵢᵢ, Xⱼⱼ is S, and another is G.

In some embodiments, the A-chain comprises a sequence selected from SEQ ID NOs 1 and 3 to 33, and is optionally appended at the N-terminus and/or at the C-terminus by at least one selected from KA, KD, KE, KF, KG, KH, KI, KL, KN, KP, KQ, KR, KS, KT, KY, KAA, KAD, KAE, KAF, KAG, KAH, KAI, KAL, KAN, KAQ, KAR, KAS, KAT, KAY, KDA, KDD, KDE, KDF, KDG, KDH, KDI, KDL, KDN, KDQ, KDR, KDS, KDT, KDY, KEA, KED, KEE, KEF, KEG, KEH, KEI, KEL, KEN, KEQ, KER, KES, KET, KEY, KFA, KFD, KFE, KFF, KFG, KFH, KFI, KFL, KFN, KFQ, KFR, KFS, KFT, KFY, KGA, KGD, KGE, KGF, KGG, KGH, KGI, KGL, KGN, KGQ, KGR, KGS, KGT, KGY, KHA, KHD, KHE, KHF, KHG, KHH, KHI, KHL, KHN, KHQ, KHR, KHS, KHT, KHY, KIA, KID, KIE, KIF, KIG, KIH, KII, KIL, KIN, KIQ, KIR, KIS, KIT, KIY, KLA, KLD, KLE, KLF, KLG, KLH, KLI, KLL, KLN, KLQ, KLR, KLS, KLT, KLY, KNA, KND, KNE, KNF, KNG, KNH, KNI, KNL, KNN, KNQ, KNR, KNS, KNT, KNY, KPA, KPD, KPE, KPF, KPG, KPH, KPI, KPL, KPN, KPQ, KPR, KPS, KPT, KPY, KQA, KQD, KQE, KQF, KQG, KQH, KQI, KQL, KQN, KQQ, KQR, KQS, KQT, KQY, KRA, KRD, KRE, KRF, KRG, KRH, KRI, KRL, KRN, KRQ, KRR, KRS, KRT, KRY, KSA, KSD, KSE, KSF, KSG, KSH, KSI, KSL, KSN, KSQ, KSR, KSS, KST, KSY, KTA, KTD, KTE, KTF, KTG, KTH, KTI, KTL, KTN, KTQ, KTR, KTS, KTT, KTY, KYA, KYD, KYE, KYF, KYG, KYH, KYI, KYL, KYN, KYQ, KYR, KYS, KYT, KYY and SEQ ID NOs 75 to 24014, 24037-24046, and wherein the B-chain comprises at least one of SEQ ID NOs 2 and 34 to 74, 24047, and 24048, and is optionally appended at the N-terminus and/or at the C-terminus by at least one selected from KA, KD, KE, KF, KG, KH, KI, KL, KN, KP, KQ, KR, KS, KT, KY, KAA, KAD, KAE, KAF, KAG, KAH, KAI, KAL, KAN, KAQ, KAR, KAS, KAT, KAY, KDA, KDD, KDE, KDF, KDG, KDH, KDI, KDL, KDN, KDQ, KDR, KDS, KDT, KDY, KEA, KED, KEE, KEF, KEG, KEH, KEI, KEL, KEN, KEQ, KER, KES, KET, KEY, KFA, KFD, KFE, KFF, KFG, KFH, KFI, KFL, KFN, KFQ, KFR, KFS, KFT, KFY, KGA, KGD, KGE, KGF, KGG, KGH, KGI, KGL, KGN, KGQ, KGR, KGS, KGT, KGY, KHA, KHD, KHE, KHF, KHG, KHH, KHI, KHL, KHN, KHQ, KHR, KHS, KHT, KHY, KIA, KID, KIE, KIF, KIG, KIH, KII, KIL, KIN, KIQ, KIR, KIS, KIT, KIY, KLA, KLD, KLE, KLF, KLG, KLH, KLI, KLL, KLN, KLQ, KLR, KLS, KLT, KLY, KNA, KND, KNE, KNF, KNG, KNH, KNI, KNL, KNN, KNQ, KNR, KNS, KNT, KNY, KPA, KPD, KPE, KPF, KPG, KPH, KPI, KPL, KPN, KPQ, KPR, KPS, KPT, KPY, KQA, KQD, KQE, KQF, KQG, KQH, KQI, KQL, KQN, KQQ, KQR, KQS, KQT, KQY, KRA, KRD, KRE, KRF, KRG, KRH, KRI, KRL, KRN, KRQ, KRR, KRS, KRT, KRY, KSA, KSD, KSE, KSF, KSG, KSH, KSI, KSL, KSN, KSQ, KSR, KSS, KST, KSY, KTA, KTD, KTE, KTF, KTG, KTH, KTI, KTL, KTN, KTQ, KTR, KTS, KTT, KTY, KYA, KYD, KYE, KYF, KYG, KYH, KYI, KYL, KYN, KYQ, KYR, KYS, KYT, KYY and SEQ ID NOs 75 to 24014, 24037-24046.

In some embodiments, the A-chain comprises a sequence selected from SEQ ID NOs 1 and 3 to 33, the B-chain comprises at least one of SEQ ID NOs 2 and 34 to 74, 24047, and 24048, and
(a)
the A-chain and the B-chain are each independently and optionally appended at the N-terminus and/or at the C-terminus by at least one selected from:
KA, KD, KE, KF, KG, KH, KI, KL, KN, KP, KQ, KR, KS, KT, KY, KAA, KAD, KAE, KAF, KAG, KAH, KAI, KAL, KAN, KAQ, KAR, KAS, KAT, KAY, KDA, KDD, KDE, KDF, KDG, KDH, KDI, KDL, KDN, KDQ, KDR, KDS, KDT, KDY, KEA, KED, KEE, KEF, KEG, KEH, KEI, KEL, KEN, KEQ, KER, KES, KET, KEY, KFA, KFD, KFE, KFF, KFG, KFH, KFI, KFL, KFN, KFQ, KFR, KFS, KFT, KFY, KGA, KGD, KGE, KGF, KGG, KGH, KGI, KGL, KGN, KGQ, KGR, KGS, KGT, KGY, KHA, KHD, KHE, KHF, KHG, KHH, KHI, KHL, KHN, KHQ, KHR, KHS, KHT, KHY, KIA, KID, KIE, KIF, KIG, KIH, KII, KIL, KIN, KIQ, KIR, KIS, KIT, KIY, KLA, KLD, KLE, KLF, KLG, KLH, KLI, KLL, KLN, KLQ, KLR, KLS, KLT, KLY, KNA, KND, KNE, KNF, KNG, KNH, KNI, KNL, KNN, KNQ, KNR, KNS, KNT, KNY, KPA, KPD, KPE, KPF, KPG, KPH, KPI, KPL, KPN, KPQ, KPR, KPS, KPT, KPY, KQA, KQD, KQE, KQF, KQG, KQH, KQI, KQL, KQN, KQQ, KQR, KQS, KQT, KQY, KRA, KRD, KRE, KRF, KRG, KRH, KRI, KRL, KRN, KRQ, KRR, KRS, KRT, KRY, KSA, KSD, KSE, KSF, KSG, KSH, KSI, KSL, KSN, KSQ, KSR, KSS, KST, KSY, KTA, KTD, KTE, KTF, KTG, KTH, KTI, KTL, KTN, KTQ, KTR, KTS, KTT, KTY, KYA, KYD, KYE, KYF, KYG, KYH, KYI, KYL, KYN, KYQ, KYR, KYS, KYT, KYY,
SEQ ID NOs 75 to 24014, 24037-24046,
KA, KD, KE, KF, KG, KH, KI, KL, KN, KP, KQ, KR, KS, KT, KY, KAA, KAD, KAE, KAF, KAG, KAH, KAI, KAL, KAN, KAQ, KAR, KAS, KAT, KAY, KDA, KDD, KDE, KDF, KDG, KDH, KDI, KDL, KDN, KDQ, KDR, KDS, KDT, KDY, KEA, KED, KEE, KEF, KEG, KEH, KEI, KEL, KEN, KEQ, KER, KES, KET, KEY, KFA, KFD, KFE, KFF, KFG, KFH, KFI, KFL, KFN, KFQ, KFR, KFS, KFT, KFY, KGA, KGD, KGE, KGF, KGG, KGH, KGI, KGL, KGN, KGQ, KGR, KGS, KGT, KGY, KHA, KHD, KHE, KHF, KHG, KHH, KHI, KHL, KHN, KHQ, KHR, KHS, KHT, KHY, KIA, KID, KIE, KIF, KIG, KIH, KII, KIL, KIN, KIQ, KIR, KIS, KIT, KIY, KLA, KLD, KLE, KLF, KLG, KLH, KLI, KLL, KLN, KLQ, KLR, KLS, KLT, KLY, KNA, KND, KNE, KNF, KNG, KNH, KNI, KNL, KNN, KNQ, KNR, KNS, KNT, KNY, KPA, KPD, KPE, KPF, KPG, KPH, KPI, KPL, KPN, KPQ, KPR, KPS, KPT, KPY, KQA, KQD, KQE, KQF, KQG, KQH, KQI, KQL, KQN, KQQ, KQR, KQS, KQT, KQY, KRA, KRD, KRE, KRF, KRG, KRH, KRI, KRL, KRN, KRQ, KRR, KRS, KRT, KRY, KSA, KSD, KSE, KSF, KSG, KSH, KSI, KSL, KSN, KSQ, KSR, KSS, KST, KSY, KTA, KTD, KTE, KTF, KTG, KTH, KTI, KTL, KTN, KTQ, KTR, KTS, KTT, KTY, KYA, KYD, KYE, KYF, KYG, KYH, KYI, KYL, KYN, KYQ, KYR, KYS, KYT, KYY and,
KA, KD, KE, KF, KG, KH, KI, KL, KN, KP, KQ, KR, KS, KT, KY and SEQ ID NOs 75 to 3224,
KA, KD, KE, KF, KG, KH, KI, KL, KN, KP, KQ, KR, KS, KT, KY and SEQ ID NOs 3225 to 6374,
KA, KD, KE, KF, KG, KH, KI, KL, KN, KP, KQ, KR, KS, KT, KY and SEQ ID NOs 6375 to 15194,
KA, KD, KE, KF, KG, KH, KI, KL, KN, KP, KQ, KR, KS, KT, KY and SEQ ID NOs 15195 to 18134,
KA, KD, KE, KF, KG, KH, KI, KL, KN, KP, KQ, KR, KS, KT, KY and SEQ ID NOs 18135 to 21074, and
KA, KD, KE, KF, KG, KH, KI, KL, KN, KP, KQ, KR, KS, KT, KY and SEQ ID NOs 21075 to 24014, 24037-24046, or
(b)
both the N-terminus and the C-terminus of the B-chain are independently covalently conjugated, via a peptide bond, to one selected from:
KA, KD, KE, KF, KG, KH, KI, KL, KN, KP, KQ, KR, KS, KT, KY, KAA, KAD, KAE, KAF, KAG, KAH, KAI, KAL, KAN, KAQ, KAR, KAS, KAT, KAY, KDA, KDD, KDE, KDF, KDG, KDH, KDI, KDL, KDN, KDQ, KDR, KDS, KDT, KDY, KEA, KED, KEE, KEF, KEG, KEH, KEI, KEL, KEN, KEQ, KER, KES, KET, KEY, KFA, KFD, KFE, KFF, KFG, KFH, KFI, KFL, KFN, KFQ, KFR, KFS, KFT, KFY, KGA, KGD, KGE, KGF, KGG, KGH, KGI, KGL, KGN, KGQ, KGR, KGS, KGT, KGY, KHA, KHD, KHE, KHF, KHG, KHH, KHI, KHL, KHN, KHQ, KHR, KHS, KHT, KHY, KIA, KID, KIE, KIF, KIG, KIH, KII, KIL, KIN, KIQ, KIR, KIS, KIT, KIY, KLA, KLD, KLE, KLF, KLG, KLH, KLI, KLL, KLN, KLQ, KLR, KLS, KLT, KLY, KNA, KND, KNE, KNF, KNG, KNH, KNI, KNL, KNN, KNQ, KNR, KNS, KNT, KNY, KPA, KPD, KPE, KPF, KPG, KPH, KPI, KPL, KPN, KPQ, KPR, KPS, KPT, KPY, KQA, KQD, KQE, KQF, KQG, KQH, KQI, KQL, KQN, KQQ, KQR, KQS, KQT, KQY, KRA, KRD, KRE, KRF, KRG, KRH, KRI, KRL, KRN, KRQ, KRR, KRS, KRT, KRY, KSA, KSD, KSE, KSF, KSG, KSH, KSI, KSL, KSN, KSQ, KSR, KSS, KST, KSY, KTA, KTD, KTE, KTF, KTG, KTH, KTI, KTL, KTN, KTQ, KTR, KTS, KTT, KTY, KYA, KYD, KYE, KYF, KYG, KYH, KYI, KYL, KYN, KYQ, KYR, KYS, KYT, KYY, SEQ ID NOs 75 to 24014, 24037-24036,
KA, KD, KE, KF, KG, KH, KI, KL, KN, KP, KQ, KR, KS, KT, KY, KAA, KAD, KAE, KAF, KAG, KAH, KAI, KAL, KAN, KAQ, KAR, KAS, KAT, KAY, KDA, KDD, KDE, KDF, KDG, KDH, KDI, KDL, KDN, KDQ, KDR, KDS, KDT, KDY, KEA, KED, KEE, KEF, KEG, KEH, KEI, KEL, KEN, KEQ, KER, KES, KET, KEY, KFA, KFD, KFE, KFF, KFG, KFH, KFI, KFL, KFN, KFQ, KFR, KFS, KFT, KFY, KGA, KGD, KGE, KGF, KGG, KGH, KGI, KGL, KGN, KGQ, KGR, KGS, KGT, KGY, KHA, KHD, KHE, KHF, KHG, KHH, KHI, KHL, KHN, KHQ, KHR, KHS, KHT, KHY, KIA, KID, KIE, KIF, KIG, KIH, KII, KIL, KIN, KIQ, KIR, KIS, KIT, KIY, KLA, KLD, KLE, KLF, KLG, KLH, KLI, KLL, KLN, KLQ, KLR, KLS, KLT, KLY, KNA, KND, KNE, KNF, KNG, KNH, KNI, KNL, KNN, KNQ, KNR, KNS, KNT, KNY, KPA, KPD, KPE, KPF, KPG, KPH, KPI, KPL, KPN, KPQ, KPR, KPS, KPT, KPY, KQA, KQD, KQE, KQF, KQG, KQH, KQI, KQL, KQN, KQQ, KQR, KQS, KQT, KQY, KRA, KRD, KRE, KRF, KRG, KRH, KRI, KRL, KRN, KRQ, KRR, KRS, KRT, KRY, KSA, KSD, KSE, KSF, KSG, KSH, KSI, KSL, KSN, KSQ, KSR, KSS, KST, KSY, KTA, KTD, KTE, KTF, KTG, KTH, KTI, KTL, KTN, KTQ, KTR, KTS, KTT, KTY, KYA, KYD, KYE, KYF, KYG, KYH, KYI, KYL, KYN, KYQ, KYR, KYS, KYT, KYY,
KA, KD, KE, KF, KG, KH, KI, KL, KN, KP, KQ, KR, KS, KT, KY and SEQ ID NOs 75 to 3224,
KA, KD, KE, KF, KG, KH, KI, KL, KN, KP, KQ, KR, KS, KT, KY and SEQ ID NOs 3225 to 6374,
KA, KD, KE, KF, KG, KH, KI, KL, KN, KP, KQ, KR, KS, KT, KY and SEQ ID NOs 6375 to 15194,
KA, KD, KE, KF, KG, KH, KI, KL, KN, KP, KQ, KR, KS, KT, KY and SEQ ID NOs 15195 to 18134,
KA, KD, KE, KF, KG, KH, KI, KL, KN, KP, KQ, KR, KS, KT, KY and SEQ ID NOs 18135 to 21074, and
KA, KD, KE, KF, KG, KH, KI, KL, KN, KP, KQ, KR, KS, KT, KY and SEQ ID NOs 21075 to 24014, 24037-24036.

In some embodiments, the A-chain comprises a sequence selected from SEQ ID NOs 1 and 3 to 33, and is optionally appended at the N-terminus and/or at the C-terminus by at least one selected from KA, KD, KE, KF, KG, KH, KI, KL, KN, KP, KQ, KR, KS, KT, KY, KAA, KAD, KAE, KAF, KAG, KAH, KAI, KAL, KAN, KAQ, KAR, KAS, KAT, KAY, KDA, KDD, KDE, KDF, KDG, KDH, KDI, KDL, KDN, KDQ, KDR, KDS, KDT, KDY, KEA, KED, KEE, KEF, KEG, KEH, KEI, KEL, KEN, KEQ, KER, KES, KET, KEY, KFA, KFD, KFE, KFF, KFG, KFH, KFI, KFL, KFN, KFQ, KFR, KFS, KFT, KFY, KGA, KGD, KGE, KGF, KGG, KGH, KGI, KGL, KGN, KGQ, KGR, KGS, KGT, KGY, KHA, KHD, KHE, KHF, KHG, KHH, KHI, KHL, KHN, KHQ, KHR, KHS, KHT, KHY, KIA, KID, KIE, KIF, KIG, KIH, KII, KIL, KIN, KIQ, KIR, KIS, KIT, KIY, KLA, KLD, KLE, KLF, KLG, KLH, KLI, KLL, KLN, KLQ, KLR, KLS, KLT, KLY, KNA, KND, KNE, KNF, KNG, KNH, KNI, KNL, KNN, KNQ, KNR, KNS, KNT, KNY, KPA, KPD, KPE, KPF, KPG, KPH, KPI, KPL, KPN, KPQ, KPR, KPS, KPT, KPY, KQA, KQD, KQE, KQF, KQG, KQH, KQI, KQL, KQN, KQQ, KQR, KQS, KQT, KQY, KRA, KRD, KRE, KRF, KRG, KRH, KRI, KRL, KRN, KRQ, KRR, KRS, KRT, KRY, KSA, KSD, KSE, KSF, KSG, KSH, KSI, KSL, KSN, KSQ, KSR, KSS, KST, KSY, KTA, KTD, KTE, KTF, KTG, KTH, KTI, KTL, KTN, KTQ, KTR, KTS, KTT, KTY, KYA, KYD, KYE, KYF, KYG, KYH, KYI, KYL, KYN, KYQ, KYR, KYS, KYT, KYY, SEQ ID NOs 75 to 24014, KGSH (SEQ ID NO:24049), GKGSH (SEQ ID NO:24050), GKGSKK (SEQ ID NO:24045), GKKPGKK (SEQ ID NO:24046), GKGPSK (SEQ ID NO:24044), GKPSHKP (SEQ ID NO:24043), and GSHKGSHK (SEQ ID NO:24042); and
wherein the B-chain comprises a sequence selected from SEQ ID NOs 2 and 34 to 74, 24047, and 24048, and is optionally appended at the N-terminus and/or at the C-terminus by at least one selected from KA, KD, KE, KF, KG, KH, KI, KL, KN, KP, KQ, KR, KS, KT, KY, KAA, KAD, KAE, KAF, KAG, KAH, KAI, KAL, KAN, KAQ, KAR, KAS, KAT, KAY, KDA, KDD, KDE, KDF, KDG, KDH, KDI, KDL, KDN, KDQ, KDR, KDS, KDT, KDY, KEA, KED, KEE, KEF, KEG, KEH, KEI, KEL, KEN, KEQ, KER, KES, KET, KEY, KFA, KFD, KFE, KFF, KFG, KFH, KFI, KFL, KFN, KFQ, KFR, KFS, KFT, KFY, KGA, KGD, KGE, KGF, KGG, KGH, KGI, KGL, KGN, KGQ, KGR, KGS, KGT, KGY, KHA, KHD, KHE, KHF, KHG, KHH, KHI, KHL, KHN, KHQ, KHR, KHS, KHT, KHY, KIA, KID, KIE, KIF, KIG, KIH, KII, KIL, KIN, KIQ, KIR, KIS, KIT, KIY, KLA, KLD, KLE, KLF, KLG, KLH, KLI, KLL, KLN, KLQ, KLR, KLS, KLT, KLY, KNA, KND, KNE, KNF, KNG, KNH, KNI, KNL, KNN, KNQ, KNR, KNS, KNT, KNY, KPA, KPD, KPE, KPF, KPG, KPH, KPI, KPL, KPN, KPQ, KPR, KPS, KPT, KPY, KQA, KQD, KQE, KQF, KQG, KQH, KQI, KQL, KQN, KQQ, KQR, KQS, KQT, KQY, KRA, KRD, KRE, KRF, KRG, KRH, KRI, KRL, KRN, KRQ, KRR, KRS, KRT, KRY, KSA, KSD, KSE, KSF, KSG, KSH, KSI, KSL, KSN, KSQ, KSR, KSS, KST, KSY, KTA, KTD, KTE, KTF, KTG, KTH, KTI, KTL, KTN, KTQ, KTR, KTS, KTT, KTY, KYA, KYD, KYE, KYF, KYG, KYH, KYI, KYL, KYN, KYQ, KYR, KYS, KYT, KYY, SEQ ID NOs 75 to 24014, KGSH (SEQ ID NO:24049), GKGSH (SEQ ID NO:24050), GKGSKK (SEQ ID NO:24045), GKKPGKK (SEQ ID NO:24046), GKGPSK (SEQ ID NO:24044), GKPSHKP (SEQ ID NO:24043), and GSHKGSHK (SEQ ID NO:24042).

In some embodiments, no more than 4 residues are added or deleted from the A-chain and/or the B-chain of the insulin.

In some embodiments, a K residue is present at the N-terminus of the A-chain and/or the B-chain, and/or wherein no more than three K residues are present at the N-terminus of the A-chain and/or the B-chain, and/or wherein in (i) the tyrosine at A14 is replaced with glutamic acid, and/or (ii) the tyrosine at B16 is replaced with histidine, and/or (iii) the phenylalanine at B25 is replaced with a histidine, and/or wherein one to three residues selected from residues B20, B21, and B22-B29 of the B-chain, residues A4 or A8 of the A-chain, and residues of an optionally extended polypeptide, are lysine residues, and/or wherein only one K residue is present within 10 residues of the N-terminus of B-chain.

In some embodiments, X1 comprises an insulin and/or insulin analog as disclosed herein.

In some embodiments, X1 comprises an insulin and/or insulin analog as disclosed herein, and the insulin and/or insulin analog further comprises an optional covalent-spacer.

In some embodiments, an amino group of one or more side chain(s) of one to four lysine residues of insulin is each independently covalently conjugated as described by Formula I.

In some embodiments, the insulin comprises at least two amines that are covalently conjugated as described by Formula I, wherein one amine is the N-terminus amino group of the B-chain of insulin, and the other amine(s) are the side chain amine of a lysine that is 0 to 5 residues away from residue B22 of the B-chain of insulin, and/or the side chain amine of a lysine that is 1 to 5 residues away from residue A21 of the A-chain of insulin.

In some embodiments, an amino group at the N-terminus of the B-chain of insulin is covalently conjugated as described by Formula I, and q′ is optionally 2 or more, and the insulin includes at least one additional covalent conjugation to X1 as independently described by Formula I.

In some embodiments, the insulin is covalently conjugated as described by Formula I, such as in Examples 1-880, and wherein 1 to 4 residues are optionally added or deleted from the A-chain and/or B-chain of the insulins shown in Examples 1-880.

In another aspect, the disclosure provides an insulin (e.g., a modified insulin) that may be used as an intermediate for the manufacture of a conjugate described by Formula I.

In some embodiments, each secondary amine in Formulae FF1-FF224 that is not conjugated to any of B₁, B₂ or B₃, is optionally independently acetylated or alkylated.

In some embodiments, the N-terminus of the A-chain and/or the N-terminus of the B-chain of insulin are additionally each independently covalently conjugated to at least two aromatic boron-containing groups, and/or wherein the C-terminus of the B-chain is further extended with a polypeptide of up to 20 residues, or the C-terminus of the A-chain is further extended with a polypeptide of up to 40 residues, each polypeptide independently comprising at least one lysine residue in which the amino group of the lysine side chain is covalently conjugated as described by Formula I.

In some embodiments, X1 is insulin having a sequence comprising one selected from: a lysine at residue B21 of the B-chain and an arginine at residue B29 of the B-chain; a lysine at residue B21 of the B-chain; and a lysine at residue B29 of the B-chain; wherein an amino group of at least one lysine of the sequence is covalently conjugated as described by Formula I.

In some embodiments, X1 is insulin having a sequence comprising: a lysine at residue B21 of the B-chain; and at least one lysine at the N-terminus of the B-chain; wherein an amino group of at least one lysine of the sequence is covalently conjugated as described by Formula I.

In some embodiments, the C-terminus of Z1a is conjugated through an amide linkage to a Z1b, and the Z1b is conjugated to the N-terminus of the B-chain of insulin through an amine linkage, and wherein the insulin is optionally further conjugated as described by Formula I.

In some embodiments, the compound comprises at least one Z1a comprising at least three amino acid residues having a side chain; the side chain of two residues of Z1a are conjugated together through a covalent bond included in at least one selected from a triazole linkage, an amide linkage, a disulfide linkage, a thioether linkage, a thiolene linkage, and an amine linkage; and the two conjugated residues are at least one residue apart.

In some embodiments, the N-terminal amine of a Z1a is covalently conjugated to a Z1c.

In some embodiments, the compound comprises at least one Z1a comprising one or more amino acids selected from lysine, diaminopropionic acid, glycine, diaminobutyric acid, serine, histidine, and ornithine, and at least one or more of the side chains of the one or more amino acids, is covalently conjugated as described by Formula I.

In some embodiments, the compound comprises at least one Z1a comprising one or more glutamic or aspartic acid residues, and optionally other naturally occurring amino acids, and at least one lysine residue that is covalently conjugated as described by Formula I.

In some embodiments, the compound comprises at least one Z1a comprising at least one lysine residue that is covalently conjugated as described by Formula I, wherein the majority of the residues are negatively charged residues.

In some embodiments, the insulin is covalently conjugated as described by Formula I, and Z1b is absent and the C-terminus of Z1a is directly conjugated to the N-terminus of the B-chain of insulin through a peptide bond.

In some embodiments, the insulin is covalently conjugated as described by Formula I,
n′=0 and the C-terminus of Z1a is directly conjugated to the N-terminus of the B-chain of insulin through a peptide bond; and Z1a comprises at least one amino acid from each of groups M1 and M2 such that no two adjacent residues are from the same group and Z1a contains at least one lysine that is covalently conjugated as described by Formula I, wherein: (i) group M1 comprises lysine and alanine and group M2 comprises glycine, glutamic acid, serine, threonine, alanine and proline; or (ii) group M1 consists of lysine and alanine; and group M2 consists of glycine, glutamic acid, serine, threonine, alanine and proline. 19. In some embodiments, the insulin is covalently conjugated as described by Formula I,
n′=0 and the C-terminus of Z1a is directly conjugated to the N-terminus of the B-chain of insulin through a peptide bond;
Z1a comprises at least one amino acid selected from K, P, E, G, S, T, A, and R, such that the sequence comprises at least one lysine, at least one proline, and at least one amino acid selected from H, R, A and T; and
the amino group of least one lysine side chain in Z1a is covalently conjugated as described by Formula I.

In some embodiments, the insulin is covalently conjugated as described by Formula I, n′=0 and the C-terminus of Z1a is directly conjugated to the N-terminus of the B-chain of Insulin through a peptide bond; and Z1a comprises at least one amino acid selected from the alanine, glycine, aspartic acid, threonine, histidine, methionine, cysteine, isoleucine, leucine, valine and glutamine; and at least one lysine having a side chain amino group that is covalently conjugated as described by Formula I; and the rest of the amino acids in Z1a are each independently selected from the twenty naturally occurring amino acids.

In some embodiments, the insulin is covalently conjugated as described by Formula I, n′=0 and the C-terminus of Z1a is directly conjugated to the N-terminus of the B-chain of Insulin through a peptide bond; Z1a has a sequence selected from: KPA, KPH, GKPA, GKPS, KP, GKPSG, and GKPGS; and Z1a comprises at least one lysine having a side chain amino group that is covalently conjugated as described by Formula I.

In some embodiments, the insulin is covalently conjugated as described by Formula I, n′=0 and the C-terminus of Z1a is directly conjugated to the N-terminus of the B-chain of Insulin through a peptide bond; Z1a comprises two or more copies of a sequence selected from: EGE, SGS, GSG, KP, GEG, E, GG, S, T, A, and R, such that no two adjacent copies are the same; Z1a optionally contains one or more of H, A, N and R; and the amino group of least one lysine side chain in Z1a is covalently conjugated as described by Formula I.

In some embodiments, the insulin is covalently conjugated as described by Formula I, n′=0 and the C-terminus of Z1a is directly conjugated to the N-terminus of the B-chain of Insulin through a peptide bond; Z1a comprises one or more amino acids selected from K, P, E, G, S, T, A, and R, such the sequence comprises at least one lysine, at least one proline, and at least one amino acid selected from H, R, A and T; and the amino group of least one lysine side chain in Z1a is covalently conjugated as described by Formula I.

In some embodiments, the insulin is covalently conjugated as described by Formula I, n′=0 and the C-terminus of Z1a is directly conjugated to the N-terminus of the B-chain of Insulin through a peptide bond; and at least one copy of KP is comprised in the polypeptide sequence of Z1a or insulin, wherein the amino group of the lysine side chain in KP is covalently conjugated as described by Formula I.

In some embodiments, the insulin is covalently conjugated as described by Formula I, n′=0 and the side chains of two residues of Z1a are conjugated via a covalent bond selected from a triazole, an amide bond, a disulfide bond, a thioether, a thiolene, and an amine; the two conjugated residues are separated by at least one amino acid; and the amino group of least one lysine side chain in Z1a is covalently conjugated as described by Formula I.

In some embodiments, the insulin is covalently conjugated as described by Formula I, n′=0 and the C-terminus of Z1a is directly conjugated to the N-terminus of the B-chain of insulin through a peptide bond; Z1a is a polypeptide selected from the a polypeptide from the sequence of human insulin, a polypeptide from the sequence of human glucagon, a polypeptide from the sequence of human C-peptide, a polypeptide from the sequence of human GLP-1, a polypeptide from the sequence of human GIP, a polypeptide from the sequence of human Extendin, and a human polypeptide hormone, and wherein the polypeptide comprises at least one lysine or Z1a contains at least one copy of dipeptide KP, and wherein the amino group of at least one lysine side chain in Z1a is covalently conjugated as described by Formula I.

In some embodiments, at least one lysine residue, an inserted cysteine residue, or a residue that has been mutated to cysteine is covalently conjugated to a structure independently selected from Formulae F411-F416:

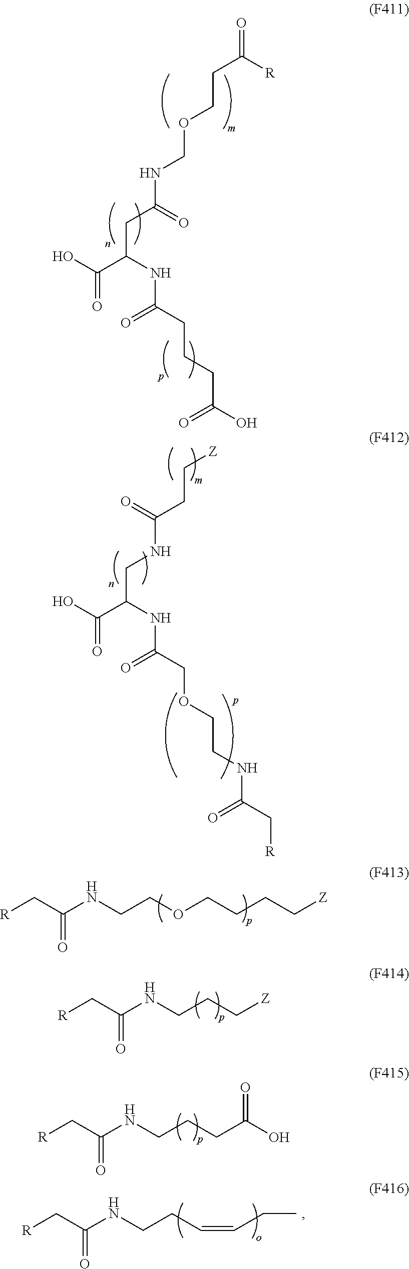

and
wherein in Formulae F411-F416, R represents an attachment point to the amine group of the lysine side chain, or the thiol group of the cysteine side chain; n is an integer in the range of 1 to 14, m is an integer between 1 and 12, o is an integer between 1 and 6, p is an integer between 1 and 12; and Z represents one of —(C=O)—OH, —NH₂, —CH₃, a cholesterol, 7-OH cholesterol, 7,25-dihydroxycholesterol, cholic acid, chenodeoxycholic acid, lithocholic acid, deoxycholic acid, glycocholic acid, glycodeoxycholic acid, glycolithocholic acid, glycochenodeoxycholic acid, α-tocopherol, β-tocopherol, γ-tocopherol, α-tocopherol, utocotrienol, β-tocotrienol, γ-tocotrienol, or δ-tocotrienol.

In some embodiments, the residue at position B29 of the B-chain of Insulin is a lysine covalently conjugated through an amide bond to the side chain of an L- or D-glutamic acid, and wherein the L- or D-glutamic acid is covalently conjugated through an amide bond to one of acid selected from hexanoic acid, myristic acid, octanoic acid, nonanoic acid, decanoic acid, lauric acid, stearic acid, and palmitic acid.

In some embodiments, the insulin is covalently conjugated as described by Formula I, Z1a comprises a polypeptide with the sequence (XA₁X)ₘ, wherein: A₁ is an L- or D-amino acid; m is an integer in the range of 1 to 4; and each X is K or KP; and the epsilon amine group of at least one lysine side chain in Z1a is further covalently conjugated as described by Formula I.

In some embodiments, the insulin is covalently conjugated as described by Formula I, Z1a comprises a polypeptide with the sequence (XA₁A₂X)ₘ(SEQ ID NO:24021), wherein: A₁ and A₂ are each independently an L- or D-amino acid; m is an integer in the range of 1 to 4; each X is K or KP; and the epsilon amine group of at least one lysine side chain in Z1a is further covalently conjugated as described by Formula I.

In some embodiments, the insulin is covalently conjugated as described by Formula I, Z1a comprises a polypeptide with the sequence (XA₁A₂A₃X)ₘ(SEQ ID NO:24022), wherein: A₁, A₂, and A₃ are each independently an L- or D-amino acid; m is an integer in the range of 1 to 4; each X is K or KP; and the epsilon amine group of at least one lysine side chain in Z1a is covalently conjugated as described by Formula I.

In some embodiments, the insulin is covalently conjugated as described by Formula I, Z1a comprises a polypeptide with a sequence selected from (XA1X)m(GGGGS)n (SEQ ID NO:24023), (XA1A2X)m (GGGGS)n (SEQ ID NO:24024), (XA1A2A3X)m(GGGGS)n (SEQ ID NO:24025), (XA1X)m(GGGGS)n (XA2X)o (SEQ ID NO:24026), and XA1A2X)m(GGGGS)n (XA3A4X)o (SEQ ID NO:24027), wherein: A₁, A₂, A₃, and A₄ are each independently an L- or D-amino acid; m is an integer in the range of 1 to 4; n is an integer in the range of 1 to 4; o is an integer in the range of 1 to 4; each X is K or KP; and the epsilon amine group of each lysine side chain of at least one lysine side chain in Z1a is further covalently conjugated as described by Formula I.

In some embodiments, the insulin is covalently conjugated as described by Formula I, Z1a comprises a polypeptide with the sequence (GX)ₘ, wherein: X is KV; m is an integer in the range of 1 to 4, and the epsilon amine group of at least one lysine side chain in Z1a is further covalently conjugated as described by Formula I.

In some embodiments, the insulin is covalently conjugated as described by Formula I, Z1a comprises a polypeptide with a sequence selected from: GXA1KGEA2XT)m(GGSGSSS)n (GXGXA3GSSSGSSSXT)o (SEQ ID NO:24028), (GXA1ESA2LYL)m (SEQ ID NO:24029), (TXEX)m(GPGS)n (SEQ ID NO:24030), (GXESA1VA)m (KA2K)n (SEQ ID NO:24031), (GXEA1A2)m(GGS)n (TYA3XXT)o (SEQ ID NO:24032), and (TXAXYT)m(TSSS)n (SEQ ID NO:24033), wherein: each X is KV or KP; A₁, A₂, A₃ are each independently an L- or D-amino acid; m is an integer in the range of 1 to 4; n is an integer in the range of 1 to 4; and o is an integer in the range of 1 to 4; and the epsilon amine group of at least one lysine side chain in Z1a is further covalently conjugated as described by Formula I.

In some embodiments, the insulin is covalently conjugated as described by Formula I, Z1a comprises a polypeptide with a sequence selected from (TKPYA1KEVETA2GSGS)m (GGGGS)n (SEQ ID NO:24034), (YTPLEA1KPYSTSYKPYSEA1L)m(GKPTSLEA2FLVEA2LYTKP)n (SEQ ID NO:24035), and (GKEALYLTPLESALYKP)m(TKPLEALYLKPEILSLKPESLA)n(GKPGSSSKPDTSSSGTP KTAAGS)o (SEQ ID NO:24036), wherein: A₁ and A₂ are each independently an L- or D-amino acid; m is an integer in the range of 1 to 4; n is an integer in the range of 1 to 4; and the epsilon amine group of at least one lysine side chain in Z1a is further covalently conjugated as described by Formula I.

In some embodiments, the compound is conjugated either directly, or via an optional covalent-spacer, to a drug molecule, an imaging agent, a contrast agent, a radioactive isotope, a radiotherapy agent, or a molecule that engages immune cells in the body.

In some embodiments, X1 is human glucagon or an analogue of human glucagon, and optionally covalently conjugated to one or more diol- or sugar-containing molecules, or X1 is an analogue of a human peptide hormone that is modified so that it binds to its cognate receptor but has diminished or null ability to activate the receptor in the body, or X1 is an analogue of a human peptide hormone that is modified so that it selectively binds or activates a subset of its cognate receptors or subsets of receptors of human polypeptide hormones.

In some embodiments, the aromatic boron-containing groups are modified to be MIDA protected, pinacol protected, or in an ester form. In some embodiments, the aromatic boron-containing group is MIDA protected or pinacol protected.

In some embodiments, the modified aromatic boron-containing groups are used as intermediates for the synthesis of a conjugate of Formula I.

In some embodiments, X1 comprises: (i) a human polypeptide hormone or an analogue of a human polypeptide hormone, wherein the covalent linkage to X1 is to an amine or via an optional covalent-spacer to an amine in X1; (ii) an amine configured to be covalently conjugated via an optional covalent-spacer to a human polypeptide hormone or an analogue of a human polypeptide hormone, or (iii) NH₂, and wherein the amine in X1 is covalently conjugated twice as described by Formula I, wherein the first covalent conjugation is through an amine bond and the second covalent conjugation is through an amide bond, and wherein each covalent conjugation is the same or different.

In some embodiments, residue B21 of the B-chain of Insulin is K, residue B22 of the B-chain of Insulin is P, and residue B29 of the B-chain of Insulin is R; and the N-terminus of the Insulin B-chain is covalently conjugated as described by Formula I to the C-terminus of Z1a, wherein: n′=0; Z1a has the sequence GKPGHKP; and one Z1c is attached to each lysine side chain in Z1a, wherein each Z1c is independently represented by Formula FF12, and each of B1 and B2 are represented by Formula F2, wherein one R1 at position 5′ is the covalent amide bond to Formula FF12, and the amino group of the lysine at B21 (residue 21 of the B-chain of Insulin) is covalently conjugated as described by Formula I, wherein n′=0; m′=0; and Z1c is described by Formula FF12, wherein each of B1 and B2 are represented by formula F2, and wherein one R1 at position 5′ is the covalent amide bond to Formula FF12.

In at least one embodiment, the present disclosure is directed to an insulin analog. In some embodiments, the insulin analog is desB30 human insulin; wherein the N-terminus of the Insulin B-chain is covalently conjugated as described by Formula I to the C-terminus of Z1a, wherein: n′=0; Z1a has the sequence KPGSEHESA, and one Z1c is attached to each lysine side chain in Z1a, wherein each Z1c is described by Formula FF1, and each of B1 and B2 are described by Formula F1, wherein one R1 at position 3′ is the covalent amide bond to Formula FF1 and wherein one R1 at position 5′ is F, and wherein the amino group of the lysine at B29 (residue 29 of the B-chain of Insulin) is covalently conjugated as described by Formula I, wherein n′=0; m′=0; and Z1c is described by Formula FF1, and each of B1 and B2 are described by Formula F1, wherein one R1 at position 3′ is the covalent amide bond to Formula FF1 and one R1 at position 5′ is F.

In some embodiments, the A- and/or B-chain sequence of the insulin is appended at the N-terminus or C-terminus by KX′K, KX′, or X′K wherein X′ represents a continuous sequence of 2, 3, 4, or 5 residues selected from within wild-type A-chain (SEQ ID NO:1) and wild-type B-chain (SEQ ID NO:2). In some embodiments, each K residue is optionally and independently covalently conjugated as described by Formula I. In some embodiments, X′ is a polypeptide of up to 30 residues with amino acids independently selected from: K, G, S, E, H, E, N, Q, D, A, P, R and C and each K residue is optionally and independently covalently conjugated as described by Formula I.

In some embodiments, the N-terminus of the A-chain and/or B-chain are optionally and independently covalently conjugated as described by Formula I.

In some embodiments, each K residue when present in insulin (insulin analog) is optionally and independently covalently conjugated as described by Formula I, wherein Z1c is any one of formulae FF1-FF224 and the B1 and B2 are each independently selected from F1 and F2.

In some embodiments, each K residue when present in insulin is optionally and independently covalently conjugated as described by Formula I, wherein Z1c is any one of formulae:
Formulae FF1-F22 and the B1 and B2 are each independently selected from F1 and F2;
Formulae FF23-FF48 and the B1 and B2 are each independently selected from F1 and F2;
Formulae FF49-FF88 and the B1 and B2 are each independently selected from F1 and F2;
Formulae FF89-FF112 and the B1 and B2 are each independently selected from F1 and F2;
Formulae FF113-FF136 and the B1 and B2 are each independently selected from F1 and F2;
Formulae FF137-FF160 and the B1 and B2 are each independently selected from F1 and F2;
Formulae FF160-FF166 and the B1 and B2 are each independently selected from F1 and F2; or
Formulae FF167-FF224 and the B1 and B2 are each independently selected from F1 and F2.

In some embodiments, Z1b is optionally selected from Formula IIa-Formula IIi; and/or optionally selected from Formula IIIa-Formula IIIi.

In at least some embodiments, the insulin does not comprise Z1a and/or Z1b. In some embodiments, Z1a is not present. In some embodiments, Z1b is not present. In at least some embodiments, Z1a and/or Z1b are not present.

In some embodiments, each K residue when present in insulin is optionally and independently covalently conjugated as described by Formula I, and wherein Z1c is any one of formulae FF1-FF224 and the B1 and B2 are each independently selected from F3 and F4.

In some embodiments, each K residue when present in insulin is optionally and independently covalently conjugated as described by Formula I, wherein Z1c is any one of formulae:
Formulae FF1-F22 and the B1 and B2 are each independently selected from F3 and F4;
Formulae FF23-FF48 and the B1 and B2 are each independently selected from F3 and F4;
Formulae FF49-FF88 and the B1 and B2 are each independently selected from F3 and F4;
Formulae FF89-FF112 and the B1 and B2 are each independently selected from F3 and F4;
Formulae FF113-FF136 and the B1 and B2 are each independently selected from F3 and F4;
Formulae FF137-FF160 and the B1 and B2 are each independently selected from F3 and F4;
Formulae FF160-FF166 and the B1 and B2 are each independently selected from F3 and F4; or
Formulae FF167-FF224 and the B1 and B2 are each independently selected from F3 and F4.

In some embodiments, Z1b is optionally selected from Formula IIa-Formula IIi and Formula IIIa-Formula IIIi.

In some embodiments, Z1a and/or Z1b are not present.

In some embodiments, each K residue is optionally and independently covalently conjugated as described by Formula I, wherein Z1c is any one of Formulae FF1-F224 and the B1 and B2 are each independently selected from F5, F6, F7, and F8.

In some embodiments, each K residue when present in insulin is optionally and independently covalently conjugated as described by Formula I, wherein Z1c is any one of formulae:
Formulae FF1-F22 and the B1 and B2 are each independently selected from F5, F6, F7, and F8;
Formulae FF23-FF48 and the B1 and B2 are each independently selected from F5, F6, F7, and F8;
Formulae FF49-FF88 and the B1 and B2 are each independently selected from F5, F6, F7, and F8;
Formulae FF89-FF112 and the B1 and B2 are each independently selected from F5, F6, F7, and F8;
Formulae FF113-FF136 and the B1 and B2 are each independently selected from F5, F6, F7, and F8;
Formulae FF137-FF160 and the B1 and B2 are each independently selected from F5, F6, F7, and F8;
Formulae FF160-FF166 and the B1 and B2 are each independently selected from F5, F6, F7, and F8; or
Formulae FF167-FF224 and the B1 and B2 are each independently selected from F5, F6, F7, and F8.

In some embodiments, Z1b is optionally selected from Formula IIa-Formula IIi and Formula IIIa-Formula IIIi.

In some embodiments, Z1a and/or Z1b are not present.

In some embodiments, each K residue when present in insulin is optionally and independently covalently conjugated as described by Formula I, wherein Z1c is any one of formulae FF1-F224and the B1 and B2 are each independently selected from F9 and F10.

In some embodiments, each K residue when present in insulin is optionally and independently covalently conjugated as described by Formula I, wherein Z1c is any one of formulae:
Formulae FF1-F22 and the B1 and B2 are each independently selected from F9 and F10;
Formulae FF23-FF48 and the B1 and B2 are each independently selected from F9 and F10;
Formulae FF49-FF88 and the B1 and B2 are each independently selected from F9 and F10;
Formulae FF89-FF112 and the B1 and B2 are each independently selected from F9 and F10;
Formulae FF113-FF136 and the B1 and B2 are each independently selected from F9 and F10;
Formulae FF137-FF166 and the B1 and B2 are each independently selected from F9 and F10; or
Formulae FF167-FF224 and the B1 and B2 are each independently selected from F9 and F10.

In some embodiments, Z1b is not present in insulin (i.e., insulin analog) and wherein Z1a is a polypeptide that is covalently linked by a peptide bond to the N-terminus of the B-chain of insulin and/or Z1a is a polypeptide that is covalently linked by a peptide bond to the N-terminus of the B-chain of insulin C-terminus of A-chain of insulin.

In some embodiments, Z1b may be present in insulin. If present in insulin, Z1b is optionally selected from Formula IIa-Formula IIi and Formula IIIa-Formula IIIi.

In some embodiments, Z1b is not present in insulin. In some embodiments, Z1a is not present. In at least some embodiments, Z1b and/or Z1a are not present.

### Methods of Preparation

In at least one embodiment, the present disclosure provides a method to prepare a compound comprising an aromatic boron-containing compound and/or an aromatic boron-containing group (e.g., Z1c, Formula I) or a pharmaceutical preparation comprising one or more compounds of the present disclosure.

In at least one embodiment, the disclosure provides a method for preparing rotationally constrained tether boron conjugates that contain scaffolds (Z1c) that are rotationally hindered by disfavored steric interactions (e.g. gauche vs anti interactions of substituents), hindered rotation due to bond hybridization (e.g., cis- vs trans-amide rotations), or through rigid covalent bonds (e.g., (E) vs (Z) configurations for alkene moieties). For example, formulae FF50-FF62, FF116, and FF121-134 contain alkyl functionalities geminal (e.g., attached to the same atom) to the amine groups that are covalently conjugated to the boronic acid functionalized moieties. As another example, Formulae FF50-FF62, FF116, and FF121-134 contain geminal alkyl substituents which may limit the accessible dihedral angles that the boron conjugated amines adopt, influencing adopted dihedral angles and placing the boronic functionalized groups closer together and allowing for increased binding of the conjugates to target molecules such as proteins or sugars.

### Methods of Treatment

In at least one embodiment, the disclosure provides a method of treating a subject suffering from, or susceptible to, a disease that is beneficially treated by a compound disclosed herein or a pharmaceutical preparation comprising one or more of the compounds disclosed herein. In some embodiments, the method comprises the step of administering to a subject in need thereof an effective amount of a pharmaceutical preparation/composition of the present disclosure. In at least one embodiment, the compound(s) and/or pharmaceutical preparations of the present disclosure may be for use in (or in the manufacture of medicaments for) the treatment or prevention of disorders, including hyperglycemia, type 2 diabetes, impaired glucose tolerance, type 1 diabetes, obesity, metabolic syndrome X, or dyslipidemia, diabetes during pregnancy, pre-diabetes, Alzheimer's disease, MODY 1, MODY 2 or MODY 3 diabetes, mood disorders, and psychiatric disorders. In at least one embodiment, a therapeutically-effective amount of a compound and/or pharmaceutical preparation of the present disclosure is administered to a subject suffering from diabetes.

The following examples and experimental data are provided for illustrative purposes only, and do not limit the scope of the embodiments of the present disclosure.

The following abbreviations have the definitions set forth below:

| Abbreviation | Full Name |
|---|---|
| Acm | s-Acetamidomethyl group |
| ACN | Acetonitrile |
| ARS | Alizarin Red S |
| Boc | tert-Butyloxycarbonyl |
| DCM | Dichloromethane |
| Dde | 1-(4,4-dimethyl-2,6-dioxocyclohex-1-ylidene)ethyl |
| DIPEA, DIEA | N,N-Diisopropylethylamine |
| DMF | Dimethylformamide |
| DMSO | Dimethyl Sulfoxide |
| DTDP | 2,2,-Dithiopyridine |
| EDC | 3-(Ethyliminomethyleneamino)-N,N-dimethylpropan-1- |
| | amine |
| Fmoc | Fluorenylmethyloxycarbonyl Chloride |
| GLP-1 | Glucagon-Like Peptide 1 |
| HATU | Hexafluorophosphate Azabenzotriazole Tetramethyl |
| | Uronium |
| HCl | Hydrochloric Acid |
| IGF1 | Insulin-like Growth Factor 1 |
| LC-MS | Liquid Chromatography-Mass Spectrometry |
| MeOH | Methanol |
| MIDA | N-methyliminodiacetic acid |
| MODY | Maturity Onset Diabetes of the Young |
| NHS | N-Hydroxysuccinimide |
| Oxyma | Ethyl cyanohydroxyiminoacetate |
| RAM | Rink Amide Matrix |
| PEG | Polyethylene Glycol |
| SDS | Sodium Dodecyl Sulfate |
| tBu | tert-Butyl |
| TFA | Trifluoroacetic Acid |

### EXAMPLES

### A. Preparation of Aromatic Boron-Containing Compounds.

The disclosed compounds can be prepared according to the following schemes. The following schemes represent the general methods used in preparing these compounds. However, the synthesis of these compounds is not limited to these representative methods, as they can also be prepared through various other methods by those skilled in the art of synthetic chemistry.

### Method 1: Synthesis of Diboronates DS01-DS48:

Chlorotrityl resin (300 mg, 0.45 mmol) was swelled in dry DCM (5 mL) for 30 mins. Solvent was removed with nitrogen and a solution of bromo acetic acid (0.139 g, 1M, 1 mL) in DCM with DIPEA (1M, 0.13 g 1 mL) was added immediately and gently mixed for 1 hr. The mixture was washed with DCM and unreacted sites were capped with a solution of 20% MeOH in a solution of DCM and DIEA (1M) and mixed for 1 hr. The resin was washed with DCM (3×5 mL) then DMF (3×5 mL). A solution of FF-diamine linker propane-1,3-diamine (0.37 g, 1M) in DMF (5 mL) was added to the resin and heated at 50° C. for 10 minutes. The resin was washed with DMF (3×5 mL) and a solution of 3-borono-5-nitrobenzoic acid (0.2M, 0.21 mg, 5 mL) in DMF with N, N′-diisopropylcarbodiimide (DIC) (0.126 g, 1M, 1 mL), Oxyma (0.5 M, 0.142 g, 2 mL) in DMF and heated at 50° C. for 30 min. The resin was washed with DMF (3×5 mL) then DCM (3×5 mL). A solution of trifluoroacetic acid with triisopropyl silane and water (95:2.5:2.5, 5 mL) was added to the resin and mixed for 90 minutes. The solution was collected and dried under vacuum, dissolved in DMSO (100 uL) and fractionated by reverse-phase (RP) flash chromatography on a C18 column with a gradient of 20% ACN in water with 0.1% TFA to 60% ACN in water with 0.1% TFA over 10 minutes. Pure fractions were isolated, combined, frozen, and lyophilized to yield N-(3-(3-borono-5-nitrobenzamido)propyl)-N-(3-borono-5-nitrobenzoyl)glycine (DS01) as a white powder (7 mg).

Similar procedures are followed for the synthesis of aromatic boron-containing groups DS02-DS48.

### Method 2: Synthesis of Symmetric Diboronates With C-Terminus Linker DS49-DS53

Tentagel-S—NH₂ resin (250 mg, 0.05 mmol) was swelled in DMF (5 mL) for 2 hr. The solution was removed under a stream of nitrogen and a solution of Boc-Gly-HMBA (0.2 mmol) was coupled using 1-[Bis(dimethylamino)methylene]-1H-1,2,3-triazolo[4,5-b]pyridinium 3-oxide hexafluorophosphate (HATU, 76 mg, 0.2 mmol), and DIPEA (70 ul) in DMF (2 mL) was added to the resin and mixed at room temperature for 45 minutes. The resin was washed with DMF (3×5 mL) and DCM (3×5 mL). A solution of 50% trifluoroacetic acid in DCM (5 mL) was added to the resin and mixed for 20 minutes to remove the Boc protecting group. This step was repeated twice. The resin was washed with DCM (3×5 mL) and DMF (3×5 mL) and was treated with a solution of 10% DIEA in DMF (5 mL) for 10 minutes, the cycle was repeated twice, and resin was washed with DMF (3×5 mL). A solution of 1-(((9H-fluoren-9-yl)methoxy)carbonyl)-4-((((9H-fluoren-9-yl)methoxy)carbonyl)amino)pyrrolidine-2-carboxylic acid (0.115 g, 0.2 mmol) with 1-[Bis(dimethylamino)methylene]-1H-1,2,3-triazolo[4,5-b]pyridinium 3-oxide hexafluorophosphate (HATU, 0.2 mmol), and DIPEA (70 ul) in DMF (2 mL) was added to the resin and mixed for 45 minutes. The resin was washed with DMF (3×5 mL) and a solution of 20% piperidine in DMF (5 mL×3) was added to resin and mixed for 5 minutes. The resin was washed with DMF (3×5 mL) and a solution of 1-hydroxy-1,3-dihydrobenzo[c][1,2]oxaborole-6-carboxylic acid (0.078 g, 0.4 mmol) with HATU (0.4 mmol) and DIPEA (140 uL) in DMF (2 mL) was added to the resin and mixed for 45 minutes. The resin was washed with DMF (3×5 mL) then DCM (3×5 mL). A solution of 0.1 M NaOH in 1:5 water:THF was added to the resin and mixed for 90 minutes. The solution was filtered and adjusted the pH-2 using 1.0 M HCl and fractionated by reverse-phase (RP) flash chromatography on a C18 column with a gradient of 20% ACN in water with 0.1% TFA to 60% ACN in water with 0.1% TFA over 10 minutes. Pure fractions were isolated, combined, frozen, and lyophilized to yield ((2S,4S)-1-(1-hydroxy-1,3-dihydrobenzo[c][1,2]oxaborole-6-carbonyl)-4-(1-hydroxy-1,3-dihydrobenzo[c][1,2]oxaborole-6-carboxamido)pyrrolidine-2-carbonyl)glycine (DS49) as a white powder (10 mg).

Similar procedures are followed for the synthesis of aromatic boron-containing groups DS50-DS53.

Method 3: Synthesis of Diboronates with Reductive Alkylation on Side Chain Amine DS54-DS63

Rink-amide resin (0.05 mmol, 263 mg) was swelled in DMF (5 mL) for 20 minutes. The solution was removed under a stream of nitrogen and a solution of 20% piperidine in DMF (5 mL) was added to resin and mixed for 5 minutes. The resin was washed with DMF (3×5 mL). A solution of ((S)-2-((((9H-fluoren-9-yl)methoxy)carbonyl)amino)-3-((diphenyl(p-tolyl)methyl)amino) propanoic acid (116 mg, 0.2 mmol) with 1-[Bis(dimethylamino)methylene]-1H-1,2,3-triazolo[4,5-b]pyridinium 3-oxide hexafluorophosphate (HATU, 76 mg, 0.2 mmol), and DIPEA (70 ul) in DMF (2 mL) was added to the resin and mixed at room temperature for 45 minutes. The resin was washed with DMF (3×5 mL) and DCM (3×5 mL). A solution of 0.2% trifluoroacetic acid in DCM (5 mL) was added to the resin and mixed for 10 minutes to remove Mtt protecting group. This step was repeated twice. The resin was washed with DCM (3×5 mL) and DMF (3×5 mL) and was treated with a solution of 10% DIEA in DMF (5 mL) for 10 minutes, the cycle was repeated twice, and resin was washed with DMF (3×5 mL). A solution of benzaldehyde (0.053 g, 0.5 mol) in trimethyl orthoformate (TMOF) (2 mL) was added to the resin and mixed for 1 hr. The solution was filtered, and resin was washed with DMF (3×5 mL) and 10% acetic acid in methanol (2×5 mL). The solution of NaCNBH₃ (10 equivalents) in 10% acetic acid in methanol was added to the resin and mixed for 1 hr, washed with DMF (3×5 mL). The resin was treated with 20% piperidine in DMF (3×5 mL) to deprotect the Fmoc, washed with DMF (3×5 mL) and coupled with 1-hydroxy-1,3-dihydrobenzo[c][1,2]oxaborole-6-carboxylic acid (0.078 g, 0.4 mmol) using 1-[Bis(dimethylamino)methylene]-1H-1,2,3-triazolo[4,5-b]pyridinium 3-oxide hexafluorophosphate (HATU, 152 mg, 0.4 mmol), and DIPEA (140 ul) in DMF (2 mL) for 45 minutes. After coupling reaction, the resin was washed with DMF (3×5 mL) then with DCM (2×5 mL). A solution of trifluoroacetic acid with triisopropyl silane and water (95:2.5:2.5, 5 mL) was added to the resin and mixed for 90 minutes. The solution was collected and dried under vacuum, dissolved in DMSO (100 uL) and fractionated by reverse-phase (RP) flash chromatography on a C18 column with a gradient of 20% ACN in water with 0.1% TFA to 60% ACN in water with 0.1% TFA over 10 minutes. Pure fractions were isolated, combined, frozen, and lyophilized to yield (S)—N-(3-amino-2-(1-hydroxy-1,3-dihydrobenzo[c][1,2]oxaborole-6-carboxamido)-3-oxopropyl)-N-benzyl-1-hydroxy-1,3-dihydrobenzo[c][1,2]oxaborole-6-carboxamide (DS54) as a white powder (7 mg).

Similar procedures are followed for the synthesis of aromatic boron-containing groups DS55-DS63.

### Method 4: Synthesis of Symmetric Diboronates Based on Amino Acids DS64-DS76, DS109-DS111

Rink-amide resin (0.05 mmol, 263 mg) was swelled in DMF (5 mL) for 20 minutes. The solution was removed under a stream of nitrogen and a solution of 20% piperidine in DMF (5 mL) was added to resin and mixed for 5 minutes. The resin was washed with DMF (3×5 mL). A solution of 1-(((9H-fluoren-9-yl)methoxy)carbonyl)-4-((((9H-fluoren-9 yl)methoxy) carbonyl) amino) pyrrolidine-2-carboxylic acid (0.115 g, 0.2 mmol) with 1-[Bis(dimethylamino)methylene]-1H-1,2,3-triazolo[4,5-b]pyridinium 3-oxide hexafluorophosphate (HATU, 0.2 mmol), and DIPEA (70 ul) in DMF (2 mL) was added to the resin and mixed at 50° C. for 20 minutes. The resin was washed with DMF (3×5 mL) and a solution of 20% piperidine in DMF (5 mL) was added to the resin and mixed for 5 minutes. The resin was washed with DMF (3×5 mL) and a solution of 1-hydroxy-1,3-dihydrobenzo[c][1,2]oxaborole-6-carboxylic acid (0.078 g, 0.4 mmol) with HATU (0.4 mmol) and DIPEA (140 uL) in DMF (2 mL) was added to the resin and mixed at 50° C. for 30 minutes. The resin was washed with DMF (3×5 mL) then with DCM (3×5 mL). A solution of trifluoroacetic acid with triisopropyl silane and water (95:2.5:2.5, 5 mL) was added to the resin and mixed for 90 minutes. The solution was collected and dried under vacuum, dissolved in DMSO (100 uL) and fractionated by reverse-phase (RP) flash chromatography on a C₁₈ column with a gradient of 20% ACN in water with 0.1% TFA to 60% ACN in water with 0.1% TFA over 10 minutes. Pure fractions were isolated, combined, frozen, and lyophilized to yield (3-((2S,4S)-4-(5-borono-2-(methylsulfonyl)benzamido)-2-carbamoylpyrrolidine-1-carbonyl)-4-(methylsulfonyl)phenyl)boronic acid (DS64) as a white powder (10 mg).

Similar procedures are followed for the synthesis of aromatic boron-containing groups DS65-DS76. Similar procedure can be followed for the synthesis of diboronate sensors DS109-DS111 noting that the resin used is 2-chlorotrityl resin and not the Amine Rink-amide resin.

### Method 5: Asymmetric Diboronate Synthesis DS77-DS79

Tentagel-S—NH₂ resin (250 mg, 0.05 mmol) was swelled in DMF (5 mL) for 2 hr. The solution was removed under a stream of nitrogen and a solution of Boc-Gly-HMBA (0.2 mmol) was coupled using 1-[Bis(dimethylamino)methylene]-1H-1,2,3-triazolo[4,5-b]pyridinium 3-oxide hexafluorophosphate (HATU, 76 mg, 0.2 mmol), and DIPEA (70 ul) in DMF (2 mL) was added to the resin and mixed at room temperature for 45 minutes. The resin was washed with DMF (3×5 mL) and DCM (3×5 mL). A solution of 50% trifluoroacetic acid in DCM (5 mL) was added to the resin and mixed for 20 minutes. To remove Boc protecting group. This step was repeated twice. The resin was washed with DCM (3×5 mL) and DMF (3×5 mL) and was treated with a solution of 10% DIEA in DMF (5 mL) for 10 minutes, the cycle was repeated twice, and resin was washed with DMF (3×5 mL). A solution of 1-(((9H-fluoren-9-yl)methoxy)carbonyl)-4-((tert-butoxycarbonyl)amino)pyrrolidine-2-carboxylic acid (0.09 g, 0.2 mmol) with 1-[Bis(dimethylamino)methylene]-1H-1,2,3-triazolo[4,5-b]pyridinium 3-oxide hexafluorophosphate (HATU, 0.2 mmol), and DIPEA (70 ul) in DMF (2 mL) was added to the resin and mixed for 45 minutes. The resin was washed with DMF (3×5 mL) and a solution of 20% piperidine in DMF (5 mL×3) was added to resin and mixed for 5 minutes. The resin was washed with DMF (3×5 mL) and a solution of 1-hydroxy-1,3-dihydrobenzo[c][1,2]oxaborole-6-carboxylic acid (0.039 g, 0.2 mmol) with HATU (0.076 g, 0.2 mmol) and DIPEA (140 uL) in DMF (2 mL) was added to the resin and mixed for 45 minutes. and DCM (3×5 mL). A solution of 50% trifluoroacetic acid in DCM (5 mL) was added to the resin and mixed for 20 minutes, to remove Boc protecting group. This step was repeated twice. The resin was washed with DCM (3×5 mL) and DMF (3×5 mL) and was treated with a solution of 10% DIEA in DMF (5 mL) for 10 minutes, the cycle was repeated twice, and resin was washed with DMF (3×5 mL). A solution of 5-borono-2-nitrobenzoic acid (0.042 g, 0.2 mmol) with HATU (0.076 g, 0.2 mmol) and DIPEA (140 uL) in DMF (2 mL) was added to the resin and mixed for 45 minutes. The resin was washed with DMF (3×5 mL) then DCM (3×5 mL). A solution of 0.1M NaOH in 1:5 water:THF was added to the resin and mixed for 90 minutes. The solution was filtered and adjusted the pH-2 using 1.0 M HCl and fractionated by reverse-phase (RP) flash chromatography on a C18 column with a gradient of 20% ACN in water with 0.1% TFA to 60% ACN in water with 0.1% TFA over 10 minutes. Pure fractions were isolated, combined, frozen, and lyophilized to yield ((2S,4S)-1-(5-borono-2-nitrobenzoyl)-4-(1-hydroxy-1,3-dihydrobenzo[c][1,2]oxaborole-6-carboxamido)pyrrolidine-2-carbonyl)glycine (DS77) as a white powder (8 mg).

Similar procedures are followed for the synthesis of aromatic boron-containing groups DS78-DS79.

Method 6: Synthesis of Diboronates with Reductive Alkylation on Side Chain DS80-DS109

Amine Rink-amide resin (0.05 mmol, 263 mg) was swelled in DMF (5 mL) for 20 minutes. The solution was removed under a stream of nitrogen and a solution of 20% piperidine in DMF (5 mL) was added to resin and mixed for 5 minutes. The resin was washed with DMF (3×5 mL). A solution of N⁶-(((9H-fluoren-9-yl)methoxy)carbonyl)-N2-(tert-butoxycarbonyl)lysine (93.6 mg, 0.2 mmol) with 1-[Bis(dimethylamino)methylene]-1H-1,2,3-triazolo[4,5-b]pyridinium 3-oxide hexafluorophosphate (HATU, 76 mg, 0.2 mmol), and DIPEA (70 ul) in DMF (2 mL) was added to the resin and mixed at room temperature for 45 minutes. The resin was washed with DMF (3×5 mL). The Fmoc was removed with 20% piperidine in DMF (2×5 mL) and then washed with additional DMF (3×10 mL) A solution of 4-methyl-3-(4,4,5,5-tetramethyl-1,3,2-dioxaborolan-2-yl)benzaldehyde (123 mg, 0.5 mol) in trimethyl orthoformate (TMOF) (2 mL) was added to the resin and mixed for 1 hr. The solution was filtered, and resin was washed with DMF (3×5 mL) and a solution of NaBH₄ (10 equivalents) in 20% methanol in DMF was added to the resin and mixed for 1 hr, washed with DMF (3×5 mL). The reduced amine was then coupled with 3-nitro-5-(4,4,5,5-tetramethyl-1,3,2-dioxaborolan-2-yl)benzoic acid (117 mg, 0.4 mmol) using 1-[Bis(dimethylamino)methylene]-1H-1,2,3-triazolo[4,5-b]pyridinium 3-oxide hexafluorophosphate (HATU, 152 mg, 0.4 mmol), and DIPEA (140 ul) in DMF (2 mL) for 45 minutes. After coupling reaction, the resin was washed with DMF (3×5 mL) then with DCM (2×5 mL). A solution of trifluoroacetic acid with triisopropyl silane and water (95:2.5:2.5, 5 mL) was added to the resin and mixed for 90 minutes. The solution was collected and dried under vacuum, dissolved in DMSO (100 uL) and fractionated by reverse-phase (RP) flash chromatography on a C₁₈ column with a gradient of 20% ACN in water with 0.1% TFA to 60% ACN in water with 0.1% TFA over 10 minutes. Pure fractions were isolated, combined, frozen, and lyophilized to yield diboronate sensor DS80 as a white powder (10 mg). Similar procedure was followed for the synthesis of diboronate sensors DS81-DS109.

The chemical structure and IUPAC name of DS1 to DS109 are summarized in Table 1 below.

TABLE 1

| Compd | | | |
|---|---|---|---|
| # | FF | F | Structure/IUPAC Name |
| DS01 | FF10 | F1 | 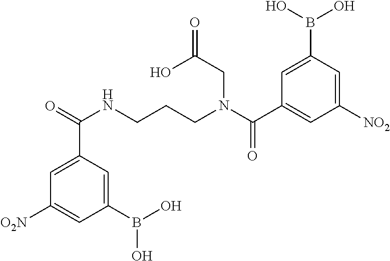 [SMILES: O=C(O)CN(CCCNC(=O)c1cc(B(O)O)cc([N+](=O)[O-])c1)C(=O)c1cc(B(O)O)cc([N+](=O)[O-])c1] |
| | | | N-(3-(3-borono-5-nitrobenzamido)propyl)-N-(3-borono-5- |
| | | | nitrobenzoyl)glycine |
| DS02 | FF217 | F1 | 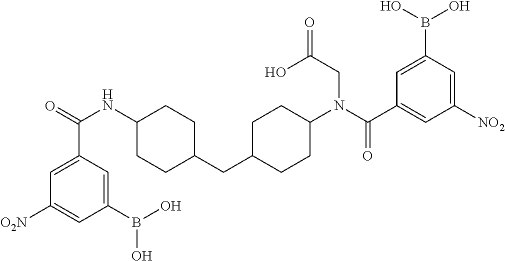 [SMILES: O=C(O)CN(C(=O)c1cc(B(O)O)cc([N+](=O)[O-])c1)C1CCC(CC2CCC(NC(=O)c3cc(B(O)O)cc([N+](=O)[O-])c3)CC2)CC1] |
| | | | N-(4-((4-(3-borono-5- |
| | | | nitrobenzamido)cyclohexyl)methyl)cyclohexyl)-N-(3-borono-5- |
| | | | nitrobenzoyl)glycine |
| DS03 | FF8 | F1 | 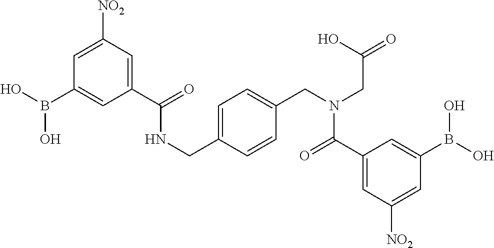 [SMILES: O=C(O)CN(Cc1ccc(CNC(=O)c2cc(B(O)O)cc([N+](=O)[O-])c2)cc1)C(=O)c1cc(B(O)O)cc([N+](=O)[O-])c1] |
| | | | N-(4-((3-borono-5-nitrobenzamido)methyl)benzyl)-N-(3-borono-5- |
| | | | nitrobenzoyl)glycine |
| DS04 | FF14 | F1 | 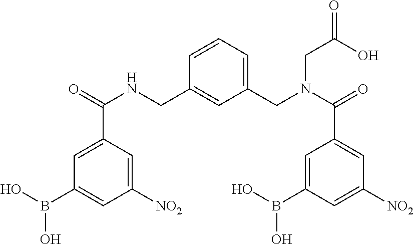 [SMILES: O=C(O)CN(Cc1cccc(CNC(=O)c2cc(B(O)O)cc([N+](=O)[O-])c2)c1)C(=O)c1cc(B(O)O)cc([N+](=O)[O-])c1] |
| | | | N-(3-((3-borono-5-nitrobenzamido)methyl)benzyl)-N-(3-borono-5- |
| | | | nitrobenzoyl)glycine |
| DS05 | FF10 | F1 | 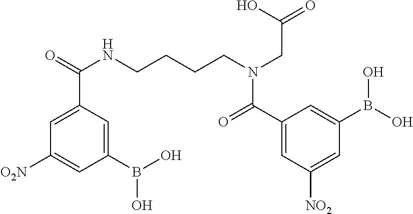 [SMILES: O=C(O)CN(CCCCNC(=O)c1cc(B(O)O)cc([N+](=O)[O-])c1)C(=O)c1cc(B(O)O)cc([N+](=O)[O-])c1] |
| | | | N-(4-(3-borono-5-nitrobenzamido)butyl)-N-(3-borono-5- |
| | | | nitrobenzoyl)glycine |
| DS06 | FF10 | F1 | 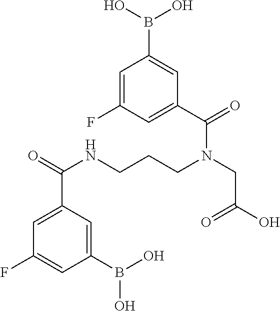 [SMILES: O=C(O)CN(CCCNC(=O)c1cc(F)cc(B(O)O)c1)C(=O)c1cc(F)cc(B(O)O)c1] |
| | | | N-(3-(3-borono-5-fluorobenzamido)propyl)-N-(3-borono-5- |
| | | | fluorobenzoyl)glycine |
| DS07 | FF213 | F1 | 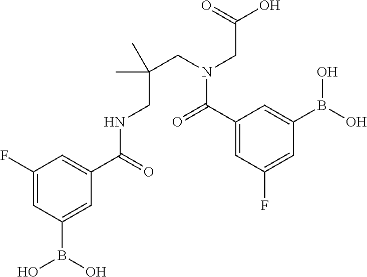 [SMILES: CC(C)(CNC(=O)c1cc(F)cc(B(O)O)c1)CN(CC(=O)O)C(=O)c1cc(F)cc(B(O)O)c1] |
| | | | N-(3-(3-borono-5-fluorobenzamido)-2,2-dimethylpropyl)-N-(3- |
| | | | borono-5-fluorobenzoyl)glycine |
| DS08 | FF214 | F1 | 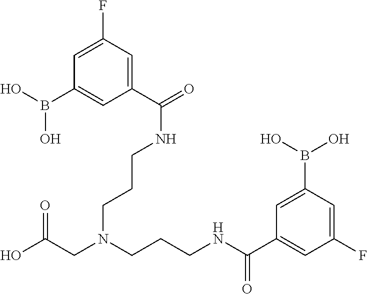 [SMILES: O=C(O)CN(CCCNC(=O)c1cc(F)cc(B(O)O)c1)CCCNC(=O)c1cc(F)cc(B(O)O)c1] |
| | | | bis(3-(3-borono-5-fluorobenzamido)propyl)glycine |
| DS09 | FF8 | F1 | 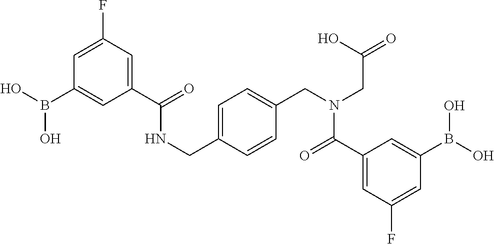 [SMILES: O=C(O)CN(Cc1ccc(CNC(=O)c2cc(F)cc(B(O)O)c2)cc1)C(=O)c1cc(F)cc(B(O)O)c1] |
| | | | N-(4-((3-borono-5-fluorobenzamido)methyl)benzyl)-N-(3-borono- |
| | | | 5-fluorobenzoyl)glycine |
| DS10 | FF14 | F | 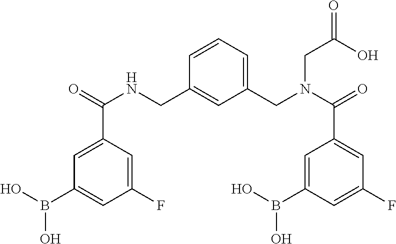 [SMILES: O=C(O)CN(Cc1cccc(CNC(=O)c2cc(F)cc(B(O)O)c2)c1)C(=O)c1cc(F)cc(B(O)O)c1] |
| | | | N-(3-((3-borono-5-fluorobenzamido)methyl)benzyl)-N-(3-borono- |
| | | | 5-fluorobenzoyl)glycine |
| DS11 | FF1 | F1 | 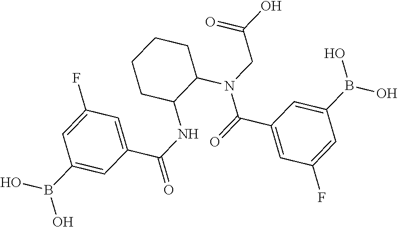 [SMILES: O=C(O)CN(C(=O)c1cc(F)cc(B(O)O)c1)C1CCCCC1NC(=O)c1cc(F)cc(B(O)O)c1] |
| | | | N-(2-(3-borono-5-fluorobenzamido)cyclohexyl)-N-(3-borono-5- |
| | | | fluorobenzoyl)glycine |
| DS12 | FF10 | F1 | 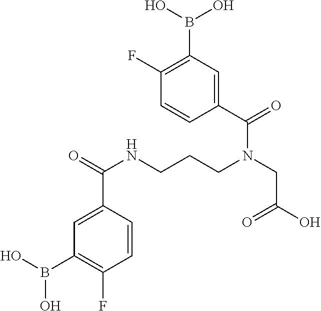 [SMILES: O=C(O)CN(CCCNC(=O)c1ccc(F)c(B(O)O)c1)C(=O)c1ccc(F)c(B(O)O)c1] |
| | | | N-(3-(3-borono-4-fluorobenzamido)propyl)-N-(3-borono-4- |
| | | | fluorobenzoyl)glycine |
| DS13 | FF217 | F1 | 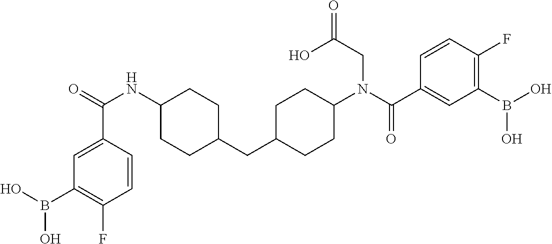 [SMILES: O=C(O)CN(C(=O)c1ccc(F)c(B(O)O)c1)C1CCC(CC2CCC(NC(=O)c3ccc(F)c(B(O)O)c3)CC2)CC1] |
| | | | N-(4-((4-(3-borono-4- |
| | | | fluorobenzamido)cyclohexyl)methyl)cyclohexyl)-N-(3-borono-4- |
| | | | fluorobenzoyl)glycine |
| DS14 | FF213 | F1 | 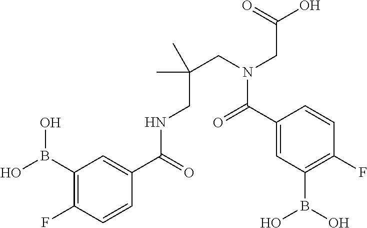 [SMILES: CC(C)(CNC(=O)c1ccc(F)c(B(O)O)c1)CN(CC(=O)O)C(=O)c1ccc(F)c(B(O)O)c1] |
| | | | N-(3-(3-borono-4-fluorobenzamido)-2,2-dimethylpropyl)-N-(3- |
| | | | borono-4-fluorobenzoyl)glycine |
| DS15 | FF8 | F1 | 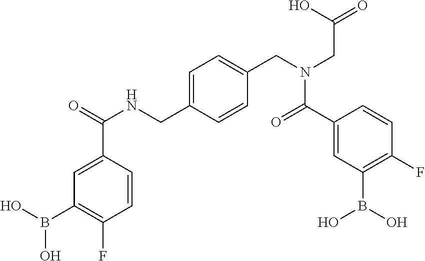 [SMILES: O=C(O)CN(Cc1ccc(CNC(=O)c2ccc(F)c(B(O)O)c2)cc1)C(=O)c1ccc(F)c(B(O)O)c1] |
| | | | N-(4-((3-borono-4-fluorobenzamido)methyl)benzyl)-N-(3-borono- |
| | | | 4-fluorobenzoyl)glycine |
| DS16 | FF14 | F1 | 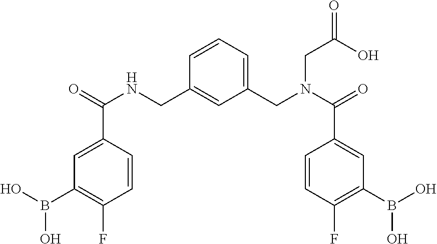 [SMILES: O=C(O)CN(Cc1cccc(CNC(=O)c2ccc(F)c(B(O)O)c2)c1)C(=O)c1ccc(F)c(B(O)O)c1] |
| | | | N-(3-((3-borono-4-fluorobenzamido)methyl)benzyl)-N-(3-borono- |
| | | | 4-fluorobenzoyl)glycine |
| DS17 | FF1 | F1 | 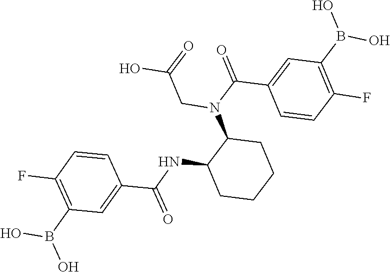 [SMILES: O=C(O)CN(C(=O)c1ccc(F)c(B(O)O)c1)[C@H]1CCCC[C@H]1NC(=O)c1ccc(F)c(B(O)O)c1] |
| | | | N-((1S,2R)-2-(3-borono-4-fluorobenzamido)cyclohexyl)-N-(3- |
| | | | borono-4-fluorobenzoyl)glycine |
| DS18 | FF1 | F1 | 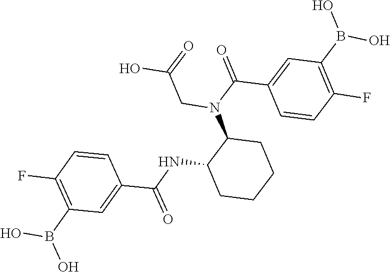 [SMILES: O=C(O)CN(C(=O)c1ccc(F)c(B(O)O)c1)[C@H]1CCCC[C@@H]1NC(=O)c1ccc(F)c(B(O)O)c1] |
| | | | N-((1S,2S)-2-(3-borono-4-fluorobenzamido)cyclohexyl)-N-(3- |
| | | | borono-4-fluorobenzoyl)glycine |
| DS19 | FF10 | F1 | 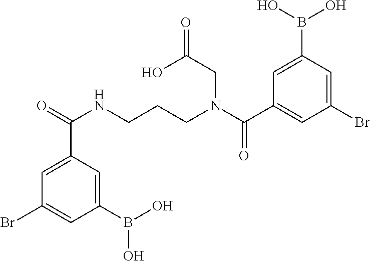 [SMILES: O=C(O)CN(CCCNC(=O)c1cc(Br)cc(B(O)O)c1)C(=O)c1cc(Br)cc(B(O)O)c1] |
| | | | N-(3-(3-borono-5-bromobenzamido)propyl)-N-(3-borono-5- |
| | | | bromobenzoyl)glycine |
| DS20 | FF217 | F1 | 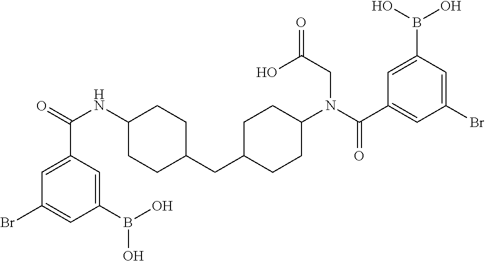 [SMILES: O=C(O)CN(C(=O)c1cc(Br)cc(B(O)O)c1)C1CCC(CC2CCC(NC(=O)c3cc(Br)cc(B(O)O)c3)CC2)CC1] |
| | | | N-(4-((4-(3-borono-5- |
| | | | bromobenzamido)cyclohexyl)methyl)cyclohexyl)-N-(3-borono-5- |
| | | | bromobenzoyl)glycine |
| DS21 | FF214 | F1 | 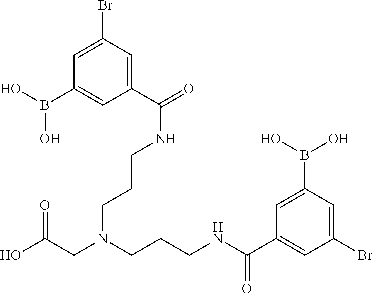 [SMILES: O=C(O)CN(CCCNC(=O)c1cc(Br)cc(B(O)O)c1)CCCNC(=O)c1cc(Br)cc(B(O)O)c1] |
| | | | bis(3-(3-borono-5-bromobenzamido)propyl)glycine |
| DS22 | FF8 | F1 | 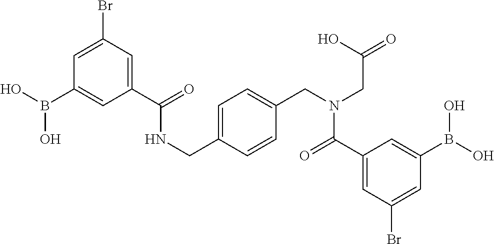 [SMILES: O=C(O)CN(Cc1ccc(CNC(=O)c2cc(Br)cc(B(O)O)c2)cc1)C(=O)c1cc(Br)cc(B(O)O)c1] |
| | | | N-(4-((3-borono-5-bromobenzamido)methyl)benzyl)-N-(3-borono- |
| | | | 5-bromobenzoyl)glycine |
| DS23 | FF14 | F1 | 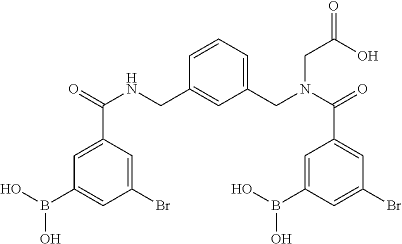 [SMILES: O=C(O)CN(Cc1cccc(CNC(=O)c2cc(Br)cc(B(O)O)c2)c1)C(=O)c1cc(Br)cc(B(O)O)c1] |
| | | | N-(3-((3-borono-5-bromobenzamido)methyl)benzyl)-N-(3-borono- |
| | | | 5-bromobenzoyl)glycine |
| DS24 | FF1 | F1 | 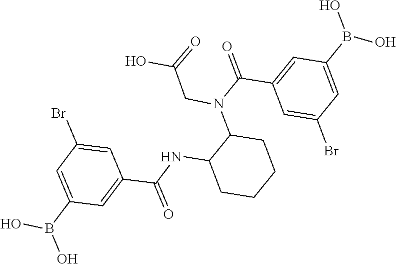 [SMILES: O=C(O)CN(C(=O)c1cc(Br)cc(B(O)O)c1)C1CCCCC1NC(=O)c1cc(Br)cc(B(O)O)c1] |
| | | | N-(2-(3-borono-5-bromobenzamido)cyclohexyl)-N-(3-borono-5- |
| | | | bromobenzoyl)glycine |
| DS25 | FF10 | F1 | 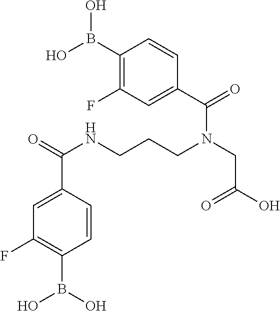 [SMILES: O=C(O)CN(CCCNC(=O)c1ccc(B(O)O)c(F)c1)C(=O)c1ccc(B(O)O)c(F)c1] |
| | | | N-(3-(4-borono-3-fluorobenzamido)propyl)-N-(4-borono-3- |
| | | | fluorobenzoyl)glycine |
| DS26 | FF217 | F1 | 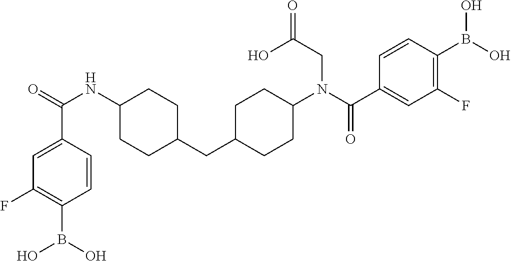 [SMILES: O=C(O)CN(C(=O)c1ccc(B(O)O)c(F)c1)C1CCC(CC2CCC(NC(=O)c3ccc(B(O)O)c(F)c3)CC2)CC1] |
| | | | N-(4-((4-(4-borono-3- |
| | | | fluorobenzamido)cyclohexyl)methyl)cyclohexyl)-N-(4-borono-3- |
| | | | fluorobenzoyl)glycine |
| DS27 | FF213 | F1 | 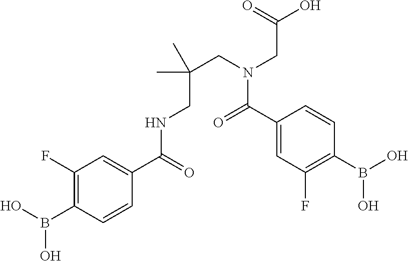 [SMILES: CC(C)(CNC(=O)c1ccc(B(O)O)c(F)c1)CN(CC(=O)O)C(=O)c1ccc(B(O)O)c(F)c1] |
| | | | N-(3-(4-borono-3-fluorobenzamido)-2,2-dimethylpropyl)-N-(4- |
| | | | borono-3-fluorobenzoyl)glycine |
| DS28 | FF214 | F1 | 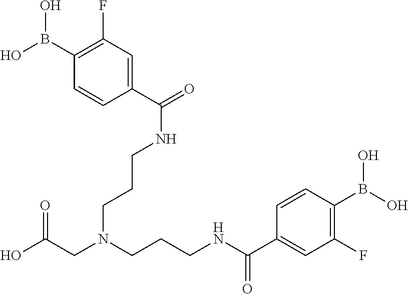 [SMILES: O=C(O)CN(CCCNC(=O)c1ccc(B(O)O)c(F)c1)CCCNC(=O)c1ccc(B(O)O)c(F)c1] |
| | | | bis(3-(4-borono-3-fluorobenzamido)propyl)glycine |
| DS29 | FF8 | F1 | 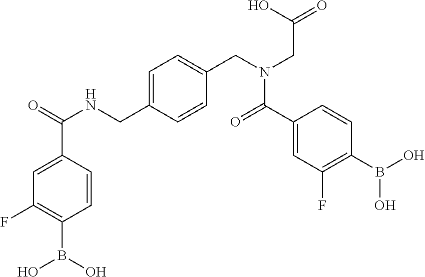 [SMILES: O=C(O)CN(Cc1ccc(CNC(=O)c2ccc(B(O)O)c(F)c2)cc1)C(=O)c1ccc(B(O)O)c(F)c1] |
| | | | N-(4-((4-borono-3-fluorobenzamido)methyl)benzyl)-N-(4-borono- |
| | | | 3-fluorobenzoyl)glycine |
| DS30 | FF14 | F1 | 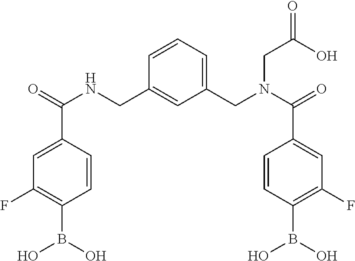 [SMILES: O=C(O)CN(Cc1cccc(CNC(=O)c2ccc(B(O)O)c(F)c2)c1)C(=O)c1ccc(B(O)O)c(F)c1] |
| | | | N-(3-((4-borono-3-fluorobenzamido)methyl)benzyl)-N-(4-borono- |
| | | | 3-fluorobenzoyl)glycine |
| DS31 | FF1 | F1 | 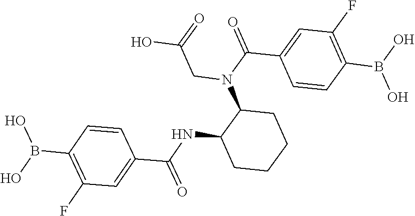 [SMILES: O=C(O)CN(C(=O)c1ccc(B(O)O)c(F)c1)[C@H]1CCCC[C@H]1NC(=O)c1ccc(B(O)O)c(F)c1] |
| | | | N-((1S,2R)-2-(4-borono-3-fluorobenzamido)cyclohexyl)-N-(4- |
| | | | borono-3-fluorobenzoyl)glycine |
| DS32 | FF10 | F2 | 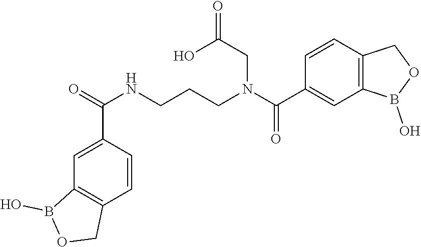 [SMILES: O=C(O)CN(CCCNC(=O)c1ccc2c(c1)B(O)OC2)C(=O)c1ccc2c(c1)B(O)OC2] |
| | | | N-(1-hydroxy-1,3-dihydrobenzo[c][1,2]oxaborole-6-carbonyl)-N- |
| | | | (3-(1-hydroxy-1,3-dihydrobenzo[c][1,2]oxaborole-6- |
| | | | carboxamido)propyl)glycine |
| DS33 | FF10 | F2 | 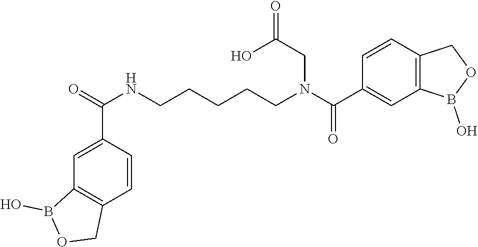 [SMILES: O=C(O)CN(CCCCCNC(=O)c1ccc2c(c1)B(O)OC2)C(=O)c1ccc2c(c1)B(O)OC2] |
| | | | N-(1-hydroxy-1,3-dihydrobenzo[c][1,2]oxaborole-6-carbonyl)-N- |
| | | | (5-(1-hydroxy-1,3-dihydrobenzo[c][1,2]oxaborole-6- |
| | | | carboxamido)pentyl)glycine |
| DS34 | FF213 | F2 | 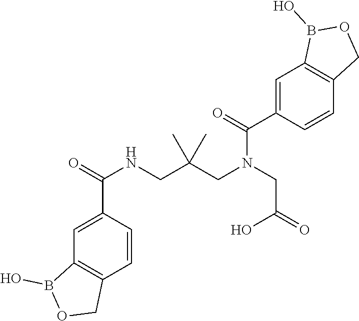 [SMILES: CC(C)(CNC(=O)c1ccc2c(c1)B(O)OC2)CN(CC(=O)O)C(=O)c1ccc2c(c1)B(O)OC2] |
| | | | N-(1-hydroxy-1,3-dihydrobenzo[c][1,2]oxaborole-6-carbonyl)-N- |
| | | | (3-(1-hydroxy-1,3-dihydrobenzo[c][1,2]oxaborole-6- |
| | | | carboxamido)-2,2-dimethylpropyl)glycine |
| DS35 | FF214 | F2 | 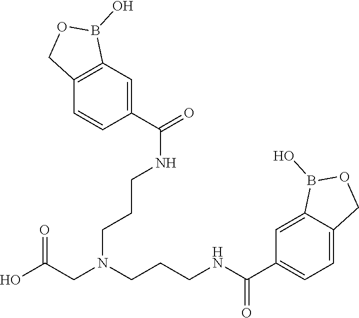 [SMILES: O=C(O)CN(CCCNC(=O)c1ccc2c(c1)B(O)OC2)CCCNC(=O)c1ccc2c(c1)B(O)OC2] |
| | | | bis(3-(1-hydroxy-1,3-dihydrobenzo[c][1,2]oxaborole-6- |
| | | | carboxamido)propyl)glycine |
| DS36 | FF14 | F2 | 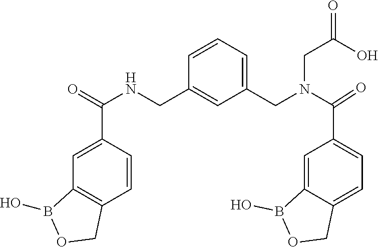 |
| | | | N-(1-hydroxy-1,3-dihydrobenzo[c][1,2]oxaborole-6-carbonyl)-N- |
| | | | (3-((1-hydroxy-1,3-dihydrobenzo[c][1,2]oxaborole-6- |
| | | | carboxamido)methyl)benzyl)glycine |
| DS37 | FF1 | F2 | 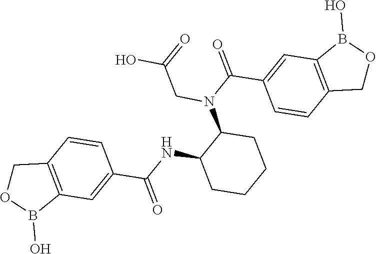 [SMILES: O=C(O)CN(C(=O)c1ccc2c(c1)B(O)OC2)[C@H]1CCCC[C@H]1NC(=O)c1ccc2c(c1)B(O)OC2] |
| | | | N-(1-hydroxy-1,3-dihydrobenzo[c][1,2]oxaborole-6-carbonyl)-N- |
| | | | ((1S,2R)-2-(1-hydroxy-1,3-dihydrobenzo[c][1,2]oxaborole-6- |
| | | | carboxamido)cyclohexyl)glycine |
| DS38 | FF10 | F2 | 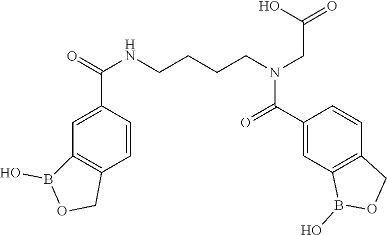 |
| | | | N-(1-hydroxy-1,3-dihydrobenzo[c][1,2]oxaborole-6-carbonyl)-N- |
| | | | (4-(1-hydroxy-1,3-dihydrobenzo[c][1,2]oxaborole-6- |
| | | | carboxamido)butyl)glycine |
| DS39 | FF1 | F2 | 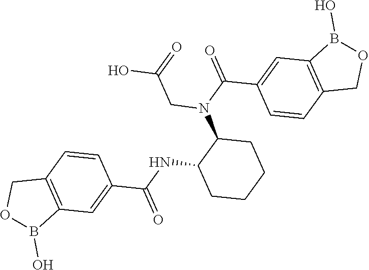 [SMILES: O=C(O)CN(C(=O)c1ccc2c(c1)B(O)OC2)[C@H]1CCCC[C@@H]1NC(=O)c1ccc2c(c1)B(O)OC2] |
| | | | N-(1-hydroxy-1,3-dihydrobenzo[c][1,2]oxaborole-6-carbonyl)-N- |
| | | | ((1S,2S)-2-(1-hydroxy-1,3-dihydrobenzo[c][1,2]oxaborole-6- |
| | | | carboxamido)cyclohexyl)glycine |
| DS40 | FF15 | F2 | 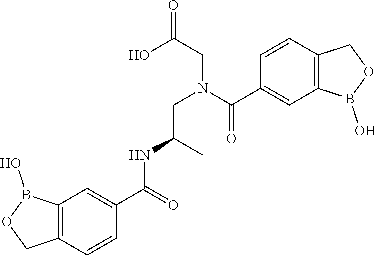 [SMILES: C[C@H](CN(CC(=O)O)C(=O)c1ccc2c(c1)B(O)OC2)NC(=O)c1ccc2c(c1)B(O)OC2] |
| | | | (R)-N-(1-hydroxy-1,3-dihydrobenzo[c][1,2]oxaborole-6-carbonyl)- |
| | | | N-(2-(1-hydroxy-1,3-dihydrobenzo[c][1,2]oxaborole-6- |
| | | | carboxamido)propyl)glycine |
| DS41 | FF15 | F2 | 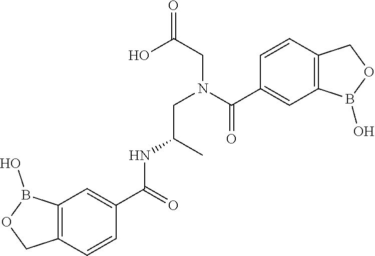 |
| | | | (S)-N-(1-hydroxy-1,3-dihydrobenzo[c][1,2]oxaborole-6-carbonyl)- |
| | | | N-(2-(1-hydroxy-1,3-dihydrobenzo[c][1,2]oxaborole-6- |
| | | | carboxamido)propyl)glycine |
| DS42 | FF1 | F2 | 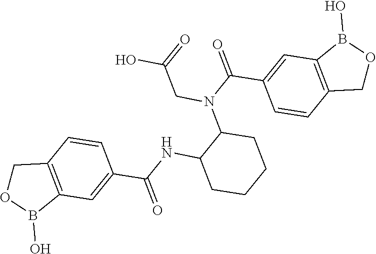 [SMILES: O=C(O)CN(C(=O)c1ccc2c(c1)B(O)OC2)C1CCCCC1NC(=O)c1ccc2c(c1)B(O)OC2] |
| | | | N-(1-hydroxy-1,3-dihydrobenzo[c][1,2]oxaborole-6-carbonyl)-N- |
| | | | (2-(1-hydroxy-1,3-dihydrobenzo[c][1,2]oxaborole-6- |
| | | | carboxamido)cyclohexyl)glycine |
| DS43 | FF10 | F1 | 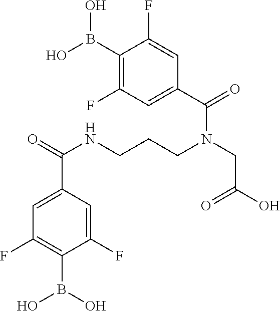 [SMILES: O=C(O)CN(CCCNC(=O)c1cc(F)c(B(O)O)c(F)c1)C(=O)c1cc(F)c(B(O)O)c(F)c1] |
| | | | N-(3-(4-borono-3,5-difluorobenzamido)propyl)-N-(4-borono-3,5- |
| | | | difluorobenzoyl)glycine |
| DS44 | FF10 | F1 | 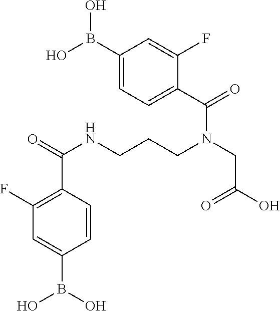 [SMILES: O=C(O)CN(CCCNC(=O)c1ccc(B(O)O)cc1F)C(=O)c1ccc(B(O)O)cc1F] |
| | | | N-(3-(4-borono-2-fluorobenzamido)propyl)-N-(4-borono-2- |
| | | | fluorobenzoyl)glycine |
| DS45 | FF215 | F2 | 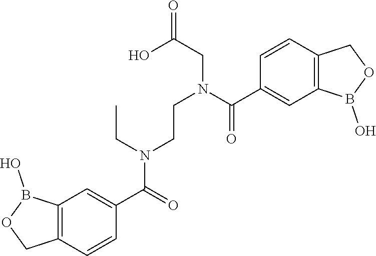 [SMILES: CCN(CCN(CC(=O)O)C(=O)c1ccc2c(c1)B(O)OC2)C(=O)c1ccc2c(c1)B(O)OC2] |
| | | | N-(2-(N-ethyl-1-hydroxy-1,3-dihydrobenzo[c][1,2]oxaborole-6- |
| | | | carboxamido)ethyl)-N-(1-hydroxy-1,3- |
| | | | dihydrobenzo[c][1,2]oxaborole-6-carbonyl)glycine |
| DS46 | FF215 | F2 | 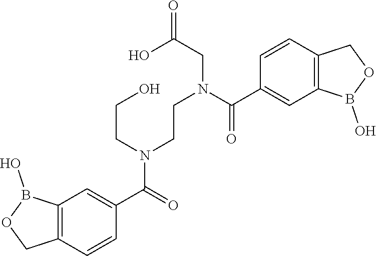 [SMILES: O=C(O)CN(CCN(CCO)C(=O)c1ccc2c(c1)B(O)OC2)C(=O)c1ccc2c(c1)B(O)OC2] |
| | | | N-(1-hydroxy-1,3-dihydrobenzo[c][1,2]oxaborole-6-carbonyl)-N- |
| | | | (2-(1-hydroxy-N-(2-hydroxyethyl)-1,3- |
| | | | dihydrobenzo[c][1,2]oxaborole-6-carboxamido)ethyl)glycine |
| DS47 | FF216 | F2 | 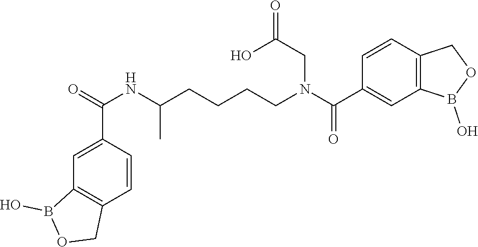 |
| | | | N-(1-hydroxy-1,3-dihydrobenzo[c][1,2]oxaborole-6-carbonyl)-N- |
| | | | (5-(1-hydroxy-1,3-dihydrobenzo[c][1,2]oxaborole-6- |
| | | | carboxamido)hexyl)glycine |
| DS48 | FF217 | F2 | 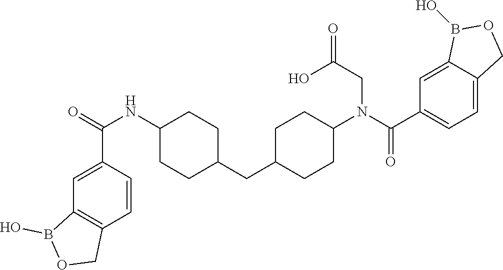 |
| | | | N-(1-hydroxy-1,3-dihydrobenzo[c][1,2]oxaborole-6-carbonyl)-N- |
| | | | (4-((4-(1-hydroxy-1,3-dihydrobenzo[c][1,2]oxaborole-6- |
| | | | carboxamido)cyclohexyl)methyl)cyclohexyl)glycine |
| DS49 | FF12 | F2 | 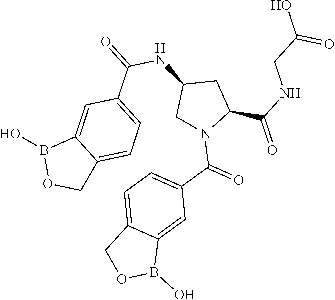 [SMILES: O=C(O)CNC(=O)[C@@H]1C[C@H](NC(=O)c2ccc3c(c2)B(O)OC3)CN1C(=O)c1ccc2c(c1)B(O)OC2] |
| | | | ((2S,4S)-1-(1-hydroxy-1,3-dihydrobenzo[c][1,2]oxaborole-6- |
| | | | carbonyl)-4-(1-hydroxy-1,3-dihydrobenzo[c][1,2]oxaborole-6- |
| | | | carboxamido)pyrrolidine-2-carbonyl)glycine |
| DS50 | FF12 | F1 | 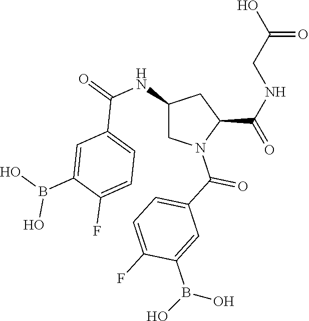 [SMILES: O=C(O)CNC(=O)[C@@H]1C[C@H](NC(=O)c2ccc(F)c(B(O)O)c2)CN1C(=O)c1ccc(F)c(B(O)O)c1] |
| | | | ((2S,4S)-4-(3-borono-4-fluorobenzamido)-1-(3-borono-4- |
| | | | fluorobenzoyl)pyrrolidine-2-carbonyl)glycine |
| DS51 | FF12 | F1 | 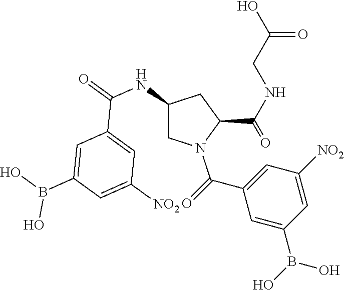 [SMILES: O=C(O)CNC(=O)[C@@H]1C[C@H](NC(=O)c2cc(B(O)O)cc([N+](=O)[O-])c2)CN1C(=O)c1cc(B(O)O)cc([N+](=O)[O-])c1] |
| | | | ((2S,4S)-4-(3-borono-5-nitrobenzamido)-1-(3-borono-5- |
| | | | nitrobenzoyl)pyrrolidine-2-carbonyl)glycine |
| DS52 | FF12 | F1 | 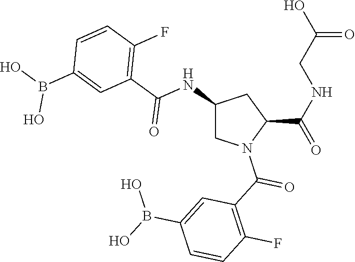 [SMILES: O=C(O)CNC(=O)[C@@H]1C[C@H](NC(=O)c2cc(B(O)O)ccc2F)CN1C(=O)c1cc(B(O)O)ccc1F] |
| | | | ((2S,4S)-4-(5-borono-2-fluorobenzamido)-1-(5-borono-2- |
| | | | fluorobenzoyl)pyrrolidine-2-carbonyl)glycine |
| DS53 | FF9 | F2 | 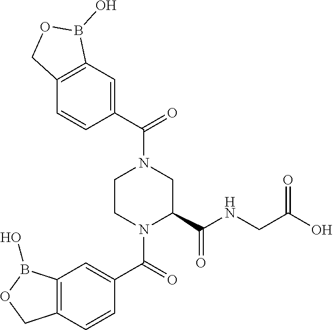 [SMILES: O=C(O)CNC(=O)[C@@H]1CN(C(=O)c2ccc3c(c2)B(O)OC3)CCN1C(=O)c1ccc2c(c1)B(O)OC2] |
| | | | (S)-(1,4-bis(1-hydroxy-1,3-dihydrobenzo[c][1,2]oxaborole-6- |
| | | | carbonyl)piperazine-2-carbonyl)glycine |
| DS54 | FF208 | F2 | 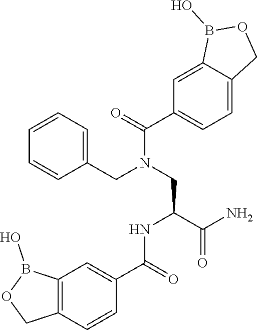 [SMILES: NC(=O)[C@H](CN(Cc1ccccc1)C(=O)c1ccc2c(c1)B(O)OC2)NC(=O)c1ccc2c(c1)B(O)OC2] |
| | | | (S)-N-(3-amino-2-(1-hydroxy-1,3-dihydrobenzo[c][1,2]oxaborole- |
| | | | 6-carboxamido)-3-oxopropyl)-N-benzyl-1-hydroxy-1,3- |
| | | | dihydrobenzo[c][1,2]oxaborole-6-carboxamide |
| DS55 | FF208 | F2 | 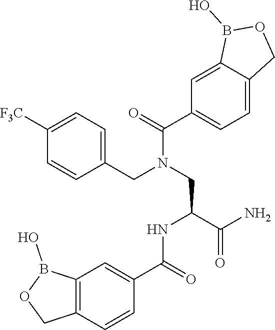 [SMILES: NC(=O)[C@H](CN(Cc1ccc(C(F)(F)F)cc1)C(=O)c1ccc2c(c1)B(O)OC2)NC(=O)c1ccc2c(c1)B(O)OC2] |
| | | | (S)-N-(3-amino-2-(1-hydroxy-1,3-dihydrobenzo[c][1,2]oxaborole- |
| | | | 6-carboxamido)-3-oxopropyl)-1-hydroxy-N-(4- |
| | | | (trifluoromethyl)benzyl)-1,3-dihydrobenzo[c][1,2]oxaborole-6- |
| | | | carboxamide |
| DS56 | FF208 | F2 | 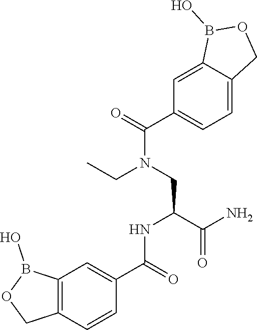 [SMILES: CCN(C[C@H](NC(=O)c1ccc2c(c1)B(O)OC2)C(N)=O)C(=O)c1ccc2c(c1)B(O)OC2] |
| | | | (S)-N-(3-amino-2-(1-hydroxy-1,3-dihydrobenzo[c][1,2]oxaborole- |
| | | | 6-carboxamido)-3-oxopropyl)-N-ethyl-1-hydroxy-1,3- |
| | | | dihydrobenzo[c][1,2]oxaborole-6-carboxamide |
| DS57 | FF208 | F2 | 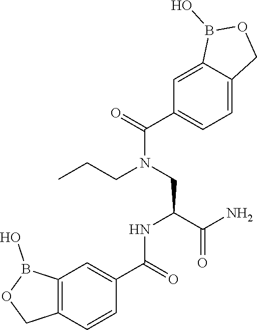 [SMILES: CCCN(C[C@H](NC(=O)c1ccc2c(c1)B(O)OC2)C(N)=O)C(=O)c1ccc2c(c1)B(O)OC2] |
| | | | (S)-N-(3-amino-2-(1-hydroxy-1,3-dihydrobenzo[c][1,2]oxaborole- |
| | | | 6-carboxamido)-3-oxopropyl)-1-hydroxy-N-propyl-1,3- |
| | | | dihydrobenzo[c][1,2]oxaborole-6-carboxamide |
| DS58 | FF208 | F2 | 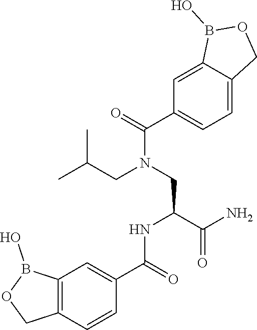 [SMILES: CC(C)CN(C[C@H](NC(=O)c1ccc2c(c1)B(O)OC2)C(N)=O)C(=O)c1ccc2c(c1)B(O)OC2] |
| | | | (S)-N-(3-amino-2-(1-hydroxy-1,3-dihydrobenzo[c][1,2]oxaborole- |
| | | | 6-carboxamido)-3-oxopropyl)-1-hydroxy-N-isobutyl-1,3- |
| | | | dihydrobenzo[c][1,2]oxaborole-6-carboxamide |
| DS59 | FF208 | F2 | 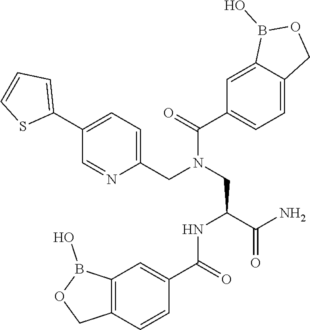 [SMILES: NC(=O)[C@H](CN(Cc1ccc(-c2cccs2)cn1)C(=O)c1ccc2c(c1)B(O)OC2)NC(=O)c1ccc2c(c1)B(O)OC2] |
| | | | (S)-N-(3-amino-2-(1-hydroxy-1,3-dihydrobenzo[c][1,2]oxaborole- |
| | | | 6-carboxamido)-3-oxopropyl)-1-hydroxy-N-((5-(thiophen-2- |
| | | | yl)pyridin-2-yl)methyl)-1,3-dihydrobenzo[c][1,2]oxaborole-6- |
| | | | carboxamide |
| DS60 | FF208 | F2 | 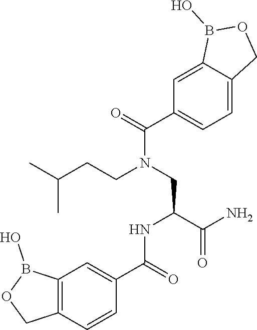 [SMILES: CC(C)CCN(C[C@H](NC(=O)c1ccc2c(c1)B(O)OC2)C(N)=O)C(=O)c1ccc2c(c1)B(O)OC2] |
| | | | (S)-N-(3-amino-2-(1-hydroxy-1,3-dihydrobenzo[c][1,2]oxaborole- |
| | | | 6-carboxamido)-3-oxopropyl)-1-hydroxy-N-isopentyl-1,3- |
| | | | dihydrobenzo[c][1,2]oxaborole-6-carboxamide |
| DS61 | FF208 | F2 | 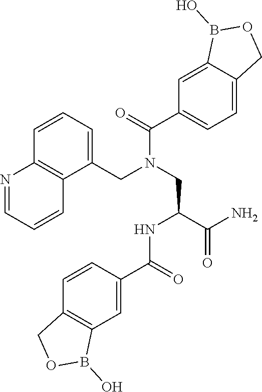 [SMILES: NC(=O)[C@H](CN(Cc1cccc2ncccc12)C(=O)c1ccc2c(c1)B(O)OC2)NC(=O)c1ccc2c(c1)B(O)OC2] |
| | | | (S)-N-(3-amino-2-(1-hydroxy-1,3-dihydrobenzo[c][1,2]oxaborole- |
| | | | 6-carboxamido)-3-oxopropyl)-1-hydroxy-N-(quinolin-5-ylmethyl)- |
| | | | 1,3-dihydrobenzo[c][1,2]oxaborole-6-carboxamide |
| DS62 | FF208 | F2 | 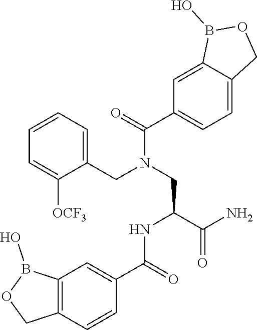 [SMILES: NC(=O)[C@H](CN(Cc1ccccc1OC(F)(F)F)C(=O)c1ccc2c(c1)B(O)OC2)NC(=O)c1ccc2c(c1)B(O)OC2] |
| | | | (S)-N-(3-amino-2-(1-hydroxy-1,3-dihydrobenzo[c][1,2]oxaborole- |
| | | | 6-carboxamido)-3-oxopropyl)-1-hydroxy-N-(2- |
| | | | (trifluoromethoxy)benzyl)-1,3-dihydrobenzo[c][1,2]oxaborole-6- |
| | | | carboxamide |
| DS63 | FF208 | F2 | 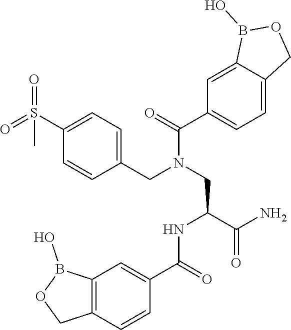 [SMILES: CS(=O)(=O)c1ccc(CN(C[C@H](NC(=O)c2ccc3c(c2)B(O)OC3)C(N)=O)C(=O)c2ccc3c(c2)B(O)OC3)cc1] |
| | | | (S)-N-(3-amino-2-(1-hydroxy-1,3-dihydrobenzo[c][1,2]oxaborole- |
| | | | 6-carboxamido)-3-oxopropyl)-1-hydroxy-N-(4- |
| | | | (methylsulfonyl)benzyl)-1,3-dihydrobenzo[c][1,2]oxaborole-6- |
| | | | carboxamide |
| DS64 | FF12 | F1 | 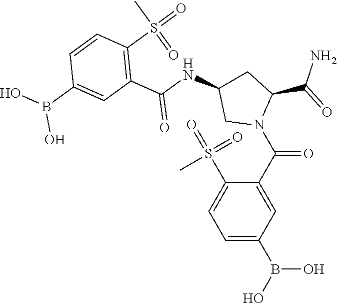 [SMILES: CS(=O)(=O)c1ccc(B(O)O)cc1C(=O)N[C@H]1C[C@@H](C(N)=O)N(C(=O)c2cc(B(O)O)ccc2S(C)(=O)=O)C1] |
| | | | (3-((2S,4S)-4-(5-borono-2-(methylsulfonyl)benzamido)-2- |
| | | | carbamoylpyrrolidine-1-carbonyl)-4- |
| | | | (methylsulfonyl)phenyl)boronic acid |
| DS65 | FF12 | F1 | 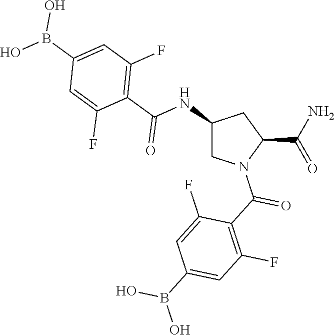 [SMILES: NC(=O)[C@@H]1C[C@H](NC(=O)c2c(F)cc(B(O)O)cc2F)CN1C(=O)c1c(F)cc(B(O)O)cc1F] |
| | | | (4-(((3S,5S)-1-(4-borono-2,6-difluorobenzoyl)-5- |
| | | | carbamoylpyrrolidin-3-yl)carbamoyl)-3,5-difluorophenyl)boronic |
| | | | acid |
| DS66 | FF220 | F2 | 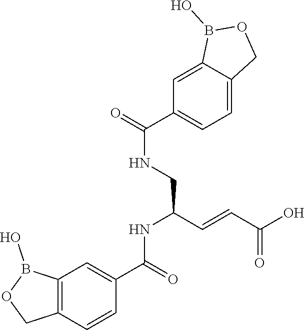 [SMILES: O=C(O)/C=C/[C@H](CNC(=O)c1ccc2c(c1)B(O)OC2)NC(=O)c1ccc2c(c1)B(O)OC2] |
| | | | (R,E)-4,5-bis(1-hydroxy-1,3-dihydrobenzo[c][1,2]oxaborole-6- |
| | | | carboxamido)pent-2-enoic acid |
| DS67 | FF12 | F2 | 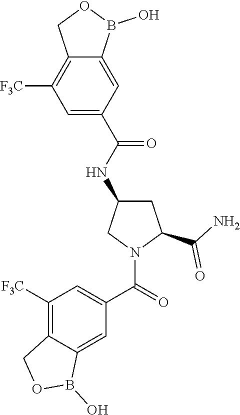 |
| | | | (2S,4S)-1-(1-hydroxy-4-(trifluoromethyl)-1,3- |
| | | | dihydrobenzo[c][1,2]oxaborole-6-carbonyl)-4-(1-hydroxy-4- |
| | | | (trifluoromethyl)-1,3-dihydrobenzo[c][1,2]oxaborole-6- |
| | | | carboxamido)pyrrolidine-2-carboxamide |
| DS68 | FF116 | F2 | 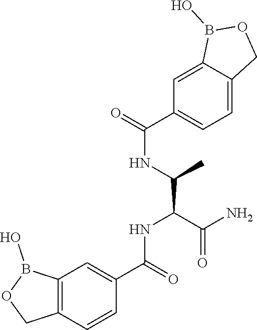 [SMILES: C[C@H](NC(=O)c1ccc2c(c1)B(O)OC2)[C@H](NC(=O)c1ccc2c(c1)B(O)OC2)C(N)=O] |
| | | | N,N′-((2S,3S)-1-amino-1-oxobutane-2,3-diyl)bis(1-hydroxy-1,3- |
| | | | dihydrobenzo[c][1,2]oxaborole-6-carboxamide) |
| DS69 | FF115 | F2 | 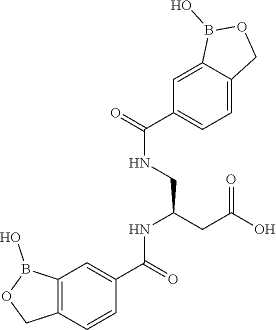 [SMILES: O=C(O)C[C@H](CNC(=O)c1ccc2c(c1)B(O)OC2)NC(=O)c1ccc2c(c1)B(O)OC2] |
| | | | (R)-3,4-bis(1-hydroxy-1,3-dihydrobenzo[c][1,2]oxaborole-6- |
| | | | carboxamido)butanoic acid |
| DS70 | FF12 | F2 | 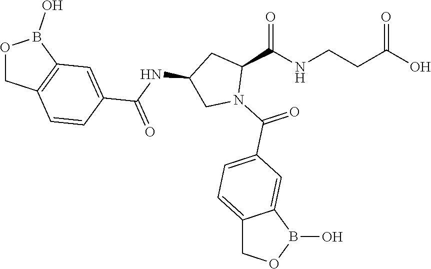 [SMILES: O=C(O)CCNC(=O)[C@@H]1C[C@H](NC(=O)c2ccc3c(c2)B(O)OC3)CN1C(=O)c1ccc2c(c1)B(O)OC2] |
| | | | 3-((2S,4S)-1-(1-hydroxy-1,3-dihydrobenzo[c][1,2]oxaborole-6- |
| | | | carbonyl)-4-(1-hydroxy-1,3-dihydrobenzo[c][1,2]oxaborole-6- |
| | | | carboxamido)pyrrolidine-2-carboxamido)propanoic acid |
| DS71 | FF115 | F2 | 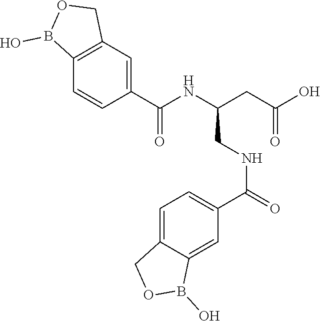 [SMILES: O=C(O)C[C@@H](CNC(=O)c1ccc2c(c1)B(O)OC2)NC(=O)c1ccc2c(c1)COB2O] |
| | | | (S)-3-(1-hydroxy-1,3-dihydrobenzo[c][1,2]oxaborole-5- |
| | | | carboxamido)-4-(1-hydroxy-1,3-dihydrobenzo[c][1,2]oxaborole-6- |
| | | | carboxamido)butanoic acid |
| DS72 | FF114 | F2 | 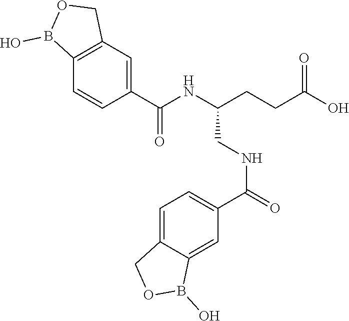 [SMILES: O=C(O)CC[C@H](CNC(=O)c1ccc2c(c1)B(O)OC2)NC(=O)c1ccc2c(c1)COB2O] |
| | | | (R)-4-(1-hydroxy-1,3-dihydrobenzo[c][1,2]oxaborole-5- |
| | | | carboxamido)-5-(1-hydroxy-1,3-dihydrobenzo[c][1,2]oxaborole-6- |
| | | | carboxamido)pentanoic acid |
| DS73 | FF12 | F2 | 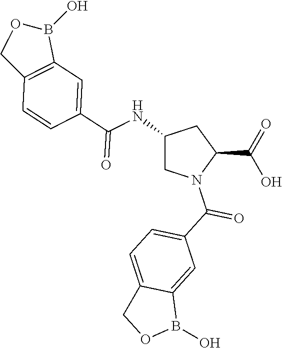 [SMILES: O=C(N[C@@H]1C[C@@H](C(=O)O)N(C(=O)c2ccc3c(c2)B(O)OC3)C1)c1ccc2c(c1)B(O)OC2] |
| | | | (2S,4R)-1-(1-hydroxy-1,3-dihydrobenzo[c][1,2]oxaborole-6- |
| | | | carbonyl)-4-(1-hydroxy-1,3-dihydrobenzo[c][1,2]oxaborole-6- |
| | | | carboxamido)pyrrolidine-2-carboxylic acid |
| DS74 | FF12 | F2 | 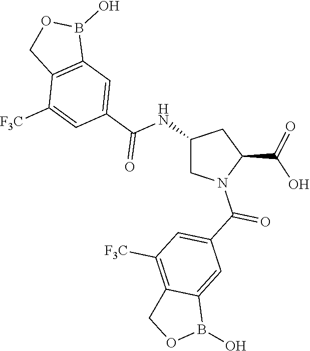 [SMILES: O=C(N[C@@H]1C[C@@H](C(=O)O)N(C(=O)c2cc3c(c(C(F)(F)F)c2)COB3O)C1)c1cc2c(c(C(F)(F)F)c1)COB2O] |
| | | | (2S,4R)-1-(1-hydroxy-4-(trifluoromethyl)-1,3- |
| | | | dihydrobenzo[c][1,2]oxaborole-6-carbonyl)-4-(1-hydroxy-4- |
| | | | (trifluoromethyl)-1,3-dihydrobenzo[c][1,2]oxaborole-6- |
| | | | carboxamido)pyrrolidine-2-carboxylic acid |
| DS75 | FF116 | F2 | 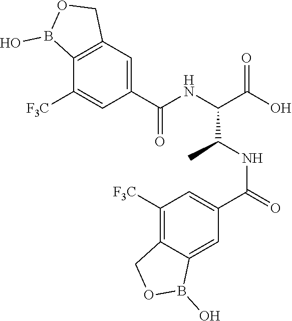 [SMILES: C[C@H](NC(=O)c1cc2c(c(C(F)(F)F)c1)COB2O)[C@H](NC(=O)c1cc2c(c(C(F)(F)F)c1)B(O)OC2)C(=O)O] |
| | | | (2S,3S)-3-(1-hydroxy-4-(trifluoromethyl)-1,3- |
| | | | dihydrobenzo[c][1,2]oxaborole-6-carboxamido)-2-(1-hydroxy-7- |
| | | | (trifluoromethyl)-1,3-dihydrobenzo[c][1,2]oxaborole-5- |
| | | | carboxamido)butanoic acid |
| DS76 | FF114 | F2 | 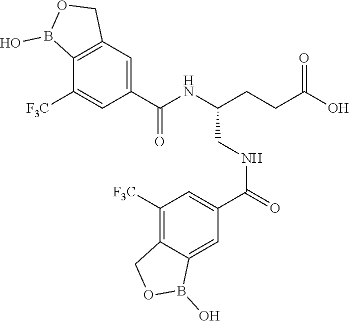 [SMILES: O=C(O)CC[C@H](CNC(=O)c1cc2c(c(C(F)(F)F)c1)COB2O)NC(=O)c1cc2c(c(C(F)(F)F)c1)B(O)OC2] |
| | | | (R)-5-(1-hydroxy-4-(trifluoromethyl)-1,3- |
| | | | dihydrobenzo[c][1,2]oxaborole-6-carboxamido)-4-(1-hydroxy-7- |
| | | | (trifluoromethyl)-1,3-dihydrobenzo[c][1,2]oxaborole-5- |
| | | | carboxamido)pentanoic acid |
| DS77 | FF12 | F1 and F2 | 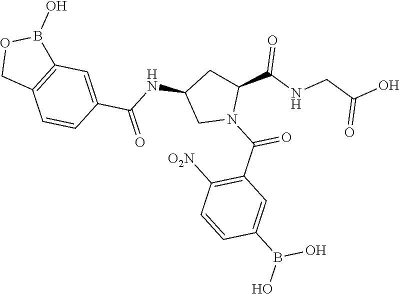 [SMILES: O=C(O)CNC(=O)[C@@H]1C[C@H](NC(=O)c2ccc3c(c2)B(O)OC3)CN1C(=O)c1cc(B(O)O)ccc1[N+](=O)[O-]] |
| | | | ((2S,4S)-1-(5-borono-2-nitrobenzoyl)-4-(1-hydroxy-1,3- |
| | | | dihydrobenzo[c][1,2]oxaborole-6-carboxamido)pyrrolidine-2- |
| | | | carbonyl)glycine |
| DS78 | FF12 | F1 and F2 | 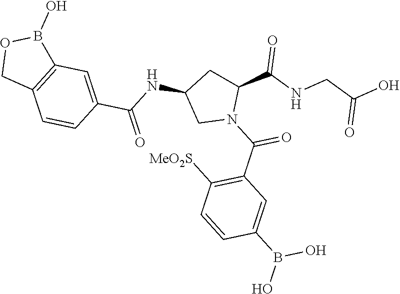 [SMILES: CS(=O)(=O)c1ccc(B(O)O)cc1C(=O)N1C[C@@H](NC(=O)c2ccc3c(c2)B(O)OC3)C[C@H]1C(=O)NCC(=O)O] |
| | | | ((2S,4S)-1-(5-borono-2-(methylsulfonyl)benzoyl)-4-(1-hydroxy- |
| | | | 1,3-dihydrobenzo[c][1,2]oxaborole-6-carboxamido)pyrrolidine-2- |
| | | | carbonyl)glycine |
| DS79 | FF12 | F1 and F2 | 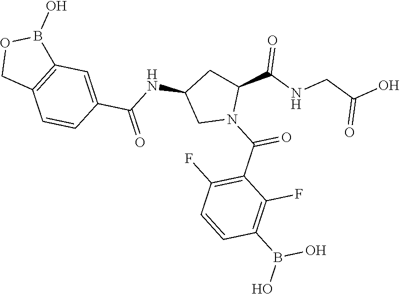 [SMILES: O=C(O)CNC(=O)[C@@H]1C[C@H](NC(=O)c2ccc3c(c2)B(O)OC3)CN1C(=O)c1c(F)ccc(B(O)O)c1F] |
| | | | ((2S,4S)-1-(3-borono-2,6-difluorobenzoyl)-4-(1-hydroxy-1,3- |
| | | | dihydrobenzo[c][1,2]oxaborole-6-carboxamido)pyrrolidine-2- |
| | | | carbonyl)glycine |
| DS80 | FF221 | F1 | 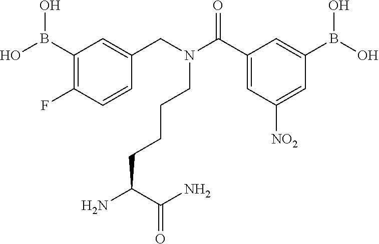 [SMILES: NC(=O)[C@@H](N)CCCCN(Cc1ccc(F)c(B(O)O)c1)C(=O)c1cc(B(O)O)cc([N+](=O)[O-])c1] |
| | | | (S)-(3-((3-borono-4-fluorobenzyl)(5,6-diamino-6- |
| | | | oxohexyl)carbamoyl)-5-nitrophenyl)boronic acid |
| DS81 | FF221 | F1 | 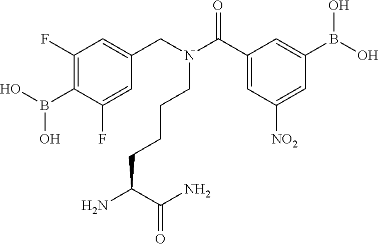 [SMILES: NC(=O)[C@@H](N)CCCCN(Cc1cc(F)c(B(O)O)c(F)c1)C(=O)c1cc(B(O)O)cc([N+](=O)[O-])c1] |
| | | | (S)-(3-((4-borono-3,5-difluorobenzyl)(5,6-diamino-6- |
| | | | oxohexyl)carbamoyl)-5-nitrophenyl)boronic acid |
| DS82 | FF221 | F1 | 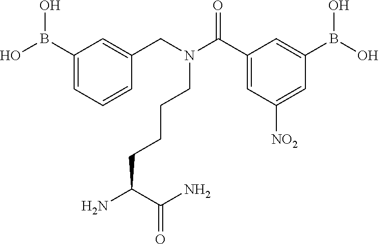 [SMILES: NC(=O)[C@@H](N)CCCCN(Cc1cccc(B(O)O)c1)C(=O)c1cc(B(O)O)cc([N+](=O)[O-])c1] |
| | | | (S)-(3-((3-boronobenzyl)(5,6-diamino-6-oxohexyl)carbamoyl)-5- |
| | | | nitrophenyl)boronic acid |
| DS83 | FF221 | F1 | 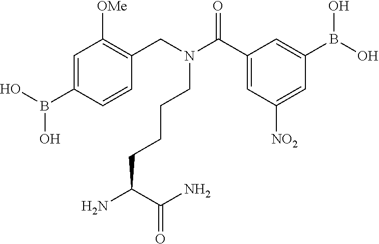 [SMILES: COc1cc(B(O)O)ccc1CN(CCCC[C@H](N)C(N)=O)C(=O)c1cc(B(O)O)cc([N+](=O)[O-])c1] |
| | | | (S)-(3-((4-borono-2-methoxybenzyl)(5,6-diamino-6- |
| | | | oxohexyl)carbamoyl)-5-nitrophenyl)boronic acid |
| DS84 | FF221 | F1 | 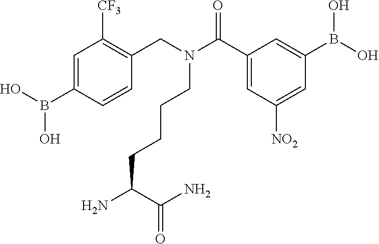 [SMILES: NC(=O)[C@@H](N)CCCCN(Cc1ccc(B(O)O)cc1C(F)(F)F)C(=O)c1cc(B(O)O)cc([N+](=O)[O-])c1] |
| | | | (S)-(3-((4-borono-2-(trifluoromethyl)benzyl)(5,6-diamino-6- |
| | | | oxohexyl)carbamoyl)-5-nitrophenyl)boronic acid |
| DS85 | FF221 | F1 | 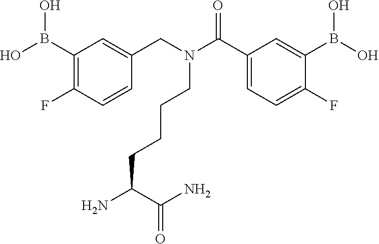 [SMILES: NC(=O)[C@@H](N)CCCCN(Cc1ccc(F)c(B(O)O)c1)C(=O)c1ccc(F)c(B(O)O)c1] |
| | | | (S)-(5-((3-borono-N-(5,6-diamino-6-oxohexyl)-4- |
| | | | fluorobenzamido)methyl)-2-fluorophenyl)boronic acid |
| DS86 | FF221 | F1 | 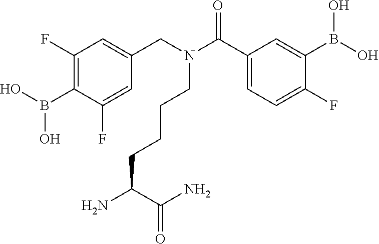 [SMILES: NC(=O)[C@@H](N)CCCCN(Cc1cc(F)c(B(O)O)c(F)c1)C(=O)c1ccc(F)c(B(O)O)c1] |
| | | | (S)-(5-((4-borono-3,5-difluorobenzyl)(5,6-diamino-6- |
| | | | oxohexyl)carbamoyl)-2-fluorophenyl)boronic acid |
| DS87 | FF221 | F1 | 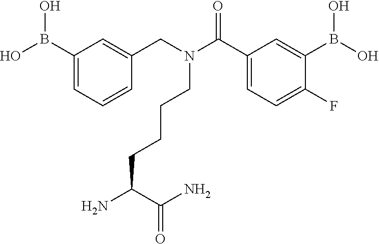 [SMILES: NC(=O)[C@@H](N)CCCCN(Cc1cccc(B(O)O)c1)C(=O)c1ccc(F)c(B(O)O)c1] |
| | | | (S)-(3-((3-borono-N-(5,6-diamino-6-oxohexyl)-4- |
| | | | fluorobenzamido)methyl)phenyl)boronic acid |
| DS88 | FF221 | F1 | 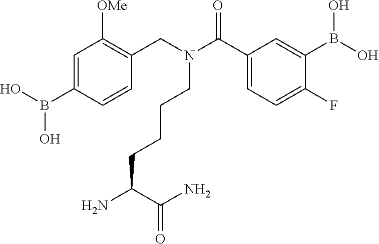 [SMILES: COc1cc(B(O)O)ccc1CN(CCCC[C@H](N)C(N)=O)C(=O)c1ccc(F)c(B(O)O)c1] |
| | | | (S)-(5-((4-borono-2-methoxybenzyl)(5,6-diamino-6- |
| | | | oxohexyl)carbamoyl)-2-fluorophenyl)boronic acid |
| DS89 | FF221 | F1 | 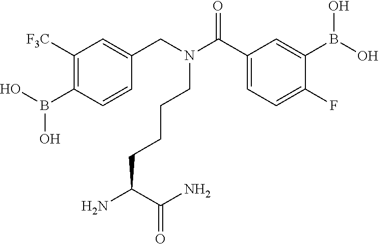 [SMILES: NC(=O)[C@@H](N)CCCCN(Cc1ccc(B(O)O)c(C(F)(F)F)c1)C(=O)c1ccc(F)c(B(O)O)c1] |
| | | | (S)-(5-((4-borono-3-(trifluoromethyl)benzyl)(5,6-diamino-6- |
| | | | oxohexyl)carbamoyl)-2-fluorophenyl)boronic acid |
| DS90 | FF221 | F1 | 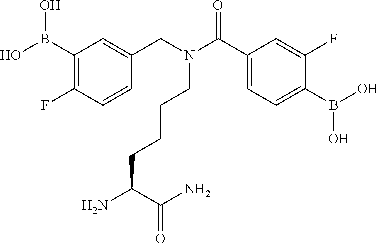 [SMILES: NC(=O)[C@@H](N)CCCCN(Cc1ccc(F)c(B(O)O)c1)C(=O)c1ccc(B(O)O)c(F)c1] |
| | | | (S)-(4-((3-borono-4-fluorobenzyl)(5,6-diamino-6- |
| | | | oxohexyl)carbamoyl)-2-fluorophenyl)boronic acid |
| DS91 | FF221 | F1 | 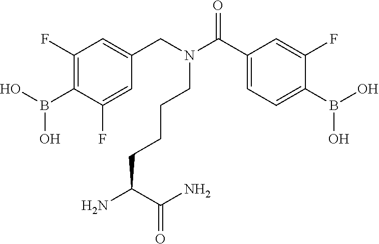 [SMILES: NC(=O)[C@@H](N)CCCCN(Cc1cc(F)c(B(O)O)c(F)c1)C(=O)c1ccc(B(O)O)c(F)c1] |
| | | | (S)-(4-((4-borono-3,5-difluorobenzyl)(5,6-diamino-6- |
| | | | oxohexyl)carbamoyl)-2-fluorophenyl)boronic acid |
| DS92 | FF221 | F1 | 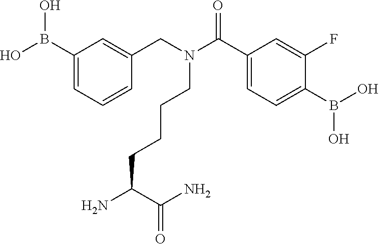 [SMILES: NC(=O)[C@@H](N)CCCCN(Cc1cccc(B(O)O)c1)C(=O)c1ccc(B(O)O)c(F)c1] |
| | | | (S)-(4-((3-boronobenzyl)(5,6-diamino-6-oxohexyl)carbamoyl)-2- |
| | | | fluorophenyl)boronic acid |
| DS93 | FF221 | F1 | 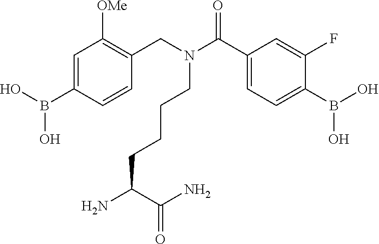 [SMILES: COc1cc(B(O)O)ccc1CN(CCCC[C@H](N)C(N)=O)C(=O)c1ccc(B(O)O)c(F)c1] |
| | | | (S)-(4-((4-borono-2-methoxybenzyl)(5,6-diamino-6- |
| | | | oxohexyl)carbamoyl)-2-fluorophenyl)boronic acid |
| DS94 | FF221 | F1 | 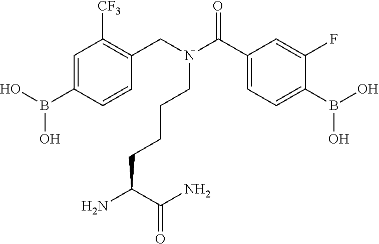 [SMILES: NC(=O)[C@@H](N)CCCCN(Cc1ccc(B(O)O)cc1C(F)(F)F)C(=O)c1ccc(B(O)O)c(F)c1] |
| | | | (S)-(4-((4-borono-2-(trifluoromethyl)benzyl)(5,6-diamino-6- |
| | | | oxohexyl)carbamoyl)-2-fluorophenyl)boronic acid |
| DS95 | FF221 | F1 | 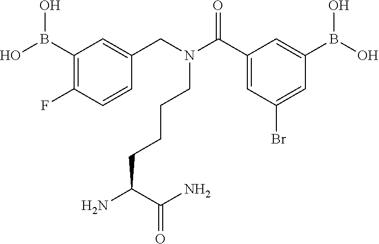 [SMILES: NC(=O)[C@@H](N)CCCCN(Cc1ccc(F)c(B(O)O)c1)C(=O)c1cc(Br)cc(B(O)O)c1] |
| | | | (S)-(5-((3-borono-5-bromo-N-(5,6-diamino-6- |
| | | | oxohexyl)benzamido)methyl)-2-fluorophenyl)boronic acid |
| DS96 | FF221 | F1 | 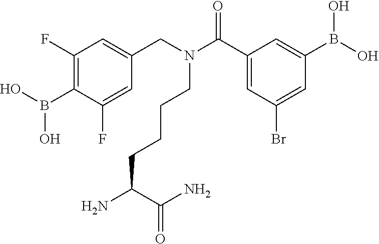 [SMILES: NC(=O)[C@@H](N)CCCCN(Cc1cc(F)c(B(O)O)c(F)c1)C(=O)c1cc(Br)cc(B(O)O)c1] |
| | | | (S)-(3-((4-borono-3,5-difluorobenzyl)(5,6-diamino-6- |
| | | | oxohexyl)carbamoyl)-5-bromophenyl)boronic acid |
| DS97 | FF221 | F1 | 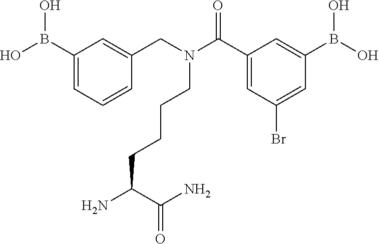 [SMILES: NC(=O)[C@@H](N)CCCCN(Cc1cccc(B(O)O)c1)C(=O)c1cc(Br)cc(B(O)O)c1] |
| | | | (S)-(3-((3-borono-5-bromo-N-(5,6-diamino-6- |
| | | | oxohexyl)benzamido)methyl)phenyl)boronic acid |
| DS98 | FF221 | F1 | 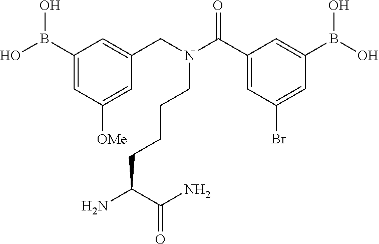 [SMILES: COc1cc(CN(CCCC[C@H](N)C(N)=O)C(=O)c2cc(Br)cc(B(O)O)c2)cc(B(O)O)c1] |
| | | | (S)-(3-((3-borono-5-bromo-N-(5,6-diamino-6- |
| | | | oxohexyl)benzamido)methyl)-5-methoxyphenyl)boronic acid |
| DS99 | FF221 | F1 | 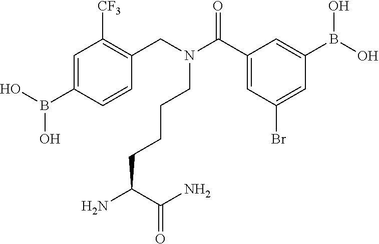 [SMILES: NC(=O)[C@@H](N)CCCCN(Cc1ccc(B(O)O)cc1C(F)(F)F)C(=O)c1cc(Br)cc(B(O)O)c1] |
| | | | (S)-(3-((4-borono-2-(trifluoromethyl)benzyl)(5,6-diamino-6- |
| | | | oxohexyl)carbamoyl)-5-bromophenyl)boronic acid |
| DS100 | FF221 | F1 | 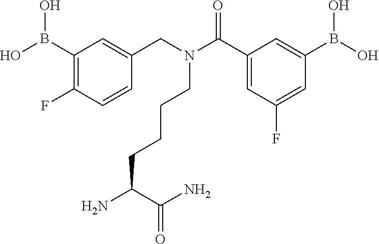 [SMILES: NC(=O)[C@@H](N)CCCCN(Cc1ccc(F)c(B(O)O)c1)C(=O)c1cc(F)cc(B(O)O)c1] |
| | | | (S)-(3-((3-borono-4-fluorobenzyl)(5,6-diamino-6- |
| | | | oxohexyl)carbamoyl)-5-fluorophenyl)boronic acid |
| DS101 | FF221 | F1 | 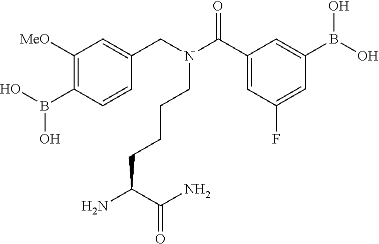 [SMILES: COc1cc(CN(CCCC[C@H](N)C(N)=O)C(=O)c2cc(F)cc(B(O)O)c2)ccc1B(O)O] |
| | | | (S)-(3-((4-borono-3-methoxybenzyl)(5,6-diamino-6- |
| | | | oxohexyl)carbamoyl)-5-fluorophenyl)boronic acid |
| DS102 | FF221 | F1 | 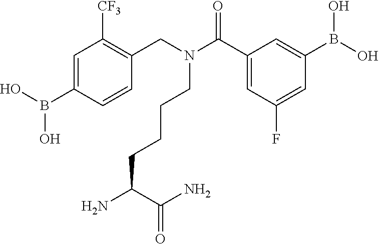 [SMILES: NC(=O)[C@@H](N)CCCCN(Cc1ccc(B(O)O)cc1C(F)(F)F)C(=O)c1cc(F)cc(B(O)O)c1] |
| | | | (S)-(3-((4-borono-2-(trifluoromethyl)benzyl)(5,6-diamino-6- |
| | | | oxohexyl)carbamoyl)-5-fluorophenyl)boronic acid |
| DS103 | FF221 | F1 and F2 | 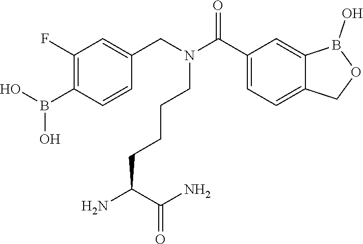 [SMILES: NC(=O)[C@@H](N)CCCCN(Cc1ccc(B(O)O)c(F)c1)C(=O)c1ccc2c(c1)B(O)OC2] |
| | | | (S)-(4-((N-(5,6-diamino-6-oxohexyl)-1-hydroxy-1,3- |
| | | | dihydrobenzo[c][1,2]oxaborole-6-carboxamido)methyl)-2- |
| | | | fluorophenyl)boronic acid |
| DS104 | FF221 | F1 and F2 | 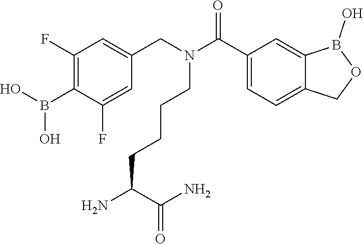 [SMILES: NC(=O)[C@@H](N)CCCCN(Cc1cc(F)c(B(O)O)c(F)c1)C(=O)c1ccc2c(c1)B(O)OC2] |
| | | | (S)-(4-((N-(5,6-diamino-6-oxohexyl)-1-hydroxy-1,3- |
| | | | dihydrobenzo[c][1,2]oxaborole-6-carboxamido)methyl)-2,6- |
| | | | difluorophenyl)boronic acid |
| DS105 | FF221 | F1 and F2 | 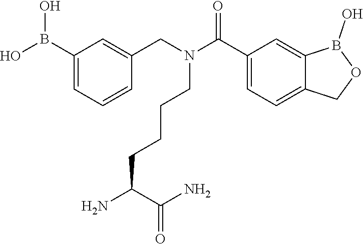 [SMILES: NC(=O)[C@@H](N)CCCCN(Cc1cccc(B(O)O)c1)C(=O)c1ccc2c(c1)B(O)OC2] |
| | | | (S)-(3-((N-(5,6-diamino-6-oxohexyl)-1-hydroxy-1,3- |
| | | | dihydrobenzo[c][1,2]oxaborole-6- |
| | | | carboxamido)methyl)phenyl)boronic acid |
| DS106 | FF221 | F1 and F2 | 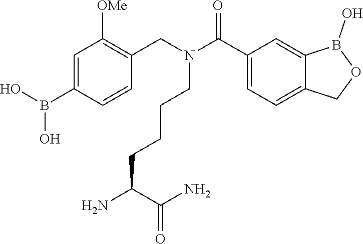 [SMILES: COc1cc(B(O)O)ccc1CN(CCCC[C@H](N)C(N)=O)C(=O)c1ccc2c(c1)B(O)OC2] |
| | | | (S)-(4-((N-(5,6-diamino-6-oxohexyl)-1-hydroxy-1,3- |
| | | | dihydrobenzo[c][1,2]oxaborole-6-carboxamido)methyl)-3- |
| | | | methoxyphenyl)boronic acid |
| DS107 | FF221 | F2 | 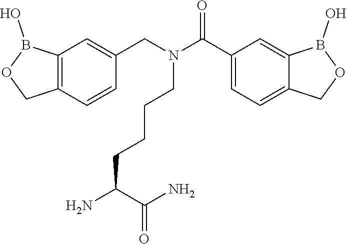 [SMILES: NC(=O)[C@@H](N)CCCCN(Cc1ccc2c(c1)B(O)OC2)C(=O)c1ccc2c(c1)B(O)OC2] |
| | | | (S)-N-(5,6-diamino-6-oxohexyl)-1-hydroxy-N-((1-hydroxy-1,3- |
| | | | dihydrobenzo[c][1,2]oxaborol-6-yl)methyl)-1,3- |
| | | | dihydrobenzo[c][1,2]oxaborole-6-carboxamide |
| DS108 | FF221 | F2 | 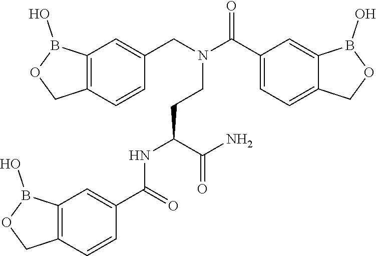 [SMILES: NC(=O)[C@H](CCN(Cc1ccc2c(c1)B(O)OC2)C(=O)c1ccc2c(c1)B(O)OC2)NC(=O)c1ccc2c(c1)B(O)OC2] |
| | | | (S)-N-(4-amino-3-(1-hydroxy-1,3-dihydrobenzo[c][1,2]oxaborole- |
| | | | 6-carboxamido)-4-oxobutyl)-1-hydroxy-N-((1-hydroxy-1,3- |
| | | | dihydrobenzo[c][1,2]oxaborol-6-yl)methyl)-1,3- |
| | | | dihydrobenzo[c][1,2]oxaborole-6-carboxamide |
| DS109 | FF221 | F2 | 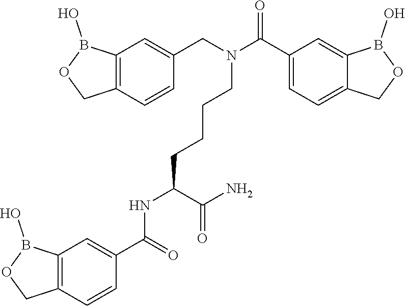 [SMILES: NC(=O)[C@H](CCCCN(Cc1ccc2c(c1)B(O)OC2)C(=O)c1ccc2c(c1)B(O)OC2)NC(=O)c1ccc2c(c1)B(O)OC2] |
| | | | (S)-N-(6-amino-5-(1-hydroxy-1,3-dihydrobenzo[c][1,2]oxaborole- |
| | | | 6-carboxamido)-6-oxohexyl)-1-hydroxy-N-((1-hydroxy-1,3- |
| | | | dihydrobenzo[c][1,2]oxaborol-6-yl)methyl)-1,3- |
| | | | dihydrobenzo[c][1,2]oxaborole-6-carboxamide |

### Synthesis of Compounds of Formula I

An illustrative synthesis protocol is provided that can be used to synthesize any of the examples described.

The lines connecting cysteine residues are disulfide bonds. For the sake of clarity, the H— at the N-terminus of the A- and B-chain of insulin is not histidine, it is the hydrogen of the N-terminus. The —OH shown at the C-terminal end of the A- and B-chain is the C-terminus of the respective chain.

Synthesis of Modified Insulins

### A-Chain Synthesis:

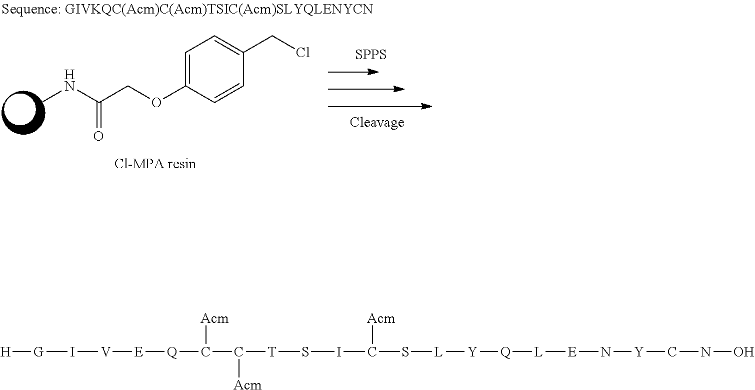

### Synthesis of A-Chain and Modified A-Chain of Example 870 was Accomplished Using Conventional Solid-Phase Peptide Synthesis (SPPS)

MPA resin (0.22 mmol/eq) was swelled in a mixture of DMF:DCM (50:50, v:v). A solution of potassium iodide with DIPEA (1 M) in DMF was added to the reaction vessel along with Fmoc-Asn(Trt)-OH (0.2 M). The reaction vessel was heated to 75° C. Each amino acid coupling step involved i) deprotection with 20% piperidine in DMF at 90° C.; ii) washing with DMF; iii) activation and coupling of Fmoc protected amino acids with 0.5 M N,N′-diisopropylcarbodiimide (DIC, 1 mL), 0.5 M Oxyma, and 0.2 M Fmoc-amino acid in DMF at 90° C.; iv) washing with DMF.

### Global Deprotection and Isolation.

Crude peptide was globally deprotected in TFA:TIPS:H₂O (95:2.5:2.5) and gently agitated for 2 h. Crude solution was filtered and peptide was precipitated in cold ether, centrifuged and washed with additional cold ether. Supernatant was decanted and the crude peptide was dried under a gentle stream of nitrogen gas. Crude peptide was dissolved in 20% ACN in water and fractionated by RP-HPLC on a C18 column.

### B-Chain Synthesis:

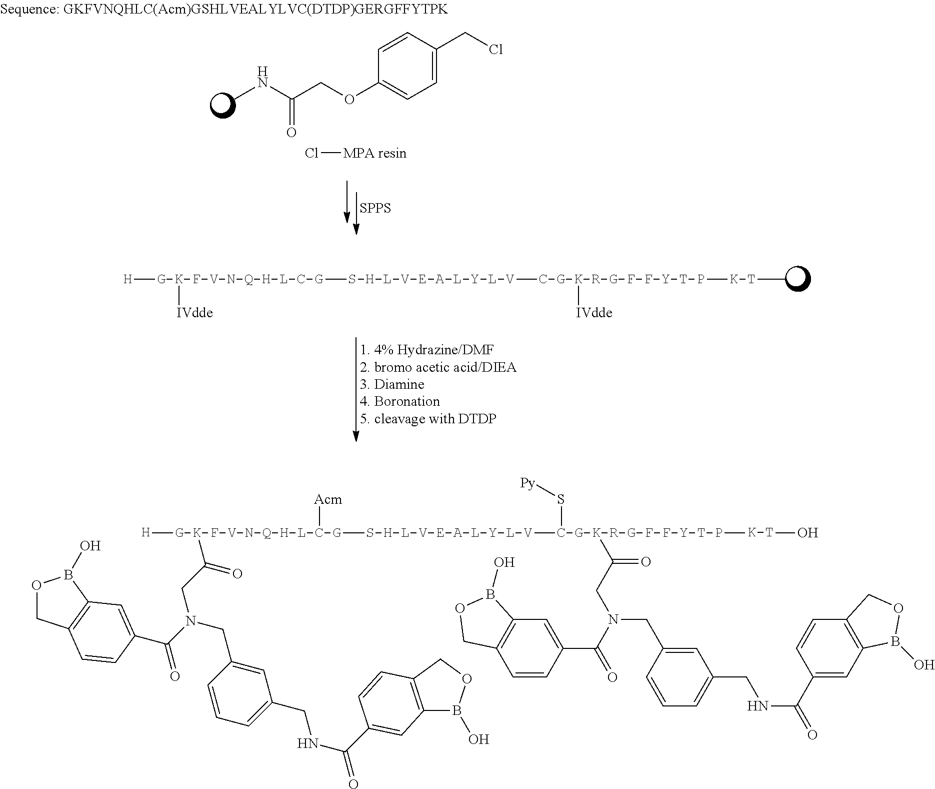

### Synthesis of Modified B-Chain Insulins Using Solid-Phase Peptide Synthesis (SPPS).

MPA resin (0.22 mmol/eq) was swelled in a mixture of DMF:DCM (50:50, v:v). A solution of potassium iodide (125 mM) with DIPEA (1 M) in DMF was added to the reaction vessel along with Fmoc-Lys(ivDde)-OH (0.2 M). The reaction vessel was heated to 75° C. Each amino acid coupling step involved i) deprotection with 20% piperidine in DMF at 90° C.; ii) washing with DMF; iii) activation and coupling of Fmoc protected amino acids with 0.5 M N,N′-diisopropylcarbodiimide (DIC), 0.5 M Oxyma, and 0.2 M Fmoc-amino acid in DMF at 90° C.; iv) washing with DMF. Fmoc-Arg(Pbf)-OH was coupled twice using the methods described above.

### Deprotection IVdde and Add Diboronates to B Chain.

The ivDde protecting group on the lysine residue was removed with 4% hydrazine in DMF, then washed with DMF. A solution of bromo acetic acid (0.2 M, 2 mL) in DMF with DIC (0.5 M, 2 mL) was added immediately and gently mixed for 4 hr. The resin was washed with DMF (3×5 mL). A solution of 1,3-phenylenedimethanamine (1M) in DMF (5 mL) was added to the resin and heated at 50 C for 10 minutes. The resin was washed with DMF (3×5 mL) and a solution of 1-hydroxy-1,3-dihydrobenzo[c][1,2]oxaborole-6-carboxylic acid (0.2 M, 5 mL) in DMF with 1 M N,N′-diisopropylcarbodiimide (DIC, 1M, 1 mL), Oxyma (0.5 M, 2 mL) in DMF and heated at 50 C for 30 min.

### Cleavage and Addition of DTDP to B-Chain.

Crude peptide was globally deprotected with 2,2′-Dithiopyridine (DTDP) in TFA:TIPS:H₂O (95:2.5:2.5) and gently agitated. Crude solution was filtered and peptide was precipitated in cold ether, centrifuged, decanted, washed with additional cold ether, and centrifuged again. Supernatant was decanted and the crude peptide was dried under a gentle stream of nitrogen gas. Crude peptide was dissolved in 20% ACN in water and fractionated by RP-HPLC on a C18 column.

### Combination of A and B Chains of Insulin and Modified Insulins.

A chain of insulin was combined with B chain in 0.2 M NH₄HCO₃ with 6M urea and at pH 8. Mixture was gently agitated, diluted with water and fractionated by RP-HPLC on a C18 column.

### Deprotection of Acm Protecting Groups and Final Oxidation.

The combined intermediate was dissolved in glacial acetic acid and water and vortexed vigorously. A solution of iodine in glacial acetic acid (20 equiv) was added to the reaction mixture and gently agitated. A solution of ascorbic acid (5 mM) was added directly to the reaction mixture. The mixture was diluted in 20% ACN in water and fractionated by RP-HPLC on a Higgins C18 column to give Example 870.

Similar procedures can be followed for the synthesis of Examples 1 to 869 and 871 to 876.

### B. Testing of Compounds for Activity in Biological Assays

Exemplary compounds DS01-DS79 of the present disclosure were tested using an alizarin red S (ARS) displacement assay.

### Procedure for Determination of the Glucose, Fructose, and Lactate Binding (Kd) Using ARS Displacement Assay

The association constant for the binding event between Alizarin Red S (ARS) and the exemplary compounds tested was determined using standard methods in the art. Triplicate titrations of 10⁻⁵ M ARS in 0.1 M phosphate buffer, pH 7.4, were performed in a 96-well plate against serial dilutions of example compounds, ranging in concentration from 0-0.1M at 25° C. The example compound-ARS solution was incubated for 5-45 minutes at 25° C., and absorbance intensity was measured using excitation wavelength 468 nm and emission wavelength 585 nm. Changes in intensity were plotted against the concentration of the example compound, and the intensity data was fitted to yield an association constant for ARS binding.

The association constant for the binding between a target sugar compound (e.g., glucose) and the tested aromatic boron-containing groups was determined via the displacement of ARS bound to the example compounds. Triplicate wells of 10⁻⁵ M ARS and 0.1 M example compounds in 0.1 M phosphate buffer, pH 7.4, were titrated in a 96-well plate against serial dilutions of the target sugar compound, ranging in concentration from 0-2.0 M at 25° C. The boron-ARS-carbohydrate solution was incubated for 30-60 minutes at 25° C. and the intensity of each well was measured in a plate reader at excitation wavelength 468 nm and emission wavelength 585 nm.

Changes in intensity were plotted against concentration of the target sugar compound, and the data was fitted to a one-site competition equation:

$$y = min ( y ) + ( max ( y ) - min ( y ) ) / ( 1 + 1 {0}^{x - logEC 50} )$$

to yield an association constant for the boron compound-target sugar compound binding event.

The binding constants of DS01-DS109 to glucose, fructose, and/or lactate were tested and were calculated, except DS76 was not tested for glucose binding, DS57 and DS75 were not tested for fructose or lactate binding. The tested compounds had Kd values ranging from ˜0.8 mM to ˜486 mM for glucose, −0.9 mM to ˜52 mM for fructose, and ˜24 to ˜425 mM for lactate.

### In Vitro Demonstration of Activity for Compounds of Formula I

CHO cells constitutively expressing Human Insulin Receptor Isoform β were plated in a 96-well tissue culture microplate at 35,000 cells/well and grown overnight in RPMI media supplemented with Glutamine and 10% Fetal Bovine Serum (growth media). The next morning, growth media was replaced with fresh growth media.

A separate microplate was prepared with a stepwise serial dilution of glucose-responsive insulin in DMEM media, without glucose, without phenol red, with 4% w/v serum albumin; wells of serially diluted compounds of Formula I were prepared in triplicate with an appropriate “high” and “low” concentration of glucose to determine change in potency of compounds of Formula I at various potential blood glucose levels.

Growth media on cells was then replaced with DMEM media, no glucose, no phenol red for 5 minutes. The media was aspirated and replaced with the contents of the prepared plate (spiking media) for 10 minutes. The spiking media was aspirated and the cells were fixed with 10% neutral buffered formalin for 10 minutes. The neutral buffered formalin was aspirated, and the microplate was stringently washed with PBS, pH 7.4. The microplate was then blocked with PBS, pH 7.4 supplemented with 10% v/v Fetal Bovine Serum and 0.1% Triton X-100 for 30 minutes. The plate was then stained at 4° C. overnight with 5% FBS in PBS+1:680 v:v of Rb α-phospho-Y1150/Y1151 IR antibody (Cell Signaling Technologies #3024). After stringent washes with PBS, pH 7.4, the microplate was incubated at 37° C. in 5% FBS in PBS+1:1000 of 2° Ab, HRP α-Rabbit (Cell Signaling Technologies, #7074) for 100 minutes. The plate was stringently washed with PBS, pH 7.4, and colorimetric readout was developed for 15 minutes at 37° C. using TMB substrate. Color development was stopped with the addition of 0.1 M hydrochloric acid and absorbance measured at 450 nm. Triplicate absorbance values were plotted in GraphPad Prism and analyzed using a four-parameter logistic regression to generate dose-response curves, and the EC50 of the dose-response curves were compared to assess fold activation of the exemplary compounds of Formula I from low to high glucose concentration.

Examples 315, 318, 320, 565, 590, 606, 611, and 803-880 had an IR Phosphorylation (fold change) ranging from ≥1.2 to 45.

### In Vivo Demonstration of Activity for Compounds of Formula I

Diabetes was induced in 12-week-old B6 mice using streptozocin (STZ) treatment. Three weeks after the final STZ treatment, blood glucose of the mice was sampled from lateral tail vein to confirm diabetes. Mice with blood glucose of >200 mg/dl were deemed to be diabetic and fasted for 1-6 hours prior to injection. Human insulin and a compound of Formula I in sterile phosphate buffered saline, pH 7.4 were injected either subcutaneously via neck scruff or intraperitoneally, depending on the experiment. Blood glucose was sampled with a glucometer via lateral tail vein at 15-minute intervals. After one hour of initial stabilization of blood glucose levels post-insulin injection, mice were challenged with an intraperitoneal injection of glucose (e.g., 2 g/kg, 4 g/kg, or 6 g/kg; the actual dose depends on the example and experiment) in sterile phosphate buffered saline. The exemplary compound activated to lower blood glucose upon the introduction of a glucose bolus, while human insulin did not activate in a glucose-dependent manner.

The above experiment showed in vivo preferential activity response of an exemplary compound of the disclosure to glucose.

Streptozotocin-treated mice (55 mg/kg, 5 days) undergo surgical catheterization of a carotid artery and jugular vein for blood sampling and infusions. After a recovery period of 3-4 days, mice are placed in an experimental chamber, connected to sampling/infusion lines, and briefly fasted. Somatostatin (5 mg/kg/min) is continuously infused throughout the study. At time 0 min, biosynthetic human insulin (BHI) or a compound of Formula I are infused at 4 mU/kg/min and glucose is infused at variable rates to achieve steady state (“clamped”) at pre-determined glycemic levels. Blood glucose (BG) is clamped in windows of stepwise increasing blood glucose concentration. Steady-state Glucose Infusion Rate (GIR) is measured for each step increase in clamped blood glucose and plotted to assess the increase in GIR as a function of increasing blood glucose. As BG increases, compounds of Formula I require greater GIR to maintain clamped BG than does BHI, demonstrating a glucose-responsive increase in compound glucose-lowering action with increased BG concentrations.

It is also observed in cell-based experiments on compounds containing formulae FF50-FF62, FF116, and FF121-FF134 that sensors with geminal alkyl substituent on the same carbon as the nitrogen conjugated to the boroxole or boronates provided between 5-56% higher glucose responsiveness in the range of 3-20 mM glucose in comparison to variants that do not have the geminal alkyl substituents. For example, when a Z1C represented by one of formulae FF50-FF62, FF116, and FF121-134 is conjugated to lysine residues in insulin wherein the boronates (B1,B2) in formulae FF50-FF62, FF116, and FF121-134 are represented by F2, the resulting insulin is observed to be between 11-56% more responsive to changes in glucose levels between 3-20 mM glucose than if instead of one of formulae FF50-FF62, FF116, and FF121-134, one uses 2,3-diaminopropionic acid. This data shows that the presence of the geminal alkyl substituent on the same carbon as the nitrogen conjugated to the boroxole or boronates improves glucose responsiveness of the resulting insulin conjugate, and in tested variations in the 3-20 mM glucose range. Without wishing to be bound by theory, it is believed that this general principle extends to other formulae FF50-FF62, FF116, and FF121-FF134 providing a framework for enhancement of glucose responsiveness by at least 5%, at least 10%, at least 20%, or at least 40% in the 3-20 mM or 2-50 mM glucose ranges. Without wishing to be bound by theory, it is believed based on observations of glucose responsiveness trends, that the presence of the carbonyl group adjacent to- or within less than two carbon centers away from the amine groups in FF formulae (to which aromatic boron-containing groups are conjugated) enhances glucose responsiveness through impacting ability to turn off activity of drug substance through plasma protein interactions such as with albumin and that this is independent of glucose affinity such that glucose affinity is not impacted by the position of this carbonyl group. Without wishing to be bound by theory, it is believed that the pharmacokinetics of the molecules and potential albumin or blood proteins binding is impacted by the position of this carbonyl group, and thereby enhances overall glucose responsiveness whilst the absolute glucose affinity is maintained or nearly identical. Therefore, in certain embodiments of the present invention, the carbonyl group (as part of an acid, amid or linkage to X in FF formulae) is placed within less than three-, or within less than two-carbon center away from one of the two amines to which the boron-containing compounds are conjugated. In certain embodiments, the placement of amines within two carbon centers from each other enables the spatial and geometric constraining of the aromatic boron containing groups to enhance glucose binding and selectively, and furthermore the presence of a carbonyl group (for example, as part of an amide linkage) which is within less than two carbon centers, from one of the two amines (to which aromatic boron containing groups are attached) ensures differential albumin binding in a manner that results in the compound exhibiting glucose responsiveness in the blood and in the body. In some embodiments, the combination of geometrical constraining of the two amines to which the aromatic boron containing groups are conjugated, as well as the presence of the carbonyl within one to two carbon centers from one of the amines provides the necessary requirements for glucose responsiveness in physiological blood and plasma glucose levels. Experiments on cell-based assays of insulins with lysines conjugated with one of formulae FF50-FF62, FF116, and FF121-134 demonstrated that the enhanced glucose responsiveness of the insulins is increased when one or more lysines are modified as described by Formula I using one of formulae FF50-FF62, FF116, and FF121-134, and wherein the lysines are present in insulin (as insertions or mutations) or in a polypeptide that is appended to the N- or C-terminus of the B-chain of insulin or the C-terminus of the A-chain of insulin, and wherein there are additional lysine residues within the insulin sequence that are similarly modified. The results are further corroborated by testing of the compounds of Formula I in STZ diabetic mouse models wherein the activity of the insulin is measured through bolus injections of the compounds of Formula I followed by glucose challenges and measurements of blood glucose, or through glucose clamp assays in which activity of the insulins is measured as a function of blood glucose levels. The results further showed that exemplary compounds of Formula I disclosed herein function in the body and are responsive to physiological changes in blood glucose and provide dynamic insulin action in the body in response to changes in blood glucose levels.

In certain embodiments a sequence is appended to the N-terminus and/or C-terminus, and/or inserted into the sequence of the A-chain of insulin, wherein the A-chain of insulin comprises one of the following sequences, optionally with up to four additional deletions and/or mutations:

(SEQ ID NO: 1)
GIVEQCCTSICSLYQLENYCN,
(SEQ ID NO: 3)
GIVKQCCTSICSLYQLENYCN,
(SEQ ID NO: 4)
GIVEQCCHSICSLYQLENYCN,
(SEQ ID NO: 5)
GIVEQCCASICSLYQLENYCN,
(SEQ ID NO: 6)
GIVEQCCTRICSLYQLENYCN,
(SEQ ID NO: 7)
GIVEQCCTKICSLYQLENYCN,
(SEQ ID NO: 8)
GIVEQCCTSICSEYQENYCN,
(SEQ ID NO: 9)
GIVKQCCTSICSLYQLENYCG,
(SEQ ID NO: 10)
GIVEQCCHSICSLYQLENYCG,
(SEQ ID NO: 11)
GIVEQCCASICSLYQLENYCG,
(SEQ ID NO: 12)
GIVEQCCTRICSLYQLENYCG,
(SEQ ID NO: 13)
GIVEQCCTKICSLYQLENYCG,
(SEQ ID NO: 14)
GIVEQCCTSICSEYQENYCG,
(SEQ ID NO: 15)
GIVEQCCTSICSEYQENYC,
(SEQ ID NO: 16)
GIVEQCCTSICSLYQLENYCNK,
(SEQ ID NO: 17)
KPGIVEQCCTSICSLYQLENYCN,
(SEQ ID NO: 18)
KPIVEQCCTSICSLYQLENYCN,
(SEQ ID NO: 19)
KPVEQCCTSICSLYQLENYCN,
(SEQ ID NO: 20)
KPGVEQCCTSICSLYQLENYCN,
(SEQ ID NO: 21)
GEKPVEQCCTSICSLYQLENYCN,
(SEQ ID NO: 22)
KPGEKPVEQCCTSICSLYQLENYCN,
(SEQ ID NO: 23)
KPVEQCCTSICSLYQLENYCNK,
(SEQ ID NO: 24)
KPVEQCCTSICSLYQLENYCNEKP,
(SEQ ID NO: 25)
GIVEQCCTSICSLYQLENYCGK,
(SEQ ID NO: 26)
KPGIVEQCCTSICSLYQLENYCG,
(SEQ ID NO: 27)
KPIVEQCCTSICSLYQLENYCG,
(SEQ ID NO: 28)
KPVEQCCTSICSLYQLENYCG,
(SEQ ID NO: 29)
KPGVEQCCTSICSLYQLENYCG,
(SEQ ID NO: 30)
GEKPVEQCCTSICSLYQLENYCG,
(SEQ ID NO: 31)
KPGEKPVEQCCTSICSLYQLENYCG,
(SEQ ID NO: 32)
KPVEQCCTSICSLYQLENYCGK,
(SEQ ID NO: 33)
KPVEQCCTSICSLYQLENYCGEKP,

and/or a sequence is appended to the N-terminus and/or C-terminus, and/or inserted into the sequence of the B-chain of insulin, wherein the B-chain of insulin comprises one of following sequences, and optionally with up to four additional deletions and/or mutations:

(SEQ ID NO: 2)
FVNQHLCGSHLVEALYLVCGERGFFYTPKT,
(SEQ ID NO: 34)
FVNQHLCGSHLVEALYLVCGERGFFYTP,
(SEQ ID NO: 35)
FVNQHLCGSHLVEALYLVCGKRGFFYTP,
(SEQ ID NO: 36)
FVNQHLCGSHLVEALYLVCGKRGFFYTPRT,
(SEQ ID NO: 37)
FVNQHLCGSHLVEALYLVCGKRGFFYT,
(SEQ ID NO: 38)
VNQHLCGSHLVEALYLVCGERGFFYTPKT,
(SEQ ID NO: 39)
NQHLCGSHLVEALYLVCGERGFFYTPKT,
(SEQ ID NO: 40)
QHLCGSHLVEALYLVCGERGFFYTPKT,
(SEQ ID NO: 41)
PFVNQHLCGSHLVEALYLVCGERGFFYTPKT,
(SEQ ID NO: 42)
PFVNQHLCGSHLVEALYLVCGKRGFFYTPRT,
(SEQ ID NO: 43)
PFVNQHLCGSHLVEALYLVCGKEGFFYTPRT,
(SEQ ID NO: 44)
PFVNQHLCGSHLVEALYLVCGKRGFFYTPR,
(SEQ ID NO: 45)
PFVNQHLCGSHLVEALYLVCGKRGFFYTRPT,
(SEQ ID NO: 46)
PFVNQHLCGSHLVEALYLVCGKRGFFYTRP,
(SEQ ID NO: 47)
PFVNQHLCGSHLVEALYLVCGKNGFFYTPRT,
(SEQ ID NO: 48)
PFVNQHLCGSHLVEALYLVCGKNGFFYTPRT,
(SEQ ID NO: 49)
PFVNQHLCGSHLVEALYLVCGKNGFFYTPR,
(SEQ ID NO: 50)
PFVNQHLCGSHLVEALYLVCGKNGFFYTRPT,
(SEQ ID NO: 51)
PFVNQHLCGSHLVEALYLVCGKNGFFYTRP,
(SEQ ID NO: 52)
PFVNQHLCGSHLVEALYLVCGKEGFFYTPRT,
(SEQ ID NO: 53)
PFVNQHLCGSHLVEALYLVCGKEGFFYTPRT,
(SEQ ID NO: 54)
PFVNQHLCGSHLVEALYLVCGKEGFFYTPR,
(SEQ ID NO: 55)
PFVNQHLCGSHLVEALYLVCGKEGFFYTRPT,
(SEQ ID NO: 56)
PFVNQHLCGSHLVEALYLVCGKEGFFYTRP,
(SEQ ID NO: 57)
PFVNQHLCGSHLVEALYLVCGKRGFFYTPR,
(SEQ ID NO: 58)
PVNQHLCGSHLVEALYLVCGERGFFYTPKT,
(SEQ ID NO: 59)
PVNQHLCGSHLVEALYLVCGKRGFFYTPRT,
(SEQ ID NO: 60)
PVNQHLCGSHLVEALYLVCGKRGFFYTPR,
(SEQ ID NO: 61)
PNQHLCGSHLVEALYLVCGERGFFYTPKT,
(SEQ ID NO: 62)
PNQHLCGSHLVEALYLVCGKRGFFYTPRT,
(SEQ ID NO: 63)
PNQHLCGSHLVEALYLVCGKRGFFYTPR,
(SEQ ID NO: 64)
PQHLCGSHLVEALYLVCGERGFFYTPKT,
(SEQ ID NO: 65)
PQHLCGSHLVEALYLVCGKRGFFYTPRT,
(SEQ ID NO: 66)
PQHLCGSHLVEALYLVCGKRGFFYTPR,
(SEQ ID NO: 67)
PFVNQHLCGSHLVEALYLVCGERGFFYTKPT,
(SEQ ID NO: 68)
PFVNQHLCGSHLVEALYLVCGERGFFYTKP,
(SEQ ID NO: 69)
PVNQHLCGSHLVEALYLVCGERGFFYTKPT,
(SEQ ID NO: 70)
PVNQHLCGSHLVEALYLVCGERGFFYTKP,
(SEQ ID NO: 71)
PNQHLCGSHLVEALYLVCGERGFFYTKPT,
(SEQ ID NO: 72)
PNQHLCGSHLVEALYLVCGERGFFYTKP,
(SEQ ID NO: 73)
PQHLCGSHLVEALYLVCGERGFFYTKPT,
(SEQ ID NO: 74)
FVNQHLCGSHLVEALYLVCGERGFFYTPK,
(SEQ ID NO: 24047)
FVNQHLCGSHLVEALYLVCGKRGFFYTPKT,
and
(SEQ ID NO: 24048)
FVNQHLCGSHLVEALYLVCGKRGFFYTPR.

### C. Exemplary Compounds

The following are non-limiting examples of compounds of Formula I, that can be prepared according to the methods described herein.

1. A compound comprising X1 and one or more Z1c, or a tautomer, stereoisomer or a mixture of stereoisomers, or a pharmaceutically acceptable salt, or hydrate, or isotopic derivative thereof,
wherein:
X1 comprises:
(i) NH₂ or OH;
(ii) a drug substance comprising an amine;
(iii) a drug substance that is covalently conjugated to an amine containing linker; or
(iv) an amine configured to be covalently conjugated to a drug substance;
wherein each Z1c is independently selected from Formulae FF1-FF224;
wherein each Z1c is covalently conjugated, directly or indirectly, to an amine in X1 or to OH when X1 is OH, and
wherein Formulae FF1-FF48 are: 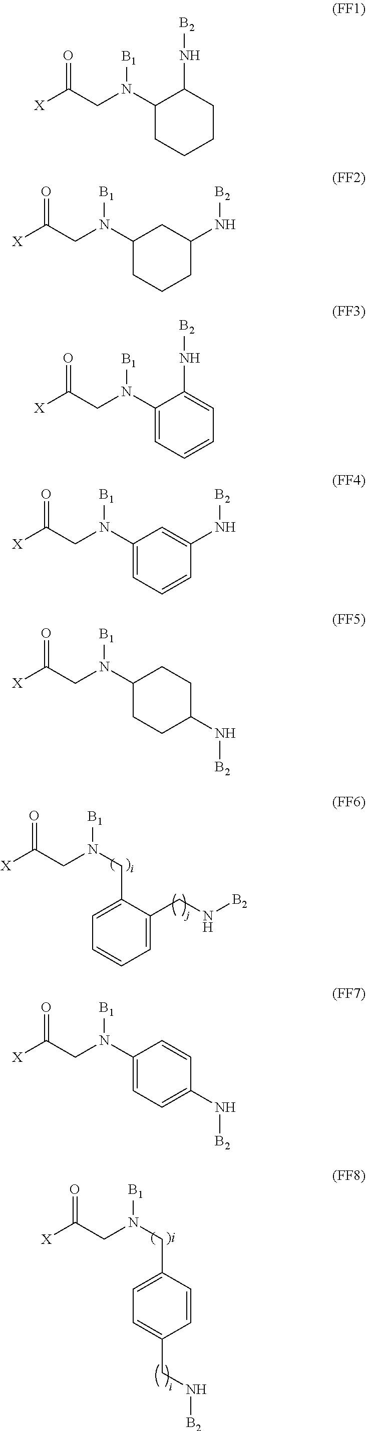 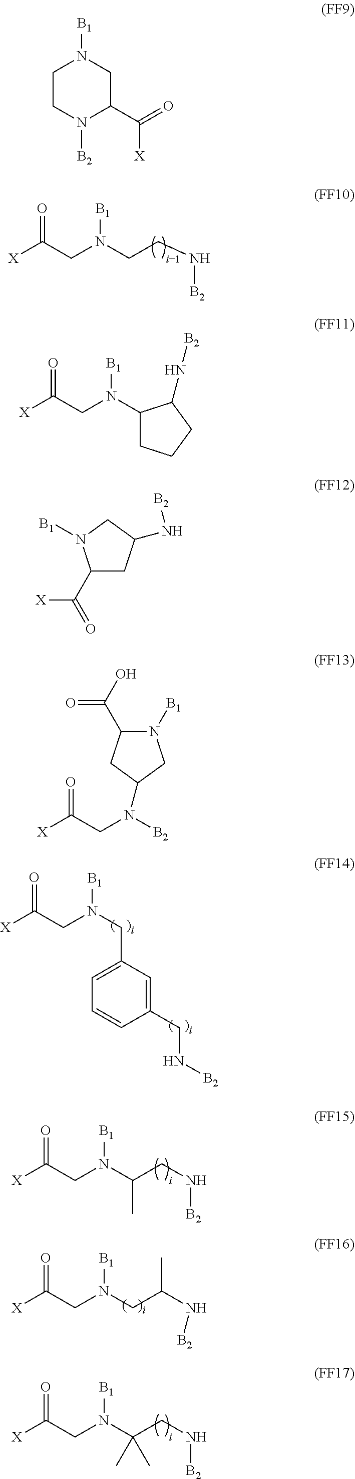 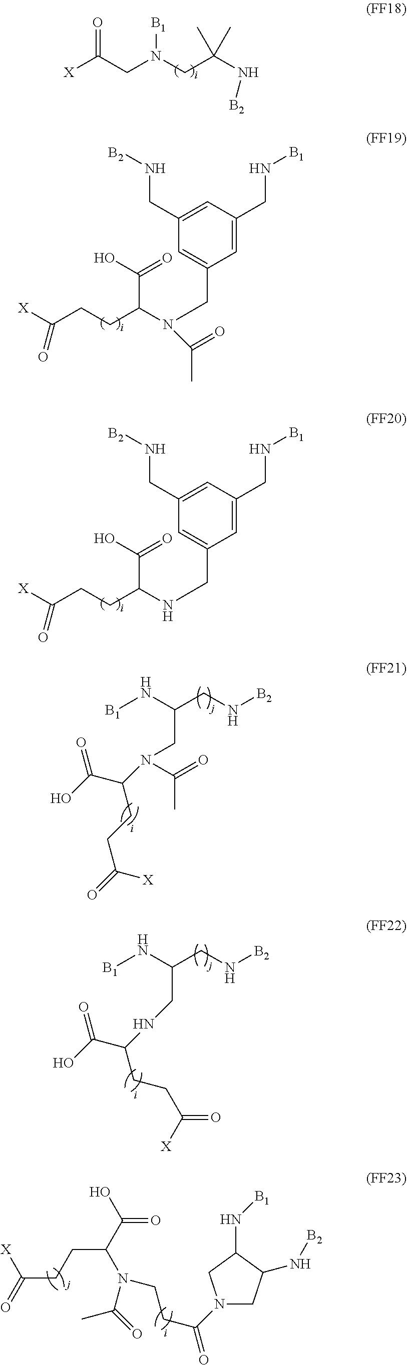 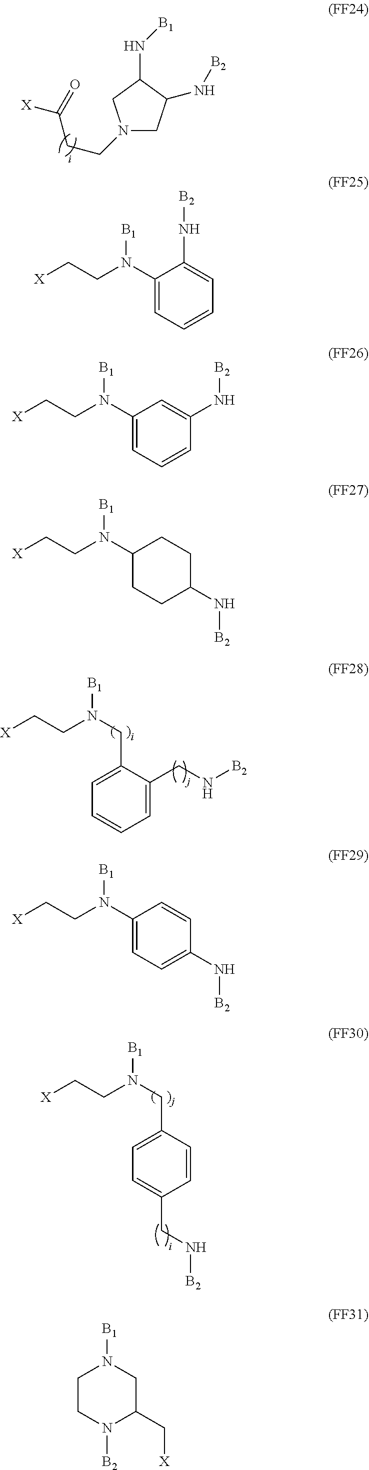 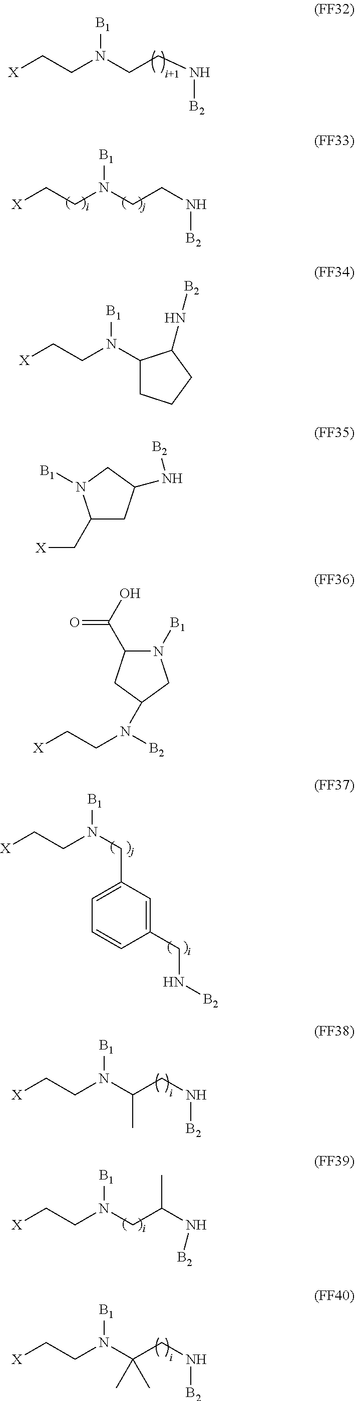 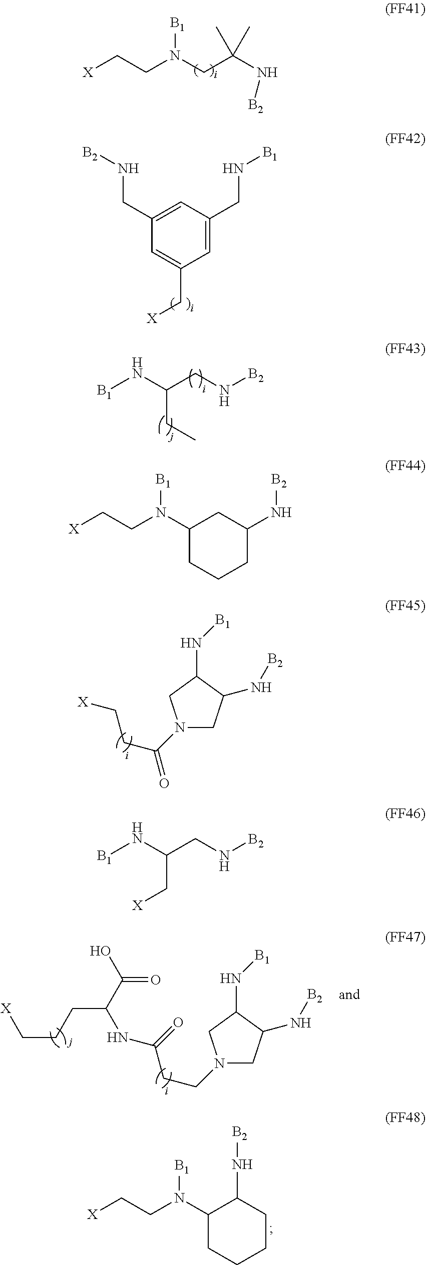
wherein X represents a point of covalent attachment either directly to an amine in X1 or to an amine that is covalently conjugated directly or indirectly to X1, or to OH when X1 is OH;
i is 1, 2, 3, 4, 5, 6, or 7;
j is 1, 2, 3, 4, 5, 6, or 7; and
B₁ and B₂, which may be identical or different, each independently represents an aromatic boron-containing group;
wherein Formulae FF49-FF88 are: 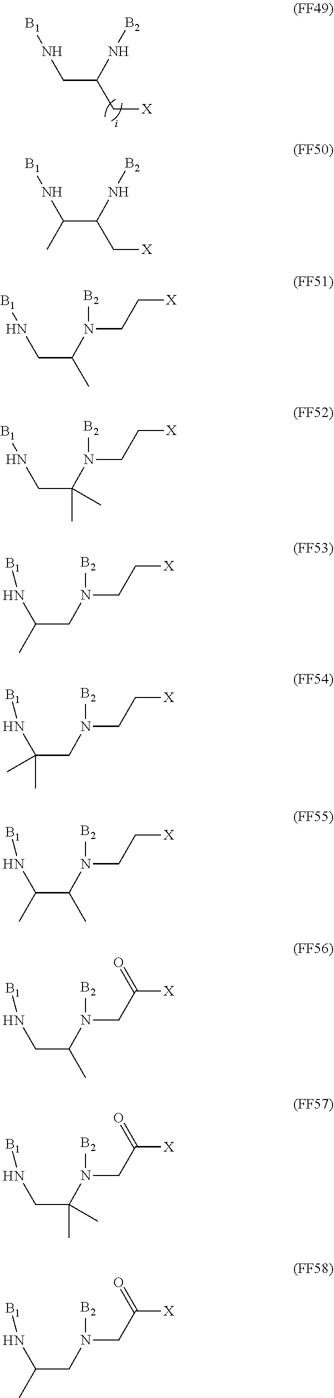 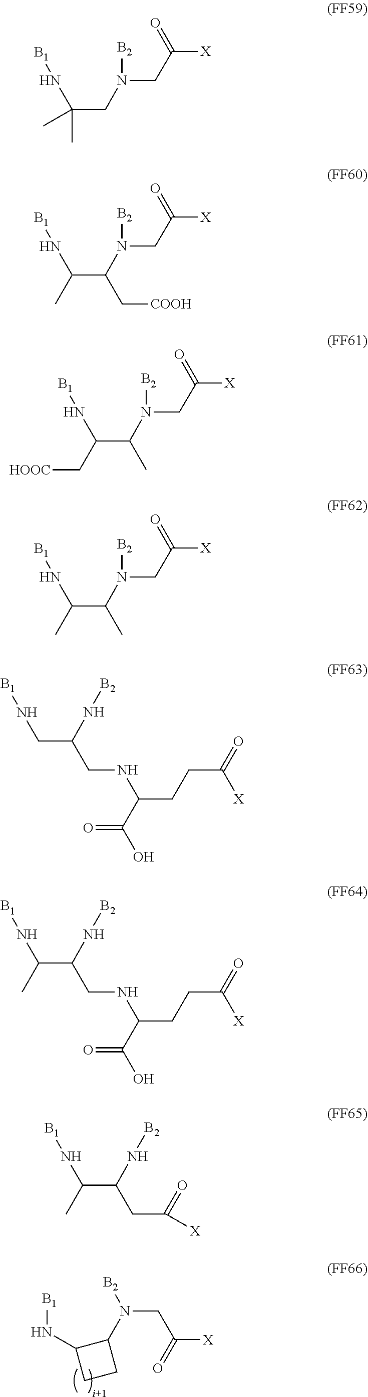 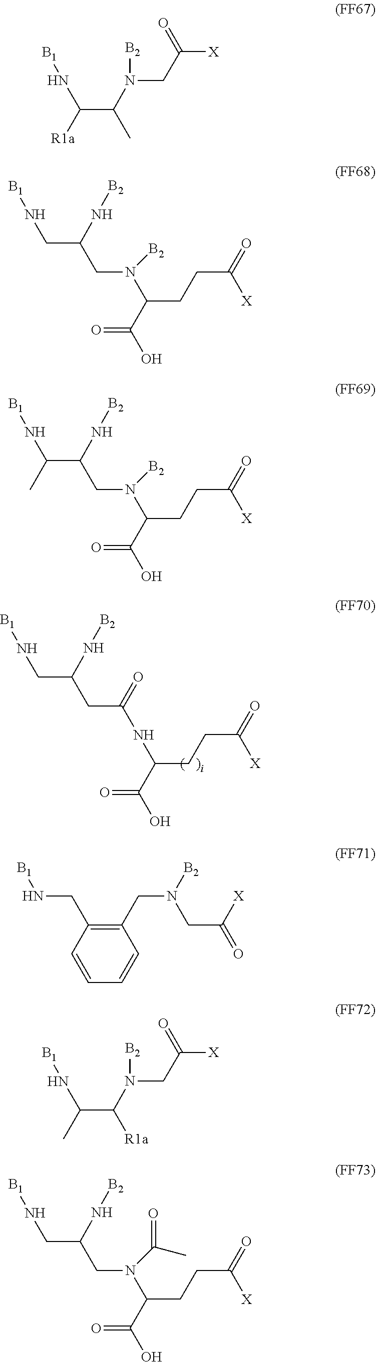 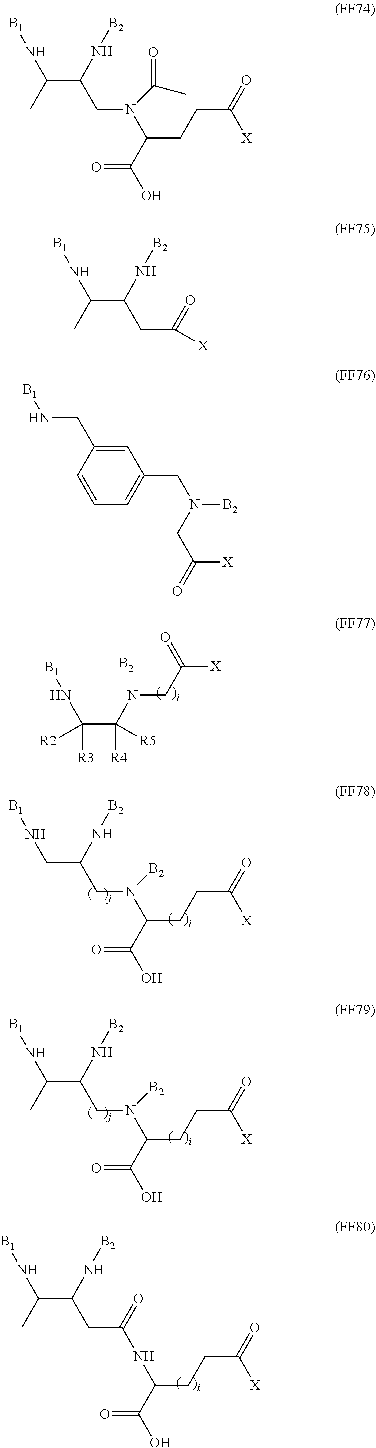 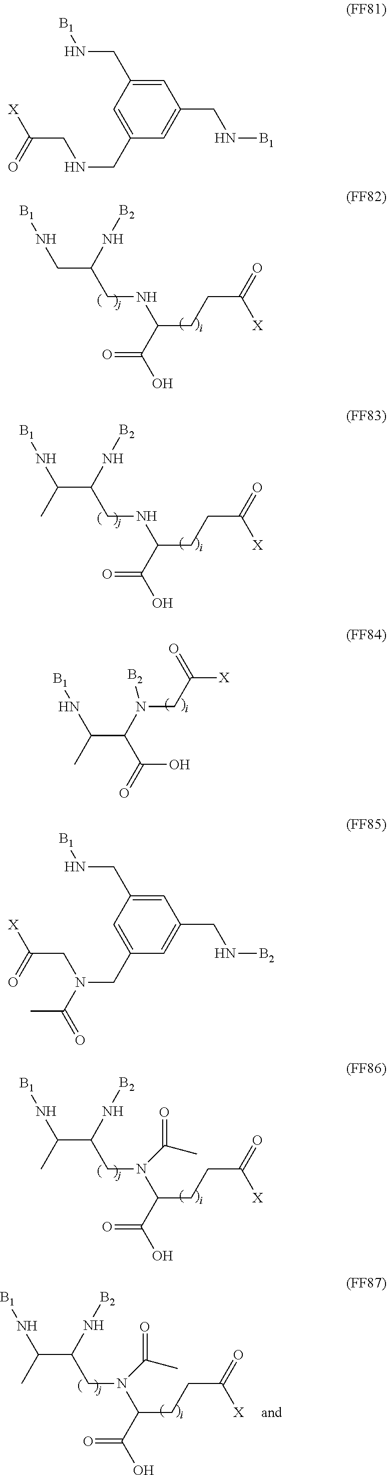 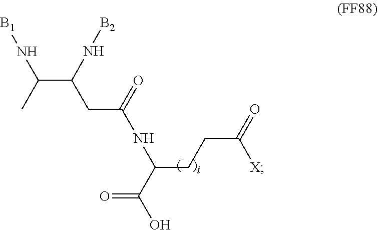
wherein X represents a point of covalent attachment either directly to an amine in X1 or to an amine that is covalently conjugated directly or indirectly to X1, or to OH when X1 is OH;
i is 1, 2, 3, 4, 5, 6, or 7;
j is 1, 2, 3, 4, 5, 6, or 7;
R1a is selected from COOH, CH₃, H, and OH;
R2, R3, R4 and R5 are each independently selected from CH₃, H, OH, and COOH, and at least one of R2, R3, R4 and R5 is CH₃ or OH; and
B₁ and B₂, which may be identical or different, are each independently an aromatic boron-containing group;
wherein Formulae FF89-FF112 are: 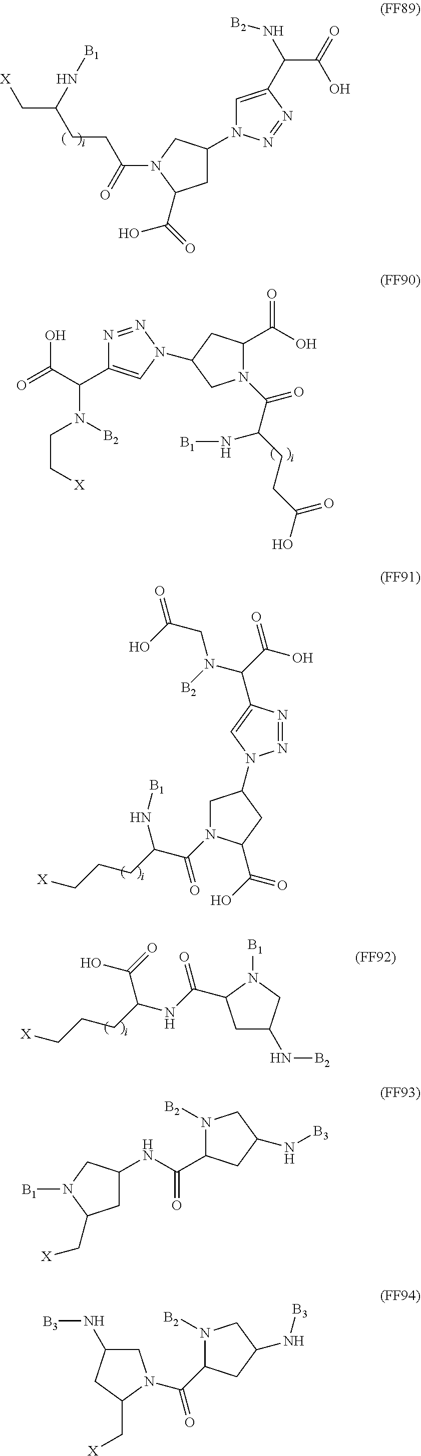 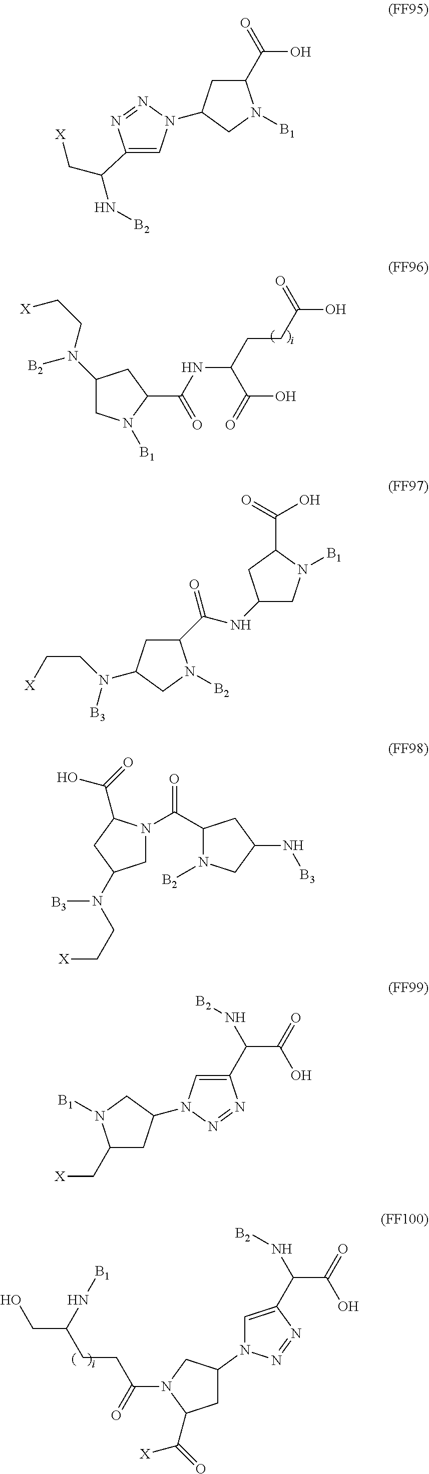 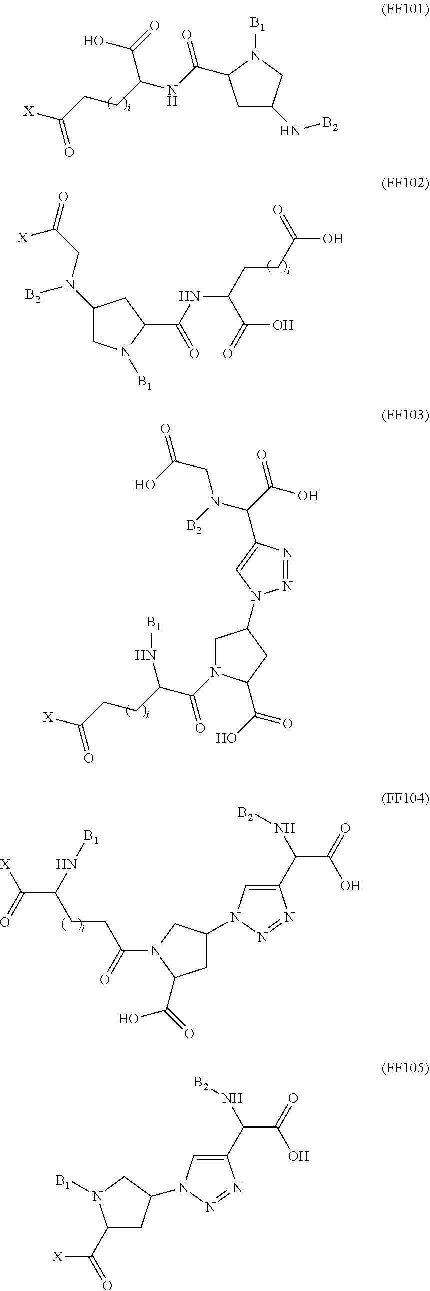 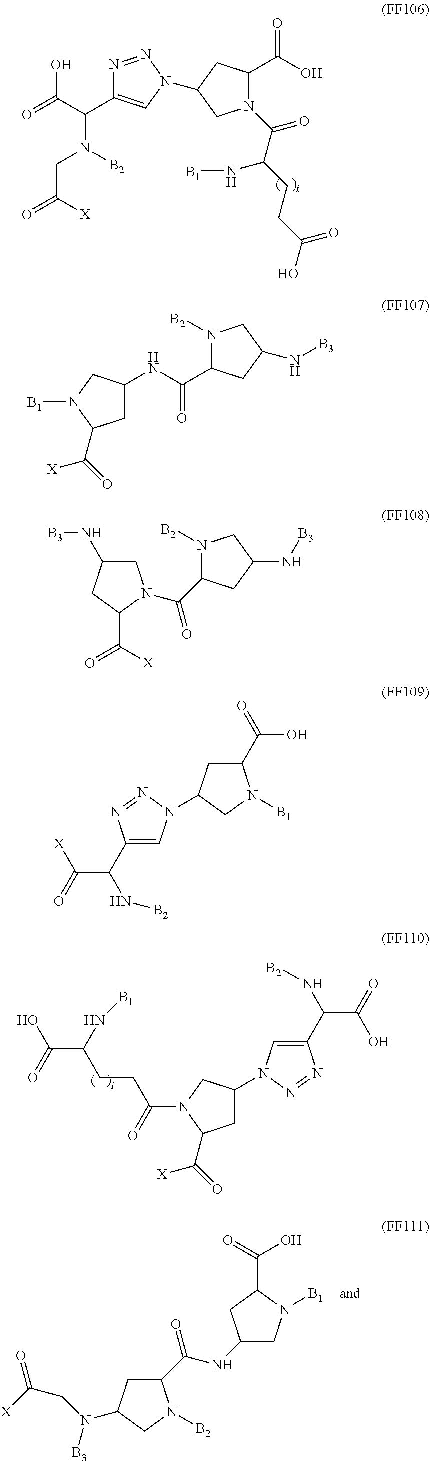 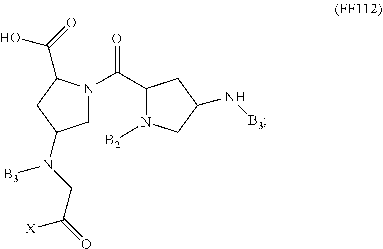
wherein X represents a point of covalent attachment either directly to an amine in X1 or to an amine that is covalently conjugated directly or indirectly to X1, or to OH when X1 is OH;
i is 1, 2, 3,4, 5,6, or 7; and
B₁, B₂ and B₃, which may be identical or different, are each independently an aromatic boron-containing group, a carboxylic acid derivative, or a H, wherein in each FF89-FF112 structure containing B1, B2 and B3 groups, at least two of the B1, B2 and B3 groups are independently an aromatic boron-containing group;
wherein Formulae FF113-FF136 are: 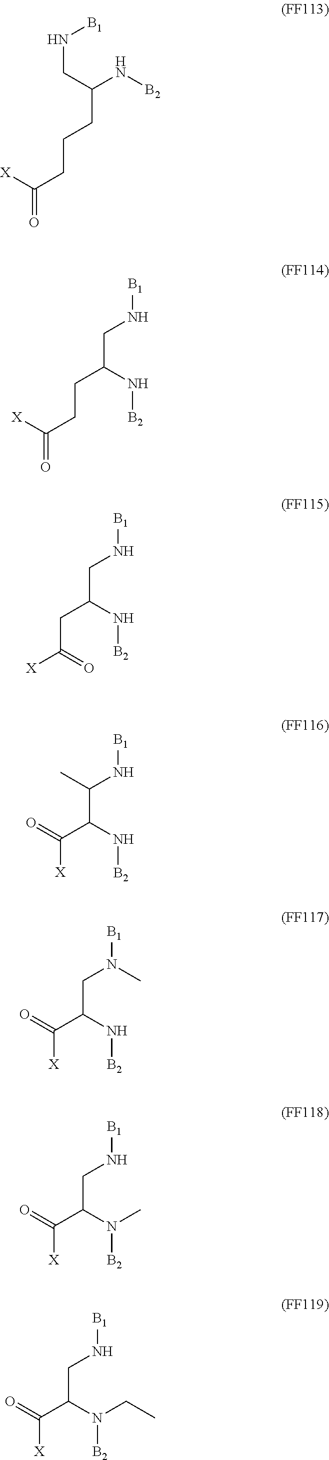 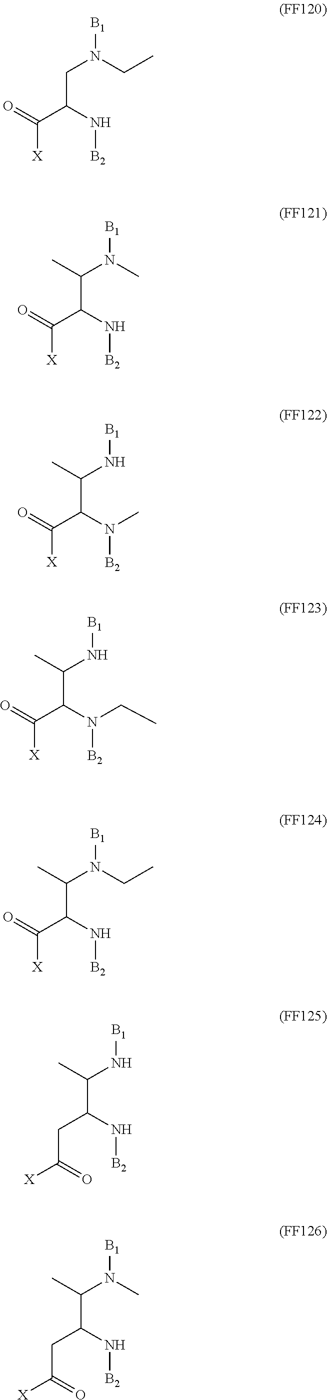 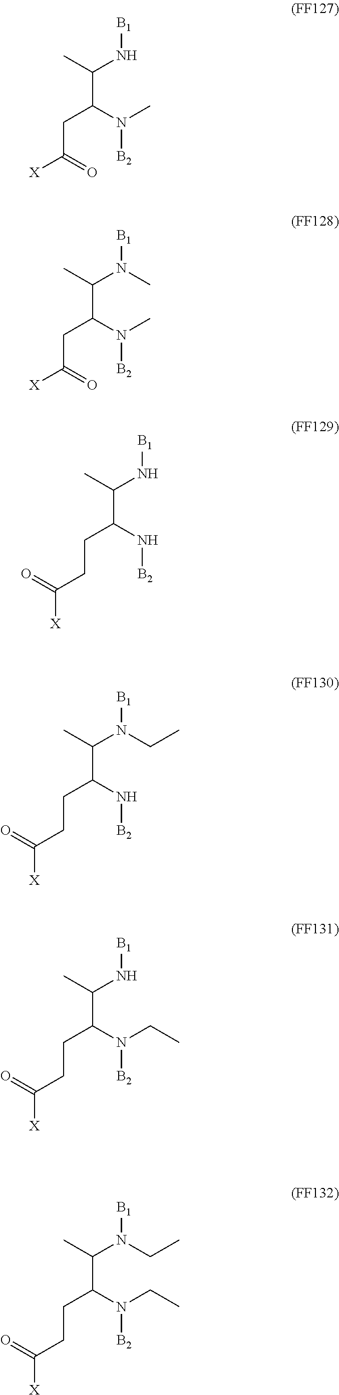 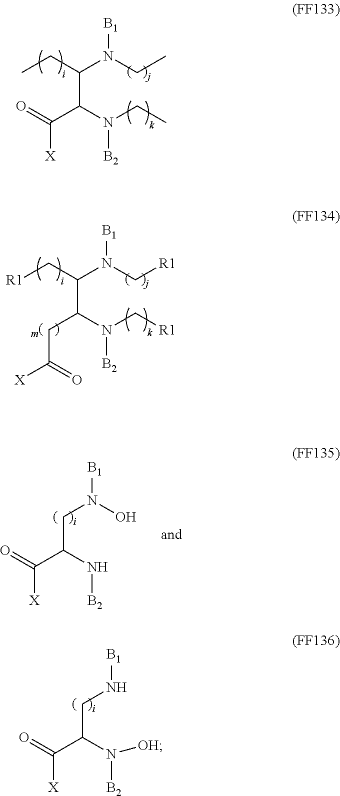
wherein X represents a point of covalent attachment either directly to an amine in X1 or to an amine that is covalently conjugated directly or indirectly to X1, or to OH when X1 is OH;
i is 1, 2, 3, 4, 5, 6, or 7;
j is 1, 2, 3, 4, 5, 6, or 7;
k is 1, 2, 3, 4, 5, 6, or 7;
m is 1, 2, 3, 4, 5, 6, or 7;
each R1 is independently selected from H, an alkyl group, an acyl group, a cycloalkyl group, a haloalkyl group, an aryl group, and a heteroaryl group, each R1 optionally comprises one or more alkyl-halide, halide, sulfhydryl, aldehyde, amine, acid, hydroxyl, alkyl, or aryl groups; and
B₁ and B₂, which may be identical or different, each independently represents an aromatic boron-containing group;
wherein Formulae FF137-FF160 are: 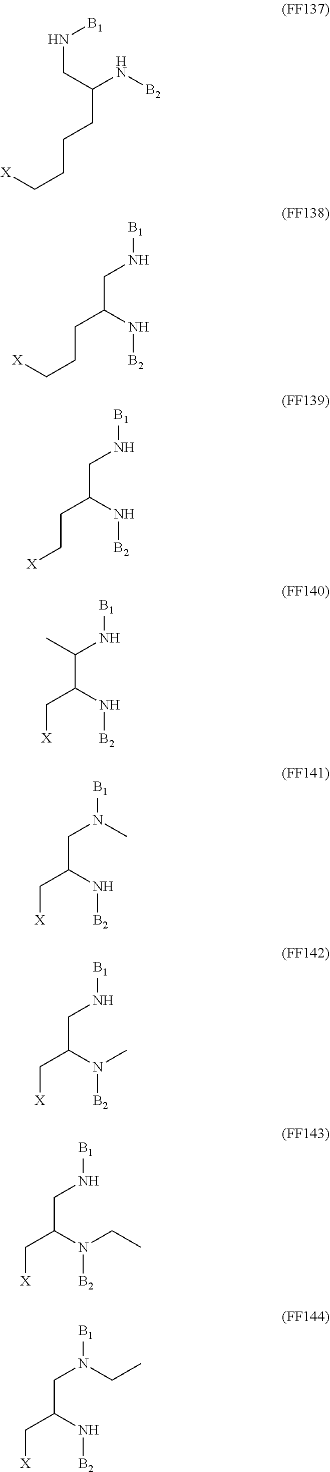 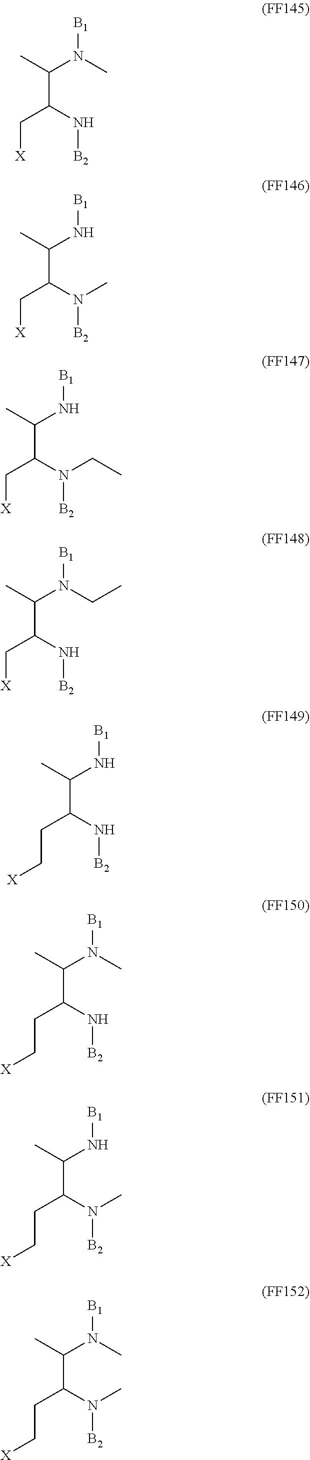 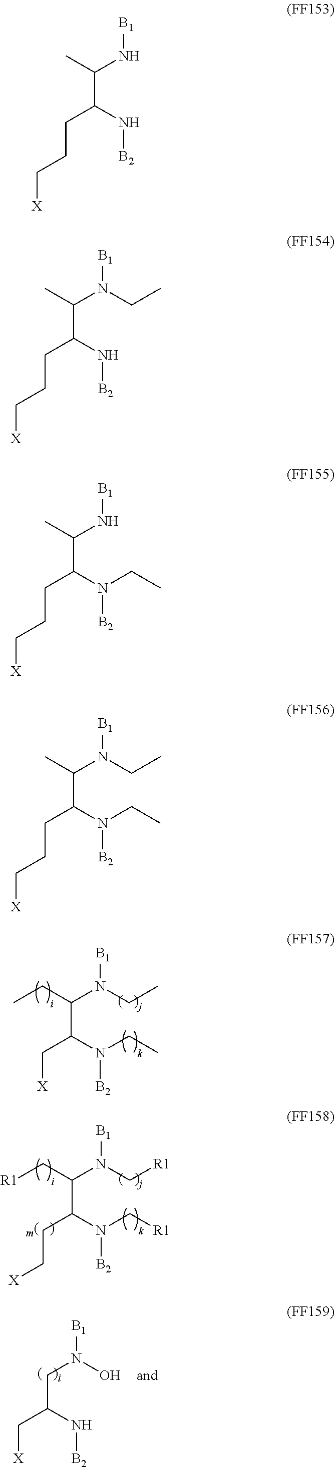 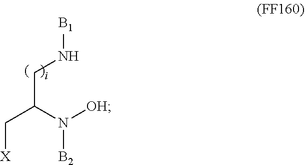
wherein X represents a point of covalent attachment either directly to an amine in X1 or to an amine that is covalently conjugated directly or indirectly to X1, or to OH when X1 is OH;
i is 1, 2, 3, 4, 5, 6, or 7;
j is 1, 2, 3, 4, 5, 6, or 7;
k is 1, 2, 3, 4, 5, 6, or 7;
m is 1, 2, 3, 4, 5, 6, or 7;
each R1 is independently selected from H, an alkyl group, an acyl group, a cycloalkyl group, a haloalkyl group, an aryl group, and a heteroaryl group, each R1 optionally comprises one or more alkyl-halide, halide, sulfhydryl, aldehyde, amine, acid, hydroxyl, alkyl, or aryl groups; and
B₁ and B₂, which may be identical or different, each independently represents an aromatic boron-containing group;
wherein Formulae FF161-FF164 are: 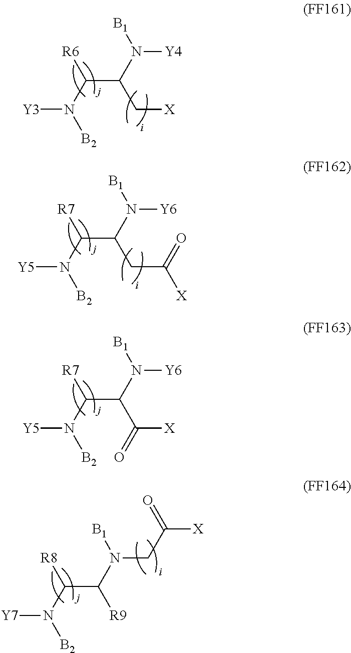
wherein X represents a point of covalent attachment either directly to an amine in X1 or to an amine that is covalently conjugated directly or indirectly to X1, or to OH when X1 is OH;
i is 1, 2, 3, 4, or 5;
j is 1, 2, 3, 4, or 5;
each R6, R7, R8, and R9 for different values of j is independently selected from H, CF₃, CH₃, CHF₂, and (CH₂)ₘCH₃, wherein m is 1, 2, 3, 4, or 5;
Y3, Y4, Y5, Y6 and Y7 are each independently selected from H, CH₂—X4, and Formulae IV-1 to IV-135;
wherein X4 is selected from —COOH, —(CH₂)ₘCOOH, an alkyl group, an acyl group, a cycloalkyl group, a haloalkyl group, an aryl group, and a heteroaryl group, each X4 optionally comprises one or more alkyl-halide, halide, sulfhydryl, aldehyde, amine, acid, hydroxyl, alkyl or aryl groups; wherein m is 1, 2, 3, 4, or 5;
wherein at least one of Y5, Y6 and Y7 in Formulae FF162 and FF163 is not H and at least one of Y7, R8 and R9 in FF164 is not H; and
wherein Formulae IV-1 to IV-135 are: 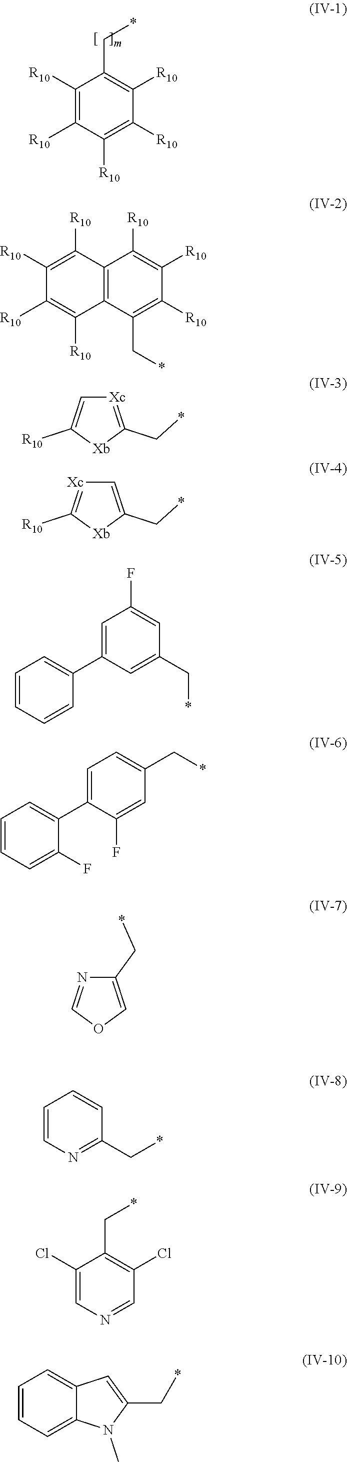 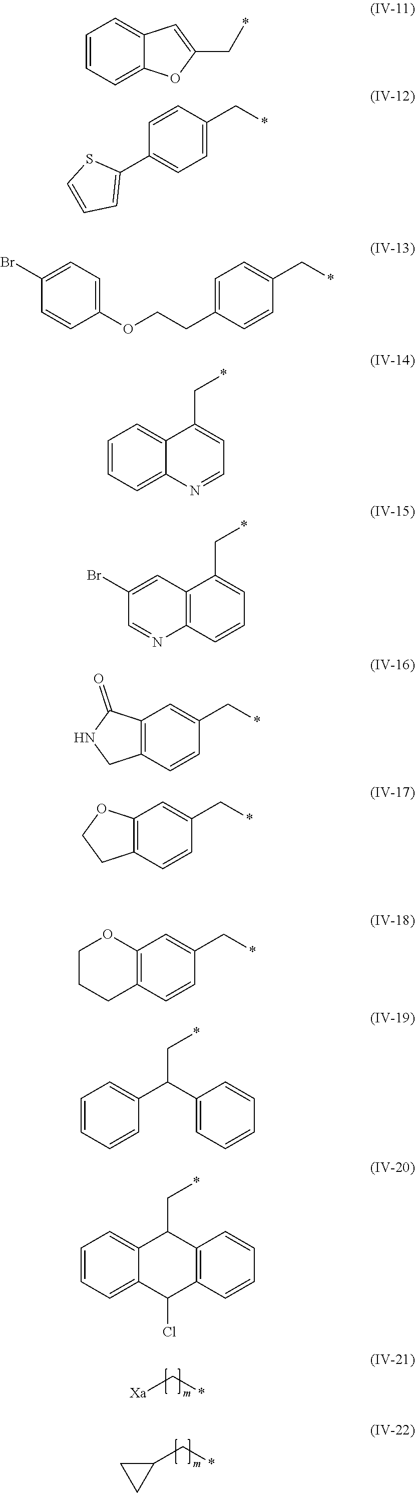 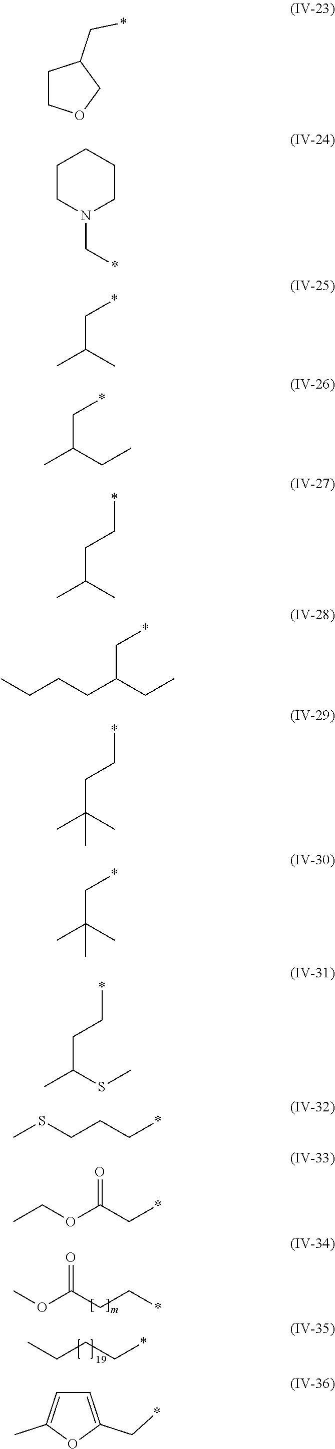 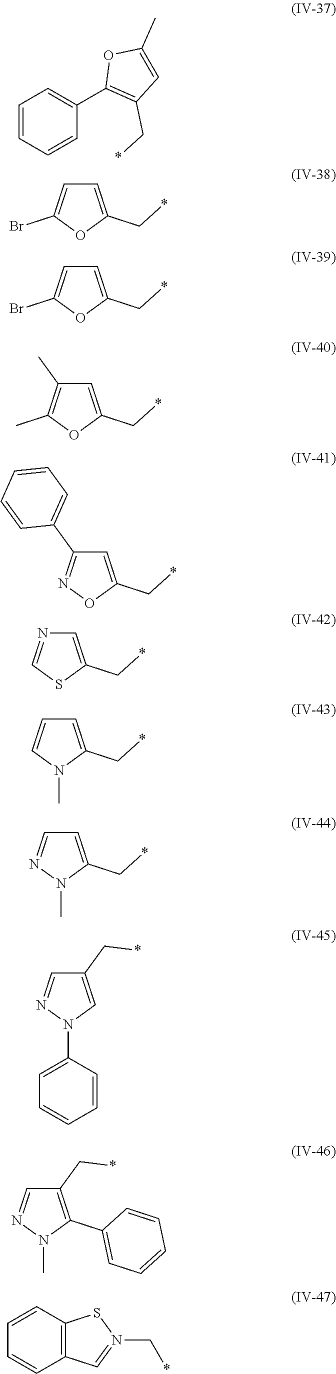 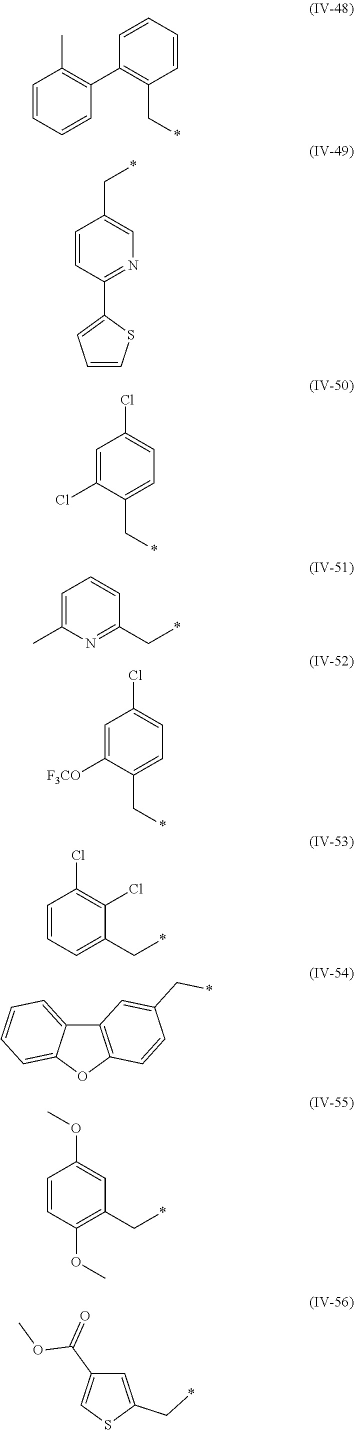 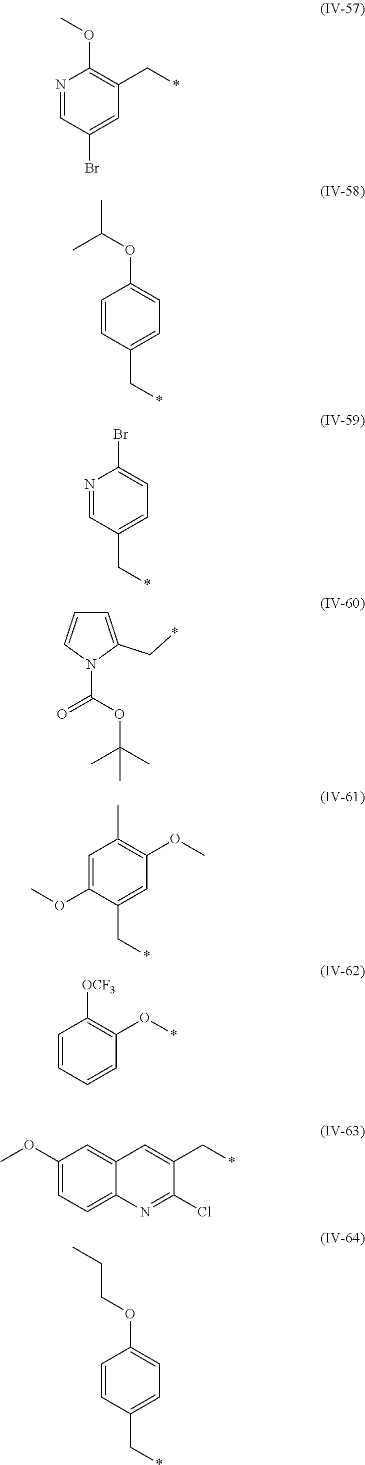 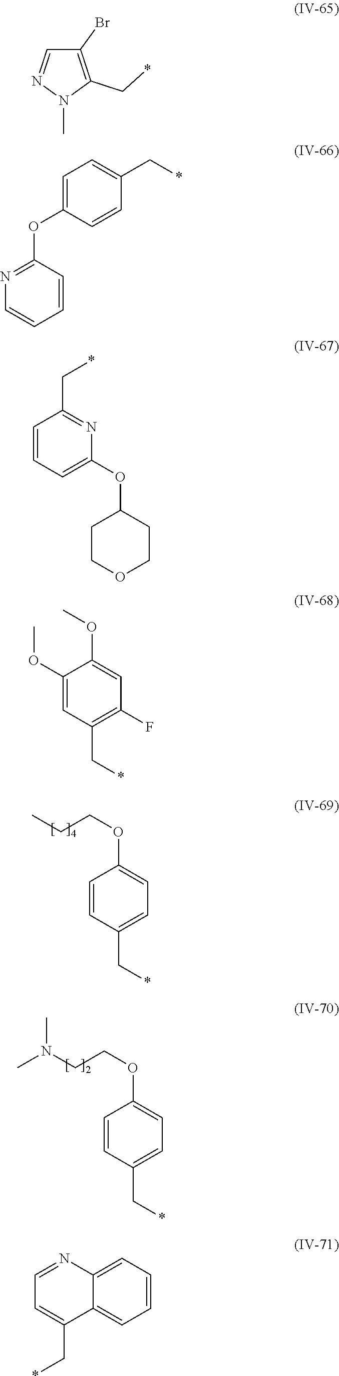 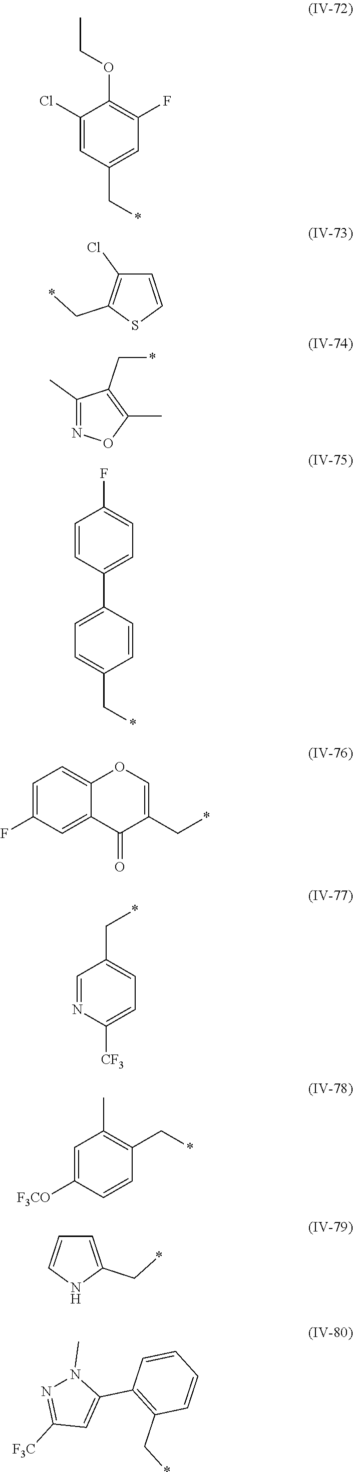 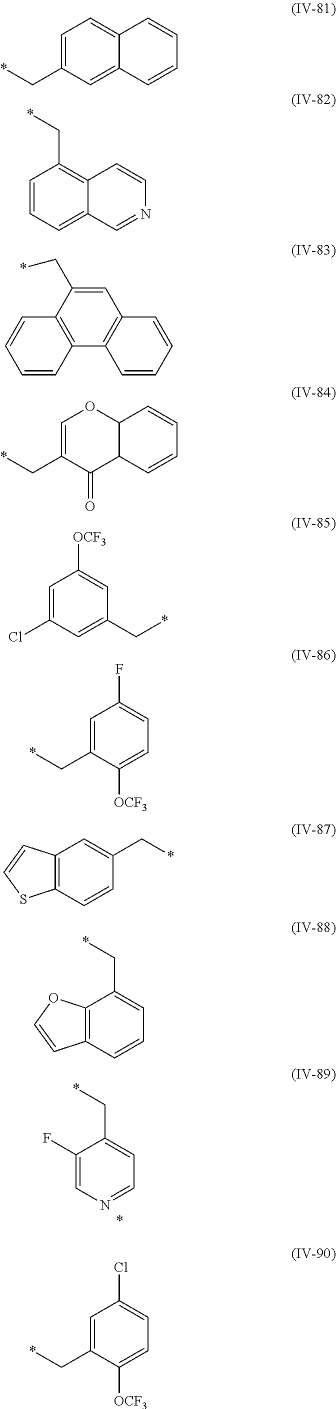 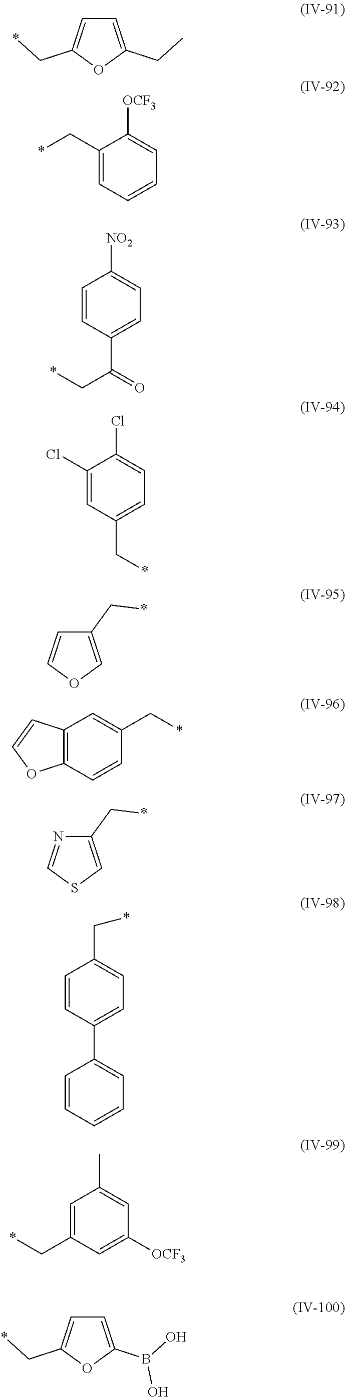 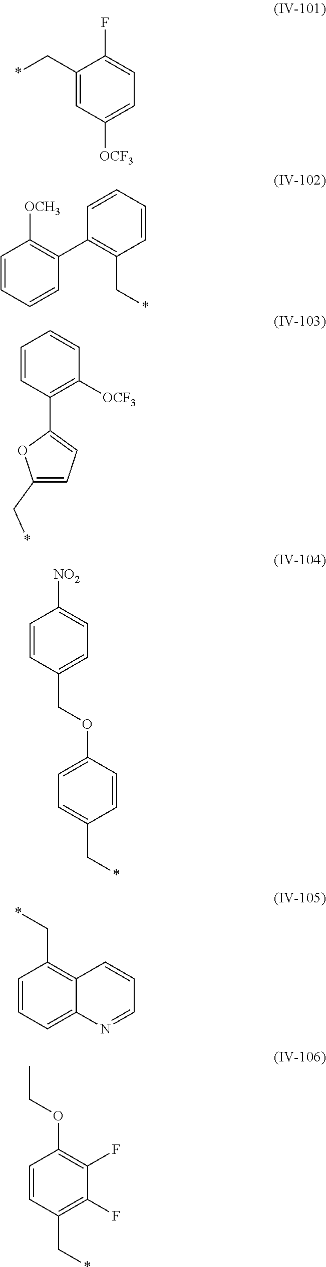 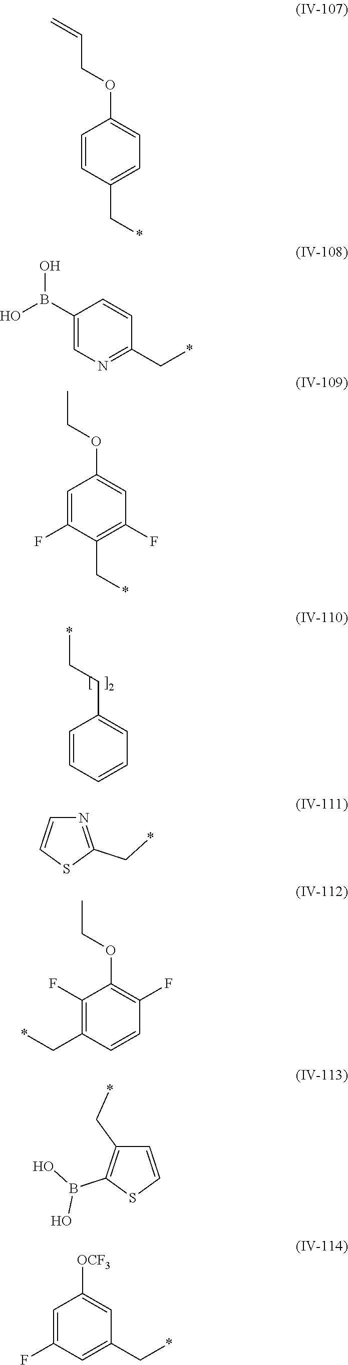 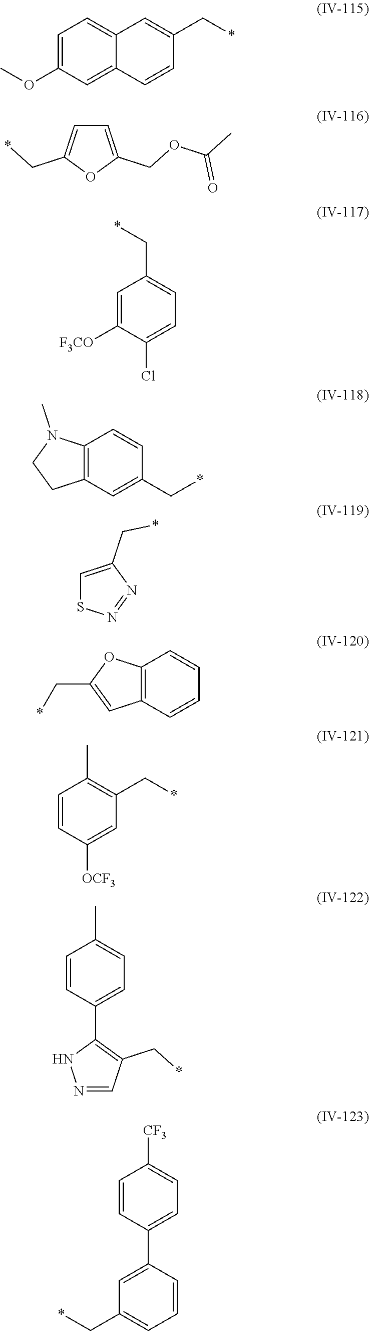 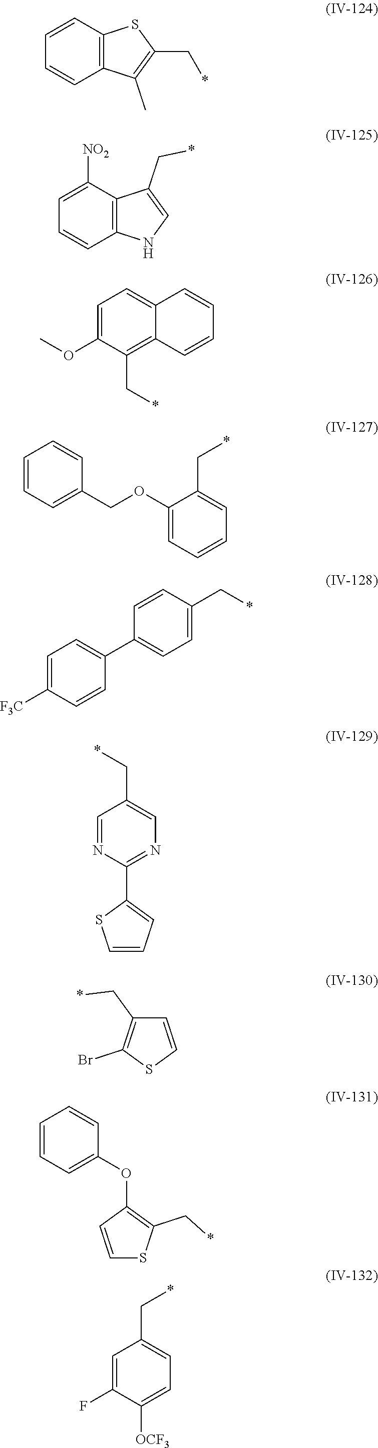 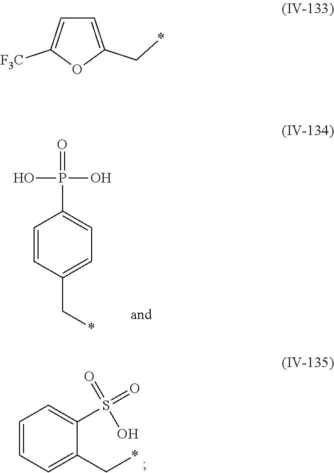
wherein Xa represents CH═O, CHF₂, CF₃, CH₂SH, COOH, CH₂OH, CH₂NO₂, CH₂NH₂, CH₃, C(CH₃)₃, CH(CH₃)₂, CH((CH₂)₃—CH₃)₂, or CH(CH₂—CH₃)₂;
Xb represents O, NH, CH₂, or S;
Xc represents CH or N;
each R₁₀ is independently selected from H, F, Cl, Br, CH₃, CF₃, CH═O, OH, COOH, and (CH₂)ₙCH₃,
m is 1, 2, 3, 4, or 5; and n is 1, 2, 3, 4, or 5;
B₁ and B₂, which may be identical or different, each independently represents an aromatic boron-containing group; and
* in Formulae IV-1 to IV-135 represents a point of attachment to corresponding Formulae FF161-164;
wherein Formulae FF165-FF166 are: 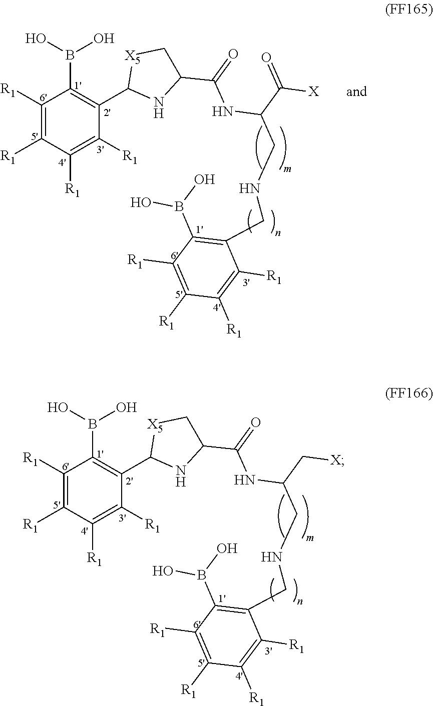
wherein X represents a point of covalent attachment either directly to an amine in X1 or to an amine that is covalently conjugated directly or indirectly to X1, or to OH when X1 is OH;
m is 1, 2, 3, 4, 5, 6, or 7;
n is 1, 2, 3, 4, 5, 6, or 7;
X5 is S, O, or NH; and
each R₁ is independently selected from H, F, Cl, Br, OH, CH₂—NH₂, NH₂, (C═O)—NH₂, CH═O, SO₂CH₃, SO₂CF₃, CF₃, CHF₂, NO₂, CH₃, OCH₃, O(CH₂)ₘCH₃, (SO₂)NH—CH₃, (SO₂)NH(CH₂)ₘCH₃, and OCF₃, wherein m is 1, 2, 3, 4, 5, 6, or 7;
wherein Formulae FF167-FF192 are: 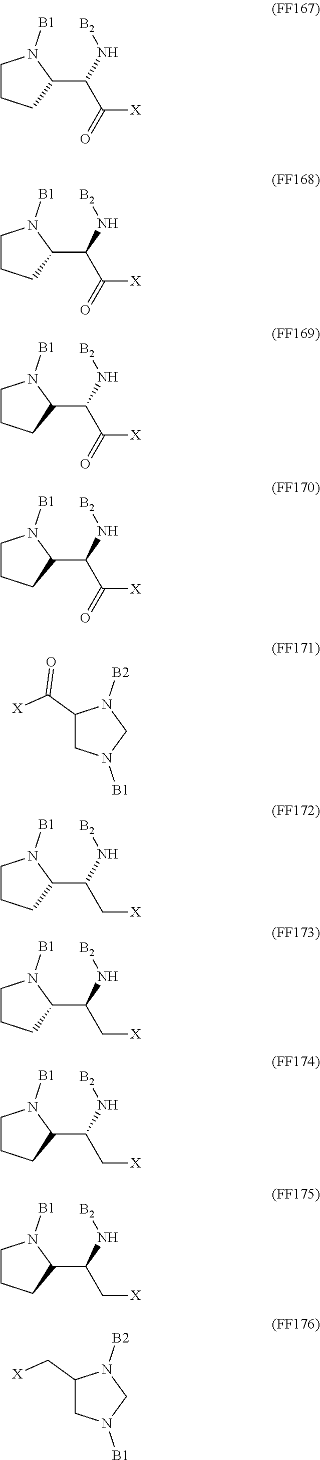 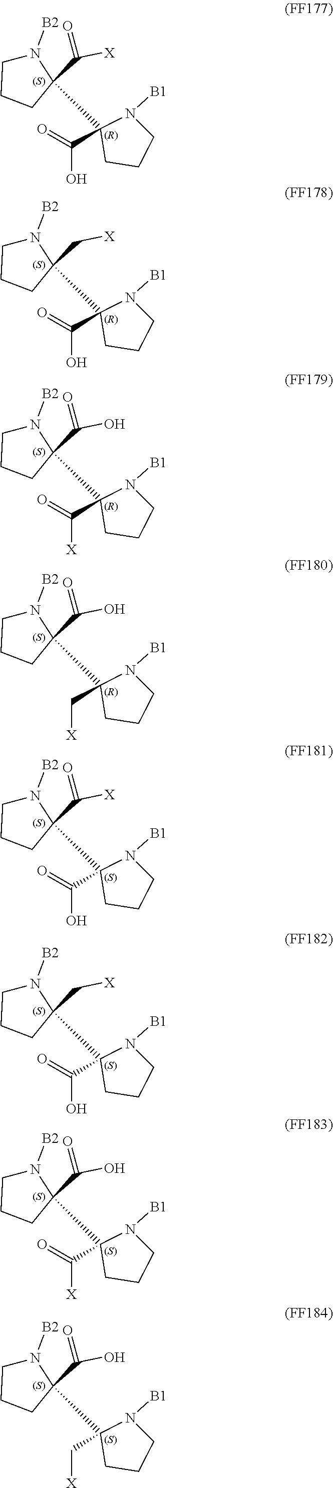 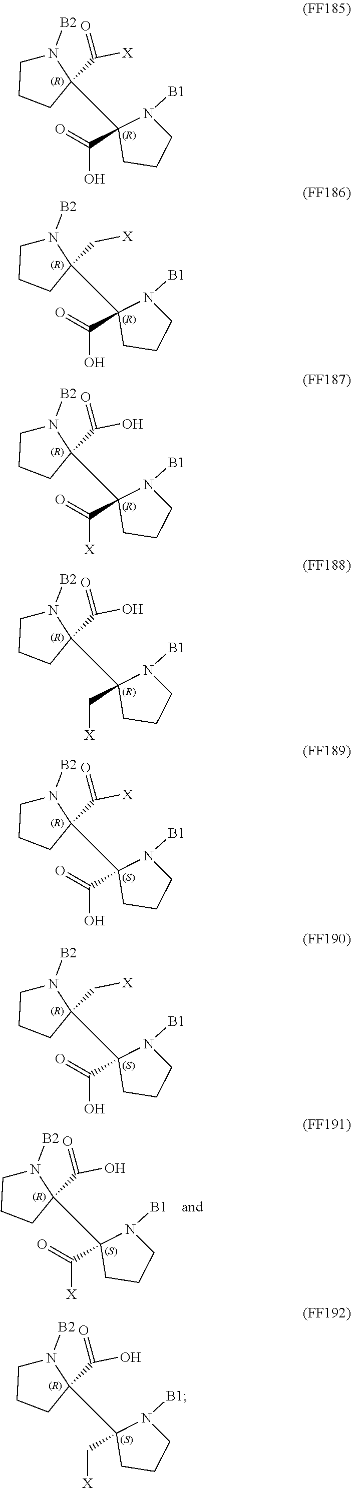
wherein X represents a point of covalent attachment either directly to an amine in X1 or to an amine that is covalently conjugated directly or indirectly to X1, or to OH when X1 is OH;
B₁ and B₂, which may be identical or different, each independently represents an aromatic boron-containing group;
wherein Formulae FF193-FF209 are: 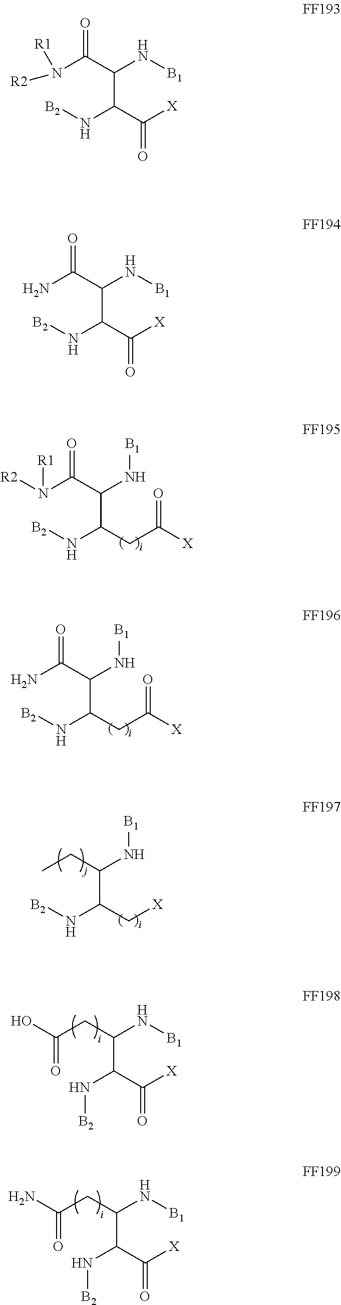 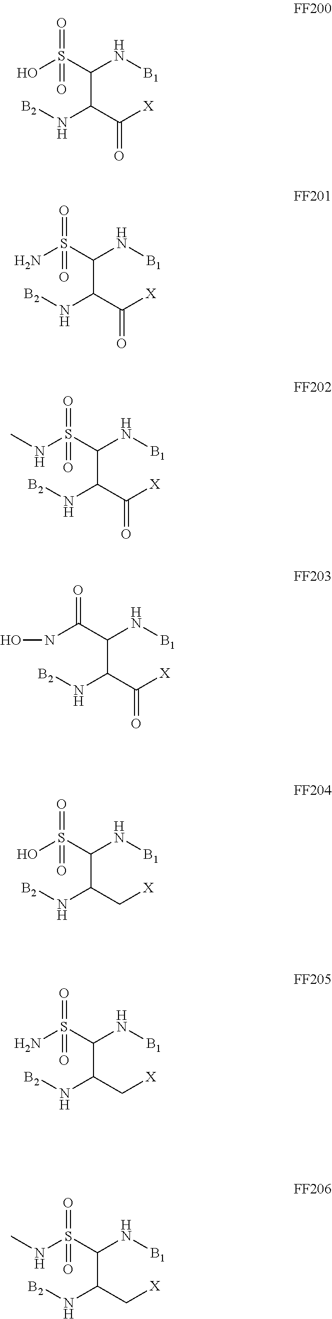 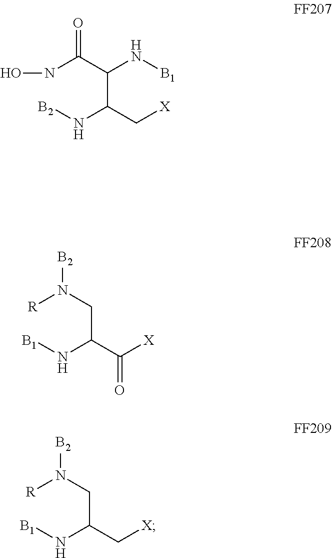
wherein R in FF208 and FF209 is an alkyl, aryl or halide that is covalently conjugated through at least one CH₂ group to the amino group in the side chain of FF208 or FF209,
R1 and R2 are independently selected from H, CH₃, alkyl, and formulae IV-1 to IV-135;
i is 1, 2, 3, 4, or 5;
j is 1, 2, 3, 4, or 5; and
wherein X represents a point of covalent attachment either directly to an amine in X1 or to an amine that is covalently conjugated directly or indirectly to X1, or to OH when X1 is OH; and
B₁ and B₂, which may be identical or different, each independently represents an aromatic boron-containing group, and
wherein Formulae FF210-FF224 are: 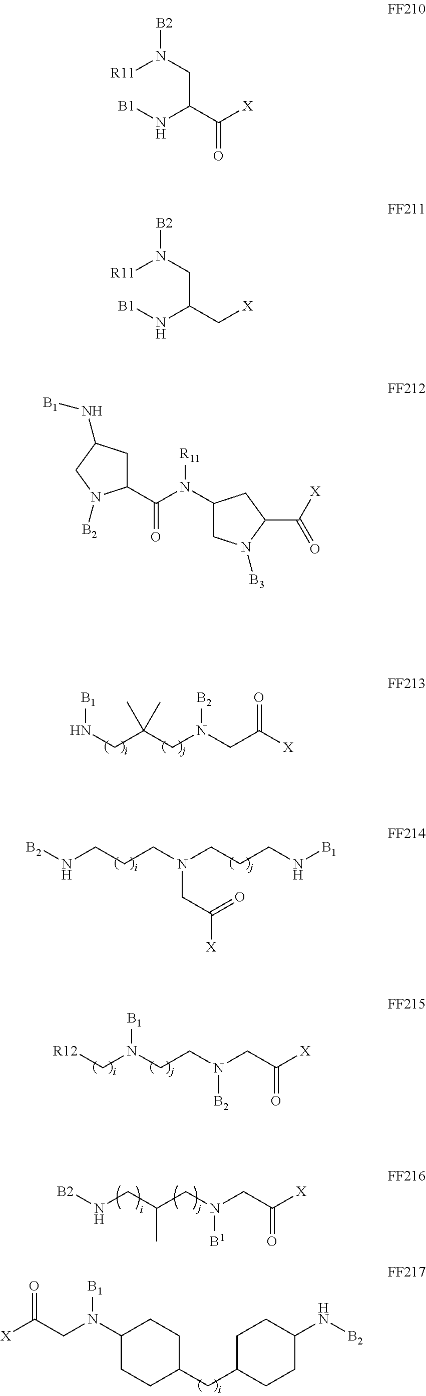 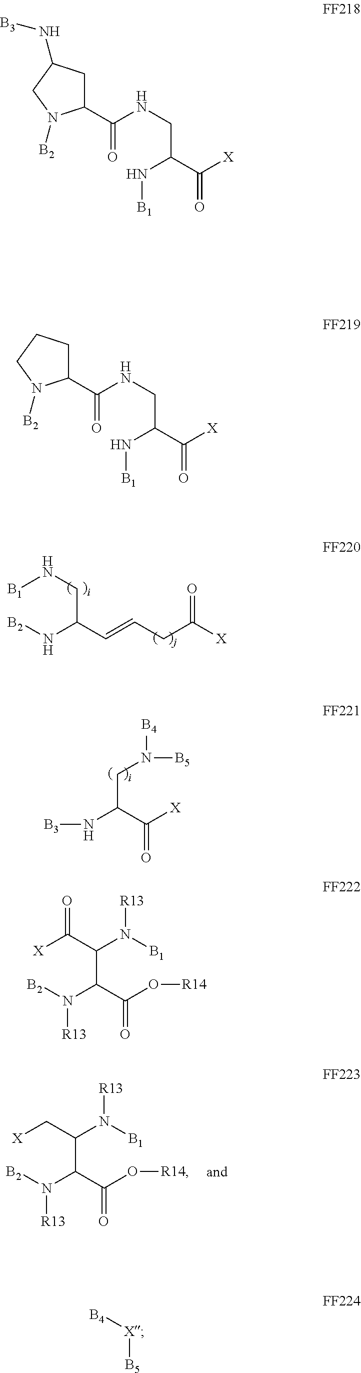
wherein R11 in FF210 to FF212 is selected from Formulae IV-1 to IV-135 and R12 is selected from an amine, a hydroxyl, an alkyl, and a halide group;
wherein each R13 is independently selected from H, CH₃, alkyl, aryl and Formulae IV-1 to IV-135; R14 is selected from H, CH₃, alkyl, aryl and heteroaryl;
wherein X represents a point of covalent attachment either directly to an amine in X1 or to an amine that is covalently conjugated directly or indirectly to X1, or to OH when X1 is OH;
X″ represents a point of covalent attachment to an amine —N in the compound, wherein — represents a single covalent bond to a CH₂ or CH group in the compound;
i is 1, 2, 3, 4, or 5;
j is 1, 2, 3, 4, or 5; and
wherein at least one primary or secondary amine in FF1-FF223 is optionally covalently conjugated to B₆; and
B₁, B₂, B₃, B₄, B₅, and B₆ each independently represents an aromatic boron-containing group, wherein in each FF structure containing B₁, B₂ and B₃ groups, at least two of the B₁, B₂ and B₃ groups are independently an aromatic boron-containing group.

2. The compound of claim 1, wherein the compound is a molecular conjugate represented by Formula I, or a stereoisomer or a mixture of stereoisomers, or pharmaceutically acceptable salt thereof: 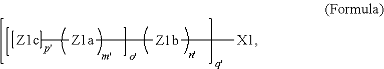
wherein
X1 comprises:
(i) NH₂ or OH,
(ii) a polypeptide drug substance comprising an amine,
(iii) a polypeptide drug substance that is covalently conjugated to an amine containing linker, or
(iv) an amine configured to be covalently conjugated to a polypeptide drug substance;
each Z1c is independently selected from Formulae FF1-FF224 and covalently conjugated either directly, or via Z1a and/or Z1b, to X1;
each Z1a independently comprises 1 to 50 amino acids connected together using amide or peptide bonds;
each Z1b is independently a small-molecule linker;
each m′ is independently 0 or 1;
each n′ is independently 0 or a positive integer;
each o′ is independently an integer greater than or equal to 1;
each p′ is a positive integer; and
q′ is a positive integer of at least 1 and not more than two times the total number of amine groups in X1, wherein when any of n′, o′, p′, or q′ is 2 or more, the corresponding groups Z1a, Z1b, and Z1c are independently selected and may be the same or different;
wherein each Z1c is independently covalently conjugated, directly or indirectly, to an amine of Z1a, to an amine of Z1b, or to X1; and
wherein optionally the molecular conjugate may comprise one or more isotopes at any position of the molecular conjugate of Formula I.

3. The compound of claim 1, wherein the compound comprises at least one of B₁, B₂ and B₃ independently selected from Formulae F1-F12 or wherein the compound comprises at least one of B₄, B₅ and B₆ independently selected from Formulae F1-F10,
wherein Formulae F1-F10 are: 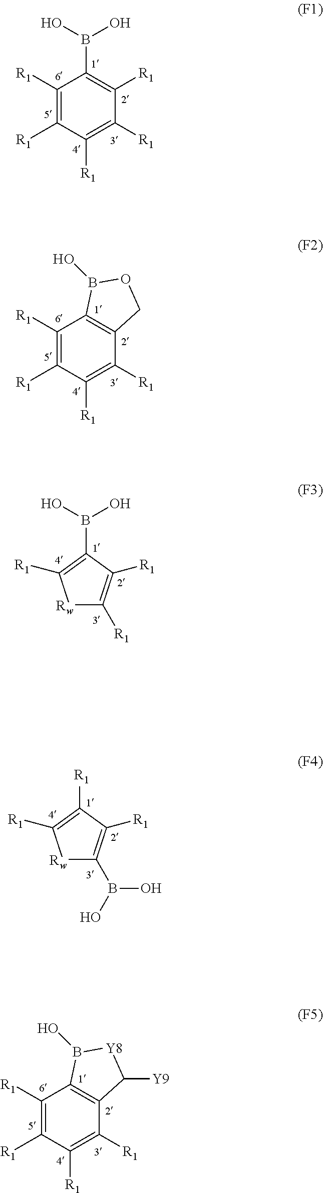 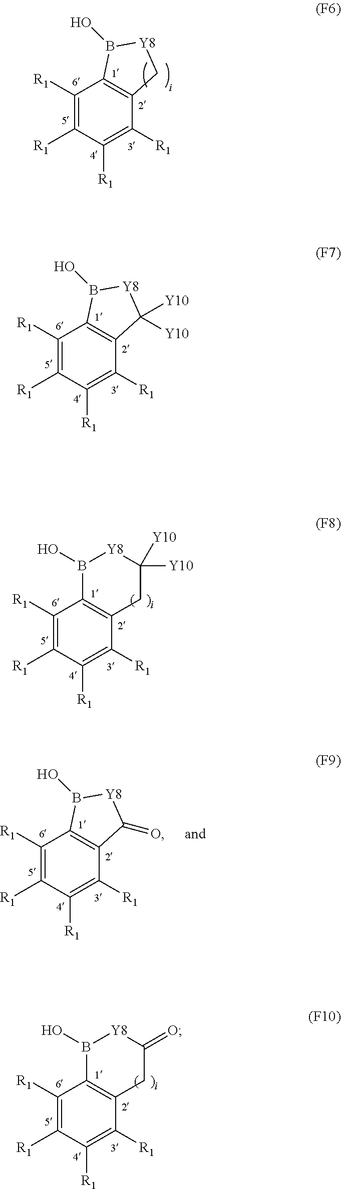
wherein for B₁, B₂, and B₃:
one R₁ represents (C═O)—*, S(═O)(═O)—*, (CH₂)ₘ(C═O)—*, or (CH₂)ₘ—*, wherein —* represents the attachment point to the rest of Z1c, and m is 1, 2, 3, 4, 5, 6, or 7; and
each remaining R₁ is independently selected from H, F, Cl, Br, OH, CH₂—NH₂, NH₂, (C═O)—NH₂, CH═O, SO₂CH₃, SO₂CF₃, CF₃, CHF₂, NO₂, CH₃, OCH₃, O(CH₂)ₘCH₃, (SO₂)NH—CH₃, (SO₂)NH(CH₂)ₘCH₃, and OCF₃, wherein m is 1, 2, 3,4, 5, 6, or 7;
wherein for B₄ and B₅:
one R₁ for B₄ represents (CH₂)ₘ—Ø, wherein —Ø represents an attachment point to the rest of Z1c and one R₁ for B₅ represents (C═O)—*, S(═O)(═O)—*, (CH₂)ₘ(C═O)—*, or (CH₂)ₘ—*, wherein —* represents an attachment point to the rest of Z1c, and m is 1, 2, 3, 4, 5, 6, or 7; and
each remaining R₁ is independently selected from H, F, Cl, Br, OH, CH₂—NH₂, NH₂, (C═O)—NH₂, CH═O, SO₂CH₃, SO₂CF₃, CF₃, CHF₂, NO₂, CH₃, OCH₃, O(CH₂)ₘCH₃, (SO₂)NH—CH₃, —(SO₂)NH(CH₂)ₘCH₃, and OCF₃, wherein m is 1, 2, 3,4, 5, 6, or 7;
wherein for B₆:
one R₁ for B₆ represents (CH₂)ₘ—Ø, wherein —Ø represents an attachment point to the rest of the compound, and m is 1, 2, 3, 4, 5, 6, or 7; and
each remaining R₁ is independently selected from H, F, Cl, Br, OH, CH₂—NH₂, NH₂, (C═O)—NH₂, CH═O, SO₂CH₃, SO₂CF₃, CF₃, CHF₂, NO₂, CH₃, OCH₃, O(CH₂)ₘCH₃, (SO₂)NH—CH₃, (SO₂)NH(CH₂)ₘCH₃, and OCF₃, wherein m is 1, 2, 3,4, 5, 6, or 7;
wherein,
for Formulae F3-F4:
R_{w} is O or S;
for Formulae F5-F10:
Y8 is selected from O, N, and NR, wherein R is an alkyl group or H;
Y9 is H, CH₃, or an alkyl group, provided that when Y8 is O, Y9 is a CH₃ or an alkyl group;
each Y10 is independently selected from H, CH₃, F, CF₃, and OCH₃; and
i is 1, 2, or 3; and
wherein Formulae F11-F12 are: 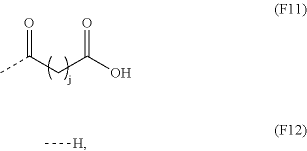
j is 1, 2, 3, 4, 5, 6, or 7; and
— represents an attachment point to the rest of Z1c.

4. The compound of claim 2, wherein the compound comprises at least one Z1b selected from Formulae IIa-IIai and Formulae IIIa-IIIai, wherein Formulae IIa-IIai are: 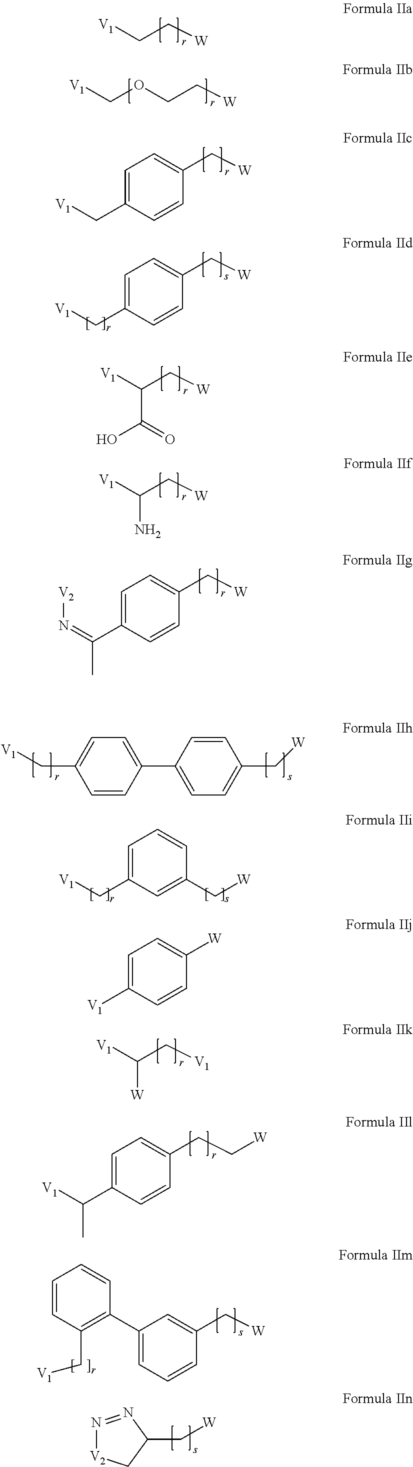 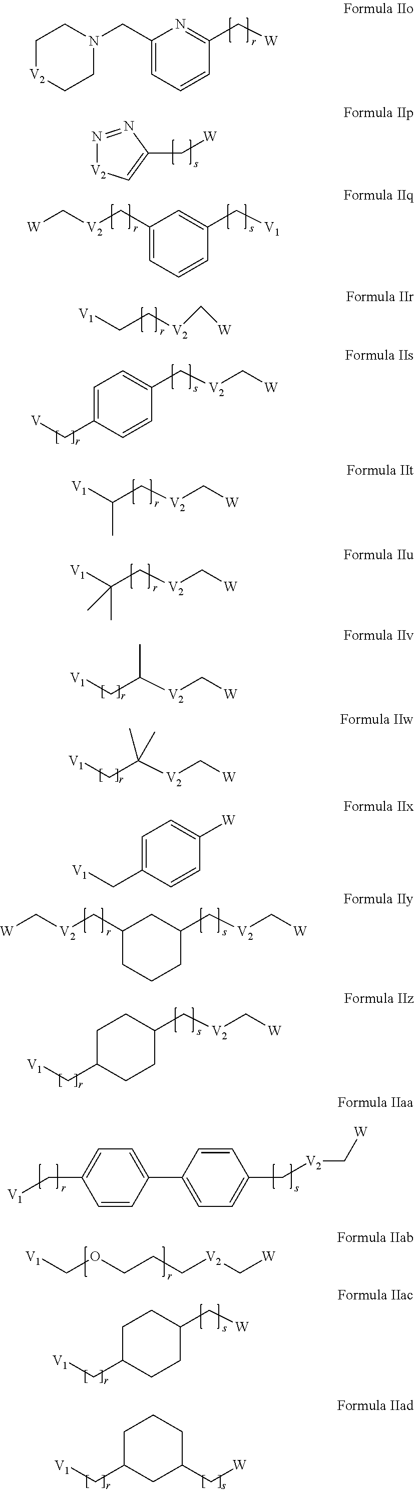 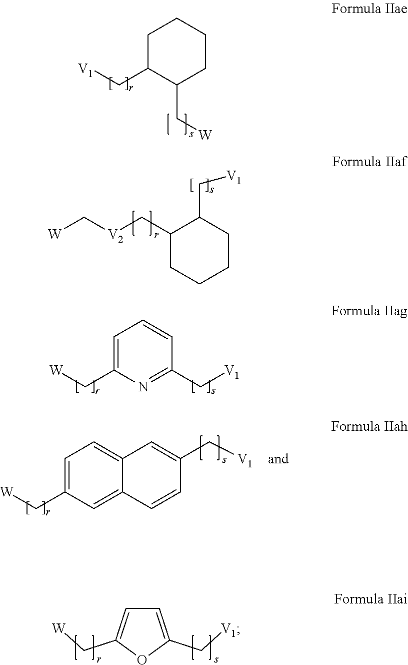
wherein:
r is, 1, 2, 3, 4, or 5;
s is 0, 1, 2, 3, 4, or 5;
W represents CH₂—˜ or (C═O)—˜, wherein —˜ is a covalent linkage to X1; and
each V₁ is independently selected from NH—†, CH₂—†, and (C═O)—† and each V₂ is N—†, wherein —† is a covalent linkage towards successive Z1b, Z1a or Z1c, provided that V₁ is NH—† when connected to Z1c; and the covalent linkages between Z1a and Z1b units each independently comprise an amine linkage or an amide linkage; and when n′=0 and m′=1, Z1a is directly conjugated to X1 by an amine linkage or amide linkage, and
wherein Formulae IIIa-IIIai are: 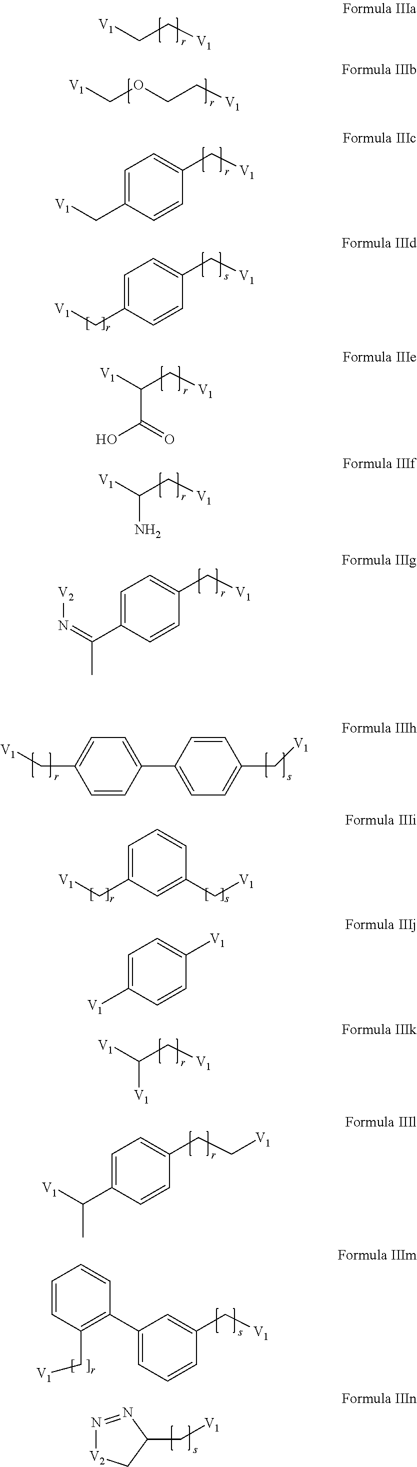 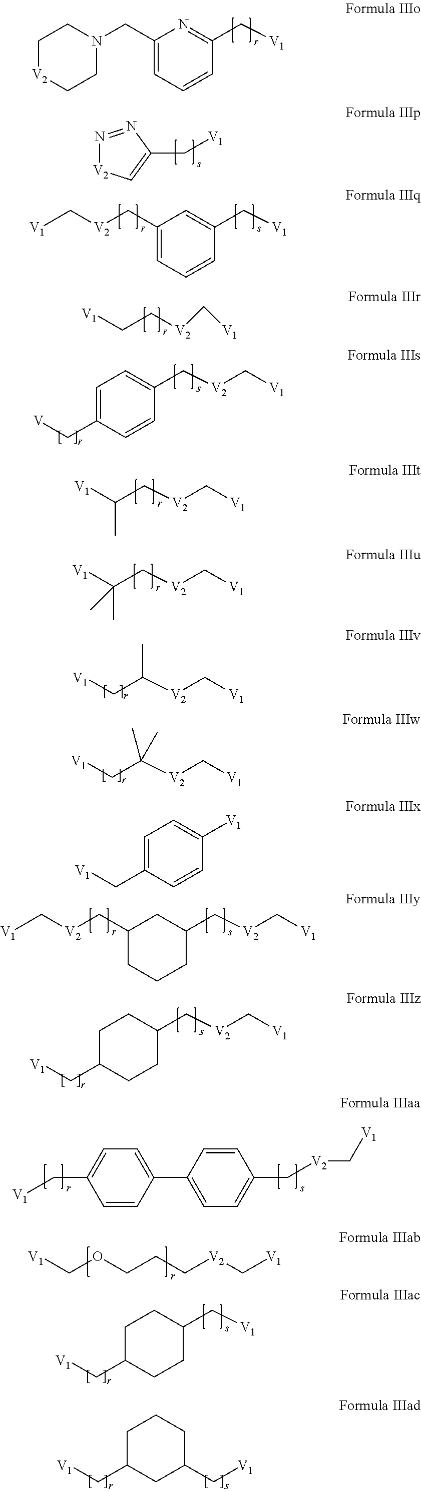 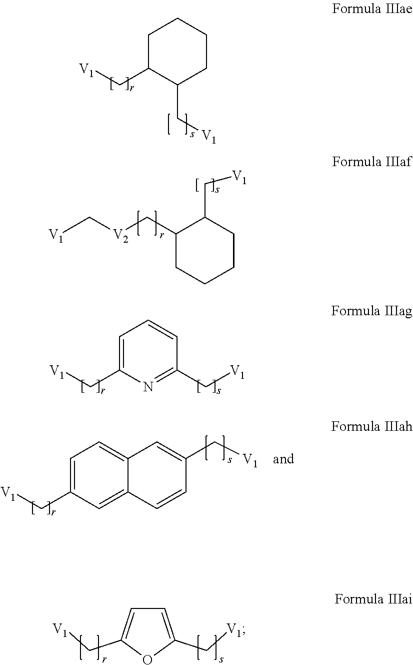
wherein:
r is 1, 2, 3, 4, or 5;
s is 1, 2, 3, 4, or 5; and
each V₁ is independently selected from NH—†, CH₂—†, and (C═O)—† and each V₂ is N—†, wherein —† is a covalent linkage towards successive Z1b, Z1a or Z1c, provided that V₁ is NH—† when connected to Z1c; and the covalent linkages between Z1a and Z1b units each independently comprise an amine linkage or an amide linkage; and when n′=0 and m′=1, Z1a is directly conjugated to X1 by an amine linkage or amide linkage.

5. The compound of claim 1, wherein the at least one Z1c is covalently conjugated indirectly via a linker selected from (i) Formulae FL1-FL19: 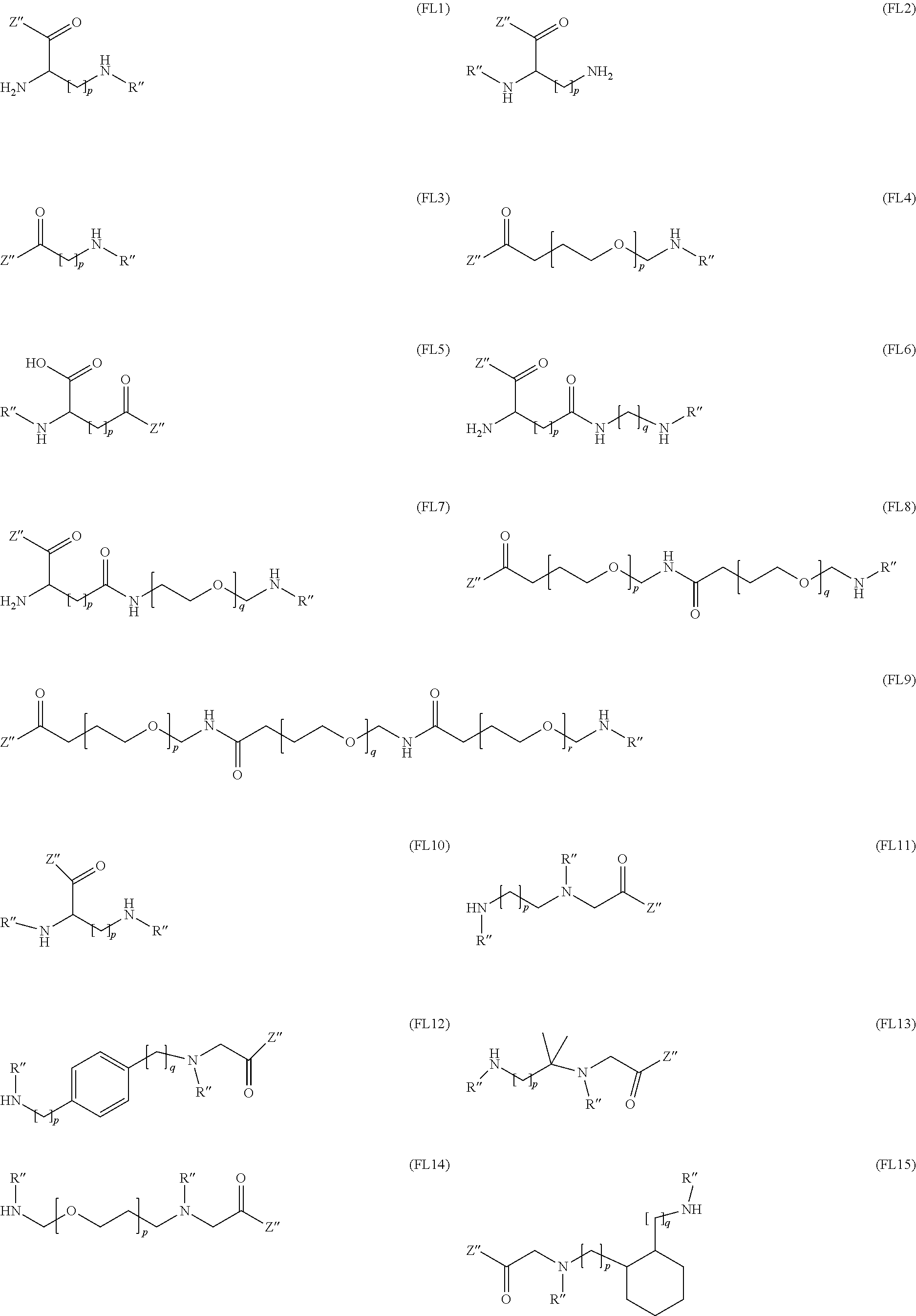 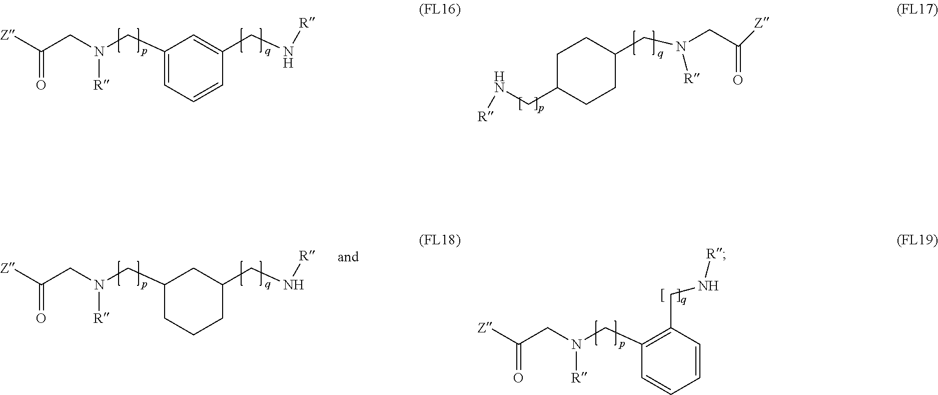
wherein, in Formulae FL1 to FL19:
Z″ represents an attachment point toward X1;
R″ represents an attachment point toward Z1c;
p is 1, 2, 3, 4, or 5,
q is 1, 2, 3, 4, or 5,
r is 1, 2, 3, 4, or 5; and
any primary amine is optionally acetylated or alkylated; and
(ii) an L- or D-amino acid comprising at least one amine group directly conjugated to Z1c, wherein an acid functional group of the amino acid is conjugated toward X1 in Formula I.

6. The compound of claim 2, wherein n′ is 1 and each of the Z1b is independently selected from (i) Formulae FL1-FL19: 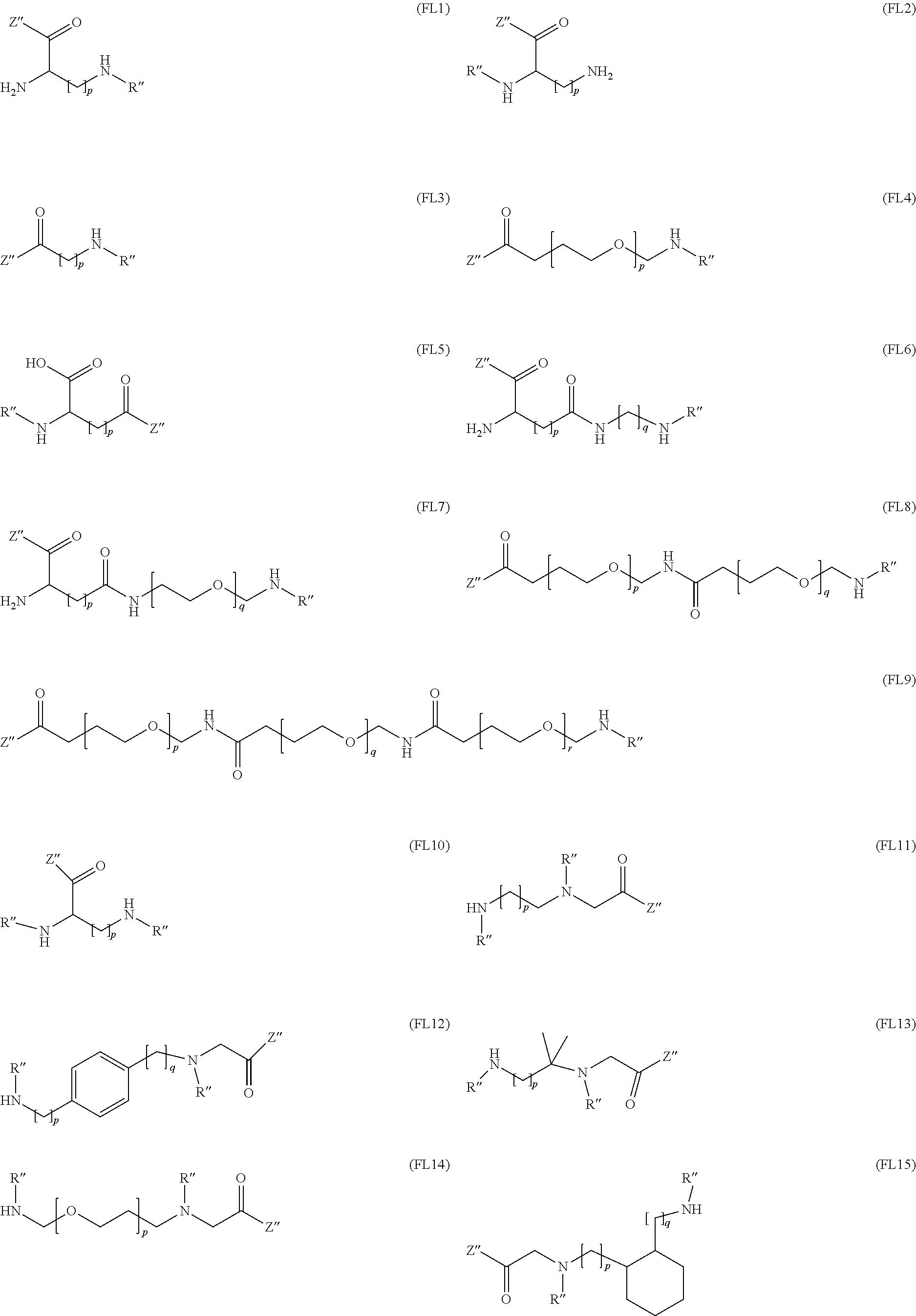 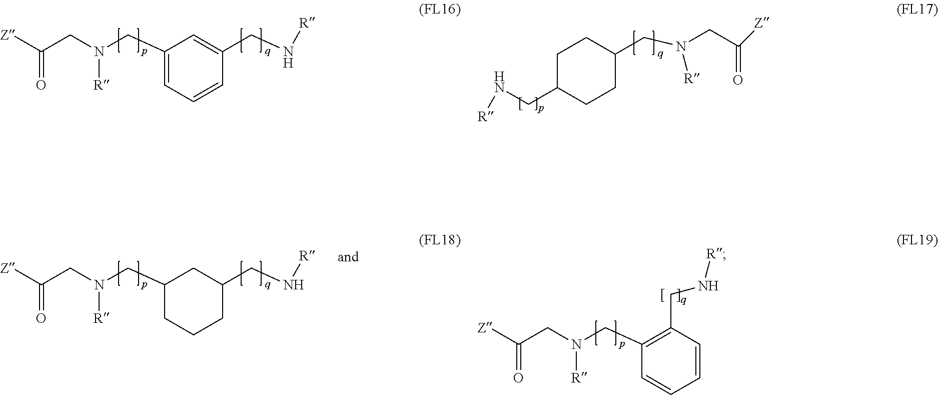
wherein, in Formulae FL1 to FL19:
Z″ represents an attachment point toward X1;
R″ represents an attachment point toward Z1c;
p is 1, 2, 3, 4, or 5,
q is 1, 2, 3, 4, or 5,
r is 1, 2, 3, 4, or 5; and
any primary amine is optionally acetylated or alkylated; and
(ii) an L- or D-amino acid comprising at least one amine group directly conjugated to Z1c, wherein an acid functional group of the amino acid is conjugated toward X1 in Formula I.

7. The compound of claim 1, wherein the compound comprises a drug substance comprising a human polypeptide hormone of the human pancreas, insulin, glucagon, GLP-1, a somatostatin, a gastric inhibitory polypeptide, a glucose-dependent insulinotropic polypeptide, a hybrid peptide comprising sequences from two or more human polypeptide hormones, or an analogue thereof.

8. The compound of claim 5, wherein:
X1 comprises human insulin or a human insulin analogue comprising an A-chain and a B-chain, wherein the A-chain comprises a sequence selected from SEQ ID NOs 1 and 3 to 33, and the B-chain comprises a sequence selected from SEQ ID NOs 2 and 34 to 74, 24047, and 24048;
each Z1c is independently selected from FF1, FF10, FF12, FF14, FF15, FF114, FF115, FF116, FF163, FF193, FF194, FF203, and FF221-FF224 and covalently conjugated either directly, or indirectly via the linker, to Z1a and/or Z1b, or to X1;
each Z1a is independently absent or independently comprises a sequence selected from K, GK, KGSH (SEQ ID NO:24049), KGSHK (SEQ ID NO:4238), KNSTK (SEQ ID NO:5085), GKASHK (SEQ ID NO:12414), GKEEEK (SEQ ID NO:12677), GKEEHK (SEQ ID NO:12680), GKGHSK (SEQ ID NO:13120), GKGSH (SEQ ID NO:24050), GKGSHK (SEQ ID NO:13198), GKGSTK (SEQ ID NO:13205), GKHENK (SEQ ID NO:13271), GKNSHK (SEQ ID NO:13982), GKNSTK (SEQ ID NO:13989), GKQSSK (SEQ ID NO:14380), GKYQFK (SEQ ID NO:15128), GKGSKK (SEQ ID NO:24045), GKKPGKK (SEQ ID NO:24046), GKGPSK (SEQ ID NO:24044), GKPSHKP (SEQ ID NO:24043), and GSHKGSHK (SEQ ID NO:24042);
each said linker is selected from FL1, FL3, FL4, and FL5;
each m′ is independently 0 or 1;
each n′ is independently 0, 1, 2, or 3;
each o′ is independently 1, 2, 3, 4, or 5;
each p′ is 1, 2, 3, 4, or 5; and
q′ is 1, 2, 3, or 4, wherein when any of n′, o′, p′, or q′ is 2 or more, the corresponding groups Z1a, Z1b, and Z1c are independently selected and may be the same or different; and
wherein each Z1c is independently covalently conjugated, directly or indirectly, to an amine of Z1a, to an amine of Z1b, or to X1.

9. (canceled)

10. The compound of claim 2, wherein each of the Z1a is independently absent or independently comprises a sequence selected from K, GK, KGSH (SEQ ID NO:24049), GKGSH (SEQ ID NO:24050), KGSHK (SEQ ID NO:4238), and GKGSHK (SEQ ID NO:13198).

11. The compound of claim 2, wherein each of the Z1c is independently selected from FF1, FF10, FF12, FF14, FF15, FF114, FF115, FF116, and FF221-FF224, and wherein the B₁ and the B₂ are independently selected from Formulae F1 and F2.

12. (canceled)

13. (canceled)

14. (canceled)

15. The compound of claim 1, wherein each of the Z1c is independently selected from FF10, FF12, FF116, FF221, FF222, and FF224.

16. The compound of claim 2, wherein:
each B₁ and B₂ is F2 and is covalently conjugated to Z1c using an amide linkage,
each Z1b is independently absent; FL3 wherein p is 1, 2, or 3; or FL5 wherein p is 2, 3, or 4;
each FF is independently selected from FF10, FF12, FF116, FF134, FF163, FF193, FF203, FF221, FF222 and FF224; wherein each FF12 and FF222 has either (S,R) or (S,S) stereochemistry;
each Z1c is conjugated either directly or indirectly through FL3 or FL5 to the amine group in one or more lysine side chain in X1 or the N-terminus in X1; and
X1 is a polypeptide drug substance and/or an insulin optionally having from 0 to 4 residues replaced, inserted, or mutated to lysines, and wherein the lysines are each conjugated directly or indirectly to a Z1c.

17. (canceled)

18. The compound of claim 2, wherein the compound is selected from:

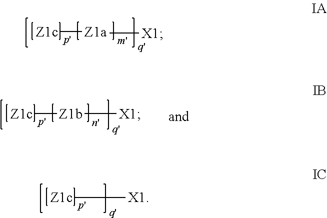

19. (canceled)

20. (canceled)

21. The compound of claim 1, wherein Z1c is covalently conjugated directly to X1 via a linker, and wherein the linker is independently selected from gamma-glutamic acid, beta-alanine, and 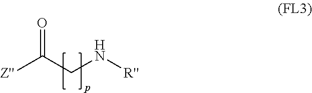
Formula FL3,
wherein p is 1, 2, or 3; and 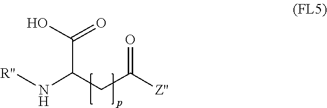
Formula FL5,
wherein p is 2, 3, or 4.

22. (canceled)

23. The compound of claim 1, wherein the compound is selected from:

[symbol]

24.-49. (canceled)

50. A compound selected from Formulae FF1-FF224:
wherein Formulae FF1-FF48 are: 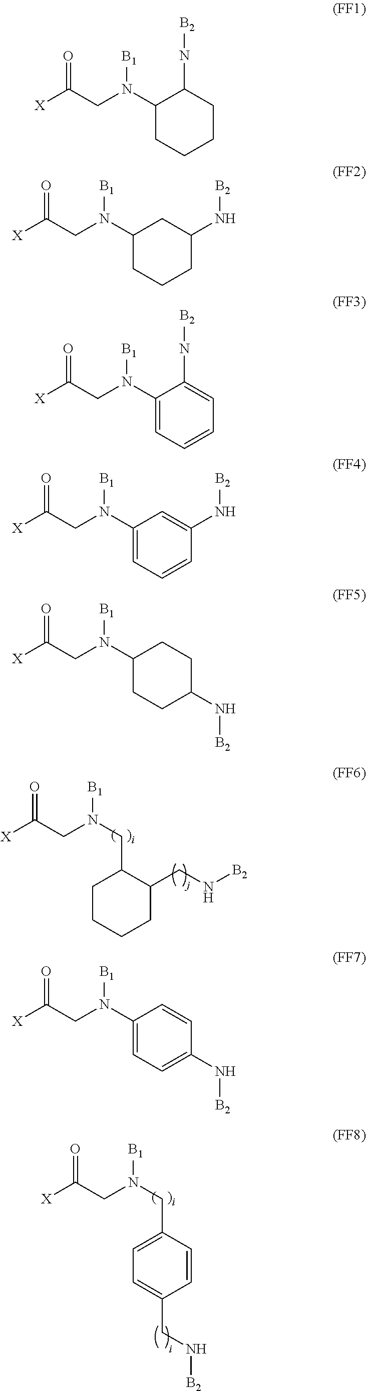 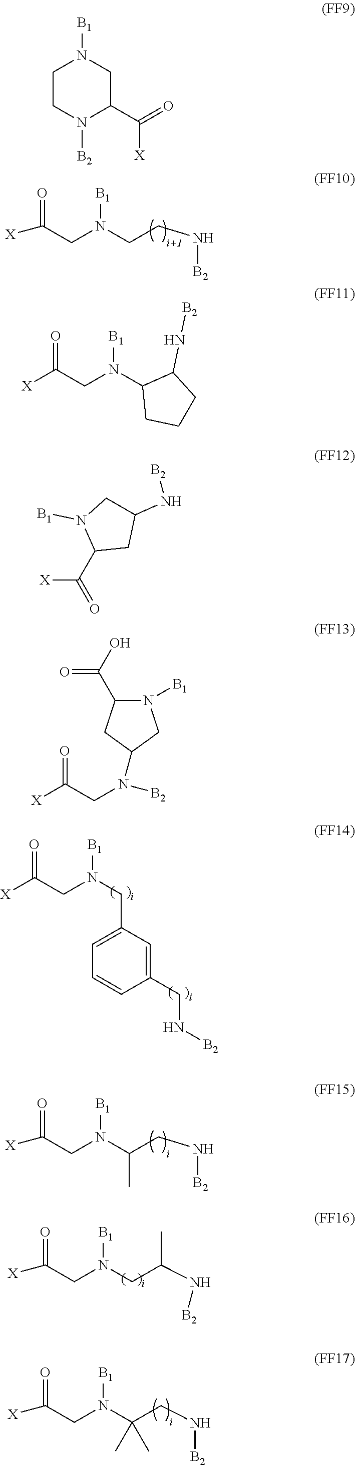 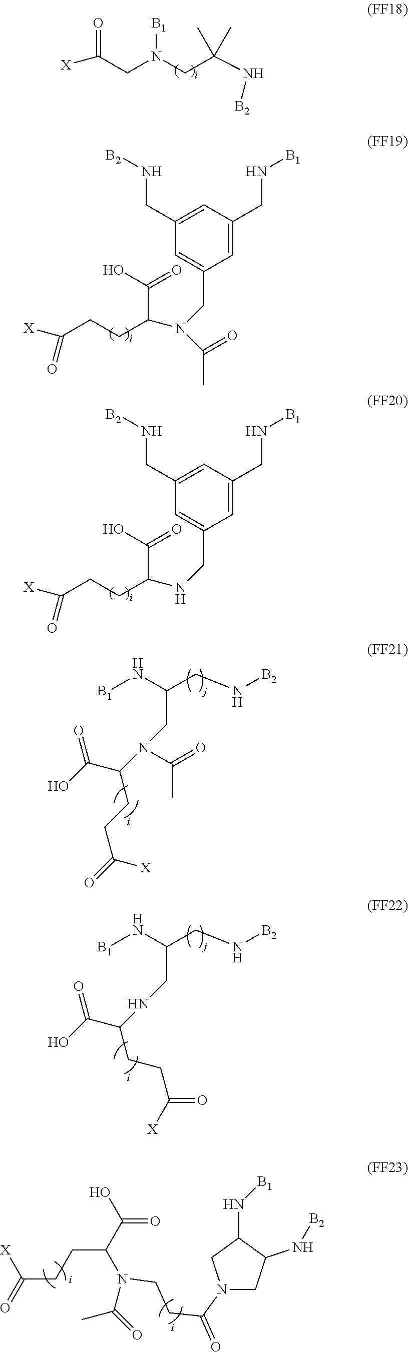 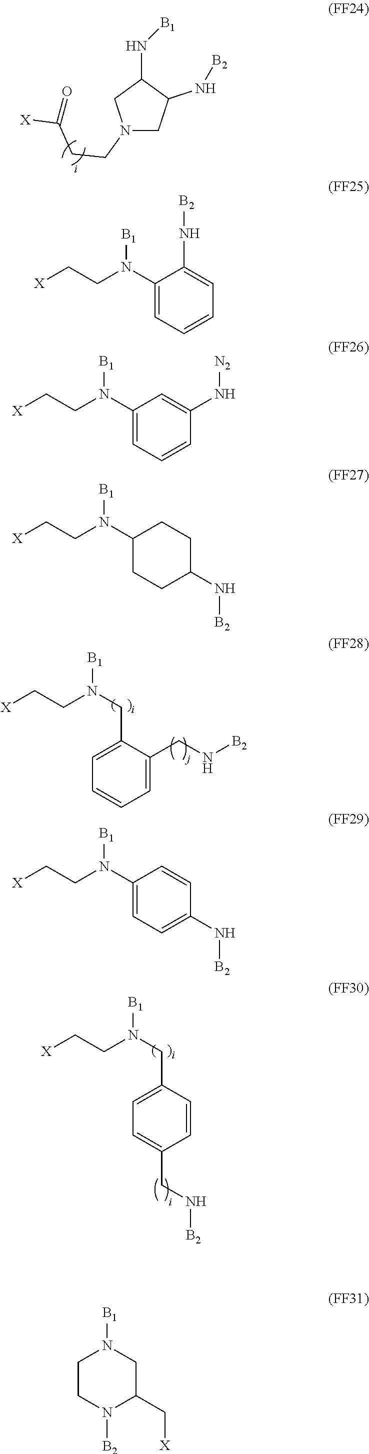 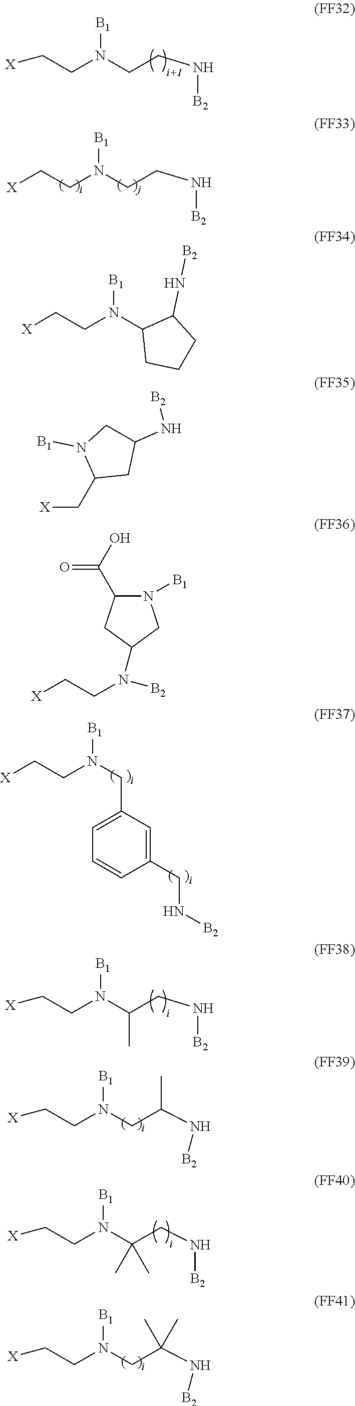 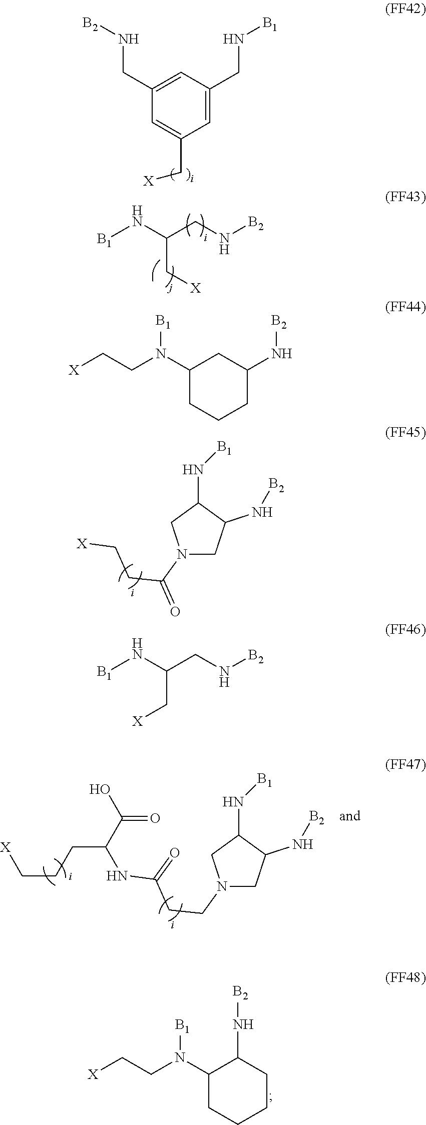
wherein X is selected from an amine, OH, and halogen; and
i is 1, 2, 3, 4, 5, 6, or 7;
j is 1, 2, 3, 4, 5, 6, or 7; and
B₁ and B₂, which may be identical or different, each independently represents an aromatic boron-containing group; and
wherein Formulae FF49-FF88 are: 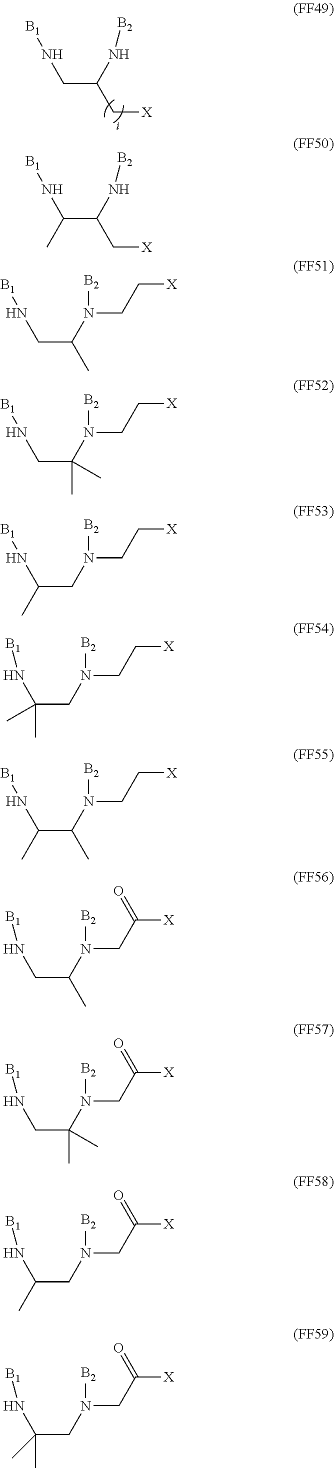 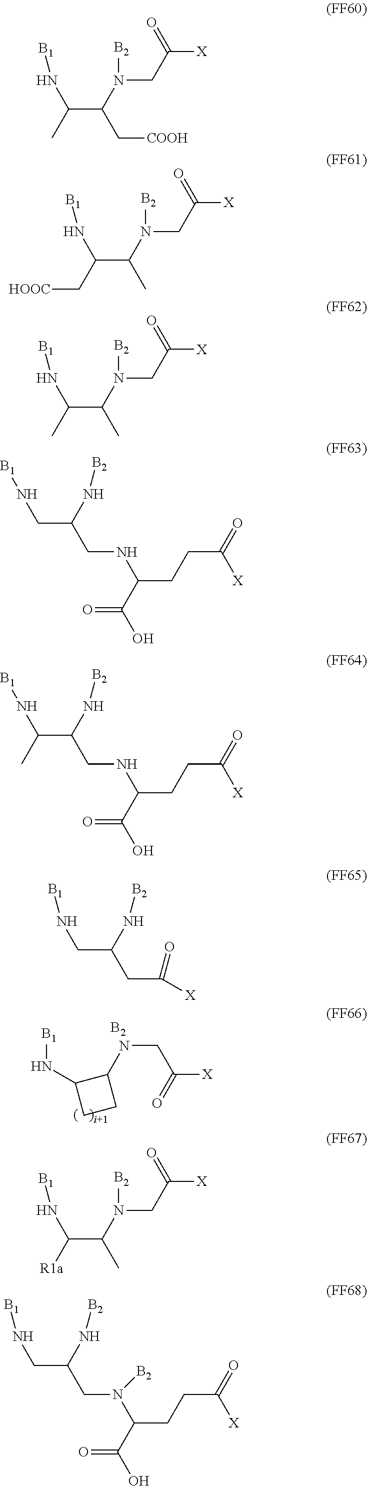 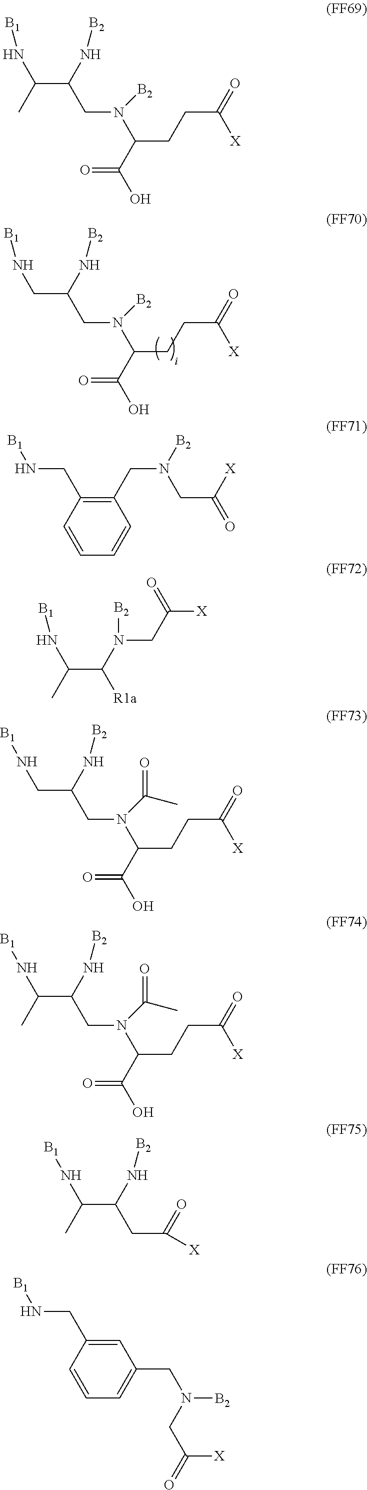 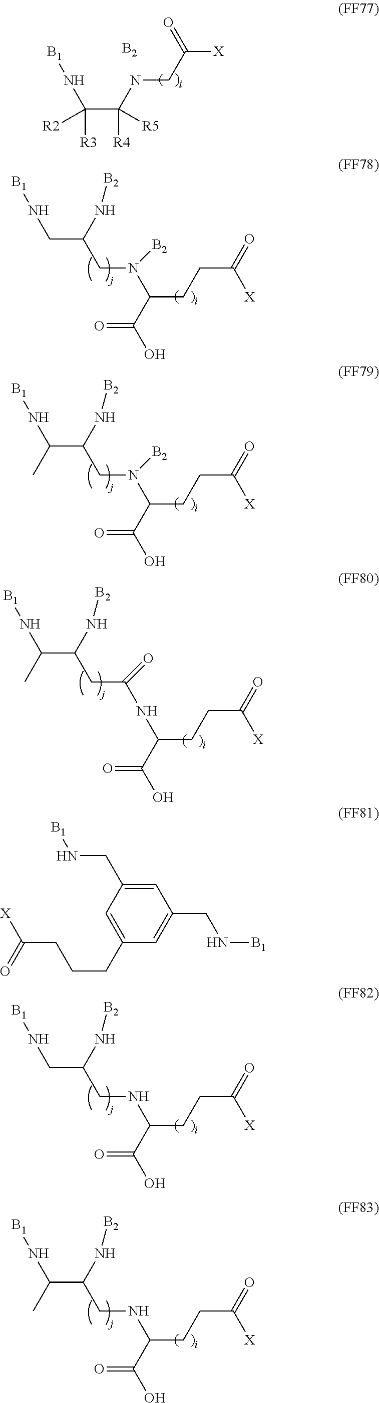 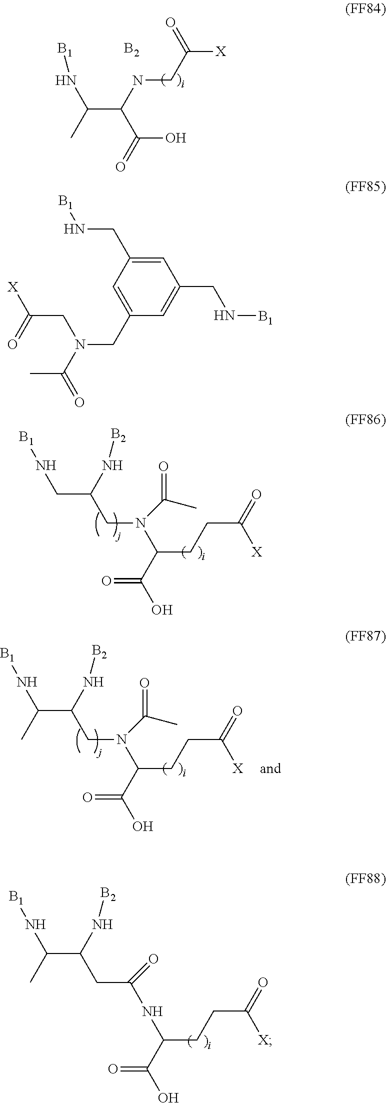
wherein X is selected from an amine, OH, and halogen;
i is 1, 2, 3, 4, 5, 6, or 7;
j is 1, 2, 3, 4, 5, 6, or 7;
R1a is selected from COOH, CH₃, H, and OH;
R2, R3, R4 and R5 is each independently selected from CH₃, H, OH, and COOH, and at least one of R2, R3, R4 and R5 is CH₃ or OH; and
B₁ and B₂, which may be identical or different, are each independently an aromatic boron-containing group; and
wherein Formulae FF89-FF112 are: 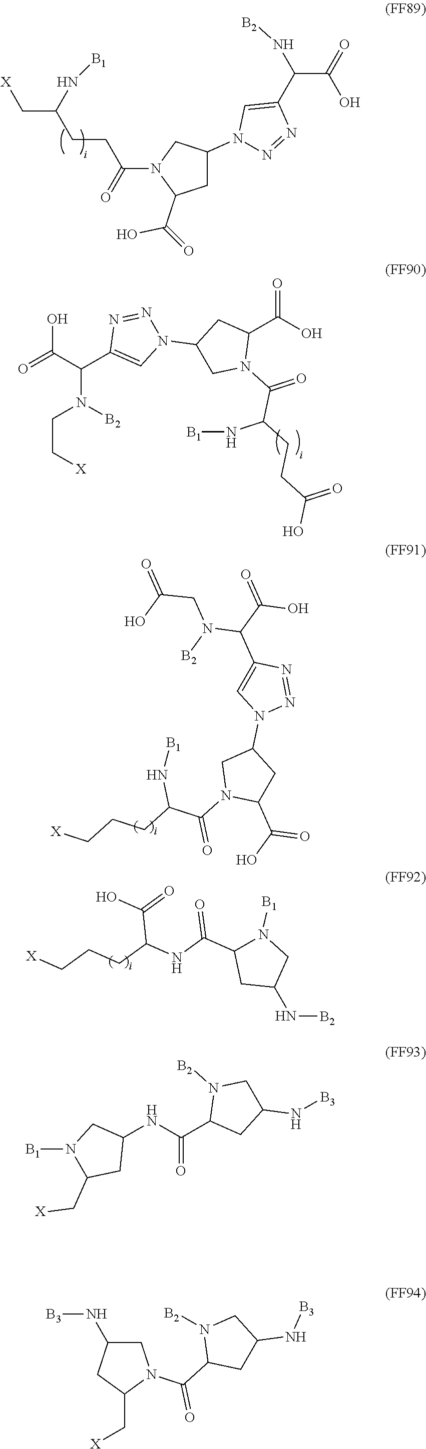 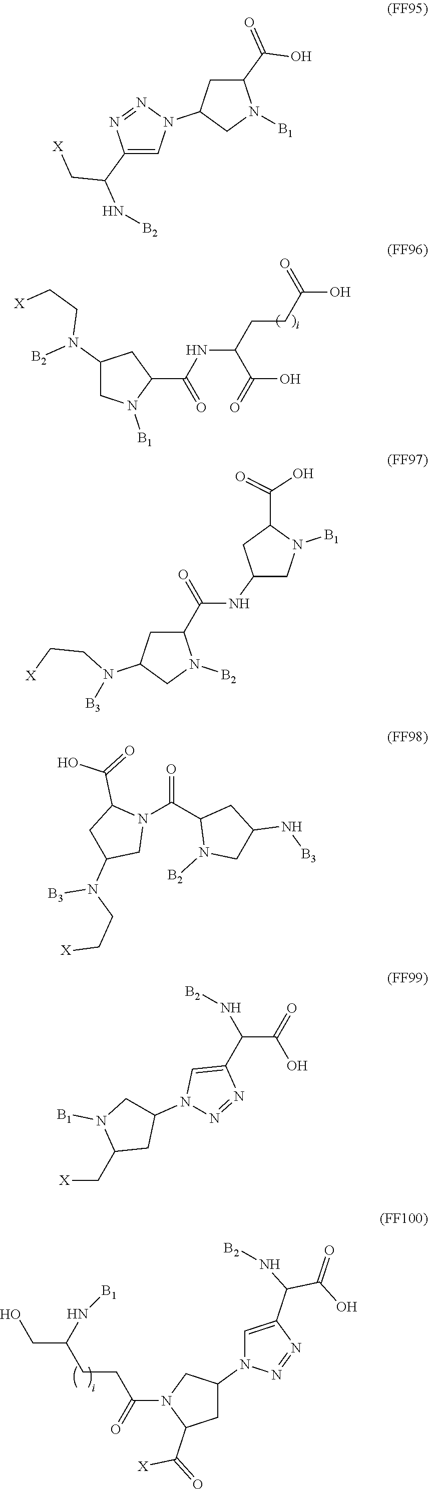 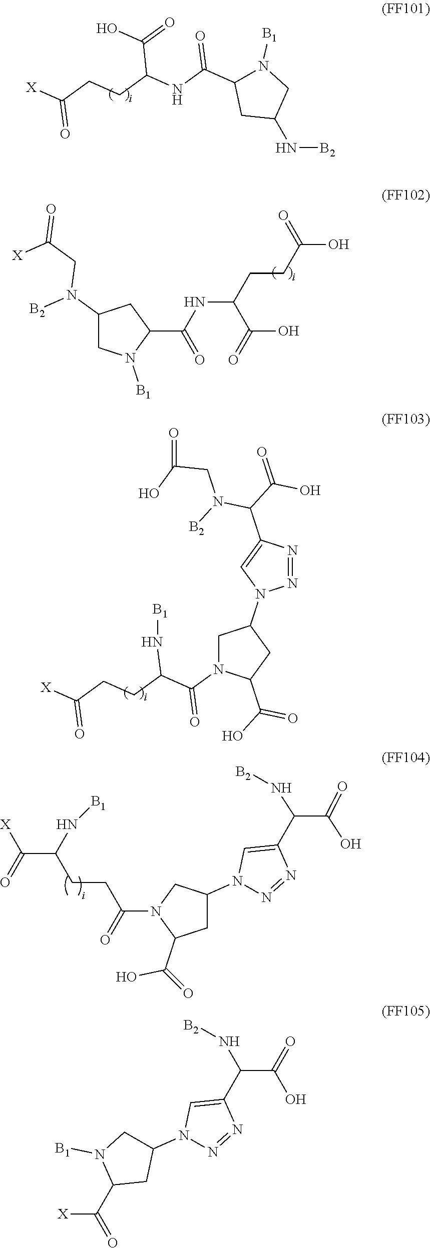 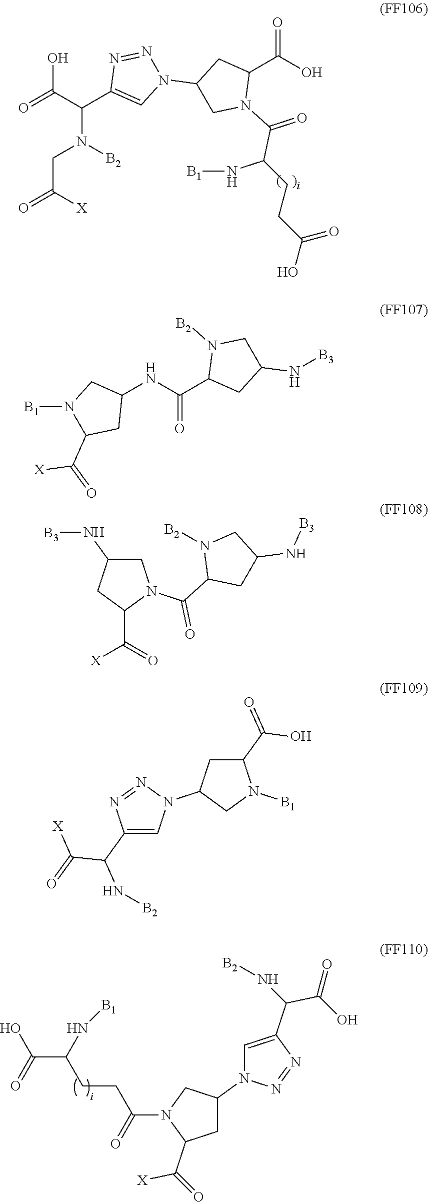 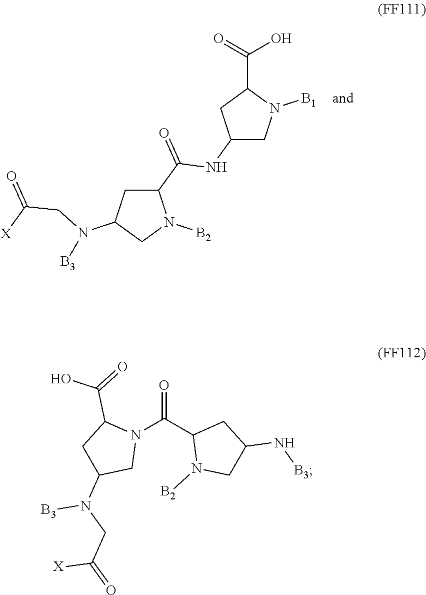
wherein X is selected from an amine, OH, and halogen;
i is 1, 2, 3, 4, 5, 6, or 7; and
B₁, B₂ and B₃, which may be identical or different, each independently represents an aromatic boron-containing group, a carboxylic acid derivative, or a H, wherein at least two of B₁, B₂ and B₃ in each FF structure are independently an aromatic boron-containing group; and
wherein Formulae FF113-FF136 are: 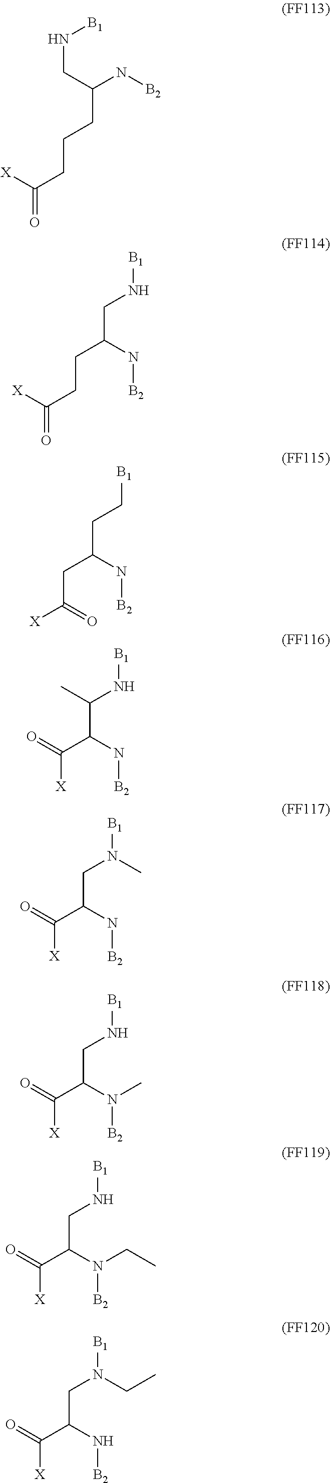 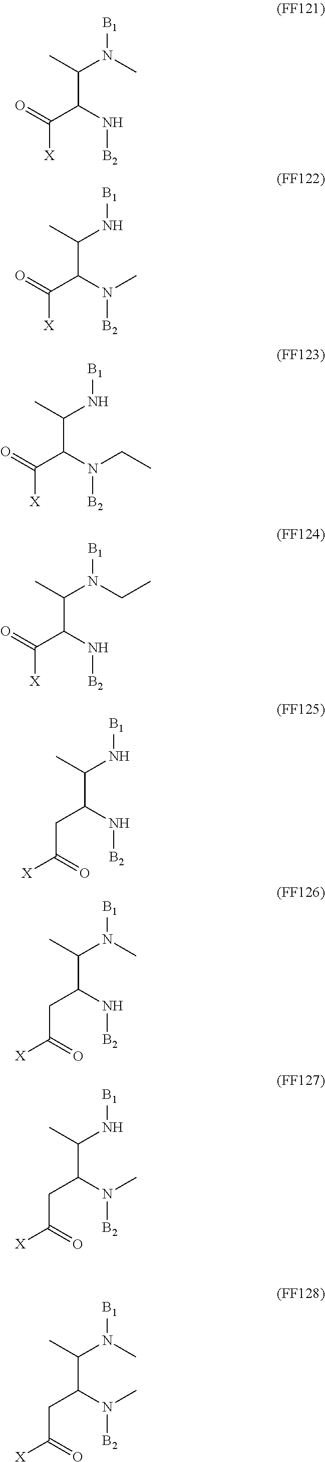 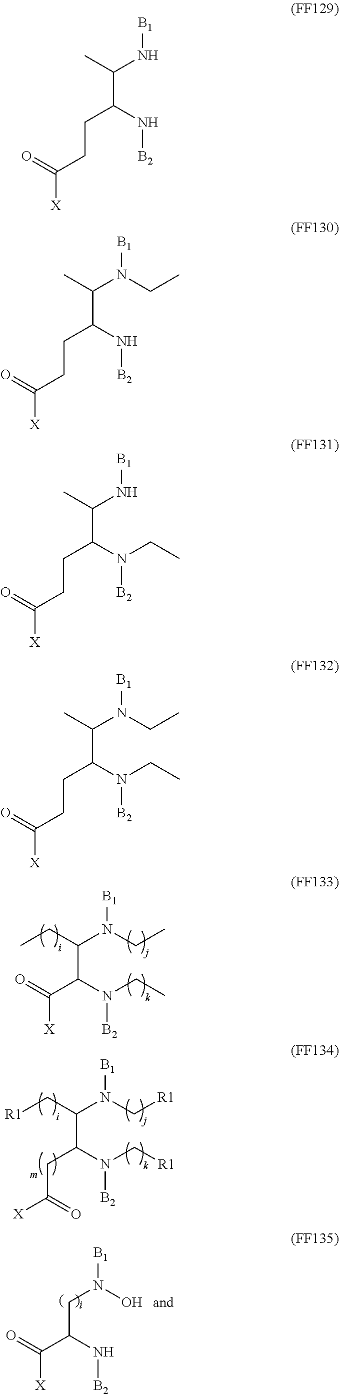 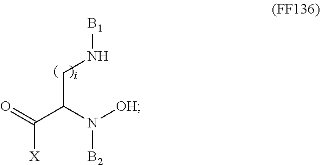
wherein X is selected from an amine, OH, and halogen;
i is 1, 2, 3, 4, 5, 6, or 7;
j is 1, 2, 3, 4, 5, 6, or 7;
k is 1, 2, 3, 4, 5, 6, or 7;
m is 1, 2, 3, 4, 5, 6, or 7;
each R1 is independently selected from H, an alkyl group, an acyl group, a cycloalkyl group, a haloalkyl group, an aryl group, and a heteroaryl group, each R1 optionally comprises one or more alkyl-halide, halide, sulfhydryl, aldehyde, amine, acid, hydroxyl, alkyl or aryl groups; and
B₁ and B₂, which may be identical or different, each independently represents an aromatic boron-containing group; and
wherein Formulae FF137-FF160 are: 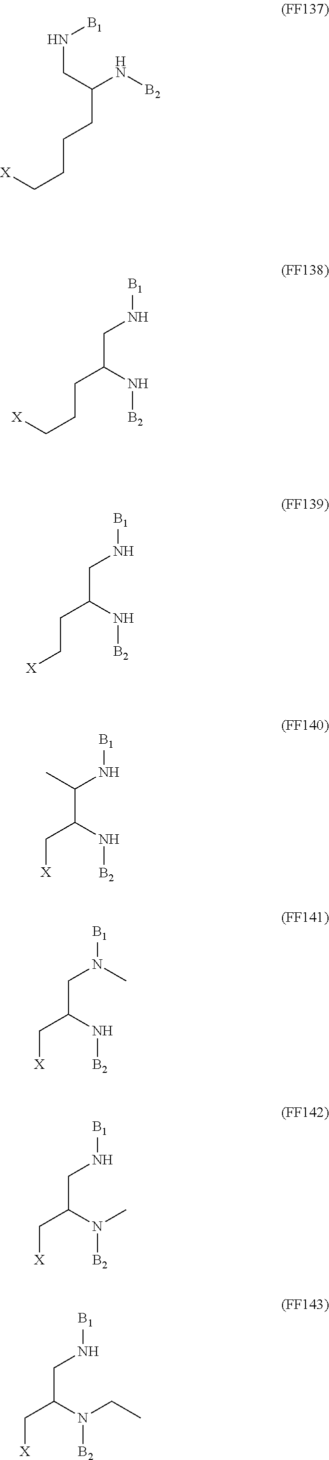 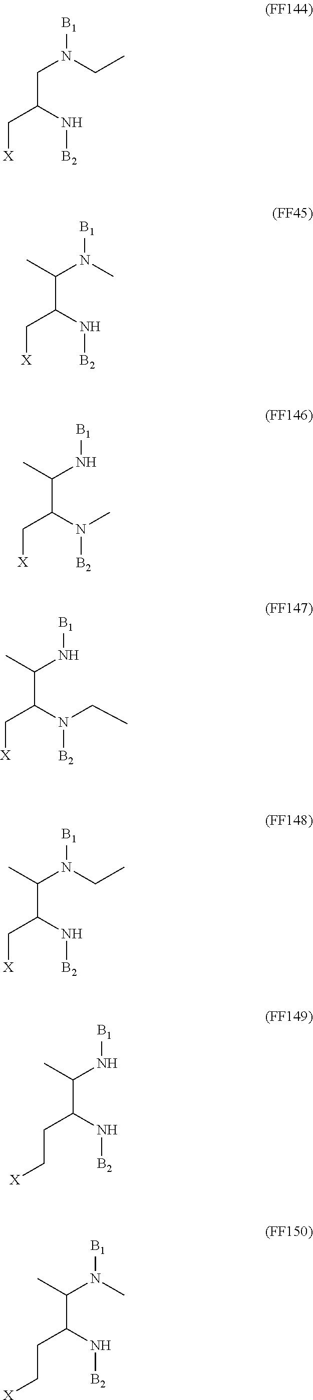 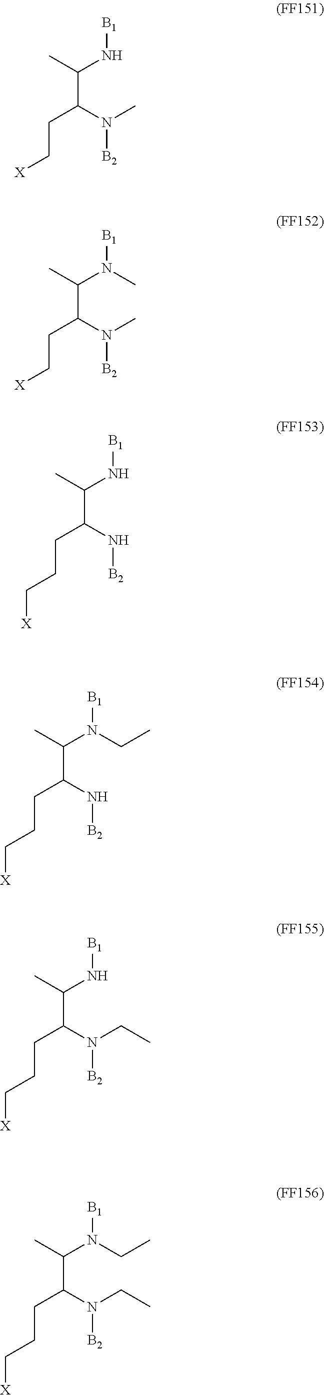 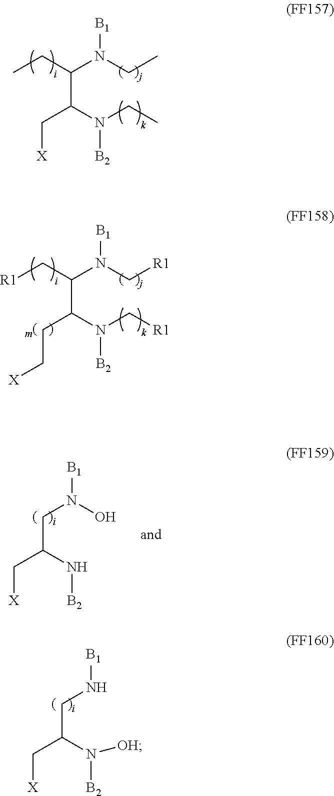
wherein X is selected from an amine, OH, and halogen;
i is 1, 2, 3, 4, 5, 6, or 7;
j is 1, 2, 3, 4, 5, 6, or 7;
k is 1, 2, 3, 4, 5, 6, or 7;
m is 1, 2, 3, 4, 5, 6, or 7;
each R1 is independently selected from H, an alkyl group, an acyl group, a cycloalkyl group, a haloalkyl group, an aryl group, and a heteroaryl group, each R1 optionally comprises one or more alkyl-halide, halide, sulfhydryl, aldehyde, amine, acid, hydroxyl, alkyl or aryl groups; and
B₁ and B₂, which may be identical or different, each independently represents an aromatic boron-containing group; and
wherein Formulae FF161-FF164 are: 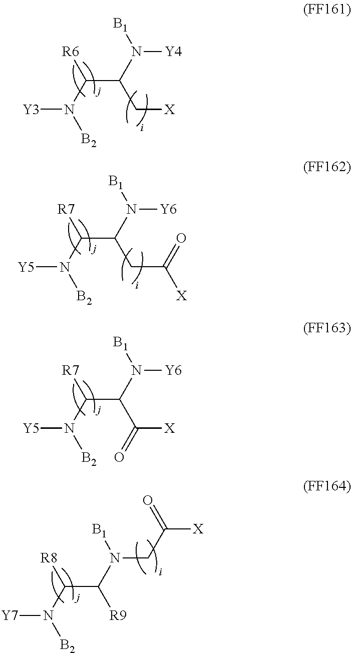
wherein X is selected from an amine, OH, and halogen;
i is 1, 2, 3, 4, or 5;
j is 1, 2, 3, 4, or 5;
each R6, R7, R8, and R9 for different values of j is independently selected from H, CF₃, CH₃, CHF₂, and (CH₂)ₘCH₃, wherein m is 1, 2, 3, 4, or 5;
Y3, Y4, Y5, Y6 and Y7 are each independently selected from H, CH₂—X4, and Formulae IV-1 to IV-135;
wherein X4 is selected from —COOH, —(CH₂)ₘCOOH, an alkyl group, an acyl group, a cycloalkyl group, a haloalkyl group, an aryl group, and a heteroaryl group, each optionally comprising one or more alkyl-halide, halide, sulfhydryl, aldehyde, amine, acid, hydroxyl, alkyl or aryl groups; wherein m is 1, 2, 3, 4, or 5;
wherein at least one of Y5, Y6, and Y7 in Formulae FF162 and FF163 is not H and at least one of Y7, R8 and R9 in FF164 is not H; and
wherein Formulae IV-1 to IV-135 are: 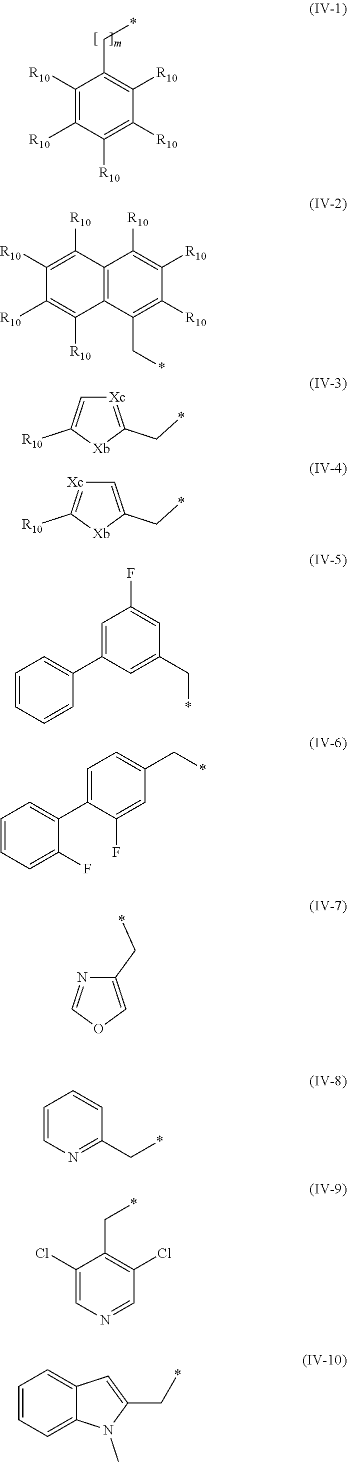 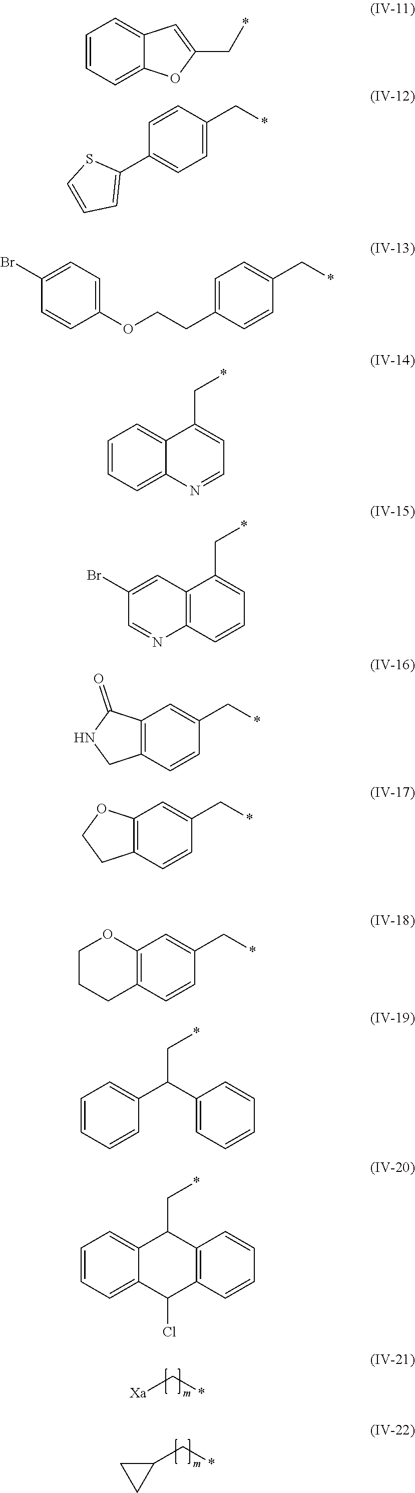 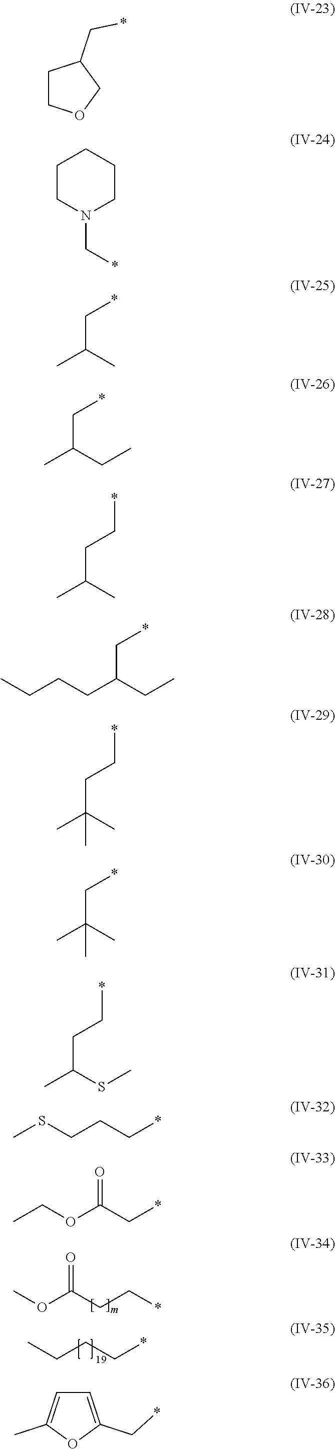 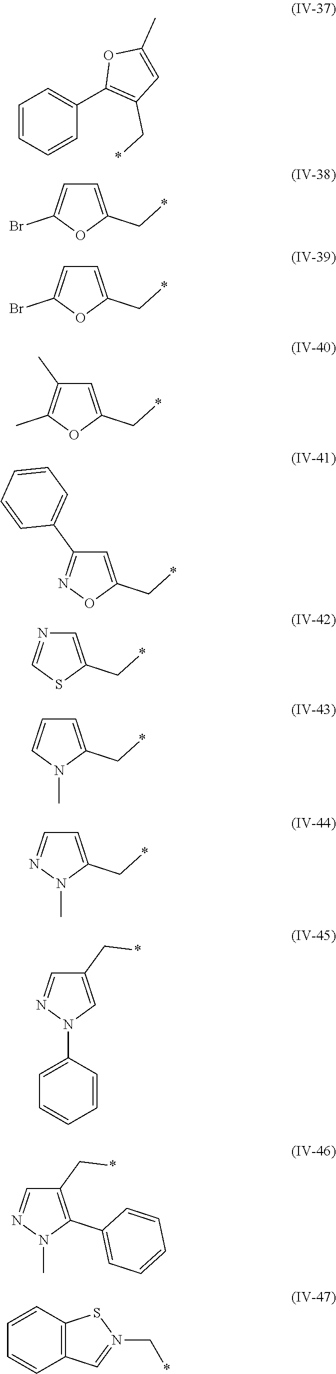 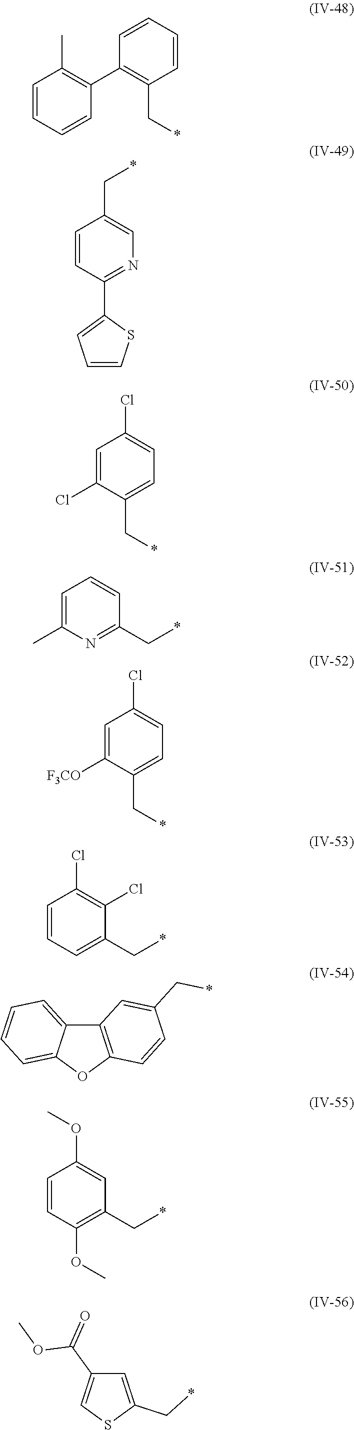 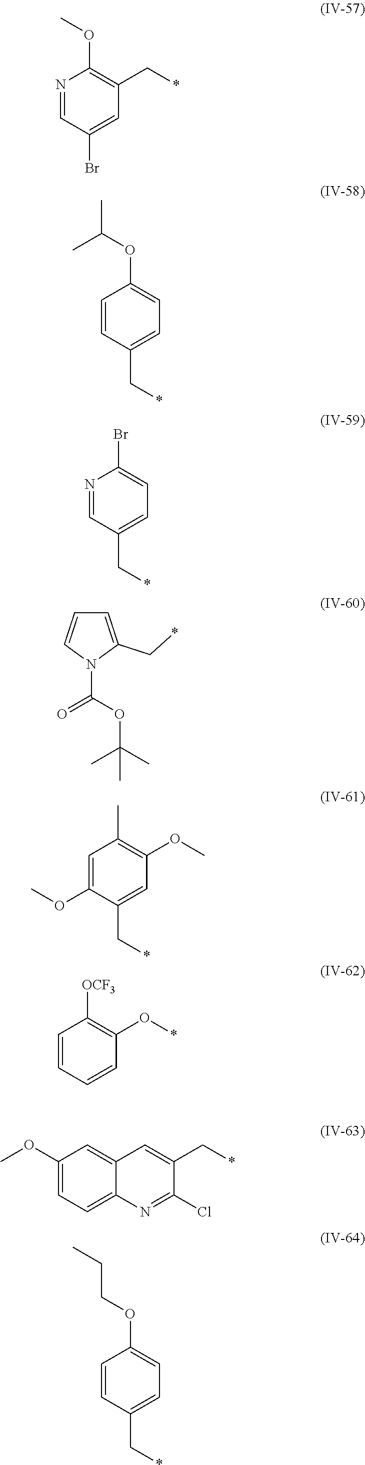 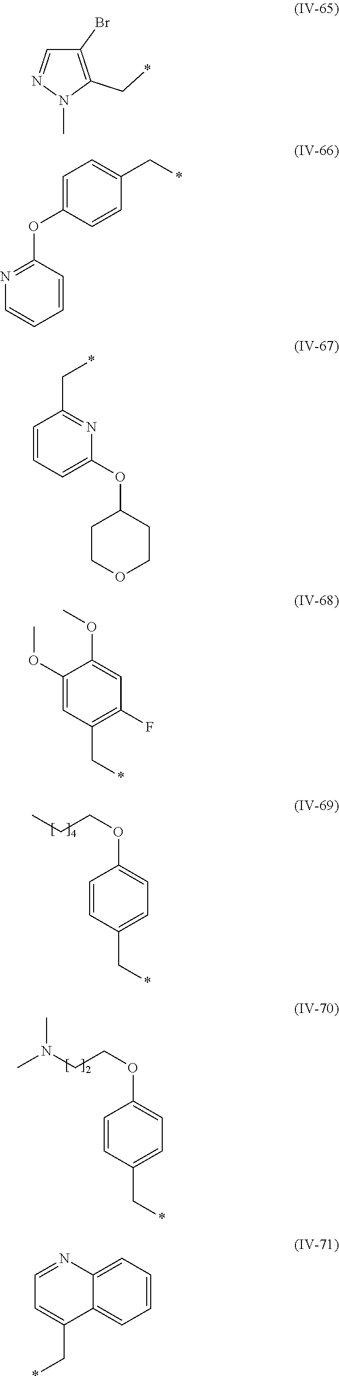 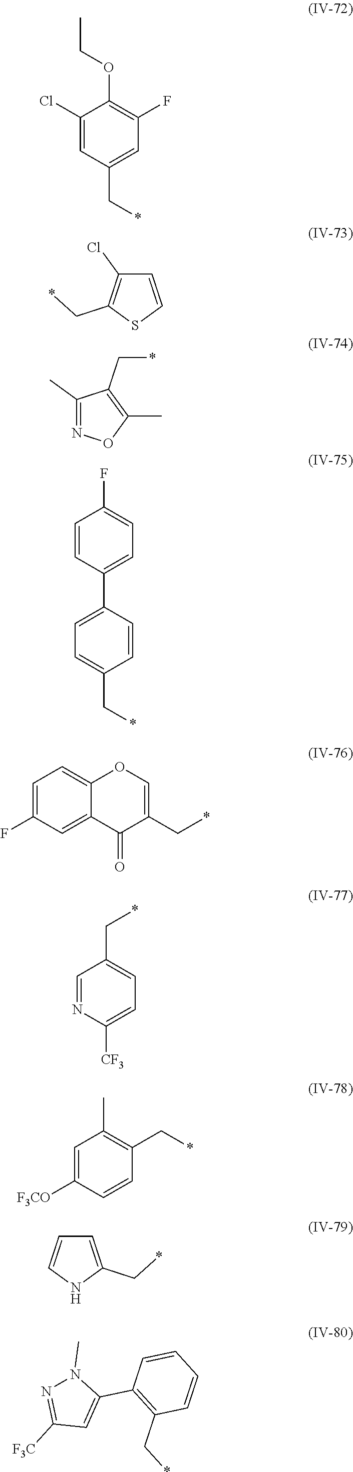 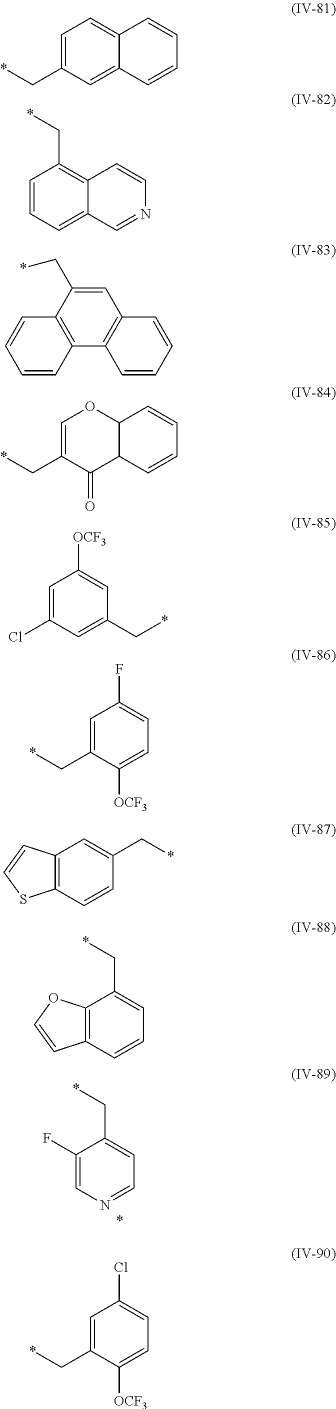 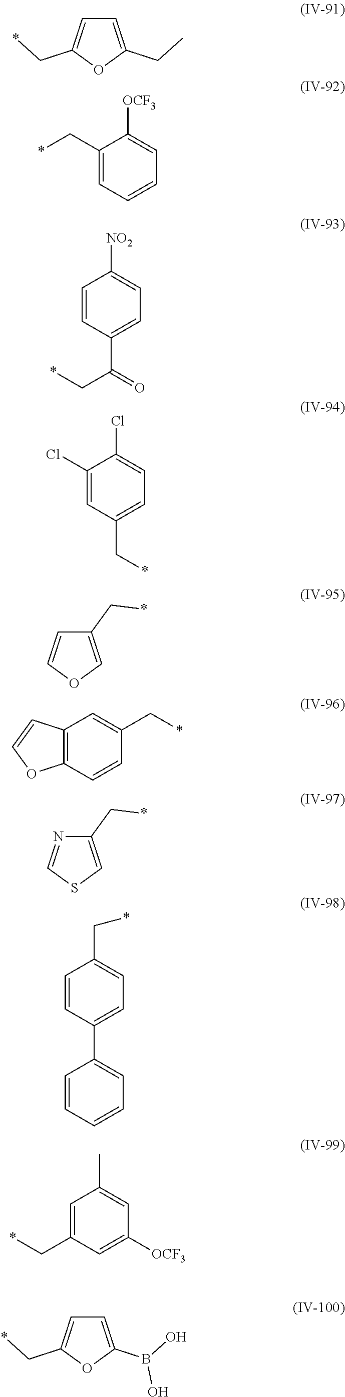 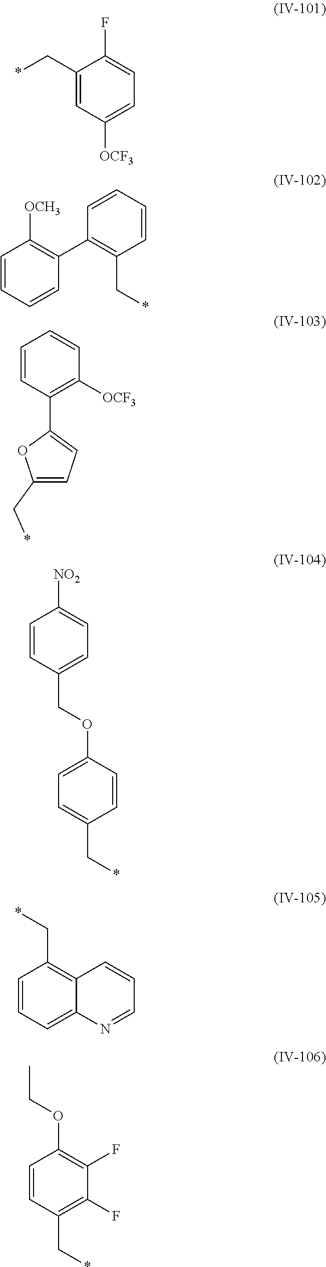 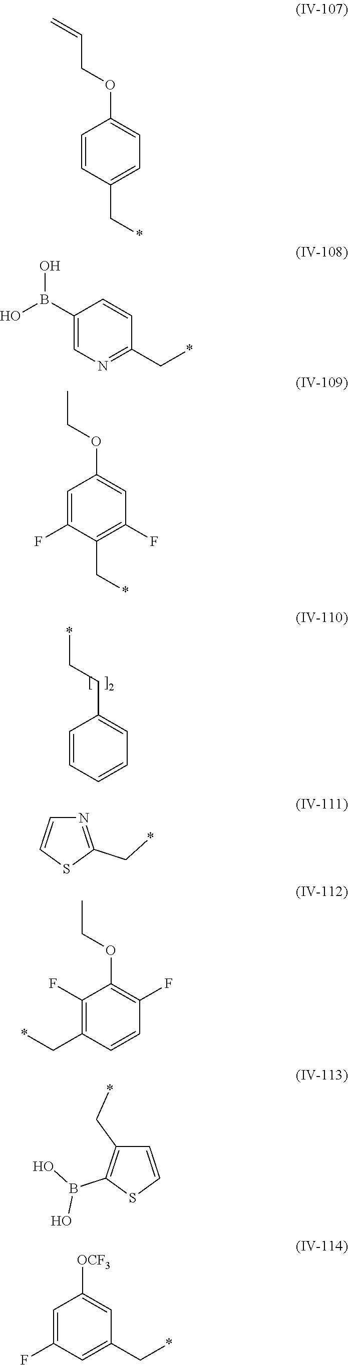 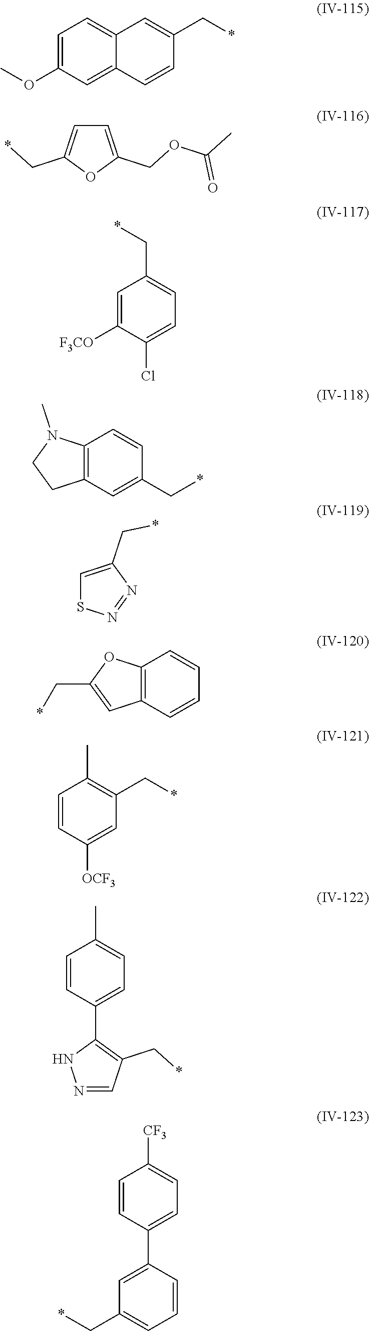 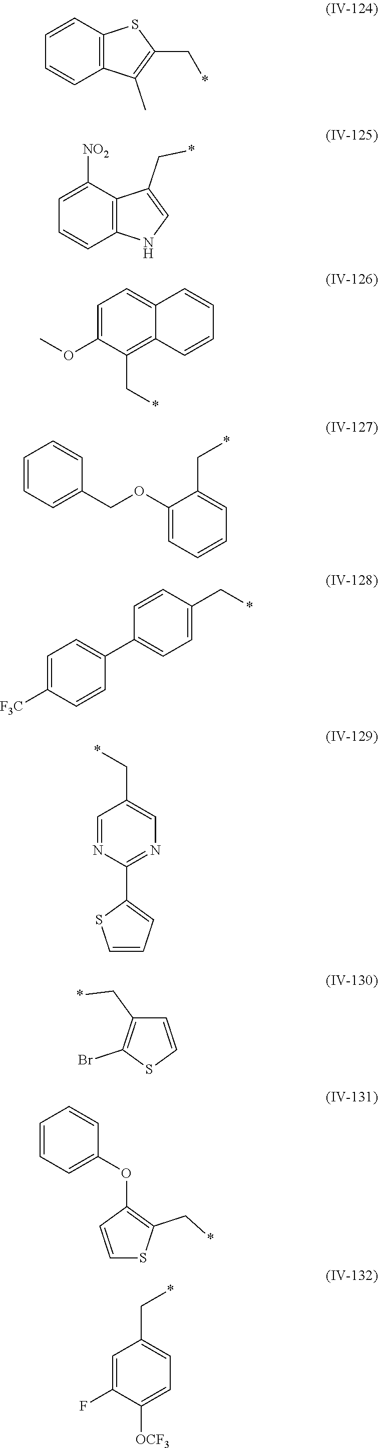 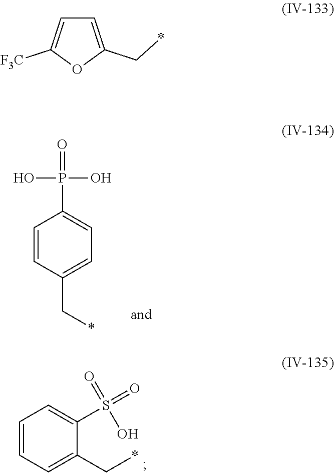
wherein:
Xa represents CH═O, CHF₂, CF₃, CH₂SH, COOH, CH₂OH, CH₂NO₂, CH₂NH₂, CH₃, C(CH₃)₃, CH(CH₃)₂, CH((CH₂)₃CH₃)₂, or CH(CH₂CH₃)₂;
Xb represents O, NH, CH₂, or S;
Xc represents CH or N;
each R₁₀ is independently selected from H, F, Cl, Br, CH₃, CF₃, CH═O, OH, COOH, and (CH₂)ₙCH₃,
m is 1, 2, 3, 4, or 5; and n is 1, 2, 3, 4, or 5;
B₁ and B₂, which may be identical or different, each independently represents an aromatic boron-containing group; and
* in Formulae IV-1 to IV-135 represents the point of attachment to corresponding Formulae FF161-164; and
wherein Formulae FF165-FF166 are: 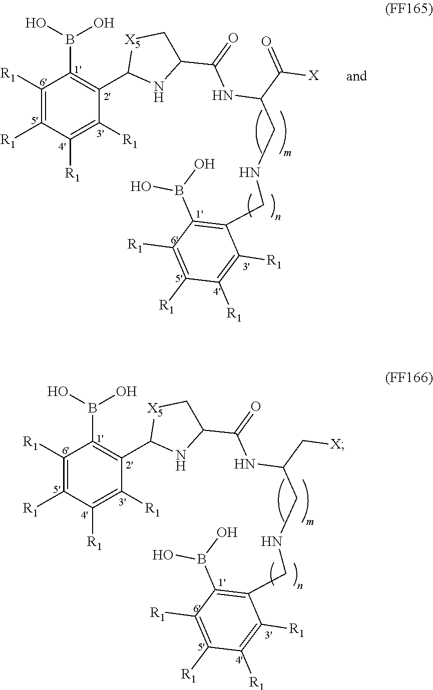
wherein X is selected from an amine, OH, and halogen;
m is 1, 2, 3, 4, 5, 6, or 7;
n is 1, 2, 3, 4, 5, 6, or 7;
X5 is S, O, or NH; and
each R₁ is independently selected from H, F, Cl, Br, OH, CH₂—NH₂, NH₂, (C═O)—NH₂, CH═O, SO₂CH₃, SO₂CF₃, CF₃, CHF₂, NO₂, CH₃, OCH₃, O(CH₂)ₘCH₃, (SO₂)NH CH₃, (SO₂)NH(CH₂)ₘCH₃, and OCF₃, wherein m is 1, 2, 3, 4, 5, 6, or 7; and
wherein Formulae FF167-FF192 are: 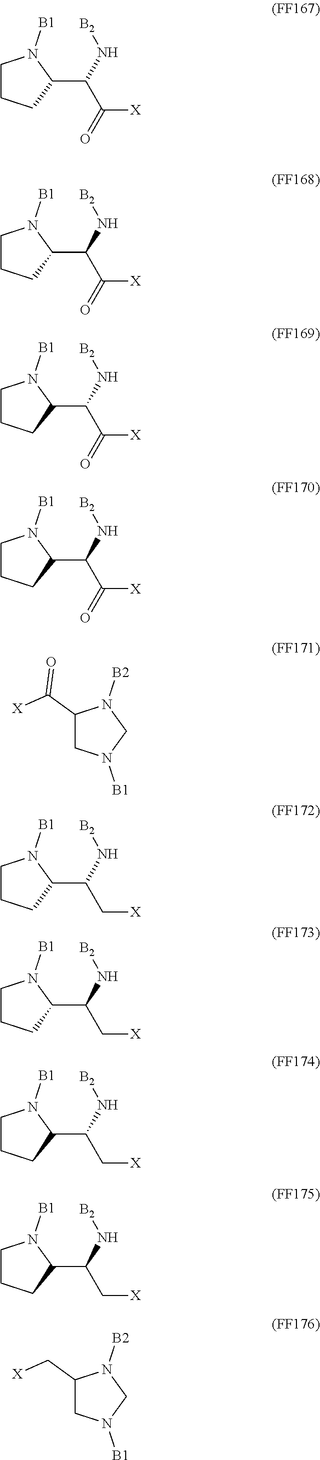 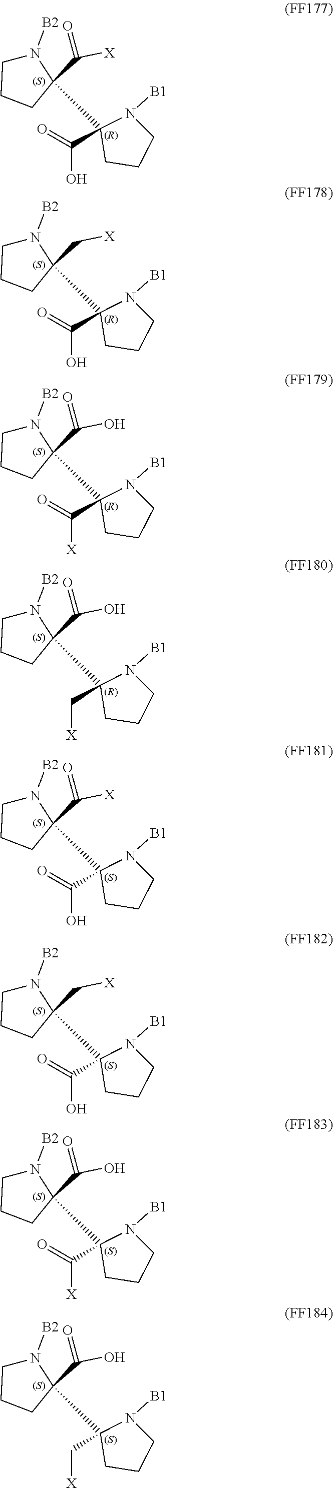 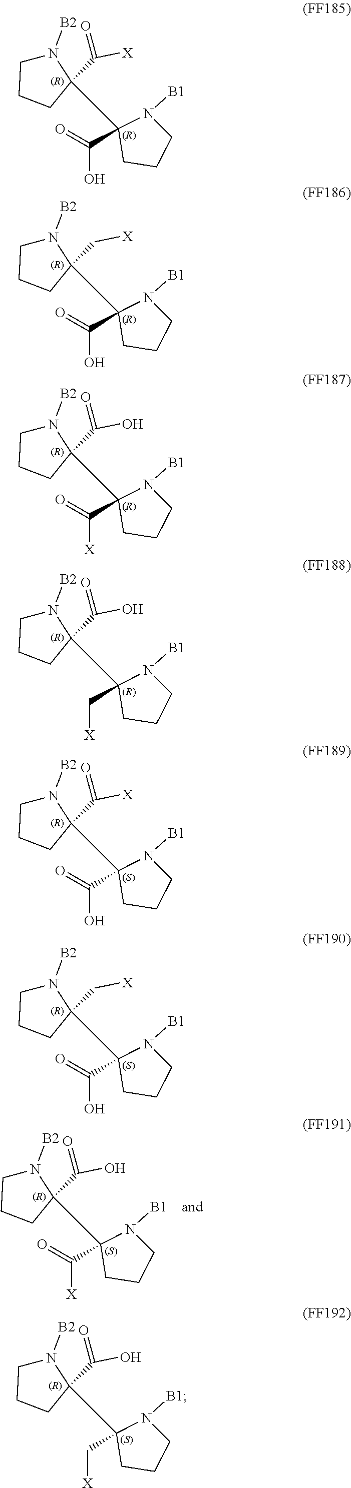
wherein X is selected from an amine, OH, and halogen;
B₁ and B₂, which may be identical or different, each independently represents an aromatic boron-containing group, and
wherein Formulae FF193-FF209 are: 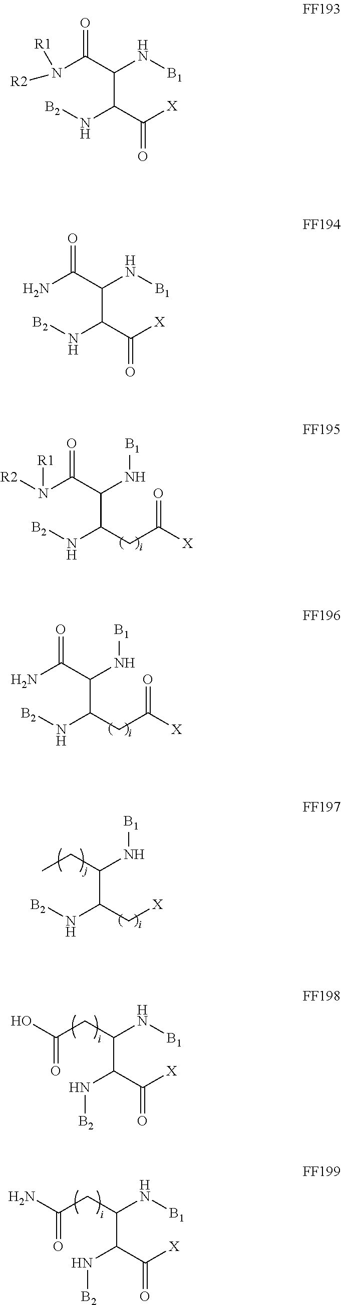 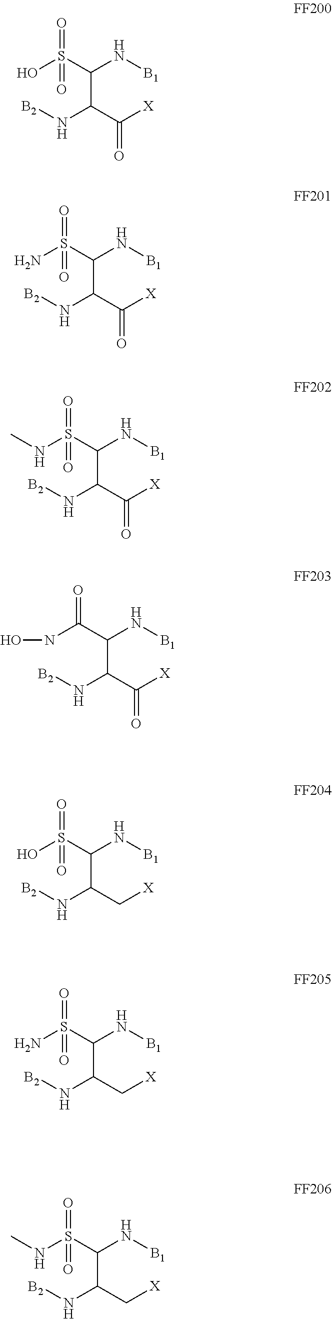 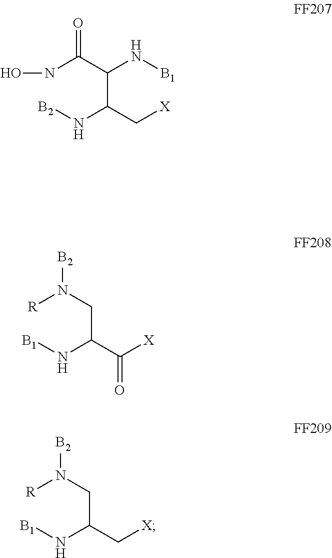
wherein R in FF208 and FF209 is an alkyl, aryl or halide that is covalently conjugated through at least one CH₂ group to the amino group in the side chain of FF208 or FF209;
R1 and R2 are independently selected from H, CH₃, alkyl, and formulae IV-1 to IV-135;
i is 1, 2, 3, 4, or 5;
j is 1, 2, 3, 4, or 5; and
wherein X is selected from an amine, OH, and halogen; and
B₁ and B₂, which may be identical or different, each independently represents an aromatic boron-containing group; and
wherein Formulae FF210-FF224 are: 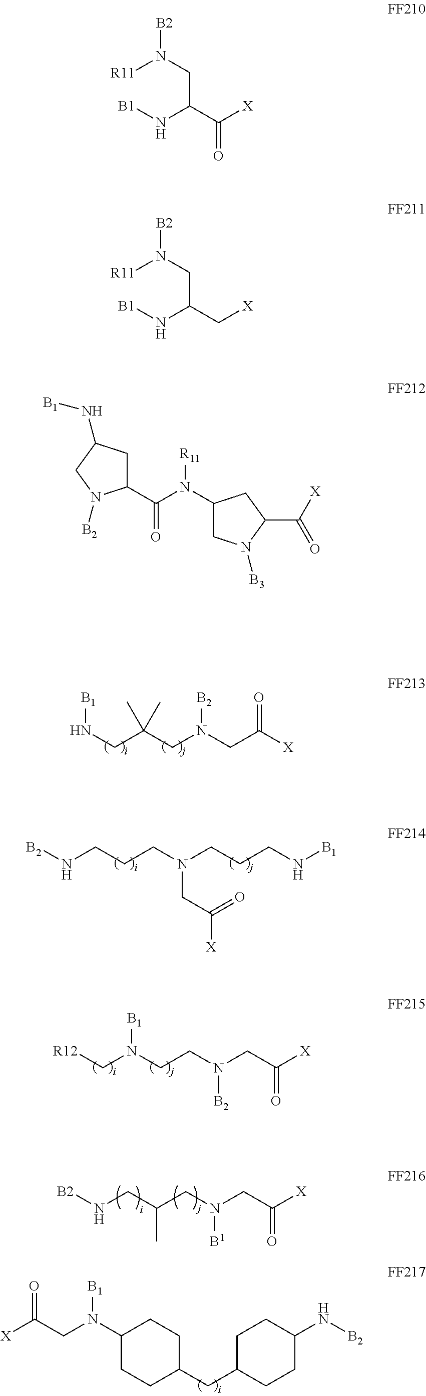 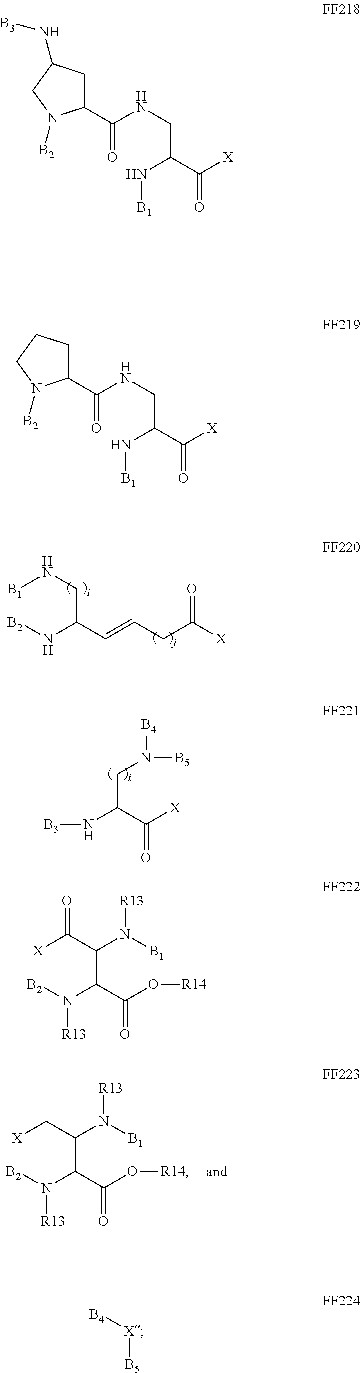
wherein R11 in FF210 to FF212 is independently selected from Formulae IV-1 to IV-135 and R12 is selected from an amine, a hydroxyl, an alkyl, and a halide group;
wherein each R13 is independently selected from H, CH₃, alkyl, aryl, and formulae IV-1 to IV-135; R14 is selected from H, CH₃, alkyl, aryl, and heteroaryl;
wherein X is independently selected from an amine, OH, and halogen;
X″ is an amine;
i is 1, 2, 3, 4, or 5;
j is 1, 2, 3, 4, or 5; and
wherein at least one primary or secondary amine in FF1-FF223 is optionally covalently conjugated to B₆; and
B₁, B₂, B₃, B₄, B₅ and B₆ each independently represents an aromatic boron-containing group, wherein in each FF structure containing B₁, B₂ and B₃ groups, at least two of the B₁, B₂ and B₃ groups are independently an aromatic boron-containing group; and
when X is an amine in any one of Formulae FF1 to FF223, X is optionally acetylated or alkylated.

51. The compound of claim 50, wherein the compound comprises at least one of B₁, B₂ and B₃ independently selected from Formulae F1-F12 or wherein the compound comprises at least one of B₄, B₅ and B₆ independently selected from Formulae F1-F10,
wherein Formulae F1-F10 are: 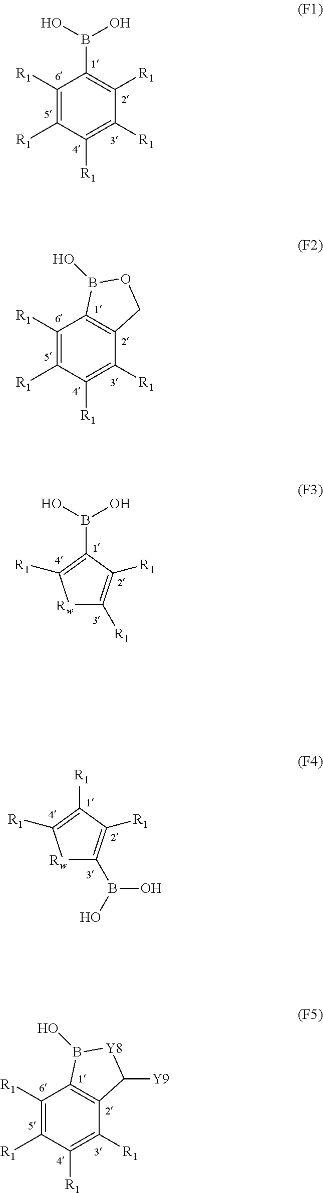 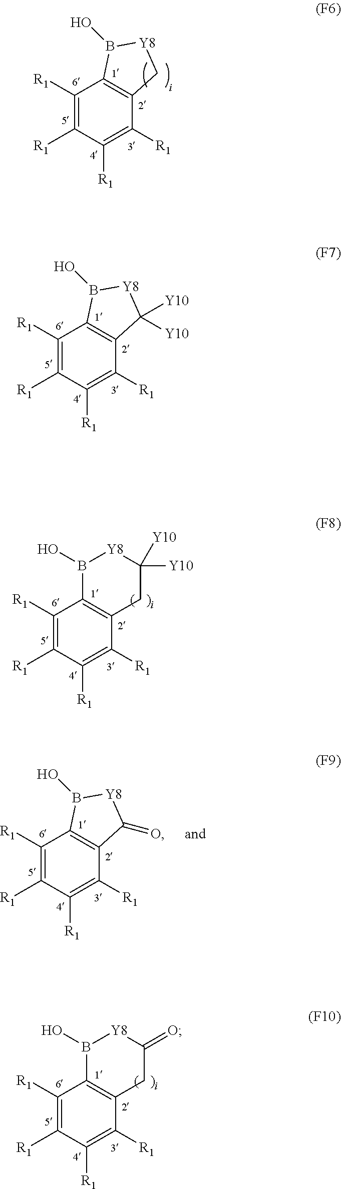
wherein for B₁, B₂, B₃:
one R₁ represents (C═O)—*, S(═O)(═O)—*, (CH₂)ₘ(C═O)—*, or (CH₂)ₘ—*, wherein —* represents an attachment point to the rest of Z1c, and m is 1, 2, 3, 4, 5, 6, or 7;
each remaining R₁ is independently selected from H, F, Cl, Br, OH, CH₂—NH₂, NH₂, (C═O)—NH₂, CH═O, SO₂CH₃, SO₂CF₃, CF₃, CHF₂, NO₂, CH₃, OCH₃, O(CH₂)ₘCH₃, —(SO₂)NH CH₃, —(SO₂)NH(CH₂)ₘCH₃, and OCF₃, wherein m is 1, 2, 3,4, 5, 6, or 7;
wherein for B₄, B₅:
one R₁ for B₄ represents (CH₂)ₘ—Ø, wherein —Ø represents the attachment point (representing a covalent bond) to an amine in X1 and one R₁ for B₅ represents (C═O)—*, S(═O)(═O)—*, (CH₂)ₘ(C═O)—*, or (CH₂)ₘ—*, wherein —* represents the attachment point to the same amine in X1, and m is 1, 2, 3, 4, 5, 6, or 7;
each remaining R₁ is independently selected from H, F, Cl, Br, OH, CH₂—NH₂, NH₂, (C═O)—NH₂, CH═O, SO₂CH₃, SO₂CF₃, CF₃, CHF₂, NO₂, CH₃, OCH₃, O(CH₂)ₘCH₃, —(SO₂)NH CH₃, —(SO₂)NH(CH₂)ₘCH₃, and OCF₃, wherein m is 1, 2, 3,4, 5, 6, or 7;
wherein for B₆:
one R₁ for B₆ represents (CH₂)ₘ—Ø, wherein —Ø represents the attachment point (representing a covalent bond) to the rest of the compound, and m is 1, 2, 3, 4, 5, 6, or 7;
each remaining R₁ is independently selected from H, F, Cl, Br, OH, CH₂—NH₂, NH₂, (C═O)—NH₂, CH═O, SO₂CH₃, SO₂CF₃, CF₃, CHF₂, NO₂, CH₃, OCH₃, O(CH₂)ₘCH₃, —(SO₂)NH CH₃, —(SO₂)NH(CH₂)ₘCH₃, and OCF₃, wherein m is 1, 2, 3,4, 5, 6, or 7;
for Formulae F3-F4:
R_{w} is 0 or S;
for Formulae F5-F10:
Y8 is selected from O, N, and NR, wherein R is an alkyl group or H;
Y9 is H, CH₃, or an alkyl group, provided that when Y8 is O, Y9 is a CH₃ or higher alkyl group;
each Y10 is independently selected from H, CH₃, F, CF₃, and OCH₃; and
i is 1, 2, or 3; and
wherein Formulae F11-F12 are: 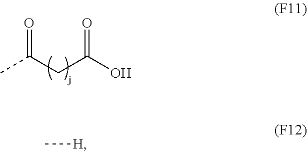
j is 1, 2, 3, 4, 5, 6, or 7; and
— represents the attachment point to the rest of Z1c.

52. The compound of claim 50, wherein the compound is selected from:
N-(3-(3-borono-5-nitrobenzamido)propyl)-N-(3-borono-5-nitrobenzoyl)glycine (DS01);
N-(4-((4-(3-borono-5-nitrobenzamido)cyclohexyl)methyl)cyclohexyl)-N-(3-borono-5-nitrobenzoyl)glycine (DS02);
N-(4-((3-borono-5-nitrobenzamido)methyl)benzyl)-N-(3-borono-5-nitrobenzoyl)glycine (DS03);
N-(3-((3-borono-5-nitrobenzamido)methyl)benzyl)-N-(3-borono-5-nitrobenzoyl)glycine (DS04);
N-(4-(3-borono-5-nitrobenzamido)butyl)-N-(3-borono-5-nitrobenzoyl)glycine (DS05);
N-(3-(3-borono-5-fluorobenzamido)propyl)-N-(3-borono-5-fluorobenzoyl)glycine (DS06);
N-(3-(3-borono-5-fluorobenzamido)-2,2-dimethylpropyl)-N-(3-borono-5-fluorobenzoyl)glycine (DS07);
bis(3-(3-borono-5-fluorobenzamido)propyl)glycine (DS08);
N-(4-((3-borono-5-fluorobenzamido)methyl)benzyl)-N-(3-borono-5-fluorobenzoyl)glycine (DS09);
N-(3-((3-borono-5-fluorobenzamido)methyl)benzyl)-N-(3-borono-5-fluorobenzoyl)glycine (DS10);
N-(2-(3-borono-5-fluorobenzamido)cyclohexyl)-N-(3-borono-5-fluorobenzoyl)glycine (DS11);
N-(3-(3-borono-4-fluorobenzamido)propyl)-N-(3-borono-4-fluorobenzoyl)glycine (DS12);
N-(4-((4-(3-borono-4-fluorobenzamido)cyclohexyl)methyl)cyclohexyl)-N-(3-borono-4-fluorobenzoyl)glycine (DS13);
N-(3-(3-borono-4-fluorobenzamido)-2,2-dimethylpropyl)-N-(3-borono-4-fluorobenzoyl)glycine (DS14);
N-(4-((3-borono-4-fluorobenzamido)methyl)benzyl)-N-(3-borono-4-fluorobenzoyl)glycine (DS15);
N-(3-((3-borono-4-fluorobenzamido)methyl)benzyl)-N-(3-borono-4-fluorobenzoyl)glycine (DS16);
N-((1S,2R)-2-(3-borono-4-fluorobenzamido)cyclohexyl)-N-(3-borono-4-fluorobenzoyl)glycine (DS17);
N-((1S,2S)-2-(3-borono-4-fluorobenzamido)cyclohexyl)-N-(3-borono-4-fluorobenzoyl)glycine (DS18);
N-(3-(3-borono-5-bromobenzamido)propyl)-N-(3-borono-5-bromobenzoyl)glycine (DS19);
N-(4-((4-(3-borono-5-bromobenzamido)cyclohexyl)methyl)cyclohexyl)-N-(3-borono-5-bromobenzoyl)glycine (DS20);
bis(3-(3-borono-5-bromobenzamido)propyl)glycine (DS21);
N-(4-((3-borono-5-bromobenzamido)methyl)benzyl)-N-(3-borono-5-bromobenzoyl)glycine (DS22);
N-(3-((3-borono-5-bromobenzamido)methyl)benzyl)-N-(3-borono-5-bromobenzoyl)glycine (DS23);
N-(2-(3-borono-5-bromobenzamido)cyclohexyl)-N-(3-borono-5-bromobenzoyl)glycine (DS24);
N-(3-(4-borono-3-fluorobenzamido)propyl)-N-(4-borono-3-fluorobenzoyl)glycine (DS25);
N-(4-((4-(4-borono-3-fluorobenzamido)cyclohexyl)methyl)cyclohexyl)-N-(4-borono-3-fluorobenzoyl)glycine (DS26);
N-(3-(4-borono-3-fluorobenzamido)-2,2-dimethylpropyl)-N-(4-borono-3-fluorobenzoyl)glycine (DS27);
bis(3-(4-borono-3-fluorobenzamido)propyl)glycine (DS28);
N-(4-((4-borono-3-fluorobenzamido)methyl)benzyl)-N-(4-borono-3-fluorobenzoyl)glycine (DS29);
N-(3-((4-borono-3-fluorobenzamido)methyl)benzyl)-N-(4-borono-3-fluorobenzoyl)glycine (DS30);
N-((1S,2R)-2-(4-borono-3-fluorobenzamido)cyclohexyl)-N-(4-borono-3-fluorobenzoyl)glycine (DS31);
N-(1-hydroxy-1,3-dihydrobenzo[c][1,2]oxaborole-6-carbonyl)-N-(3-(1-hydroxy-1,3-dihydrobenzo[c][1,2]oxaborole-6-carboxamido)propyl)glycine (DS32);
N-(1-hydroxy-1,3-dihydrobenzo[c][1,2]oxaborole-6-carbonyl)-N-(5-(1-hydroxy-1,3-dihydrobenzo[c][1,2]oxaborole-6-carboxamido)pentyl)glycine (DS33);
N-(1-hydroxy-1,3-dihydrobenzo[c][1,2]oxaborole-6-carbonyl)-N-(3-(1-hydroxy-1,3-dihydrobenzo[c][1,2]oxaborole-6-carboxamido)-2,2-dimethylpropyl)glycine (DS34);
bis(3-(1-hydroxy-1,3-dihydrobenzo[c][1,2]oxaborole-6-carboxamido)propyl)glycine (DS35);
N-(1-hydroxy-1,3-dihydrobenzo[c][1,2]oxaborole-6-carbonyl)-N-(3-((1-hydroxy-1,3-dihydrobenzo[c][1,2]oxaborole-6-carboxamido)methyl)benzyl)glycine (DS36);
N-(1-hydroxy-1,3-dihydrobenzo[c][1,2]oxaborole-6-carbonyl)-N-((1S,2R)-2-(1-hydroxy-1,3-dihydrobenzo[c][1,2]oxaborole-6-carboxamido)cyclohexyl)glycine (DS37);
N-(1-hydroxy-1,3-dihydrobenzo[c][1,2]oxaborole-6-carbonyl)-N-(4-(1-hydroxy-1,3-dihydrobenzo[c][1,2]oxaborole-6-carboxamido)butyl)glycine (DS38);
N-(1-hydroxy-1,3-dihydrobenzo[c][1,2]oxaborole-6-carbonyl)-N-((1S,2S)-2-(1-hydroxy-1,3-dihydrobenzo[c][1,2]oxaborole-6-carboxamido)cyclohexyl)glycine (DS39);
(R)—N-(1-hydroxy-1,3-dihydrobenzo[c][1,2]oxaborole-6-carbonyl)-N-(2-(1-hydroxy-1,3-dihydrobenzo[c][1,2]oxaborole-6-carboxamido)propyl)glycine (DS40);
(S)—N-(1-hydroxy-1,3-dihydrobenzo[c][1,2]oxaborole-6-carbonyl)-N-(2-(1-hydroxy-1,3-dihydrobenzo[c][1,2]oxaborole-6-carboxamido)propyl)glycine (DS41);
N-(1-hydroxy-1,3-dihydrobenzo[c][1,2]oxaborole-6-carbonyl)-N-(2-(1-hydroxy-1,3-dihydrobenzo[c][1,2]oxaborole-6-carboxamido)cyclohexyl)glycine (DS42);
N-(3-(4-borono-3, 5-difluorobenzamido)propyl)-N-(4-borono-3, 5-difluorobenzoyl)glycine (DS43);
N-(3-(4-borono-2-fluorobenzamido)propyl)-N-(4-borono-2-fluorobenzoyl)glycine (DS44);
N-(2-(N-ethyl-1-hydroxy-1,3-dihydrobenzo[c][1,2]oxaborole-6-carboxamido)ethyl)-N-(1-hydroxy-1,3-dihydrobenzo[c][1,2]oxaborole-6-carbonyl)glycine (DS45);
N-(1-hydroxy-1,3-dihydrobenzo[c][1,2]oxaborole-6-carbonyl)-N-(2-(1-hydroxy-N-(2-hydroxyethyl)-1,3-dihydrobenzo[c][1,2]oxaborole-6-carboxamido)ethyl)glycine (DS46);
N-(1-hydroxy-1,3-dihydrobenzo[c][1,2]oxaborole-6-carbonyl)-N-(5-(1-hydroxy-1,3-dihydrobenzo[c][1,2]oxaborole-6-carboxamido)hexyl)glycine (DS47);
N-(1-hydroxy-1,3-dihydrobenzo[c][1,2]oxaborole-6-carbonyl)-N-(4-((4-(1-hydroxy-1,3-dihydrobenzo[c][1,2]oxaborole-6-carboxamido)cyclohexyl)methyl)cyclohexyl)glycine (DS48);
((2S,4S)-1-(1-hydroxy-1,3-dihydrobenzo[c][1,2]oxaborole-6-carbonyl)-4-(1-hydroxy-1,3-dihydrobenzo[c][1,2]oxaborole-6-carboxamido)pyrrolidine-2-carbonyl)glycine (DS49);
((2S,4S)-4-(3-borono-4-fluorobenzamido)-1-(3-borono-4-fluorobenzoyl)pyrrolidine-2-carbonyl)glycine (DS50);
((2S,4S)-4-(3-borono-5-nitrobenzamido)-1-(3-borono-5-nitrobenzoyl)pyrrolidine-2-carbonyl)glycine (DS51);
((2S,4S)-4-(5-borono-2-fluorobenzamido)-1-(5-borono-2-fluorobenzoyl)pyrrolidine-2-carbonyl)glycine (DS52);
(S)-(1,4-bis(1-hydroxy-1,3-dihydrobenzo[c][1,2]oxaborole-6-carbonyl)piperazine-2-carbonyl)glycine (DS53);
(S)—N-(3-amino-2-(1-hydroxy-1,3-dihydrobenzo[c][1,2]oxaborole-6-carboxamido)-3-oxopropyl)-N-benzyl-1-hydroxy-1,3-dihydrobenzo[c][1,2]oxaborole-6-carboxamide (DS54);
(S)—N-(3-amino-2-(1-hydroxy-1,3-dihydrobenzo[c][1,2]oxaborole-6-carboxamido)-3-oxopropyl)-1-hydroxy-N-(4-(trifluoromethyl)benzyl)-1,3-dihydrobenzo[c][1,2]oxaborole-6-carboxamide (DS55);
(S)—N-(3-amino-2-(1-hydroxy-1,3-dihydrobenzo[c][1,2]oxaborole-6-carboxamido)-3-oxopropyl)-N-ethyl-1-hydroxy-1,3-dihydrobenzo[c][1,2]oxaborole-6-carboxamide (DS56);
(S)—N-(3-amino-2-(1-hydroxy-1,3-dihydrobenzo[c][1,2]oxaborole-6-carboxamido)-3-oxopropyl)-1-hydroxy-N-propyl-1,3-dihydrobenzo[c][1,2]oxaborole-6-carboxamide (DS57);
(S)—N-(3-amino-2-(1-hydroxy-1,3-dihydrobenzo[c][1,2]oxaborole-6-carboxamido)-3-oxopropyl)-1-hydroxy-N-isobutyl-1,3-dihydrobenzo[c][1,2]oxaborole-6-carboxamide (DS58);
(S)—N-(3-amino-2-(1-hydroxy-1,3-dihydrobenzo[c][1,2]oxaborole-6-carboxamido)-3-oxopropyl)-1-hydroxy-N-((5-(thiophen-2-yl)pyridin-2-yl)methyl)-1,3-dihydrobenzo[c][1,2]oxaborole-6-carboxamide (DS59);
(S)—N-(3-amino-2-(1-hydroxy-1,3-dihydrobenzo[c][1,2]oxaborole-6-carboxamido)-3-oxopropyl)-1-hydroxy-N-isopentyl-1,3-dihydrobenzo[c][1,2]oxaborole-6-carboxamide (DS60);
(S)—N-(3-amino-2-(1-hydroxy-1,3-dihydrobenzo[c][1,2]oxaborole-6-carboxamido)-3-oxopropyl)-1-hydroxy-N-(quinolin-5-ylmethyl)-1,3-dihydrobenzo[c][1,2]oxaborole-6-carboxamide (DS61);
(S)—N-(3-amino-2-(1-hydroxy-1,3-dihydrobenzo[c][1,2]oxaborole-6-carboxamido)-3-oxopropyl)-1-hydroxy-N-(2-(trifluoromethoxy)benzyl)-1,3-dihydrobenzo[c][1,2]oxaborole-6-carboxamide (DS62);
(S)—N-(3-amino-2-(1-hydroxy-1,3-dihydrobenzo[c][1,2]oxaborole-6-carboxamido)-3-oxopropyl)-1-hydroxy-N-(4-(methylsulfonyl)benzyl)-1,3-dihydrobenzo[c][1,2]oxaborole-6-carboxamide (DS63);
(3-((2S,4S)-4-(5-borono-2-(methylsulfonyl)benzamido)-2-carbamoylpyrrolidine-1-carbonyl)-4-(methylsulfonyl)phenyl)boronic acid (DS64);
(4-(((3S,5S)-1-(4-borono-2,6-difluorobenzoyl)-5-carbamoylpyrrolidin-3-yl)carbamoyl)-3, 5-difluorophenyl)boronic acid (DS65);
(R,E)-4,5-bis(1-hydroxy-1,3-dihydrobenzo[c][1,2]oxaborole-6-carboxamido)pent-2-enoic acid (DS66);
(2S,4S)-1-(1-hydroxy-4-(trifluoromethyl)-1,3-dihydrobenzo[c][1,2]oxaborole-6-carbonyl)-4-(1-hydroxy-4-(trifluoromethyl)-1,3-dihydrobenzo[c][1,2]oxaborole-6-carboxamido)pyrrolidine-2-carboxamide (DS67);
N,N′-((2S,3S)-1-amino-1-oxobutane-2,3-diyl)bis(1-hydroxy-1,3-dihydrobenzo[c][1,2]oxaborole-6-carboxamide) (DS68);
(R)-3,4-bis(1-hydroxy-1,3-dihydrobenzo[c][1,2]oxaborole-6-carboxamido)butanoic acid (DS69);
3-((2S,4S)-1-(1-hydroxy-1,3-dihydrobenzo[c][1,2]oxaborole-6-carbonyl)-4-(1-hydroxy-1,3-dihydrobenzo[c][1,2]oxaborole-6-carboxamido)pyrrolidine-2-carboxamido)propanoic acid (DS70);
(S)-3-(1-hydroxy-1,3-dihydrobenzo[c][1,2]oxaborole-5-carboxamido)-4-(1-hydroxy-1,3-dihydrobenzo[c][1,2]oxaborole-6-carboxamido)butanoic acid (DS71);
(R)-4-(1-hydroxy-1,3-dihydrobenzo[c][1,2]oxaborole-5-carboxamido)-5-(1-hydroxy-1,3-dihydrobenzo[c][1,2]oxaborole-6-carboxamido)pentanoic acid (DS72);
(2S,4R)-1-(1-hydroxy-1,3-dihydrobenzo[c][1,2]oxaborole-6-carbonyl)-4-(1-hydroxy-1,3-dihydrobenzo[c][1,2]oxaborole-6-carboxamido)pyrrolidine-2-carboxylic acid (DS73);
(2S,4R)-1-(1-hydroxy-4-(trifluoromethyl)-1,3-dihydrobenzo[c][1,2]oxaborole-6-carbonyl)-4-(1-hydroxy-4-(trifluoromethyl)-1,3-dihydrobenzo[c][1,2]oxaborole-6-carboxamido)pyrrolidine-2-carboxylic acid (DS74);
(2S,3S)-3-(1-hydroxy-4-(trifluoromethyl)-1,3-dihydrobenzo[c][1,2]oxaborole-6-carboxamido)-2-(1-hydroxy-7-(trifluoromethyl)-1,3-dihydrobenzo[c][1,2]oxaborole-5-carboxamido)butanoic acid (DS75);
(R)-5-(1-hydroxy-4-(trifluoromethyl)-1,3-dihydrobenzo[c][1,2]oxaborole-6-carboxamido)-4-(1-hydroxy-7-(trifluoromethyl)-1,3-dihydrobenzo[c][1,2]oxaborole-5-carboxamido)pentanoic acid (DS76);
((2S,4S)-1-(5-borono-2-nitrobenzoyl)-4-(1-hydroxy-1,3-dihydrobenzo[c][1,2]oxaborole-6-carboxamido)pyrrolidine-2-carbonyl)glycine (DS77);
((2S,4S)-1-(5-borono-2-(methylsulfonyl)benzoyl)-4-(1-hydroxy-1,3-dihydrobenzo[c][1,2]oxaborole-6-carboxamido)pyrrolidine-2-carbonyl)glycine (DS78);
((2S,4S)-1-(3-borono-2,6-difluorobenzoyl)-4-(1-hydroxy-1,3-dihydrobenzo[c][1,2]oxaborole-6-carboxamido)pyrrolidine-2-carbonyl)glycine (DS79);
(S)-(3-((3-borono-4-fluorobenzyl)(5,6-diamino-6-oxohexyl)carbamoyl)-5-nitrophenyl)boronic acid (DS80);
(S)-(3-((4-borono-3, 5-difluorobenzyl)(5,6-diamino-6-oxohexyl)carbamoyl)-5-nitrophenyl)boronic acid (DS81);
(S)-(3-((3-boronobenzyl)(5,6-diamino-6-oxohexyl)carbamoyl)-5-nitrophenyl)boronic acid (DS82);
(S)-(3-((4-borono-2-methoxybenzyl)(5,6-diamino-6-oxohexyl)carbamoyl)-5-nitrophenyl)boronic acid (DS83);
(S)-(3-((4-borono-2-(trifluoromethyl)benzyl)(5,6-diamino-6-oxohexyl)carbamoyl)-5-nitrophenyl)boronic acid (DS84);
(S)-(5-((3-borono-N-(5,6-diamino-6-oxohexyl)-4-fluorobenzamido)methyl)-2-fluorophenyl)boronic acid (DS85);
(S)-(5-((4-borono-3, 5-difluorobenzyl)(5,6-diamino-6-oxohexyl)carbamoyl)-2-fluorophenyl)boronic acid (DS86);
(S)-(3-((3-borono-N-(5,6-diamino-6-oxohexyl)-4-fluorobenzamido)methyl) phenyl)boronic acid (DS87);
(S)-(5-((4-borono-2-methoxybenzyl)(5,6-diamino-6-oxohexyl)carbamoyl)-2-fluorophenyl)boronic acid (DS88);
(S)-(5-((4-borono-3-(trifluoromethyl)benzyl)(5,6-diamino-6-oxohexyl)carbamoyl)-2-fluorophenyl)boronic acid (DS89);
(S)-(4-((3-borono-4-fluorobenzyl)(5,6-diamino-6-oxohexyl)carbamoyl)-2-fluorophenyl)boronic acid (DS90);
(S)-(4-((4-borono-3, 5-difluorobenzyl)(5, 6-diamino-6-oxohexyl)carbamoyl)-2-fluorophenyl)boronic acid (DS91);
(S)-(4-((3-boronobenzyl)(5,6-diamino-6-oxohexyl)carbamoyl)-2-fluorophenyl)boronic acid (DS92);
(S)-(4-((4-borono-2-methoxybenzyl)(5,6-diamino-6-oxohexyl)carbamoyl)-2-fluorophenyl)boronic acid (DS93);
(S)-(4-((4-borono-2-(trifluoromethyl)benzyl)(5,6-diamino-6-oxohexyl)carbamoyl)-2-fluorophenyl)boronic acid (DS94);
(S)-(5-((3-borono-5-bromo-N-(5,6-diamino-6-oxohexyl)benzamido)methyl)-2-fluorophenyl)boronic acid (DS95);
(S)-(3-((4-borono-3, 5-difluorobenzyl)(5,6-diamino-6-oxohexyl)carbamoyl)-5-bromophenyl)boronic acid (DS96);
(S)-(3-((3-borono-5-bromo-N-(5,6-diamino-6-oxohexyl)benzamido)methyl) phenyl)boronic acid (DS97);
(S)-(3-((3-borono-5-bromo-N-(5,6-diamino-6-oxohexyl)benzamido)methyl)-5-methoxyphenyl)boronic acid (DS98);
(S)-(3-((4-borono-2-(trifluoromethyl)benzyl)(5,6-diamino-6-oxohexyl)carbamoyl)-5-bromophenyl)boronic acid (DS99);
(S)-(3-((3-borono-4-fluorobenzyl)(5,6-diamino-6-oxohexyl)carbamoyl)-5-fluorophenyl)boronic acid (DS100);
(S)-(3-((4-borono-3-methoxybenzyl)(5,6-diamino-6-oxohexyl)carbamoyl)-5-fluorophenyl)boronic acid (DS101);
(S)-(3-((4-borono-2-(trifluoromethyl)benzyl)(5,6-diamino-6-oxohexyl)carbamoyl)-5-fluorophenyl)boronic acid (DS102);
(S)-(4-((N-(5,6-diamino-6-oxohexyl)-1-hydroxy-1,3-dihydrobenzo[c][1,2]oxaborole-6-carboxamido)methyl)-2-fluorophenyl)boronic acid (DS103);
(S)-(4-((N-(5,6-diamino-6-oxohexyl)-1-hydroxy-1,3-dihydrobenzo[c][1,2]oxaborole-6-carboxamido)methyl)-2,6-difluorophenyl)boronic acid (DS104);
(S)-(3-((N-(5,6-diamino-6-oxohexyl)-1-hydroxy-1,3-dihydrobenzo[c][1,2]oxaborole-6-carboxamido)methyl)phenyl)boronic acid (DS105);
(S)-(4-((N-(5,6-diamino-6-oxohexyl)-1-hydroxy-1,3-dihydrobenzo[c][1,2]oxaborole-6-carboxamido)methyl)-3-methoxyphenyl)boronic acid (DS106);
(S)—N-(5,6-diamino-6-oxohexyl)-1-hydroxy-N-((1-hydroxy-1,3-dihydrobenzo[c][1,2]oxaborol-6-yl)methyl)-1,3-dihydrobenzo[c][1,2]oxaborole-6-carboxamide (DS107);
(S)—N-(4-amino-3-(1-hydroxy-1,3-dihydrobenzo[c][1,2]oxaborole-6-carboxamido)-4-oxobutyl)-1-hydroxy-N-((1-hydroxy-1,3-dihydrobenzo[c][1,2]oxaborol-6-yl)methyl)-1,3-dihydrobenzo[c][1,2]oxaborole-6-carboxamide (DS108);
(S)—N-(6-amino-5-(1-hydroxy-1,3-dihydrobenzo[c][1,2]oxaborole-6-carboxamido)-6-oxohexyl)-1-hydroxy-N-((1-hydroxy-1,3-dihydrobenzo[c][1,2]oxaborol-6-yl)methyl)-1,3-dihydrobenzo[c][1,2]oxaborole-6-carboxamide (DS109);
(2S,4S)-1-(1-hydroxy-1,3-dihydrobenzo[c][1,2]oxaborole-6-carbonyl)-4-(1-hydroxy-1,3-dihydrobenzo[c][1,2]oxaborole-6-carboxamido)pyrrolidine-2-carboxylic acid (DS110);
(2S,3S)-2-(1-hydroxy-1,3-dihydrobenzo[c][1,2]oxaborole-5-carboxamido)-3-(1-hydroxy-1,3-dihydrobenzo[c][1,2]oxaborole-6-carboxamido)butanoic acid (DS111); and
(2S,4R)-1-(1-hydroxy-1,3-dihydrobenzo[c][1,2]oxaborole-6-carbonyl)-4-(1-hydroxy-1,3-dihydrobenzo[c][1,2]oxaborole-6-carboxamido)pyrrolidine-2-carboxylic acid (DS112).

53. (canceled)

54. A human insulin analog, comprising an A-chain and a B-chain,
wherein the sequence of the A-chain comprises:
X_{aa′}X_{bb′}X_{cc′}X_{dd′}X_{ee′}X_{ff′}X_{gg′}VEQCCX_{hh′}X_{ii′}ICSLYQLENYCNX_{jj′}X_{kk′}X_{ll′}X_{mm′}X_{nn′}X_{oo′}X_{pp′} (SEQ ID NO:24015); and
wherein the sequence of the B-chain comprises:
(i)
(SEQ ID NO: 24016)
XₐₐX_{bb}X_{cc}X_{dd}KXₑₑX_{ff}X_{gg}XₕₕXᵢᵢXⱼⱼKXₖₖXₗₗXₘₘXₙₙQHLCGS
HLVEALYLVCXₒₒXₚₚX_{qq}GFFYTXᵣᵣTXₛₛXₜₜXᵤᵤXᵥᵥX_{ww},
wherein X_{aa′}, X_{bb′}, X_{cc′}, X_{dd′}, X_{ee′}, X_{ff′}, X_{gg′}, X_{hh′}, X_{ii′}, X_{jj′}, X_{kk′}, X_{ll′}, X_{mm′}, X_{nn′}, X_{oo′}, X_{pp′}, Xₐₐ, X_{bb}, X_{cc}, X_{dd}, Xₑₑ, X_{ff}, X_{gg}, Xₕₕ, Xᵢᵢ, Xⱼⱼ, Xₖₖ, Xₗₗ, Xₘₘ, Xₙₙ, Xₒₒ, Xₚₚ, X_{qq}, Xᵣᵣ, Xₛₛ, Xₜₜ, Xᵤᵤ, Xᵥᵥ, and X_{ww} are each independently either absent or selected from amino acid residues A, D, E, F, G, H, I, K, L, N, P, Q, R, S, T, V, Y and W,
(ii)
(SEQ ID NO: 24017)
XₐₐX_{bb}X_{cc}X_{dd}KPXₑₑX_{ff}X_{gg}XₕₕXᵢᵢXⱼⱼXₖₖXₗₗXₘₘXₙₙQHLCGS
HLVEALYLVCXₒₒXₚₚX_{qq}GFFYTXᵣᵣXₛₛXₜₜXᵤᵤXᵥᵥX_{ww},
wherein X_{aa′}, X_{bb′}, X_{cc′}, X_{dd′}, X_{ee′}, X_{ff′}, X_{gg′}, X_{hh′}, X_{ii′}, X_{jj′}, X_{kk′}, X_{ll′}, X_{mm′}, X_{nn′}, X_{oo′}, X_{pp′}, Xₐₐ, X_{bb}, X_{cc}, X_{dd}, X_{ff}, X_{gg}, Xₕₕ, Xᵢᵢ, Xⱼⱼ, Xₖₖ, Xₗₗ, Xₘₘ, Xₙₙ, Xₒₒ, Xₚₚ, X_{qq}, Xᵣᵣ, Xₛₛ, Xₜₜ, Xᵤᵤ, Xᵥᵥ, and X_{ww} are each independently either absent or selected from amino acid residues A, D, E, F, G, H, I, K, L, N, P, Q, R, S, T, V, Y and W, and wherein Xₑₑ is selected from amino acid residues A, E, F, H, I, K, L, N, P, Q, R, S, T, V, Y and W,
(iii)
(SEQ ID NO: 24018)
XₐₐX_{bb}X_{cc}X_{dd}KXₑₑX_{ff}X_{gg}XₕₕXᵢᵢXⱼⱼKXₖₖXₗₗXₘₘXₙₙQHLCGS
HLVEALYLVCXₒₒXₚₚX_{qq}GFFYTXᵣᵣXₛₛXₜₜXᵤᵤXᵥᵥX_{ww},
wherein X_{aa′}, X_{bb′}, X_{cc′}, X_{dd′}, X_{ee′}, X_{ff′}, X_{gg′}, X_{hh′}, X_{ii′}, X_{jj′}, X_{kk′}, X_{ll′}, X_{mm′}, X_{nn′}, X_{oo′}, X_{pp′}, Xₐₐ, X_{bb}, X_{cc}, X_{dd}, Xₑₑ, X_{ff}, X_{gg}, Xₕₕ, Xᵢᵢ, Xⱼⱼ, Xₖₖ, Xₗₗ, Xₘₘ, Xₙₙ, Xₒₒ, Xₚₚ, X_{qq}, Xᵣᵣ, Xₛₛ, Xₜₜ, Xᵤᵤ, Xᵥᵥ, and X_{ww} are each independently either absent or selected from amino acid residues A, D, E, F, G, H, I, K, L, N, P, Q, R, S, T, V, Y, W and at least one of Xₑₑ, X_{ff}, X_{gg}, Xₕₕ, Xᵢᵢ, Xⱼⱼ is present and at least one of Xₑₑ, X_{ff}, X_{gg}, Xₕₕ, Xᵢᵢ, Xⱼⱼ is G,
(iv)
(SEQ ID NO: 24019)
XₐₐX_{bb}X_{cc}X_{dd}KXₑₑX_{ff}X_{gg}XₕₕXᵢᵢXⱼⱼKXₖₖXₗₗXₘₘXₙₙQHLCGS
HLVEALYLVCXₒₒXₚₚX_{qq}GFFYTXᵣᵣXₛₛXₜₜXᵤᵤXᵥᵥXX_{ww},
wherein X_{aa′}, X_{bb′}, X_{cc′}, X_{dd′}, X_{ee′}, X_{ff′}, X_{gg′}, X_{hh′}, X_{ii′}, X_{jj′}, X_{kk′}, X_{ll′}, X_{mm′}, X_{nn′}, X_{oo′}, X_{pp′}, Xₐₐ, X_{bb}, X_{cc}, X_{dd}, Xₑₑ, X_{ff}, X_{gg}, Xₕₕ, Xᵢᵢ, Xⱼⱼ, Xₖₖ, Xₗₗ, Xₘₘ, Xₙₙ, Xₒₒ, Xₚₚ, X_{qq}, Xᵣᵣ, Xₛₛ, Xₜₜ, Xᵤᵤ, Xᵥᵥ, and X_{ww} are each independently either absent or selected from amino acid residues A, D, E, F, G, H, I, K, L, N, P, Q, R, S, T, V, Y, W and at least one of Xₑₑ, X_{ff}, X_{gg}, Xₕₕ, Xᵢᵢ, Xⱼⱼ is present and at least one of Xₑₑ, X_{ff}, X_{gg}, Xₕₕ, Xᵢᵢ, Xⱼⱼ is S, or
(v)
(SEQ ID NO: 24020)
XₐₐX_{bb}X_{cc}X_{dd}KXₑₑX_{ff}X_{gg}XₕₕXᵢᵢXⱼⱼKXₖₖXₗₗXₘₘXₙₙQHLCGS
HLVEALYLVCXₒₒXₚₚX_{qq}GFFYTXᵣᵣXₛₛXₜₜXᵤᵤXᵥᵥX_{ww},
wherein X_{aa′}, X_{bb′}, X_{cc′}, X_{dd′}, X_{ee′}, X_{ff′}, X_{gg′}, X_{hh′}, X_{ii′}, X_{jj′}, X_{kk′}, X_{ll′}, X_{mm′}, X_{nn′}, X_{oo′}, X_{pp′}, Xₐₐ, X_{bb}, X_{cc}, X_{dd}, Xₑₑ, X_{ff}, X_{gg}, Xₕₕ, Xᵢᵢ, Xⱼⱼ, Xₖₖ, Xₗₗ, Xₘₘ, Xₙₙ, Xₒₒ, Xₚₚ, X_{qq}, Xᵣᵣ, Xₛₛ, Xₜₜ, Xᵤᵤ, Xᵥᵥ, and X_{ww} are each independently either absent or selected from amino acid residues A, D, E, F, G, H, I, K, L, N, P, Q, R, S, T, V, Y, W and at least two of Xₑₑ, X_{ff}, X_{gg}, Xₕₕ, Xᵢᵢ, Xⱼⱼ are present and at least one of Xₑₑ, X_{ff}, X_{gg}, Xₕₕ, Xᵢᵢ, Xⱼⱼ is S, and another is G.

55. The insulin of claim 54, wherein the A-chain comprises a sequence selected from SEQ ID NOs 1 and 3 to 33, and is optionally appended at the N-terminus and/or at the C-terminus by at least one selected from KA, KD, KE, KF, KG, KH, KI, KL, KN, KP, KQ, KR, KS, KT, KY, KAA, KAD, KAE, KAF, KAG, KAH, KAI, KAL, KAN, KAQ, KAR, KAS, KAT, KAY, KDA, KDD, KDE, KDF, KDG, KDH, KDI, KDL, KDN, KDQ, KDR, KDS, KDT, KDY, KEA, KED, KEE, KEF, KEG, KEH, KEI, KEL, KEN, KEQ, KER, KES, KET, KEY, KFA, KFD, KFE, KFF, KFG, KFH, KFI, KFL, KFN, KFQ, KFR, KFS, KFT, KFY, KGA, KGD, KGE, KGF, KGG, KGH, KGI, KGL, KGN, KGQ, KGR, KGS, KGT, KGY, KHA, KHD, KHE, KHF, KHG, KHH, KHI, KHL, KHN, KHQ, KHR, KHS, KHT, KHY, KIA, KID, KIE, KIF, KIG, KIH, KII, KIL, KIN, KIQ, KIR, KIS, KIT, KIY, KLA, KLD, KLE, KLF, KLG, KLH, KLI, KLL, KLN, KLQ, KLR, KLS, KLT, KLY, KNA, KND, KNE, KNF, KNG, KNH, KNI, KNL, KNN, KNQ, KNR, KNS, KNT, KNY, KPA, KPD, KPE, KPF, KPG, KPH, KPI, KPL, KPN, KPQ, KPR, KPS, KPT, KPY, KQA, KQD, KQE, KQF, KQG, KQH, KQI, KQL, KQN, KQQ, KQR, KQS, KQT, KQY, KRA, KRD, KRE, KRF, KRG, KRH, KRI, KRL, KRN, KRQ, KRR, KRS, KRT, KRY, KSA, KSD, KSE, KSF, KSG, KSH, KSI, KSL, KSN, KSQ, KSR, KSS, KST, KSY, KTA, KTD, KTE, KTF, KTG, KTH, KTI, KTL, KTN, KTQ, KTR, KTS, KTT, KTY, KYA, KYD, KYE, KYF, KYG, KYH, KYI, KYL, KYN, KYQ, KYR, KYS, KYT, KYY, SEQ ID NOs 75 to 24014, KGSH (SEQ ID NO:24049), GKGSH (SEQ ID NO:24050), GKGSKK (SEQ ID NO:24045), GKKPGKK (SEQ ID NO:24046), GKGPSK (SEQ ID NO:24044), GKPSHKP (SEQ ID NO:24043), and GSHKGSHK (SEQ ID NO:24042); and
wherein the B-chain comprises a sequence selected from SEQ ID NOs 2 and 34 to 74, 24047, and 24048, and is optionally appended at the N-terminus and/or at the C-terminus by at least one selected from KA, KD, KE, KF, KG, KH, KI, KL, KN, KP, KQ, KR, KS, KT, KY, KAA, KAD, KAE, KAF, KAG, KAH, KAI, KAL, KAN, KAQ, KAR, KAS, KAT, KAY, KDA, KDD, KDE, KDF, KDG, KDH, KDI, KDL, KDN, KDQ, KDR, KDS, KDT, KDY, KEA, KED, KEE, KEF, KEG, KEH, KEI, KEL, KEN, KEQ, KER, KES, KET, KEY, KFA, KFD, KFE, KFF, KFG, KFH, KFI, KFL, KFN, KFQ, KFR, KFS, KFT, KFY, KGA, KGD, KGE, KGF, KGG, KGH, KGI, KGL, KGN, KGQ, KGR, KGS, KGT, KGY, KHA, KHD, KHE, KHF, KHG, KHH, KHI, KHL, KHN, KHQ, KHR, KHS, KHT, KHY, KIA, KID, KIE, KIF, KIG, KIH, KII, KIR, KIN, KIQ, KIR, KIS, KIT, KIY, KLA, KLD, KLE, KLF, KLG, KLH, KLI, KLL, KLN, KLQ, KLR, KLS, KLT, KLY, KNA, KND, KNE, KNF, KNG, KNH, KNI, KNL, KNN, KNQ, KNR, KNS, KNT, KNY, KPA, KPD, KPE, KPF, KPG, KPH, KPI, KPL, KPN, KPQ, KPR, KPS, KPT, KPY, KQA, KQD, KQE, KQF, KQG, KQH, KQI, KQL, KQN, KQQ, KQR, KQS, KQT, KQY, KRA, KRD, KRE, KRF, KRG, KRH, KRI, KRL, KRN, KRQ, KRR, KRS, KRT, KRY, KSA, KSD, KSE, KSF, KSG, KSH, KSI, KSL, KSN, KSQ, KSR, KSS, KST, KSY, KTA, KTD, KTE, KTF, KTG, KTH, KTI, KTL, KTN, KTQ, KTR, KTS, KTT, KTY, KYA, KYD, KYE, KYF, KYG, KYH, KYI, KYL, KYN, KYQ, KYR, KYS, KYT, KYY, SEQ ID NOs 75 to 24014, KGSH (SEQ ID NO:24049), GKGSH (SEQ ID NO:24050), GKGSKK (SEQ ID NO:24045), GKKPGKK (SEQ ID NO:24046), GKGPSK (SEQ ID NO:24044), GKPSHKP (SEQ ID NO:24043), and GSHKGSHK (SEQ ID NO:24042).

56.-66. (canceled)
